SEQUENCE LISTING (115 sequences)

SEQ ID NO: 1 - protein, 260 aa, Homo sapiens
  MISC_FEATURE: Amino acid sequence of UPK1B
MAKDNSTVRCFQGLLIFGNVIIGCCGIALTAECIFFVSDQHSLYPLLEATDNDDIYGAAWIGIFVGICLFCLSVLGIVGIMKSSRKILLAYFILMFIVYAFEVASCITAATQQDFFTPNLFLKQMLERYQNNSPPNNDDQWKNNGVTKTWDRLMLQDNCCGVNGPSDWQKYTSAFRTENNDADYPWPRQCCVMNNLKEPLNLEACKLGVPGFYHNQGCYELISGPMNRHAWGVAWFGFAILCWTFWVLLGTMFYWSRIEY

SEQ ID NO: 2 - protein, 329 aa, Homo sapiens
  MISC_FEATURE: IgG1 heavy chain constant region protein
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 329 aa, Homo sapiens
  MISC_FEATURE: C220S IgG1 heavy constant region protein
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 328 aa, Homo sapiens
  MISC_FEATURE: C220 delta IgG1 heavy constant region protein
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 107 aa, Homo sapiens [sequence omitted]

SEQ ID NO: 6 - protein, 107 aa, Homo sapiens
  MISC_FEATURE: C214S kappa light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 7 - protein, 106 aa, Homo sapiens
  MISC_FEATURE: C214 delta kappa light chain constant region protein
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 8 - protein, 105 aa, Homo sapiens
  MISC_FEATURE: lambda light chain constant region protein
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 9 - protein, 105 aa, Homo sapiens
  MISC_FEATURE: C214S lambda light chain constant region protein
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTESS

SEQ ID NO: 10 - protein, 104 aa, Homo sapiens
  MISC_FEATURE: C214 delta lambda light chain constant region protein
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTES

SEQ ID NO: 11-19 - skipped (9 entries)

SEQ ID NO: 20 - DNA, 339 bp, Mus musculus
  misc_feature: SC115.1 VL
gacattgtgatgacacagtctccatcctccctggctatgtcagtaggacagaaggtcactatgaactgcaagtccagtcagagccttttaaatagtaacaatcaaaagaactatttggcctggtaccagcagaaaccaggacagtctcctaaacttctggtatactttgcatccactagggaatctggggtccctgatcgcttcataggcagtggatctgggacagatttcactcttaccatcaccagtgttcaggctgaagacctggcagattacttctgtctgcaacattatggcactccgctcacgttcggtgctgggaccaagctggagctgaaa

SEQ ID NO: 21 - protein, 113 aa, Mus musculus
  MISC_FEATURE: SC115.1 VL
DIVMTQSPSSLAMSVGQKVTMNCKSSQSLLNSNNQKNYLAWYQQKPGQSPKLLVYFASTRESGVPDRFIGSGSGTDFTLTITSVQAEDLADYFCLQHYGTPLTFGAGTKLELK

SEQ ID NO: 22 - DNA, 354 bp, Mus musculus
  misc_feature: SC115.1 VH
caggtgctactgcagcagtctgggcctcagctggttaggcctggggcttcagtgaagatatcctgcaaggcttctggttactcattcaccagttactggatccactgggtgaagcagaggcctgggcaaggtcttgagtggattggcatgattgatccttccgatagtgaaactagctttaatcagaagttcaaggacaaggccacattgactgtagacaaatcctccagcacagcctacatgcaactcagcagcccgacatctgaggactctgcggtctattactgtgcaaggagttactacgctagtcgctttgactactggggccaaggcgccactctcacagtctcctca

SEQ ID NO: 23 - protein, 118 aa, Mus musculus
  MISC_FEATURE: SC115.1 VH
QVLLQQSGPQLVRPGASVKISCKASGYSFTSYWIHWVKQRPGQGLEWIGMIDPSDSETSFNQKFKDKATLTVDKSSSTAYMQLSSPTSEDSAVYYCARSYYASRFDYWGQGATLTVSS

SEQ ID NO: 24 - DNA, 333 bp, Mus musculus
  misc_feature: SC115.4 VL
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgcttcaacctattactgtcagcacagtagggagcttcctctcacgttcggtgctgggaccaagctggagctgaaa

SEQ ID NO: 25 - protein, 111 aa, Mus musculus
  MISC_FEATURE: SC115.4 VL
DIVLTQSPASLAVSLGQRATISCRASKSVSTSGYSYMHWYQQKPGQPPKLLIYLASNLESGVPARFSGSGSGTDFTLNIHPVEEEDASTYYCQHSRELPLTFGAGTKLELK

SEQ ID NO: 26 - DNA, 363 bp, Mus musculus
  misc_feature: SC115.4  VH
gacgtgaagctcgtggagtctgggggaggcttagtgaagcttggagggtccctgaaactctcctgtgcagcctctggattcactttcagtagctatcccatgtcttgggttcgccagactccggagatgaggctggagtgggtcgcaaccattagtagtggtggtggtatcacctactatccagacagtgtgaagggtcgattcaccatctccagagacaatgccaagaacaccctgtccctgcaaatgaacagtttgaggtctgaggacacggccatgtattactgttcaagactctactatagtgactacgagggttatatggactactggggtcaaggaacctcagtcaccgtctcctca

SEQ ID NO: 27 - protein, 121 aa, Mus musculus
  MISC_FEATURE: SC115.4 VH
DVKLVESGGGLVKLGGSLKLSCAASGFTFSSYPMSWVRQTPEMRLEWVATISSGGGITYYPDSVKGRFTISRDNAKNTLSLQMNSLRSEDTAMYYCSRLYYSDYEGYMDYWGQGTSVTVSS

SEQ ID NO: 28 - DNA, 339 bp, Mus musculus
  misc_feature: SC115.7 VL
gacattgtgatgacacagtctccatcctccctgactgtgacagcaggagagaaggtcactatgagctgcaagtccagtcagagtctgttaaacagtggaaatcaaaagaactatttgacctggtaccagcagaaactagggcagcctcctaaactgttgatctattgggcatccactagggaatctggggtccctgatcgcttcacaggcagtggatctggaacagatttcactctcaccatcagcagtgtgcaggctgaagacctggcagtttattactgtcagaatgattatagtaatccattcacgttcggctcggggacaaagttggagataaaa

SEQ ID NO: 29 - protein, 113 aa, Mus musculus
  MISC_FEATURE: SC115.7 VL
DIVMTQSPSSLTVTAGEKVTMSCKSSQSLLNSGNQKNYLTWYQQKLGQPPKLLIYWASTRESGVPDRFTGSGSGTDFTLTISSVQAEDLAVYYCQNDYSNPFTFGSGTKLEIK

SEQ ID NO: 30 - DNA, 366 bp, Mus musculus
  misc_feature: SC115.7 VH
gaggttcagctgcagcagtctggggcagagcttgtgaggccaggggcctcagtcaagttgtcctgcacagcttctggcttcaatattaaagacagttttattcactgggtgaagaagaggcctgaagagggcctggagtggattggatggattgatcctgaggatggtgaaactaaatatgccccgaagttccaggacaaggccactataactgcagacacatcctccaacacagcctacctgcagctcagcagcctgacatctgaggacactgccatctattactgtgctagaagggaacgtttctacgggatttactatgctctggactactggggtcagggaacctcagtcaccgtctcctca

SEQ ID NO: 31 - protein, 122 aa, Mus musculus
  MISC_FEATURE: SC115.7 VH
EVQLQQSGAELVRPGASVKLSCTASGFNIKDSFIHWVKKRPEEGLEWIGWIDPEDGETKYAPKFQDKATITADTSSNTAYLQLSSLTSEDTAIYYCARRERFYGIYYALDYWGQGTSVTVSS

SEQ ID NO: 32 - DNA, 339 bp, Mus musculus
  misc_feature: SC115.9 VL
gacattgtgatgacacagtctccatcctccctgactgtgacagcaggagagaaggtcactatgagctgcaagtccagtcagagtctgttaaacagtggaaatcaaaagaactacttgacctggtaccagcagaaaccaggacagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagctggagctgaaa

SEQ ID NO: 33 - protein, 113 aa, Mus musculus
  MISC_FEATURE: SC115.9 VL
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 34 - DNA, 354 bp, Mus musculus
  misc_feature: SC115.9 VH
caggtccaactgcagcagcctggggctgagctggtgaagcctggggcttcagtgaagatgtcctgcaaggcttctggctacactttcaccagcaactggatgcactgggtgaagcagaggcctggacaaggccttgagtggatcggagtgattgatccttctgatacttatattagttacaatcaaaagttcaagggcaaggccacattgactgtagacacatcctccagtacagcctacatgcagctcagcagcctgacatctgaggactctgcggtctattactgtacaagaagggattccggatggtacttcgatgtctggggcgcagggaccacggtcaccgtctcctca

SEQ ID NO: 35 - protein, 118 aa, Mus musculus
  MISC_FEATURE: SC115.9 VH
QVQLQQPGAELVKPGASVKMSCKASGYTFTSNWMHWVKQRPGQGLEWIGVIDPSDTYISYNQKFKGKATLTVDTSSSTAYMQLSSLTSEDSAVYYCTRRDSGWYFDVWGAGTTVTVSS

SEQ ID NO: 36 - DNA, 336 bp, Mus musculus
  misc_feature: SC115.13 VL
gatgttgtgatgacccagactccactcactttgtcggttaccattggacaaccagcctccatctcttgcaagtcaagtcagagcctcttagatagtgatggagagacatatttgaattggttcttacagaggccaggccagtctccaaagcgcctaatctatctggtgtctaaattggactctggagtccctgacaggttcactggcagtggatcagggacagatttcacactgaaaatcagcagagtggaggctgatgatttgggagtttattattgctgccaaggtacacattttccgctcacgttcggtgctgggaccaaactggacctgaaa

SEQ ID NO: 37 - protein, 112 aa, Mus musculus
  MISC_FEATURE: SC115.13 VL
DVVMTQTPLTLSVTIGQPASISCKSSQSLLDSDGETYLNWFLQRPGQSPKRLIYLVSKLDSGVPDRFTGSGSGTDFTLKISRVEADDLGVYYCCQGTHFPLTFGAGTKLDLK

SEQ ID NO: 38 - DNA, 354 bp, Mus musculus
  misc_feature: SC115.13 VH
cagatcctgttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaagactggtgtgccaacatatcctgatgacttcaagggccggtttgccttctctttggaaacctctgccagcactgcctatttgcagatcagcaacctcaaagatgaggacacggctacatatttctgtacaagaggggagagttttttttcgtttgctttctggggccaagggactctggtcgctgtctctgca

SEQ ID NO: 39 - protein, 118 aa, Mus musculus
  MISC_FEATURE: SC115.13 VH
QILLVQSGPELKKPGETVKISCKASGYTFTNYGMNWVKQAPGKGLKWMGWINTKTGVPTYPDDFKGRFAFSLETSASTAYLQISNLKDEDTATYFCTRGESFFSFAFWGQGTLVAVSA

SEQ ID NO: 40 - DNA, 339 bp, Mus musculus
  misc_feature: SC115.18 VL
gacattgtgatgacacagtctccatcctccctgactgtgacagcaggagagaaggtcactatgagatgcaagtccagtcagagtctgttaaacagtggaaatcaaaagaactacttgacctggtaccagcagaaaccagggcagcctcctaaactgttgatctcctgggcatccactagggactctggggtccctgatcgcttcacaggcagtggatctagaacagatttcattctcaccatcagcagtgtgcaggctgaagacctggccgtttattactgtcagaatgattatagttatccgctcacgttcggtgctgggaccaagctggagctgaaa

SEQ ID NO: 41 - protein, 113 aa, Mus musculus
  MISC_FEATURE: SC115.18 VL
DIVMTQSPSSLTVTAGEKVTMRCKSSQSLLNSGNQKNYLTWYQQKPGQPPKLLISWASTRDSGVPDRFTGSGSRTDFILTISSVQAEDLAVYYCQNDYSYPLTFGAGTKLELK

SEQ ID NO: 42 - DNA, 363 bp, Mus musculus
  misc_feature: SC115.18 VH
caggtccaactgcagcagcctggggctgagctggtgaggcctgggtcttcagtgaagctgtcctgcaaggcttctggctacaccttcaccagctactggatgcattgggtgaaacagaggcctatgcaaggccttgaatggattggtaacattgacccctctgatagtgaaatttattacaatcaaaagttcaaggacaaggccacattgactgtagacaaatcctccggcacagcctacatgcagctcagcagcctgacatctgaggactctgcggtctattactgtgcaaggaggtccggtagtagagactactatggtatggacgactggggtcaaggaacctcagtcaccgtctcctca

SEQ ID NO: 43 - protein, 121 aa, Mus musculus
  MISC_FEATURE: SC115.18 VH
QVQLQQPGAELVRPGSSVKLSCKASGYTFTSYWMHWVKQRPMQGLEWIGNIDPSDSEIYYNQKFKDKATLTVDKSSGTAYMQLSSLTSEDSAVYYCARRSGSRDYYGMDDWGQGTSVTVSS

SEQ ID NO: 44 - DNA, 336 bp, Mus musculus
  misc_feature: SC115.19 VL
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccagccttgcctcaggagtcccagacaggttcagtagcagtgggtcaggaactgatttcacactgagaatcagcagagtggaggctgaggatgtgggtgtttattactgtgctcaaagtctagaacttccgtacacgttcggaggggggaccaagctggaaataaaa

SEQ ID NO: 45 - protein, 112 aa, Mus musculus
  MISC_FEATURE: SC115.19 VL
DIVMTQAAFSNPVTLGTSASISCRSSKSLLHSNGITYLYWYLQKPGQSPQLLIYQMSSLASGVPDRFSSSGSGTDFTLRISRVEAEDVGVYYCAQSLELPYTFGGGTKLEIK

SEQ ID NO: 46 - DNA, 363 bp, Mus musculus
  misc_feature: SC115.19 VH
cagattactctgaaagagtctggccctgggatattgcagccctcccagactctcagtctgacttgttctttctctgggttttcactgagcacttctggtatgggtgtaggctggattcgtcagccttcagggaagggtctggagtggctggcaaacatttggtggaatgatgatcagtactataacccacccctgaagagccggctcacagtctccaaggatacctccaaaacccaggtgttcctcaagatcgcccatgtggacactgcagatactgccacatactactgtgctcgaatttactctggtaactacggcgctatggactactggggtcaaggaacctcagtcaccgtctcctca

SEQ ID NO: 47 - protein, 121 aa, Mus musculus
  MISC_FEATURE: SC115.19 VH
QITLKESGPGILQPSQTLSLTCSFSGFSLSTSGMGVGWIRQPSGKGLEWLANIWWNDDQYYNPPLKSRLTVSKDTSKTQVFLKIAHVDTADTATYYCARIYSGNYGAMDYWGQGTSVTVSS

SEQ ID NO: 48 - DNA, 339 bp, Mus musculus
  misc_feature: SC115.26 VL
gacattgtgatgtcacagtctccatcctccctagctgtgtcagttggagagaaggttactatgagctgcaagtccagtcagagccttttatatagtagcaatcaaaagaacttcttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcaatttattactgtcagcaatattattactatccgtacacgttcggaggggggaccaagctggaaataaaa

SEQ ID NO: 49 - protein, 113 aa, Mus musculus
  MISC_FEATURE: SC115.26 VL
DIVMSQSPSSLAVSVGEKVTMSCKSSQSLLYSSNQKNFLAWYQQKPGQSPKLLIYWASTRESGVPDRFTGSGSGTDFTLTISSVKAEDLAIYYCQQYYYYPYTFGGGTKLEIK

SEQ ID NO: 50 - DNA, 363 bp, Mus musculus
  misc_feature: SC115.26 VH
caggttactctgaaagagtctggccctgggatattgcagtcctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagaaaccaggtattcctcaagatcaccagtgtggacactgcagatactgccacatactactgtgctcgaagagccaatcgtgcttggggggctatggactactggggtcagggaacctcagtcaccgtctcctca

SEQ ID NO: 51 - protein, 121 aa, Mus musculus
  MISC_FEATURE: SC115.26 VH
QVTLKESGPGILQSSQTLSLTCSFSGFSLSTSGMGVSWIRQPSGKGLEWLAHIYWDDDKRYNPSLKSRLTISKDTSRNQVFLKITSVDTADTATYYCARRANRAWGAMDYWGQGTSVTVSS

SEQ ID NO: 52 - DNA, 339 bp, Mus musculus
  misc_feature: SC115.32 VL
gacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccattcacgttcggctcggggacaaagttggaaataaaa

SEQ ID NO: 53 - protein, 113 aa, Mus musculus
  MISC_FEATURE: SC115.32 VL
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 54 - DNA, 363 bp, Mus musculus
  misc_feature: SC115.32 VH
caggtccaactgcagcagcctggggctgagctggtgaggcctgggtcttcagtgaagctgtcctgtaaggcttctggctacaccttcaacagctactggatgcattgggtgaagcagaggcctataaaaggccttgaatggattggtaacattgacccttctgatggtgaaacttattacaatcaagccttcaaggacaaggccacattgactgtagacaaatcctccagcacagcctacatgcagctcagcaggttgacatctgaggactctgcggtctattactgtgcaagacgggggacagctcaggctacgacctggttaggttactggggccaagggactctggtcactgtctctgca

SEQ ID NO: 55 - protein, 121 aa, Mus musculus
  MISC_FEATURE: SC115.32 VH
QVQLQQPGAELVRPGSSVKLSCKASGYTFNSYWMHWVKQRPIKGLEWIGNIDPSDGETYYNQAFKDKATLTVDKSSSTAYMQLSRLTSEDSAVYYCARRGTAQATTWLGYWGQGTLVTVSA

SEQ ID NO: 56 - DNA, 333 bp, Mus musculus
  misc_feature: SC115.36 VL
gacattgtgctgacccaatctccagcttctttggctgtgtctctagggcagagggccaccatatcctgcagagccagtgaaagtgttgatggttatggcaatagttttatgcactggtaccggcagaaaccaggacagccacccaaactcctcatctatcgtgcatccaacctagaatctgggatccctgccaggttcagtggcagtgggtctaggacagacttcgccctcaccattaatcctgtggaggctgatgatgttgcaacctattactgtcagcaaagttatggggatcctcggacgttcggtggaggcaccaacctggaaatcaaa

SEQ ID NO: 57 - protein, 111 aa, Mus musculus
  MISC_FEATURE: SC115.36 VL
DIVLTQSPASLAVSLGQRATISCRASESVDGYGNSFMHWYRQKPGQPPKLLIYRASNLESGIPARFSGSGSRTDFALTINPVEADDVATYYCQQSYGDPRTFGGGTNLEIK

SEQ ID NO: 58 - DNA, 363 bp, Mus musculus
  misc_feature: SC115.36 VH
gaggttcagctccagcagtctgggactgtggtggcaaggcctggggcttccgtgaagatgtcctgcaaggcttctggctacagctttaccagctactggatgcactggataaaacagaggcctggacagggtctagattggattggtagtatttatcctggacatagtgatgccacctacaaccagaggttcaagggcaaggccaaactgactgcagtcacatccgccaacactgcctacatggagctcagcagcctgacaaatgaggattctgcggtctattactgttcaagatcggatggttactacagagggatggctatggactactggggtcaaggaacctcagtcaccgtctcctca

SEQ ID NO: 59 - protein, 121 aa, Mus musculus
  MISC_FEATURE: SC115.36 VH
EVQLQQSGTVVARPGASVKMSCKASGYSFTSYWMHWIKQRPGQGLDWIGSIYPGHSDATYNQRFKGKAKLTAVTSANTAYMELSSLTNEDSAVYYCSRSDGYYRGMAMDYWGQGTSVTVSS

SEQ ID NO: 60 - DNA, 339 bp, Mus musculus
  misc_feature: SC115.46 VL
gacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagactcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagtaatccgttcacgttcggaggggggaccaagctggaaataaaa

SEQ ID NO: 61 - protein, 113 aa, Mus musculus
  MISC_FEATURE: SC115.46 VL
DIVMTQSPSSLTVTAGEKVTMSCKSSQSLLNSGNQKNYLTWYQQKPGQTPKLLIYWASTRESGVPDRFTGSGSGTDFTLTISSVQAEDLAVYYCQNDYSNPFTFGGGTKLEIK

SEQ ID NO: 62 - DNA, 366 bp, Mus musculus
  misc_feature: SC115.46 VH
caggtgcaattgcagcagtctgggcctcagctggttaggcctggggcttcagtgaaaatatcctgcaaggcttctggttactcattcaccacctcctggatgcactggatgaagcagaggcctggacaaggtcttgagtggattggcatgattgatccttccgatagtgaaactgggttaaatcagattttcagggacaaggccacattgactgttgacaaatactccagcacagcctacatgcaactcagcagcccgacatctgaggactctgcggtctattactgtgcaagaagaggtcgattctcctatgattacggggggtttgcttactggggccaagggactctggtcactgtctctgca

SEQ ID NO: 63 - protein, 122 aa, Mus musculus
  MISC_FEATURE: SC115.46 VH
QVQLQQSGPQLVRPGASVKISCKASGYSFTTSWMHWMKQRPGQGLEWIGMIDPSDSETGLNQIFRDKATLTVDKYSSTAYMQLSSPTSEDSAVYYCARRGRFSYDYGGFAYWGQGTLVTVSA

SEQ ID NO: 64 - DNA, 345 bp, Mus musculus
  misc_feature: SC115.48 VL
caacttgtggttactcagtcatcttcagcctctttctccctgggagcctcagcaaaactcacgtgcaccttgagtagtcagcacagtacgtacaccattgaatggtatcagcaacagccactcaagcctcctaagtatgtgatggagcttaagaaagatggaagccacagcacaggtgatgggattcctgatcgcttctctggatccagctctggtgctgatcgctaccttagcatttccaacatgcagcctgaagatgaagcaatatacatctgtggtgtgggtgaagcaattaaggaacaatttgtttatgttttcggcggtggaaccaaggtcactgtccta

SEQ ID NO: 65 - protein, 115 aa, Mus musculus
  MISC_FEATURE: SC115.48 VL
QLVVTQSSSASFSLGASAKLTCTLSSQHSTYTIEWYQQQPLKPPKYVMELKKDGSHSTGDGIPDRFSGSSSGADRYLSISNMQPEDEAIYICGVGEAIKEQFVYVFGGGTKVTVL

SEQ ID NO: 66 - DNA, 348 bp, Mus musculus
  misc_feature: SC115.48 VH
gaggtgcagcttgttgagactggtggaggattggtgcagcctaaagggtcattgaaactctcatgtgcagcctctggactcagcttcaatacctatgccatgaactgggtccgccaggctccaggaaagggtttggaatgggttgctcgcataagaagtaaaagtaataattatgcaacatattatgccgattcagtgaaagacaggttcaccatctccagagatgattcacaaagcatgctctatctgcaaatgaacaacttgaaaactgaggacacagccatgtattactgtgtgacatccctctttgactattggggccaaggcaccactctcacagtctcctca

SEQ ID NO: 67 - protein, 116 aa, Mus musculus
  MISC_FEATURE: SC115.48 VH
EVQLVETGGGLVQPKGSLKLSCAASGLSFNTYAMNWVRQAPGKGLEWVARIRSKSNNYATYYADSVKDRFTISRDDSQSMLYLQMNNLKTEDTAMYYCVTSLFDYWGQGTTLTVSS

SEQ ID NO: 68 - DNA, 345 bp, Mus musculus
  misc_feature: SC115.51 VL
gacattgtgatgacccagtctccatcctccctggctgtgacagcaggagagaaggtcactatgagatgcaagtccagtcagagtcttttgtggagtgtaaaccaaaataactacttatcctggtaccagcagaaacaagggcagcctcctaaactgcttatctctggggcatccattagagaatcttgggtccctgatcgattcacaggaagtggatctgggacagacttcactctcaccattagcaatgtgcatgctgaagacctagcagtttattactgtcagcacaatcatggcagctttctcccgtacacgttcggaggggggaccaagctggaaataaaa

SEQ ID NO: 69 - protein, 115 aa, Mus musculus
  MISC_FEATURE: SC115.51 VL
DIVMTQSPSSLAVTAGEKVTMRCKSSQSLLWSVNQNNYLSWYQQKQGQPPKLLISGASIRESWVPDRFTGSGSGTDFTLTISNVHAEDLAVYYCQHNHGSFLPYTFGGGTKLEIK

SEQ ID NO: 70 - DNA, 351 bp, Mus musculus
  misc_feature: SC115.51 VH
caggtgcagctgaagcagtcaggacctggcctagtgcagccctcacagagcctgtccataacctgcacagtctctggtttctcattaactaactatggtatccactgggttcgccagtctccaggaaagggtctggagtggctgggagtgatatggagaggtggaagcacagactacaatgcaggtttcatgtccagactgaccatcaccaaggacaactccaagagccaagttttctttaaaatgaacagtctgcaaggtgatgacactgccatatactactgtgccaaaaatacgggacgcggggcccttgactactggggccaaggcaccactctcacagtctcctca

SEQ ID NO: 71 - protein, 117 aa, Mus musculus
  MISC_FEATURE: SC115.51 VH
QVQLKQSGPGLVQPSQSLSITCTVSGFSLTNYGIHWVRQSPGKGLEWLGVIWRGGSTDYNAGFMSRLTITKDNSKSQVFFKMNSLQGDDTAIYYCAKNTGRGALDYWGQGTTLTVSS

SEQ ID NO: 72 - DNA, 336 bp, Mus musculus
  misc_feature: SC115.52 VL
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatccgcagggtggaggctgaggatttgggagtttattattgctggcaaggtacacattttccgctcatgttcggttctgggaccaagctggagctgaaa

SEQ ID NO: 73 - protein, 112 aa, Mus musculus
  MISC_FEATURE: SC115.52 VL
DVVMTQTPLTLSVTIGQPASISCKSSQSLLDSDGKTYLNWLLQRPGQSPKRLIYLVSKLDSGVPDRFTGSGSGTDFTLKIRRVEAEDLGVYYCWQGTHFPLMFGSGTKLELK

SEQ ID NO: 74 - DNA, 354 bp, Mus musculus
  misc_feature: SC115.52 VH
cagatcctgttggtgcagtctggacctgagctgaagaagcctggagagacagtcaagatctcctgcaaggcttcagggtataccttcacaaaccttggaatgaactgggtgaagcaggctccaggaaagggtttaaagtggatgggctggataaacacctacactggagatccaacatatgttgatgacttcaagggacggtttgccttctctttggaaacctctgccagcacagcctatttgcagatcagcaacctcaaaaatgaggacacggctacatacttctgtacaagaggggagaaattttattcgtttgcttactggggccaagggactctggtcactgtctctgca

SEQ ID NO: 75 - protein, 118 aa, Mus musculus
  MISC_FEATURE: SC115.52 VH
QILLVQSGPELKKPGETVKISCKASGYTFTNLGMNWVKQAPGKGLKWMGWINTYTGDPTYVDDFKGRFAFSLETSASTAYLQISNLKNEDTATYFCTRGEKFYSFAYWGQGTLVTVSA

SEQ ID NO: 76 - DNA, 336 bp, Mus musculus
  misc_feature: SC115.65 VL
gatgttttgatgacccaaactccactctccctgcctgtcagtcttggagatcaagcctccatctcttgcagatctagtcagatcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatcttccgtacacgttcggtggggggaccaagctggaaataaaa

SEQ ID NO: 77 - protein, 112 aa, Mus musculus
  MISC_FEATURE: SC115.65 VL
DVLMTQTPLSLPVSLGDQASISCRSSQIIVHSNGNTYLEWYLQKPGQSPKLLIYKVSNRFSGVPDRFSGSGSGTDFTLKISRVEAEDLGVYYCFQGSHLPYTFGGGTKLEIK

SEQ ID NO: 78 - DNA, 348 bp, Mus musculus
  misc_feature: SC115.65 VH
gaggttcagctgcagcagtctggacctgagctggtgaagcctggggcttcagtgaagatatcctgcaaggcttctggttactcatttactggctactttatgaactgggtgatgcagagccatggaaagagccttgagtggattggacgtattaatccttacaatggtgatactttctacaaccagaagttcaagggcaaggccacattgactgtagacaaatcctctaacaccgcccatatggagctccggagcctggcatctgaggactctggagtctattattgtgcaggacataataactatggagtctcctggggccaaggcaccactctcacagtctcctca

SEQ ID NO: 79 - protein, 116 aa, Mus musculus
  MISC_FEATURE: SC115.65 VH
EVQLQQSGPELVKPGASVKISCKASGYSFTGYFMNWVMQSHGKSLEWIGRINPYNGDTFYNQKFKGKATLTVDKSSNTAHMELRSLASEDSGVYYCAGHNNYGVSWGQGTTLTVSS

SEQ ID NO: 80 - DNA, 324 bp, Mus musculus
  misc_feature: SC115.84 VL
caaattgttctcacccagtctccagctatcatgtctgcatctcctggggaacgggtcaccatgacctgcagtgccagctcaagtgtaagttccttctacttgtattggtaccagcagaagtcaggatcctccccaaaagtctggatttatagcacatccaacctggcttctggagtcccaactcgcttcagtggcagtgggtctgggacctcttactctctcacaatcaacagcatggaggctgaagatgctgccacttattactgccagcagtggagtagtaacccactcacgttcggtgctgggaccaagctggagctgaaa

SEQ ID NO: 81 - protein, 108 aa, Mus musculus
  MISC_FEATURE: SC115.84 VL
QIVLTQSPAIMSASPGERVTMTCSASSSVSSFYLYWYQQKSGSSPKVWIYSTSNLASGVPTRFSGSGSGTSYSLTINSMEAEDAATYYCQQWSSNPLTFGAGTKLELK

SEQ ID NO: 82 - DNA, 351 bp, Mus musculus
  misc_feature: SC115.84 VH
caggttcagctgcagcagtctggggctgagctggtgaagcctggggcctcagtgaagatttcctgcgaaacttctggctacacattcagtagctactggatgaactgggtgaagcagaggcctggaaagggtcttgagtggattggacagattgatcctggagatggtgatactaactacaacggaaagttcaggggcaaggccacactgactgcagacaaatcctccagcacagcctacatgcagctcaacaccctgacctctgaggactctgcggtctatttctgtgcaagatcaagacagctcaggcttgactactggggccaaggcaccactctcacagtctcctca

SEQ ID NO: 83 - protein, 117 aa, Mus musculus
  MISC_FEATURE: SC115.84 VH
QVQLQQSGAELVKPGASVKISCETSGYTFSSYWMNWVKQRPGKGLEWIGQIDPGDGDTNYNGKFRGKATLTADKSSSTAYMQLNTLTSEDSAVYFCARSRQLRLDYWGQGTTLTVSS

SEQ ID NO: 84 - DNA, 333 bp, Mus musculus
  misc_feature: SC115.90 VL
gacattgtgctgacacagtctcctgcttccttagctgtatctctggggcagagggccaccatctcatgcagggccagcaaaagtgtcagtacatctggctacagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttcctctcacgttcggtgctgggaccaggctggagctgaaa

SEQ ID NO: 85 - protein, 111 aa, Mus musculus
  MISC_FEATURE: SC115.90 VL
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTRLELK

SEQ ID NO: 86 - DNA, 363 bp, Mus musculus
  misc_feature: SC115.90 HC
  misc_feature: SC115.90 VL
gacgtgaagctcgtggagtctgggggaggctttgtgaggcttggagggtccctgaaactctcctgtgcagcctctggattcactttcaataactatgccatgtcttgggttcgccagactccggagaagaggctggagtgggtcgcaatcattagtcgtggtggtactaaaaactactatccagacagtgtgaagggtcgattcaccatctccagagacaatgccaagaacaccctgtacctgcaaatgagcagtttgaggtctgaggacacggccatgtattactgtgcaagactctactatgatgactacgagggttatatggactactggggtcaaggaacctcagtcaccgtctcctca

SEQ ID NO: 87 - protein, 121 aa, Mus musculus
  MISC_FEATURE: SC115.90 VH
DVKLVESGGGFVRLGGSLKLSCAASGFTFNNYAMSWVRQTPEKRLEWVAIISRGGTKNYYPDSVKGRFTISRDNAKNTLYLQMSSLRSEDTAMYYCARLYYDDYEGYMDYWGQGTSVTVSS

SEQ ID NO: 88 - DNA, 345 bp, Mus musculus
  misc_feature: SC115.94 VL
caacttgtgctcactcagtcatcttcagcctctttctccctgggagcctcagcaaaactcacgtgcaccttgagtagtcagcacagtacgtacaccattgaatggtatcagcaacagccactcaagcctcctaagtatgtgatggagcttaagaaagatggaagccacaacacaggtgatgggattcctgatcgcttctctggatccagctctggtgctgatcgctaccttagcatttccaacatccagcctgaagatgaagcaatatacatctgtggtgtgggttatacaattaaggaacaatttgtgtatgttttcggcggtggaaccaaggtcactgtccta

SEQ ID NO: 89 - protein, 115 aa, Mus musculus
  MISC_FEATURE: SC115.94 VL
QLVLTQSSSASFSLGASAKLTCTLSSQHSTYTIEWYQQQPLKPPKYVMELKKDGSHNTGDGIPDRFSGSSSGADRYLSISNIQPEDEAIYICGVGYTIKEQFVYVFGGGTKVTVL

SEQ ID NO: 90 - DNA, 351 bp, Mus musculus
  misc_feature: SC115.94 VH
caggttcagctgcagctgtctggggctgagctggtgaagcctggggcctcagtgaagatttcctgcaaagcttctggctacgcattcagtagctactggatgaactgggtgaagcagaggcctggaaagggtcttgagtggattggacagatttatcctggagatgctgatactcactacaacggaaagttcaagggcaaggccacactgactgcagacaaatcctccagcacagcctacatgcagctcagcagcctgacctctgaggactctgcggtctatttctgtgcaagattaaaactgggacgttttgactactggggccaaggcaccactctcacagtctcctca

SEQ ID NO: 91 - protein, 117 aa, Mus musculus
  MISC_FEATURE: SC115.94 VH
QVQLQLSGAELVKPGASVKISCKASGYAFSSYWMNWVKQRPGKGLEWIGQIYPGDADTHYNGKFKGKATLTADKSSSTAYMQLSSLTSEDSAVYFCARLKLGRFDYWGQGTTLTVSS

SEQ ID NO: 92-99 - skipped (8 entries)

SEQ ID NO: 100 - DNA, 339 bp, Artificial Sequence
  hSC115.9 VL
gatatccagatgacccaaagcccaagctctctgtccgcatctgttggagacagggtgactatcacctgcaagagcagccagtccctccttaacagtgggaatcagaagaattacctgacttggtaccagcagaagcctggcaaagcccccaagctgttgatctattgggctagcacacgggagtccggcgttccctccagattctctgggtcaggaagtggaaccgatttcactctcaccatctcctctcttcagcctgaagacttcgccacttattactgtcagaacgactatagttacccattgactttcggtcagggaacaaaacttgaaattaag

SEQ ID NO: 101 - protein, 113 aa, Artificial Sequence
  hSC115.9 VL
DIQMTQSPSSLSASVGDRVTITCKSSQSLLNSGNQKNYLTWYQQKPGKAPKLLIYWASTRESGVPSRFSGSGSGTDFTLTISSLQPEDFATYYCQNDYSYPLTFGQGTKLEIK

SEQ ID NO: 102 - DNA, 354 bp, Artificial Sequence
  hSC115.9 VH
gaagtacagctcgtgcagtccggggccgaggtgaaaaagcctggcgagtctttgaagatttcttgcaagggcagtggatattcctttaccagtaattggatgcactgggtccgacagatgcccggcaagggcctggaatggatgggagtaattgatccaagcgatacctacatctcctacaatcagaaatttaagggacaggtgaccatctctgccgacaagtccatcagcactgcctatctgcagtggtctagtctgaaggcttcagatacagcaatgtattactgtgctaggcgtgactccgggtggtatttcgatgtgtggggccaggggaccacagtgaccgtgtcttcc

SEQ ID NO: 103 - protein, 118 aa, Artificial Sequence
  hSC115.9 VH
EVQLVQSGAEVKKPGESLKISCKGSGYSFTSNWMHWVRQMPGKGLEWMGVIDPSDTYISYNQKFKGQVTISADKSISTAYLQWSSLKASDTAMYYCARRDSGWYFDVWGQGTTVTVSS

SEQ ID NO: 104 - DNA, 339 bp, Artificial Sequence
  hSC115.18 VL
gacattcagatgacccaatccccctccagcctgtctgcctcagtgggagaccgtgtgaccataacttgtaagtcatcccaaagcctgcttaactccgggaaccaaaagaactatcttacctggtatcaacagaaacccggcaaagcaccaaaactcttgatttactgggcttctacacgcgactctggcgttcctagtcgattcagcggctctgggtccggaaccgacttcacactgaccatcagcagtgttcaaccagaggacttcgctacttattactgtcagaacgactactcttatcctctcacctttggccaaggaactaagttggagattaag

SEQ ID NO: 105 - protein, 113 aa, Artificial Sequence
  hSC115.18 VL
DIQMTQSPSSLSASVGDRVTITCKSSQSLLNSGNQKNYLTWYQQKPGKAPKLLIYWASTRDSGVPSRFSGSGSGTDFTLTISSVQPEDFATYYCQNDYSYPLTFGQGTKLEIK

SEQ ID NO: 106 - DNA, 363 bp, Artificial Sequence
  hSC115.18 VH
caggtgcagctggtccagtctggtgctgaggtaaagaagcccggtgcatccgtcaaagtctcttgtaaggcctctgggtataccttcaccagctattggatgcattgggtgaggcaggctcctggacagcgtctggaatggatggggaacatcgaccctagcgactccgagatctactacaatcagaagtttaaagaccgggtaaccctcacacgagacacaagtgccagtacagcctacatggagctgtcatctctgcggtccgaggatactgctgtctattattgcgctcggagatccggttctcgcgactactatggcatggacgattggggtcagggaaccactgttaccgtgagttct

SEQ ID NO: 107 - protein, 121 aa, Artificial Sequence
  hSC115.18 VH
QVQLVQSGAEVKKPGASVKVSCKASGYTFTSYWMHWVRQAPGQRLEWMGNIDPSDSEIYYNQKFKDRVTLTRDTSASTAYMELSSLRSEDTAVYYCARRSGSRDYYGMDDWGQGTTVTVSS

SEQ ID NO: 108-109 - skipped (2 entries)

SEQ ID NO: 110 - protein, 220 aa, Artificial Sequence
  hSC115.9 full length light chain
DIQMTQSPSSLSASVGDRVTITCKSSQSLLNSGNQKNYLTWYQQKPGKAPKLLIYWASTRESGVPSRFSGSGSGTDFTLTISSLQPEDFATYYCQNDYSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 447 aa, Artificial Sequence
  hSC115.9 full length heavy chain
EVQLVQSGAEVKKPGESLKISCKGSGYSFTSNWMHWVRQMPGKGLEWMGVIDPSDTYISYNQKFKGQVTISADKSISTAYLQWSSLKASDTAMYYCARRDSGWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 447 aa, Artificial Sequence
  hSC115.9ss1 full length heavy chain
EVQLVQSGAEVKKPGESLKISCKGSGYSFTSNWMHWVRQMPGKGLEWMGVIDPSDTYISYNQKFKGQVTISADKSISTAYLQWSSLKASDTAMYYCARRDSGWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 220 aa, Artificial Sequence
  hSC115.18 full length light chain
DIQMTQSPSSLSASVGDRVTITCKSSQSLLNSGNQKNYLTWYQQKPGKAPKLLIYWASTRDSGVPSRFSGSGSGTDFTLTISSVQPEDFATYYCQNDYSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 450 aa, Artificial Sequence
  hSC115.18 full length heavy chain
QVQLVQSGAEVKKPGASVKVSCKASGYTFTSYWMHWVRQAPGQRLEWMGNIDPSDSEIYYNQKFKDRVTLTRDTSASTAYMELSSLRSEDTAVYYCARRSGSRDYYGMDDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 450 aa, Artificial Sequence
  hSC115.18ss1 full length heavy chain
QVQLVQSGAEVKKPGASVKVSCKASGYTFTSYWMHWVRQAPGQRLEWMGNIDPSDSEIYYNQKFKDRVTLTRDTSASTAYMELSSLRSEDTAVYYCARRSGSRDYYGMDDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2019/0000969 A1
(43) Pub. Date: Jan. 3, 2019

(54) NOVEL ANTI-UPK1B ANTIBODIES AND METHODS OF USE

(71) Applicant: ABBVIE STEMCENTRX LLC, NORTH CHICAGO, IL (US)

(72) Inventors: KRISTEN MCKNIGHT, SAN FRANCISCO, CA (US); SARAH FONG, OAKLAND, CA (US); SAMUEL A. WILLIAMS, SAN MATEO, CA (US); ZHAO HUANG, BURLINGAME, CA (US); EARL KIM, BURLINGAME, CA (US); ROBERT A. STULL, ALAMEDA, CA (US); DAVID COELHO, SAN FRANCISCO, CA (US); WADE C. ANDERSON, FAIRFIELD, CA (US); MARIANNE SANTAGUIDA, BELMONT, CA (US)

(73) Assignee: ABBVIE STEMCENTRX LLC, NORTH CHICAGO, IL (US)

(21) Appl. No.: 16/065,064

(22) PCT Filed: Dec. 21, 2016
(86) PCT No.: PCT/US2016/068146

§ 371 Date: Jun. 21, 2018

Related U.S. Application Data
(60) Provisional application No. 62/270,993, filed on Dec. 22, 2015.
(60) Provisional application No. 62/430,191, filed on Dec. 5, 2016.

(51) Int. Cl. A61K 39/395; A61P 35/00; A61K 47/68; A61K 49/00; G01N 33/574

(52) U.S. Cl. CPC A61K 39/395; A61P 35/00; G01N 33/57438; A61K 47/6849; A61K 49/0058; A61K 47/6803

(57) ABSTRACT

Provided are novel anti-UPK1B antibodies and antibody drug conjugates, and methods of using such anti-UPK1B antibodies and antibody drug conjugates to treat cancer.

### CROSS REFERENCED APPLICATIONS

This application claims the benefit of U.S. Provisional Application No. 62/270,993 filed on Dec. 22, 2015 and U.S. Provisional Application No. 62/430,191 filed on Dec. 5, 2016, which are incorporated herein by reference in their entirety.

### SEQUENCE LISTING

This application contains a sequence listing which has been submitted in ASCII format via EFS-Web and is hereby incorporated by reference in its entirety. Said ASCII copy, created on Dec. 5, 2016, is named S69697_1360WO_sc11501WO01_ST25.txt and is 104 KB (107,102 bytes) in size.

### FIELD OF THE INVENTION

This application generally relates to novel anti-UPK1B antibodies or immunoreactive fragments thereof and compositions, including antibody drug conjugates, comprising the same for the treatment, diagnosis or prophylaxis of cancer and any recurrence or metastasis thereof. Selected embodiments of the invention provide for the use of such anti-UPK1B antibodies or antibody drug conjugates for the treatment of cancer comprising a reduction in tumorigenic cell frequency.

### BACKGROUND OF THE INVENTION

Differentiation and proliferation of stem cells and progenitor cells are normal ongoing processes that act in concert to support tissue growth during organogenesis, cell repair and cell replacement. The system is tightly regulated to ensure that only appropriate signals are generated based on the needs of the organism. Cell proliferation and differentiation normally occur only as necessary for the replacement of damaged or dying cells or for growth. However, disruption of these processes can be triggered by many factors including the under- or overabundance of various signaling chemicals, the presence of altered microenvironments, genetic mutations or a combination thereof. Disruption of normal cellular proliferation and/or differentiation can lead to various disorders including proliferative diseases such as cancer.

Conventional therapeutic treatments for cancer include chemotherapy, radiotherapy and immunotherapy. Often these treatments are ineffective and surgical resection may not provide a viable clinical alternative. Limitations in the current standard of care are particularly evident in those cases where patients undergo first line treatments and subsequently relapse. In such cases refractory tumors, often aggressive and incurable, frequently arise. The overall survival rates for many tumors have remained largely unchanged over the years due, at least in part, to the failure of existing therapies to prevent relapse, tumor recurrence and metastasis. There remains therefore a great need to develop more targeted and potent therapies for proliferative disorders. The current invention addresses this need.

### SUMMARY OF THE INVENTION

In a broad aspect the present invention provides isolated antibodies, and corresponding antibody drug or diagnostic conjugates (ADCs), or compositions thereof, which specifically bind to human UPK1B determinants. In certain embodiments the UPK1B determinant is a UPK1B protein expressed on tumor cells while in other embodiments the UPK1B determinant is expressed on tumor initiating cells. In other embodiments the antibodies of the invention bind to a UPK1B protein and compete for binding with an antibody that binds to an epitope on human UPK1B protein.

In selected embodiments the invention comprises an antibody that comprises or competes for binding with an isolated antibody that binds to a cell expressing human UPK1B having SEQ ID NO: 1, wherein the isolated antibody comprises: (1) a light chain variable region (VL) of SEQ ID NO: 21 and a heavy chain variable region (VH) of SEQ ID NO: 23; or (2) a VL of SEQ ID NO: 25 and a VH of SEQ ID NO: 27; or (3) a VL of SEQ ID NO: 29 and a VH of SEQ ID NO: 31; or (4) a VL of SEQ ID NO: 33 and a VH of SEQ ID NO: 35; or (5) a VL of SEQ ID NO: 37 and a VH of SEQ ID NO: 39; or (6) a VL of SEQ ID NO: 41 and a VH of SEQ ID NO: 43; or (7) a VL of SEQ ID NO: 45 and a VH of SEQ ID NO: 47; or (8) a VL of SEQ ID NO: 49 and a VH of SEQ ID NO: 51; or (9) a VL of SEQ ID NO: 53 and a VH of SEQ ID NO: 55; or (10) a VL of SEQ ID NO: 57 and a VH of SEQ ID NO: 59; or (11) a VL of SEQ ID NO: 61 and a VH of SEQ ID NO: 63; or (12) a VL of SEQ ID NO: 65 and a VH of SEQ ID NO: 67; or (13) a VL of SEQ ID NO: 69 and a VH of SEQ ID NO: 71; or (14) a VL of SEQ ID NO: 73 and a VH of SEQ ID NO: 75; or (15) a VL of SEQ ID NO: 77 and a VH of SEQ ID NO: 79; or (16) a VL of SEQ ID NO: 81 and a VH of SEQ ID NO: 83; or (17) a VL of SEQ ID NO: 85 and a VH of SEQ ID NO: 87; or (18) a VL of SEQ ID NO: 89 and a VH of SEQ ID NO: 91.

In a further aspect, the invention comprises an antibody that binds to UPK1B comprising a light chain variable region and a heavy chain variable region, wherein the light chain variable region has three CDRs of a light chain variable region set forth as SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53 SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85 or SEQ ID NO: 89; and the heavy chain variable region has three CDRs of a heavy chain variable region set forth as SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO:59, SEQ ID NO: 63, SEQ ID NO: 67, SEQ ID NO: 71, SEQ ID NO: 75, SEQ ID NO: 79, SEQ ID N,O: 83, SEQ ID NO: 87 or SEQ ID NO: 91.

In other aspects the invention comprises humanized antibodies having a VL comprising SEQ ID NO: 101 and a VH comprising SEQ ID NO: 103 or having a VL comprising SEQ ID NO: 105 and a VH comprising SEQ ID NO: 107. In certain embodiments these humanized antibodies will comprise site-specific antibodies.

In other selected embodiments the invention will comprise a humanized antibody selected from the group consisting of hSC115.9 (SEQ ID NOS: 110 and 111), hSC115.9ss1 (SEQ ID NOS: 110 and 112), hSC115.18 (SEQ ID NOS: 113 and 114) and hSC115.18ss1 (SEQ ID NOS: 113 and 115).

In some aspects of the invention the antibody comprises a chimeric, CDR grafted, humanized or human antibody or an immunoreactive fragment thereof. In other aspects of the invention the antibody, preferably comprising all or part of the aforementioned sequences, is an internalizing antibody. In yet other embodiments the antibodies will comprise site-specific antibodies. In other selected embodiments the invention comprises antibody drug conjugates incorporating any of the aforementioned antibodies.

In certain aspects the invention comprises a nucleic acid encoding an anti-UPK1B antibody of the invention or a fragment thereof. In other embodiments the invention comprises a vector comprising one or more of the above described nucleic acids or a host cell comprising said nucleic acids or vectors.

As alluded to above the present invention further provides anti-UPK1B antibody drug conjugates where antibodies as disclosed herein are conjugated to a payload. In certain aspects the present invention comprises ADCs that immunopreferentially associate or bind to hUPK1B. Compatible anti-UPK1B antibody drug conjugates (ADCs) of the invention may generally comprise the formula:

Ab-[L-D]n or a pharmaceutically acceptable salt thereof wherein
a) Ab comprises an anti-UPK1B antibody;
b) L comprises an optional linker;
c) D comprises a drug; and
d) n is an integer from about 1 to about 20.

In certain aspects the ADCs of the invention comprise an anti-UPK1B antibody such as those described above or an immunoreactive fragment thereof. In other embodiments the ADCs of the invention comprise a cytotoxic compound selected from radioisotopes, calicheamicins, pyrrolobenzodiazepines, benzodiazepine derivatives, auristatins, dolastatins, duocarmycins, maytansinoids or an additional therapeutic moiety described herein.

In certain aspects the invention may generally comprise the formula:

Ab-[L-D]n or a pharmaceutically acceptable salt thereof wherein
a) Ab comprises an anti-UPK1B antibody;
b) L comprises an optional linker;
c) D comprises an auristatin; and

n is an integer from about 1 to about 20.

In other aspects the invention may generally comprise the formula:

Ab-[L-D]n or a pharmaceutically acceptable salt thereof wherein
a) Ab comprises an anti-UPK1B antibody;
b) L comprises an optional linker;
c) D comprises an dolastatin; and

n is an integer from about 1 to about 20.

Further provided are pharmaceutical compositions comprising an anti-UPK1B ADC as disclosed herein. In certain embodiments the compositions with comprise a selected drug-antibody ratio (DAR) of greater than about 50%, greater than about 60%, greater than about 70%, greater than about 80%, greater than about 90% or even greater than about 95%. In some embodiments the selected DAR will be two, while in other embodiments the selected DAR will be four and in other embodiments the selected DAR will be six and in yet other embodiments the selected DAR will be eight.

Another aspect of the invention is a method of treating cancer comprising administering a pharmaceutical composition such as those described herein to a subject in need thereof. In certain aspects the cancer comprises a hematologic malignancy such as, for example, acute myeloid leukemia or diffuse large B-cell lymphoma. In other aspects the subject will be suffering from a solid tumor. With regard to such embodiments the cancer is preferably selected from the group consisting of adrenal cancer, liver cancer, kidney cancer, bladder cancer, breast cancer, gastric cancer, ovarian cancer, cervical cancer, uterine cancer, esophageal cancer, colorectal cancer, prostate cancer, pancreatic cancer, lung cancer (both small cell and non-small cell), thyroid cancer, mesothelioma and glioblastoma. In certain embodiments the subject will be suffering from pancreatic cancer, bladder cancer, head and neck cancer, non-small cell lung cancer, ovarian cancer, gastric cancer, uterine cancer or endometrial cancer. In some aspects the subject will be suffering from pancreatic cancer. In other aspects the subject will be suffering from bladder cancer. Further, in selected embodiments the method of treating cancer described above comprises administering to the subject at least one additional therapeutic moiety besides the anti-UPK1B ADCs of the invention.

In still another embodiment the invention comprises a method of reducing tumor initiating cells in a tumor cell population, wherein the method comprises contacting (e.g. in vitro or in vivo) a tumor initiating cell population with an ADCs as described herein whereby the frequency of the tumor initiating cells is reduced.

In one aspect, the invention comprises a method of delivering a cytotoxin to a cell comprising contacting the cell with any of the above described ADCs.

In another aspect, the invention comprises a method of detecting, diagnosing, or monitoring cancer (e.g. lung cancer or hematologic malignancies) in a subject, the method comprising the steps of contacting (e.g. in vitro or in vivo) tumor cells with an UPK1B detection agent and detecting the UPK1B agent associated with the tumor cells. In selected embodiments the detection agent shall comprise an anti-UPK1B antibody or a nucleic acid probe that associates with an UPK1B genotypic determinant. In related embodiments the diagnostic method will comprise immunohistochemistry (IHC) or in situ hybridization (ISH). Those of skill in the art will appreciate that such agents optionally may be labeled or associated with effectors, markers or reporters as disclosed below and detected using any one of a number of standard techniques (e.g., MRI, CAT scan, PET scan, etc.).

In a similar vein the present invention also provides kits or devices and associated methods that are useful in the diagnosis, monitoring or treatment of UPK1B associated disorders such as cancer. To this end the present invention preferably provides an article of manufacture useful for detecting, diagnosing or treating UPK1B associated disorders comprising a receptacle containing a UPK1B ADC and instructional materials for using said UPK1B ADC to treat, monitor or diagnose the UPK1B associated disorder or provide a dosing regimen for the same. In selected embodiments the devices and associated methods will comprise the step of contacting at least one circulating tumor cell. In other embodiments the disclosed kits will comprise instructions, labels, inserts, readers or the like indicating that the kit or device is used for the diagnosis, monitoring or treatment of a UPK1B associated cancer or provide a dosing regimen for the same.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description.

### BRIEF DESCRIPTION OF THE FIGURES

FIGS. 1A and 1B provide, respectively, an annotated amino acid sequence of UPK1B (FIG. 1A) along with a schematic representation of the same (FIG. 1B);

FIGS. 2A and 2B show expression levels of UPK1B as measured through whole transcriptome sequencing of RNA derived from patient derived xenograft (PDX) cancer stem cells (CSC) and non-tumorigenic (NTG) cells as well as normal tissue using a SOUD platform (FIG. 2A) or an Illumina platform (FIG. 2B);

FIG. 3 depicts the relative expression levels of UPK1B transcripts as measured by qRT-PCR in RNA samples isolated from normal tissue and from a variety of PDX tumors;

FIG. 4 shows the normalized intensity value of UPK1B transcript expression measured by microarray hybridization in normal tissues and a variety of PDX cell lines;

FIG. 5 shows expression of UPK1B transcripts in normal tissues and primary tumors from The Cancer Genome Atlas (TCGA), a publically available dataset;

FIG. 6 depicts Kaplan-Meier survival curves based on high and low expression of UPK1B transcripts in primary tumors from the TCGA dataset wherein the threshold index value is determined using the arithmetic mean of the TPM values;

FIGS. 7A and 7B provide, in a tabular form and plot respectively, antibody isotype, cell killing and binning characteristics of exemplary anti-UPK1B antibodies (FIG. 7A) and the cell killing activity of the antibodies plotted as a function of their bin (FIG. 7B);

FIG. 8 illustrates the level of UPK1B protein expression in a number of exemplary PDX tumor cell lines;

FIGS. 9A and 9B show, in tabular form, UPK1B protein expression on engineered cell lines and PDX tumor cells as determined by immunohistochemistry while FIGS. 9C and 9D show UPK1B protein expression on bladder (FIG. 9C) and pancreatic (FIG. 9D) cancer tissue arrays as determined by immunohistochemistry and plotted as a function of tumor subtype;

FIGS. 10A and 10B shows UPK1B protein expression on the surface of tumor cells as determined by flow cytometry with various pancreas (FIG. 10A) and bladder (FIG. 10B) cancer PDX cell lines where an exemplary antibody of the instant invention (black line) is compared to an isotype-control stained population (solid gray);

FIGS. 11A and 11B depicts UPK1B RNA (FIG. 11A) and protein (FIG. 11B) levels as a function of CDKN2A mutation status determined by microarray or flow cytometry respectively;

FIGS. 12A-12F provide annotated amino acid and nucleic acid sequences wherein FIGS. 12A and 12B show contiguous amino acid sequences of the light chain (FIG. 12A) and heavy chain (FIG. 12B) variable regions (SEQ ID NOS: 21-91, odd numbers) of exemplary murine anti-UPK1B antibodies, FIG. 12C shows nucleic acid sequences encoding the aforementioned light and heavy chain variable regions (SEQ ID NOS: 20-90, even numbers), FIGS. 12D and 12E depict amino acid sequences and nucleic acid sequences of humanized VL and VH domains, FIG. 12F shows amino acid sequences of full length heavy and light chains and FIGS. 12G and 12H depict, respectively, CDRs of the light and heavy chain variable regions of SC115.9 and SC115.18 murine antibodies as determined using Kabat, Chothia, ABM and Contact methodology;

FIG. 13 depicts, in tabular form, the antibody affinity for parental and humanized UPK1B antibody clones;

FIGS. 14A and 14B illustrate the cell killing capacity humanized UPK1B antibodies hSC115.9 and hSC115.18 as compared to chimeric antibodies comprising the source murine VH and VL domains;

FIG. 15 depicts the ability of humanized anti-UPK1B ADCs to internalize and kill HEK293T cells overexpressing UPK1B protein;

FIGS. 16A-16C show that anti-UPK1B ADCs are able to internalize into PA tumors in vivo and cause a significant and prolonged reduction in tumor volume through the delivery of different cytotoxins; and

FIG. 17 illustrates the correlation between expression level of UPK1B and the amount of in vivo tumor growth inhibition.

### DETAILED DESCRIPTION OF THE INVENTION

The invention may be embodied in many different forms. Disclosed herein are non-limiting, illustrative embodiments of the invention that exemplify the principles thereof. Any section headings used herein are for organizational purposes only and are not to be construed as limiting the subject matter described. For the purposes of the instant disclosure all identifying sequence accession numbers may be found in the NCBI Reference Sequence (RefSeq) database and/or the NCBI GenBank® archival sequence database unless otherwise noted.

It has surprisingly been found that UPK1B phenotypic determinants are clinically associated with various proliferative disorders, including neoplasia, and that UPK1B protein and variants or isoforms thereof provide useful tumor markers which may be exploited in the treatment of related diseases. In this regard the present invention provides antibody drug conjugates comprising an engineered anti-UPK1B antibody targeting agent and cytotoxic payload. As discussed in more detail below and set forth in the appended Examples, the disclosed anti-UPK1B ADCs are particularly effective at eliminating tumorigenic cells and therefore useful for the treatment and prophylaxis of certain proliferative disorders or the progression or recurrence thereof. In addition, the disclosed ADC compositions may exhibit a relatively high DAR=2 percentage and unexpected stability that can provide for an improved therapeutic index when compared with conventional ADC compositions comprising the same components.

Moreover, it has been found that UPK1B markers or determinants such as cell surface UPK1B protein are therapeutically associated with cancer stem cells (also known as tumor perpetuating cells) and may be effectively exploited to eliminate or silence the same. The ability to selectively reduce or eliminate cancer stem cells through the use of anti-UPK1B conjugates as disclosed herein is surprising in that such cells are known to generally be resistant to many conventional treatments. That is, the effectiveness of traditional, as well as more recent targeted treatment methods, is often limited by the existence and/or emergence of resistant cancer stem cells that are capable of perpetuating tumor growth even in face of these diverse treatment methods. Further, determinants associated with cancer stem cells often make poor therapeutic targets due to low or inconsistent expression, failure to remain associated with the tumorigenic cell or failure to present at the cell surface. In sharp contrast to the teachings of the prior art, the instantly disclosed ADCs and methods effectively overcome this inherent resistance and to specifically eliminate, deplete, silence or promote the differentiation of such cancer stem cells thereby negating their ability to sustain or re-induce the underlying tumor growth.

Thus, it is particularly remarkable that UPK1B conjugates such as those disclosed herein may advantageously be used in the treatment and/or prevention of selected proliferative (e.g., neoplastic) disorders or progression or recurrence thereof. It will be appreciated that, while preferred embodiments of the invention will be discussed extensively below, particularly in terms of particular domains, regions or epitopes or in the context of cancer stem cells or tumors comprising neuroendocrine features and their interactions with the disclosed antibody drug conjugates, those skilled in the art will appreciate that the scope of the instant invention is not limited by such exemplary embodiments. Rather, the most expansive embodiments of the present invention and the appended claims are broadly and expressly directed to anti-UPK1B antibodies and conjugates, including those disclosed herein, and their use in the treatment and/or prevention of a variety of UPK1B associated or mediated disorders, including neoplastic or cell proliferative disorders, regardless of any particular mechanism of action or specifically targeted tumor, cellular or molecular component.

### I. UPK1B Physiology

The tetraspanin (TM4SF) protein family, as the name suggests, consists of proteins that contain four-transmembrane (4TM) helices. In humans, 33 genes encode for proteins of this family (Hemler, 2014; PMID:24505619). Typically, tetraspanins are distinguished from other 4TM proteins (e.g., claudins) in that tetraspanins have both the intracellular amino and carboxyl tails which are predicted to be short, and the second extracellular loop is larger than the first (Zoller, 2009; PMID:19078974). Interestingly, conservation between tetraspanin family members is highest in the TM domains themselves, with wider sequence divergence in the extracellular loops. Tetraspanins may be found in various membranes within the cell, including the plasma membrane, but the function of these proteins frequently is poorly understood. It is known that tetraspanins may associate with membrane proteins and other tetraspanins to form tetraspanin-enriched microdomains, also referred to as the tetraspanin webs. Within these microdomains, the tetraspanins may contribute to a variety of cellular processes, including by not limited to cell adhesion, migration, signaling, receptivity to viruses (including cancer-causing viruses), invasion, and cell-cell fusion (Hemler, 2014). The specific roles that various tetraspanins play in each of these processes, or in oncogenesis and cancer progression, remain unclear.

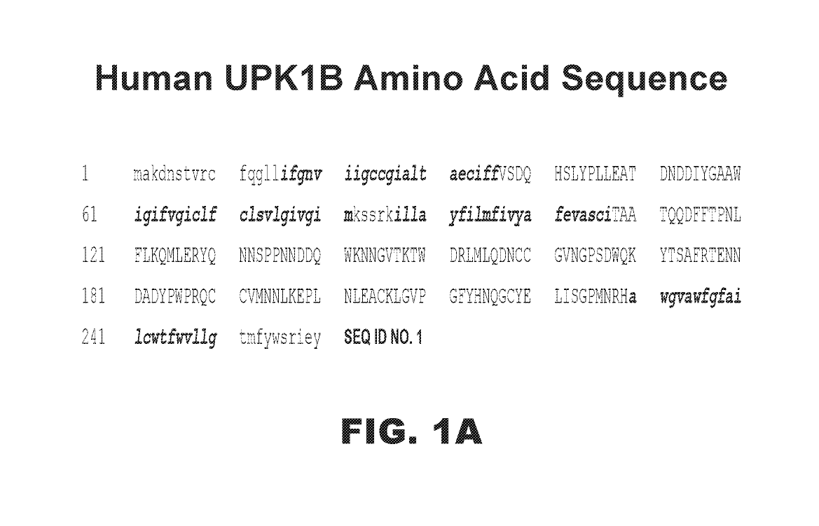

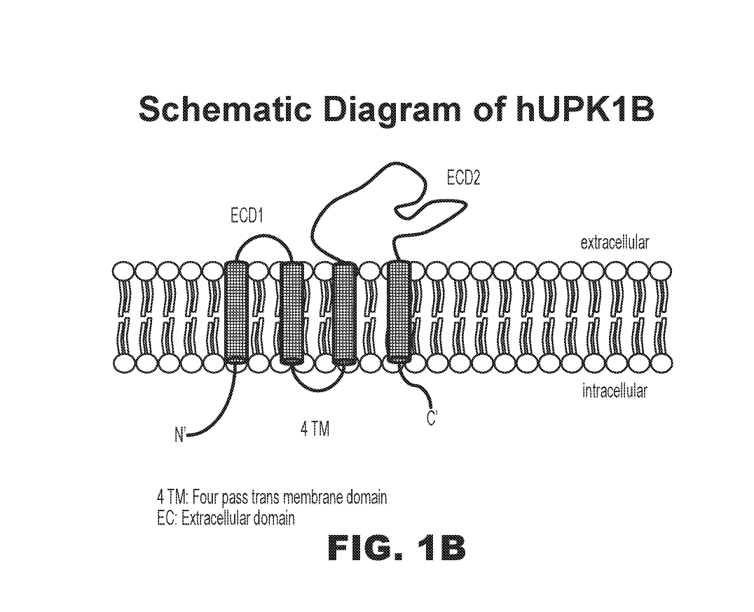

Uroplakin-1B (UPK1B; also known as TSPAN20 or UPIb) is a member of the tetraspanin family. Representative orthologues of the UPK1B protein include, but are not limited to, human (NP_006952), chimpanzee (XP_526274), mouse (NP_849255), rat (NP_001019424) and rhesus monkey (XP_001108219). In humans, the gene encoding UPK1B consists of 8 exons spanning approximately 31 kBp and is localized on chromosome 3q13.32. Transcription of the locus gives rise to one known 2060 bp transcript (NM_006952), although alternate polyadenylation sites have been reported for this gene. The transcript is subsequently translated into a 260 amino acid protein (NP_008883) that is co-translationally inserted into the cell membrane. FIG. 1A depicts the primary amino acid sequence of the human UPK1B protein, with the four transmembrane domains shown in lower case bold font, the extracellular domains shown in upper case font, and the short intracellular domains shown in standard lower case font. FIG. 1B provides a schematic illustration of the topology of the human UPK1B protein.

The epithelial lining of the urinary tract in mammals, the urothelium, is composed of several layers: a basal cell layer, an intermediate layer, and an apical layer. The apical layer of cells is formed by large hexagonal cells, termed umbrella cells, which are tightly interconnected by tight junctions and covered by crystalline plaques. These plaques give rise to an ultrastructural feature that has been termed the asymmetric unit membrane (AUM), due to it appearance in cross-section, where the outer leaflet of the plaque is nearly twice a thick as the inner leaflet. The thick outer leaflet consists of two-dimensional arrays of crystalline proteins, of which UPK1B is one component (Wu et al., 1990; PMID:229070; Yu et al., 1990; PMID:1697295). In this location, UPK1B is thought to regulate membrane permeability, to help control transcellular flux of molecules from the bladder lumen back into the blood stream, and to participate in processes to strengthen and stabilize the urothelial apical surface, avoiding membrane rupture during bladder distention. UPK1B may form heterodimers with other uroplakins, particularly UPK3A and UPK3B; the latter two proteins each seem to require chaperone functions of UPK1B to escape the endoplasmic reticulum during biosynthesis, while the UPK1B protein can “self-export” when overexpressed ectopically (Tu et al. 2002; PMID:12475947). UPK1B has also been detected in urinary exosomes (Gonzales et al., PMID:19056867).

### II. Cancer Stem Cells

According to current models, a tumor comprises non-tumorigenic cells and tumorigenic cells. Non-tumorigenic cells do not have the capacity to self-renew and are incapable of reproducibly forming tumors, even when transplanted into immunocompromised mice in excess cell numbers. Tumorigenic cells, also referred to herein as “tumor initiating cells” (TICs), which typically make up a fraction of the tumor's cell population of 0.01-10%, have the ability to form tumors. For hematopoietic malignancies TICs can be very rare ranging from 1:10⁴ to 1:10⁷ in particular in Acute Myeloid Malignancies (AML) or very abundant for example in lymphoma of the B cell lineage. Tumorigenic cells encompass both tumor perpetuating cells (TPCs), referred to interchangeably as cancer stem cells (CSCs), and tumor progenitor cells (TProgs).

CSCs, like normal stem cells that support cellular hierarchies in normal tissue, are able to self-replicate indefinitely while maintaining the capacity for multilineage differentiation. In this regard CSCs are able to generate both tumorigenic progeny and non-tumorigenic progeny and are able to completely recapitulate the heterogeneous cellular composition of the parental tumor as demonstrated by serial isolation and transplantation of low numbers of isolated CSCs into immunocompromised mice. Evidence indicates that unless these “seed cells” are eliminated tumors are much more likely to metastasize or reoccur leading to relapse and ultimate progression of the disease.

TProgs, like CSCs have the ability to fuel tumor growth in a primary transplant. However, unlike CSCs, they are not able to recapitulate the cellular heterogeneity of the parental tumor and are less efficient at reinitiating tumorigenesis in subsequent transplants because TProgs are typically only capable of a finite number of cell divisions as demonstrated by serial transplantation of low numbers of highly purified TProg into immunocompromised mice. TProgs may further be divided into early TProgs and late TProgs, which may be distinguished by phenotype (e.g., cell surface markers) and their different capacities to recapitulate tumor cell architecture. While neither can recapitulate a tumor to the same extent as CSCs, early TProgs have a greater capacity to recapitulate the parental tumor's characteristics than late TProgs. Notwithstanding the foregoing distinctions, it has been shown that some TProg populations can, on rare occasion, gain self-renewal capabilities normally attributed to CSCs and can themselves become CSCs.

CSCs exhibit higher tumorigenicity and are often relatively more quiescent than: (i) TProgs (both early and late TProgs); and (ii) non-tumorigenic cells such as terminally differentiated tumor cells and tumor-infiltrating cells, for example, fibroblasts/stroma, endothelial and hematopoietic cells that may be derived from CSCs and typically comprise the bulk of a tumor. Given that conventional therapies and regimens have, in large part, been designed to debulk tumors and attack rapidly proliferating cells, CSCs are therefore more resistant to conventional therapies and regimens than the faster proliferating TProgs and other bulk tumor cell populations such as non-tumorigenic cells. Other characteristics that may make CSCs relatively chemoresistant to conventional therapies are increased expression of multi-drug resistance transporters, enhanced DNA repair mechanisms and anti-apoptotic gene expression. Such CSC properties have been implicated in the failure of standard treatment regimens to provide a lasting response in patients with advanced stage neoplasia as standard chemotherapy does not effectively target the CSCs that actually fuel continued tumor growth and recurrence.

It has surprisingly been discovered that UPK1B expression is associated with various tumorigenic cell subpopulations in a manner which renders them susceptible to treatment as set forth herein. The invention provides anti-UPK1B antibodies that may be particularly useful for targeting tumorigenic cells and may be used to silence, sensitize, neutralize, reduce the frequency, block, abrogate, interfere with, decrease, hinder, restrain, control, deplete, moderate, mediate, diminish, reprogram, eliminate, kill or otherwise inhibit (collectively, “inhibit”) tumorigenic cells, thereby facilitating the treatment, management and/or prevention of proliferative disorders (e.g. cancer). Advantageously, the anti-UPK1B antibodies of the invention may be selected so they preferably reduce the frequency or tumorigenicity of tumorigenic cells upon administration to a subject regardless of the form of the UPK1B determinant (e.g., phenotypic or genotypic). The reduction in tumorigenic cell frequency may occur as a result of (i) inhibition or eradication of tumorigenic cells; (ii) controlling the growth, expansion or recurrence of tumorigenic cells; (iii) interrupting the initiation, propagation, maintenance, or proliferation of tumorigenic cells; or (iv) by otherwise hindering the survival, regeneration and/or metastasis of the tumorigenic cells. In some embodiments, the inhibition of tumorigenic cells may occur as a result of a change in one or more physiological pathways. The change in the pathway, whether by inhibition or elimination of the tumorigenic cells, modification of their potential (for example, by induced differentiation or niche disruption) or otherwise interfering with the ability of tumorigenic cells to influence the tumor environment or other cells, allows for the more effective treatment of UPK1B associated disorders by inhibiting tumorigenesis, tumor maintenance and/or metastasis and recurrence. It will further be appreciated that the same characteristics of the disclosed antibodies make them particularly effective at treating recurrent tumors which have proved resistant or refractory to standard treatment regimens.

Methods that can be used to assess the reduction in the frequency of tumorigenic cells, include but are not limited to, cytometric or immunohistochemical analysis, preferably by in vitro or in vivo limiting dilution analysis (Dylla et al. 2008, PMID: PMC2413402 and Hoey et al. 2009, PMID: 19664991).

In vitro limiting dilution analysis may be performed by culturing fractionated or unfractionated tumor cells (e.g. from treated and untreated tumors, respectively) on solid medium that fosters colony formation and counting and characterizing the colonies that grow. Alternatively, the tumor cells can be serially diluted onto plates with wells containing liquid medium and each well can be scored as either positive or negative for colony formation at any time after inoculation but preferably more than 10 days after inoculation.

In vivo limiting dilution is performed by transplanting tumor cells, from either untreated controls or from tumors exposed to selected therapeutic agents, into immunocompromised mice in serial dilutions and subsequently scoring each mouse as either positive or negative for tumor formation. The scoring may occur at any time after the implanted tumors are detectable but is preferably done 60 or more days after the transplant. The analysis of the results of limiting dilution experiments to determine the frequency of tumorigenic cells is preferably done using Poisson distribution statistics or assessing the frequency of predefined definitive events such as the ability to generate tumors in vivo or not (Fazekas et al., 1982, PMID: 7040548).

Flow cytometry and immunohistochemistry may also be used to determine tumorigenic cell frequency. Both techniques employ one or more antibodies or reagents that bind art recognized cell surface proteins or markers known to enrich for tumorigenic cells (see WO 2012/031280). As known in the art, flow cytometry (e.g. florescence activated cell sorting (FACS)) can also be used to characterize, isolate, purify, enrich or sort for various cell populations including tumorigenic cells. Flow cytometry measures tumorigenic cell levels by passing a stream of fluid, in which a mixed population of cells is suspended, through an electronic detection apparatus which is able to measure the physical and/or chemical characteristics of up to thousands of particles per second. Immunohistochemistry provides additional information in that it enables visualization of tumorigenic cells in situ (e.g., in a tissue section) by staining the tissue sample with labeled antibodies or reagents which bind to tumorigenic cell markers.

As such, the antibodies of the invention may be useful for identifying, characterizing, monitoring, isolating, sectioning or enriching populations or subpopulations of tumorigenic cells through methods such as, for example, flow cytometry, magnetic activated cell sorting (MACS), laser mediated sectioning or FACS. FACS is a reliable method used to isolate cell subpopulations at more than 99.5% purity based on specific cell surface markers. Other compatible techniques for the characterization and manipulation of tumorigenic cells including CSCs can be seen, for example, in U.S. patent Ser. Nos. 12/686,359, 12/669,136 and 12/757,649.

Listed below are markers that have been associated with CSC populations and have been used to isolate or characterize CSCs: ABCA1, ABCA3, ABCB5, ABCG2, ADAM9, ADCY9, ADORA2A, ALDH, AFP, AXIN1, B7H3, BCL9, Bmi-1, BMP-4, C20orf52, C4.4A, carboxypeptidase M, CAV1, CAV2, CD105, CD117, CD123, CD133, CD14, CD16, CD166, CD16a, CD16b, CD2, CD20, CD24, CD29, CD3, CD31, CD324, CD325, CD33, CD34, CD38, CD44, CD45, CD46, CD49b, CD49f, CD56, CD64, CD74, CD9, CD90, CD96, CEACAM6, CELSR1, CLEC12A, CPD, CRIM1, CX3CL1, CXCR4, DAF, decorin, easyh1, easyh2, EDG3, EGFR, ENPP1, EPCAM, EPHA1, EPHA2, FLJ10052, FLVCR, FZD1, FZD10, FZD2, FZD3, FZD4, FZD6, FZD7, FZD8, FZD9, GD2, GJA1, GLI1, GLI2, GPNMB, GPR54, GPRC5B, HAVCR2, IL1R1, IL1RAP, JAM3, Lgr5, Lgr6, LRP3, LY6E, MCP, mf2, mllt3, MPZL1, MUC1, MUC16, MYC, N33, NANOG, NB84, NES, NID2, NMA, NPC1, OSM, OCT4, OPN3, PCDH7, PCDHA10, PCDHB2, PPAP2C, PTPN3, PTS, RARRES1, SEMA4B, SLC19A2, SLC1A1, SLC39A1, SLC4A11, SLC6A14, SLC7A8, SMARCA3, SMARCD3, SMARCE1, SMARCA5, SOX1, STAT3, STEAP, TCF4, TEM8, TGFBR3, TMEPAI, TMPRSS4, TFRC, TRKA, WNT10B, WNT16, WNT2, WNT2B, WNT3, WNT5A, YY1 and CTNNB1. See, for example, Schulenburg et al., 2010, PMID: 20185329, U.S. Pat. No. 7,632,678 and U.S.P.N.s. 2007/0292414, 2008/0175870, 2010/0275280, 2010/0162416 and 2011/0020221.

Similarly, non-limiting examples of cell surface phenotypes associated with CSCs of certain tumor types include CD44^{hi}CD24^{low}, ALDH⁺, CD133⁺, CD123⁺, CD34⁺CD38⁻, CD44⁺CD24⁻, CD46^{hi}CD324⁺CD66c⁻, CD133⁺CD34⁺CD10⁻CD19⁻, CD138⁻CD34⁻CD19⁺, CD133⁺RC2⁺, CD44⁺α₂β₁^{hi}CD133⁺, CD44⁺CD24⁺ESA⁺, CD271⁺, ABCB5⁺ as well as other CSC surface phenotypes that are known in the art. See, for example, Schulenburg et al., 2010, supra, Visvader et al., 2008, PMID: 18784658 and U.S.P.N. 2008/0138313. Of particular interest with respect to the instant invention are CSC preparations comprising CD46^{hi}CD324⁺ phenotypes in solid tumors and CD34⁺CD38⁻ in leukemias.

“Positive,” “low” and “negative” expression levels as they apply to markers or marker phenotypes are defined as follows. Cells with negative expression (i.e. “−”) are herein defined as those cells expressing less than, or equal to, the 95th percentile of expression observed with an isotype control antibody in the channel of fluorescence in the presence of the complete antibody staining cocktail labeling for other proteins of interest in additional channels of fluorescence emission. Those skilled in the art will appreciate that this procedure for defining negative events is referred to as “fluorescence minus one”, or “FMO”, staining. Cells with expression greater than the 95th percentile of expression observed with an isotype control antibody using the FMO staining procedure described above are herein defined as “positive” (i.e. “+”). As defined herein there are various populations of cells broadly defined as “positive.” A cell is defined as positive if the mean observed expression of the antigen is above the 95th percentile determined using FMO staining with an isotype control antibody as described above. The positive cells may be termed cells with low expression (i.e. “lo”) if the mean observed expression is above the 95^{th} percentile determined by FMO staining and is within one standard deviation of the 95^{th} percentile. Alternatively, the positive cells may be termed cells with high expression (i.e. “hi”) if the mean observed expression is above the 95^{th} percentile determined by FMO staining and greater than one standard deviation above the 95^{th} percentile. In other embodiments the 99th percentile may preferably be used as a demarcation point between negative and positive FMO staining and in some embodiments the percentile may be greater than 99%.

The CD46^{hi}CD324⁺ or CD34⁺CD38⁻ marker phenotype and those exemplified immediately above may be used in conjunction with standard flow cytometric analysis and cell sorting techniques to characterize, isolate, purify or enrich TIC and/or TPC cells or cell populations for further analysis.

The ability of the antibodies of the current invention to reduce the frequency of tumorigenic cells can therefore be determined using the techniques and markers described above. In some instances, the anti-UPK1B antibodies may reduce the frequency of tumorigenic cells by 10%, 15%, 20%, 25%, 30% or even by 35%. In other embodiments, the reduction in frequency of tumorigenic cells may be in the order of 40%, 45%, 50%, 55%, 60% or 65%. In certain embodiments, the disclosed compounds my reduce the frequency of tumorigenic cells by 70%, 75%, 80%, 85%, 90% or even 95%. It will be appreciated that any reduction of the frequency of tumorigenic cells is likely to result in a corresponding reduction in the tumorigenicity, persistence, recurrence and aggressiveness of the neoplasia.

### III. Antibodies

A. Antibody Structure

Antibodies and variants and derivatives thereof, including accepted nomenclature and numbering systems, have been extensively described, for example, in Abbas et al. (2010), Cellular and Molecular Immunology (6^{th} Ed.), W.B. Saunders Company; or Murphey et al. (2011), Janeway's Immunobiology (8^{th} Ed.), Garland Science.

An “antibody” or “intact antibody” typically refers to a Y-shaped tetrameric protein comprising two heavy (H) and two light (L) polypeptide chains held together by covalent disulfide bonds and non-covalent interactions. Each light chain is composed of one variable domain (VL) and one constant domain (CL). Each heavy chain comprises one variable domain (VH) and a constant region, which in the case of IgG, IgA, and IgD antibodies, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (from about 10 to about 60 amino acids in various IgG subclasses). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

As used herein the term “antibody” includes polyclonal antibodies, multiclonal antibodies, monoclonal antibodies, chimeric antibodies, humanized and primatized antibodies, CDR grafted antibodies, human antibodies (including recombinantly produced human antibodies), recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. ScFv and ScFvFc); and derivatives thereof including Fc fusions and other modifications, and any other immunoreactive molecule so long as it exhibits preferential association or binding with a determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs). Non-covalent association between the VH and the VL region forms the Fv fragment (for “fragment variable”) which contains one of the two antigen-binding sites of the antibody.

As used herein, the assignment of amino acids to each domain, framework region and CDR may be in accordance with one of the schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co or AbM (Oxford Molecular/MSI Pharmacopia) unless otherwise noted. As is well known in the art variable region residue numbering is typically as set forth in Chothia or Kabat. Amino acid residues which comprise CDRs as defined by Kabat, Chothia, MacCallum (also known as Contact) and AbM as obtained from the Abysis website database (infra.) are set out below in Table 1. Note that MacCallum uses the Chothia numbering system.

TABLE 1

| | Kabat | Chothia | MacCallum | AbM |
|---|---|---|---|---|
| VH CDR1 | 31-35 | 26-32 | 30-35 | 26-35 |
| VH CDR2 | 50-65 | 52-56 | 47-58 | 50-58 |
| VH CDR3 | 95-102 | 95-102 | 93-101 | 95-102 |
| VL CDR1 | 24-34 | 24-34 | 30-36 | 24-34 |
| VL CDR2 | 50-56 | 50-56 | 46-55 | 50-56 |
| VL CDR3 | 89-97 | 89-97 | 89-96 | 89-97 |

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005).

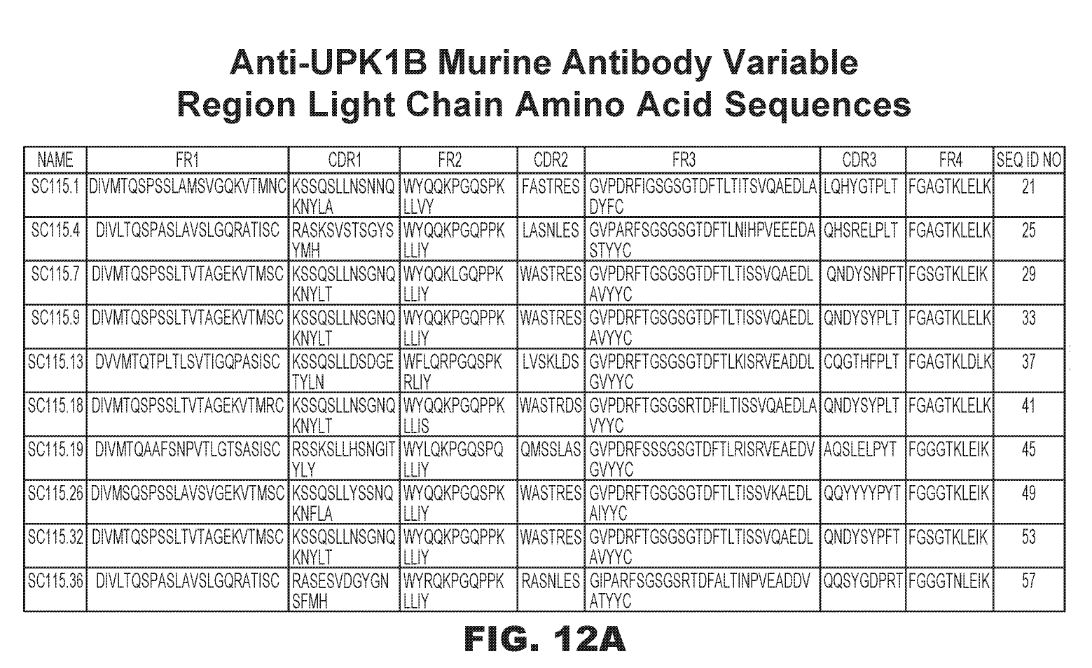

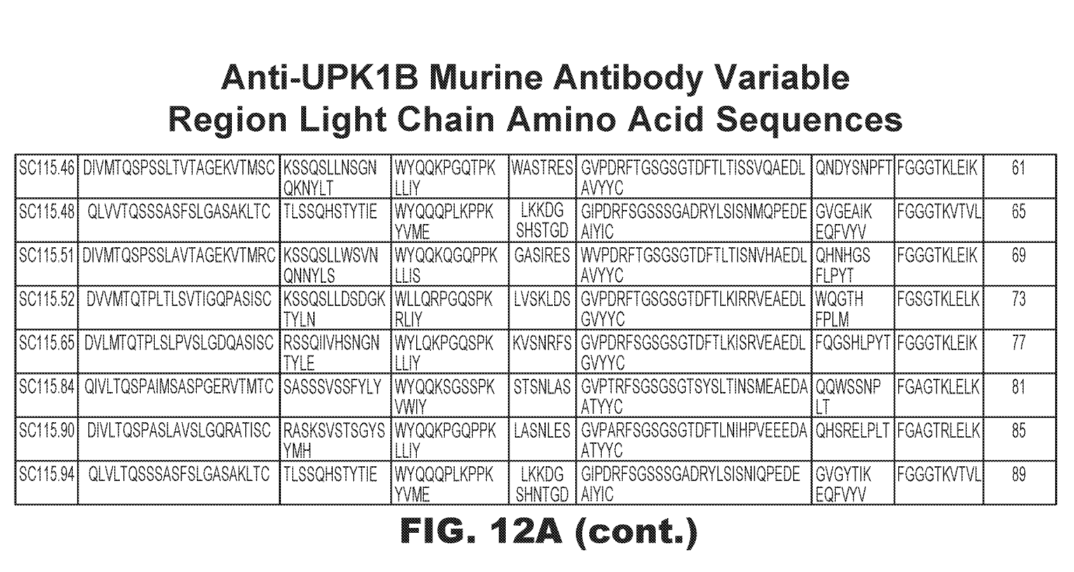

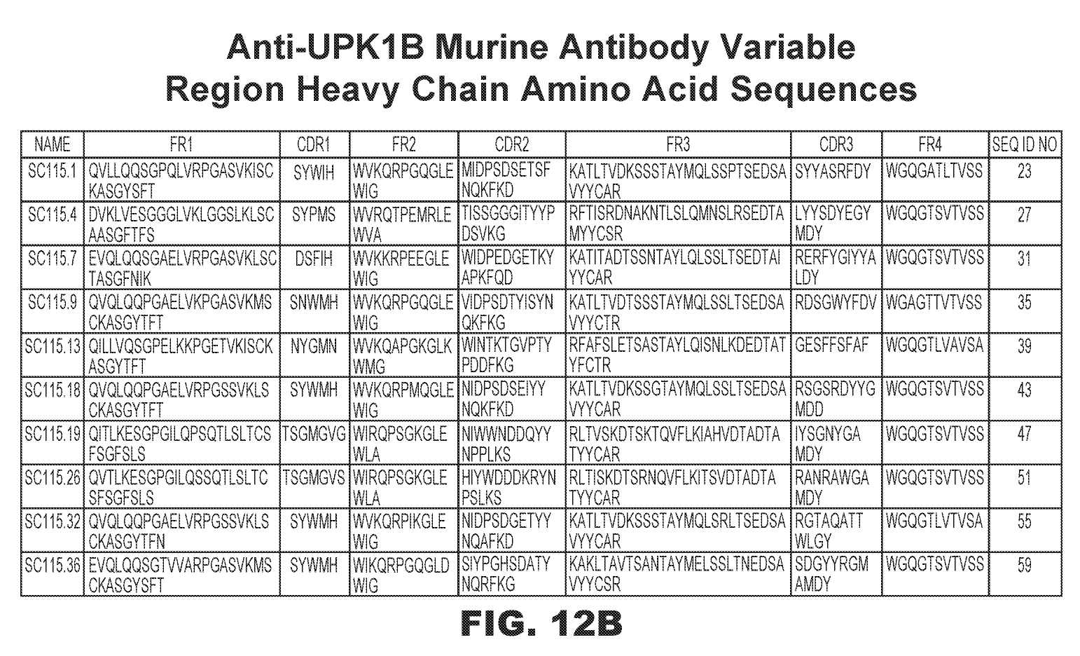

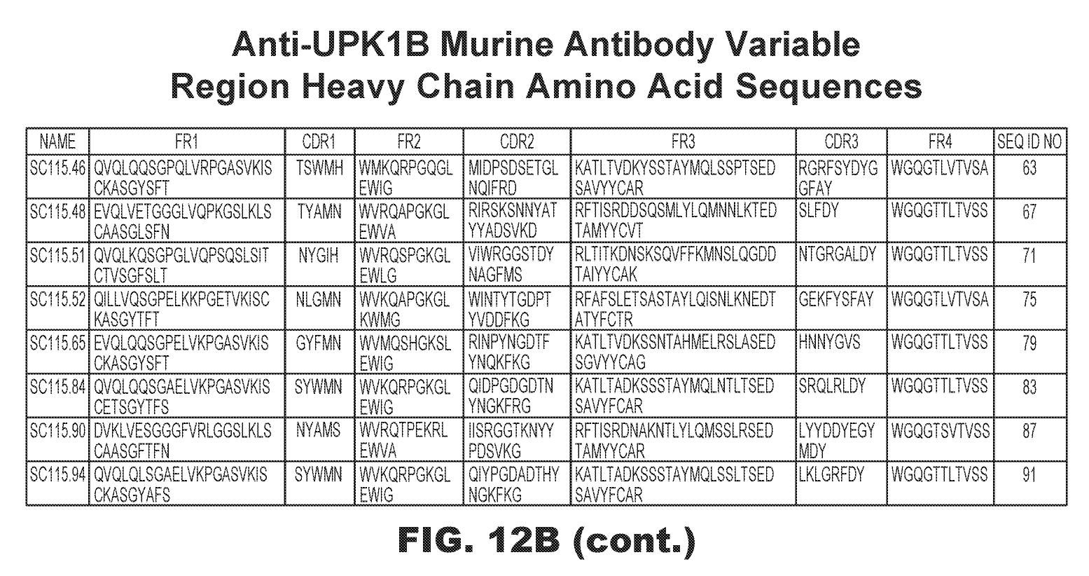

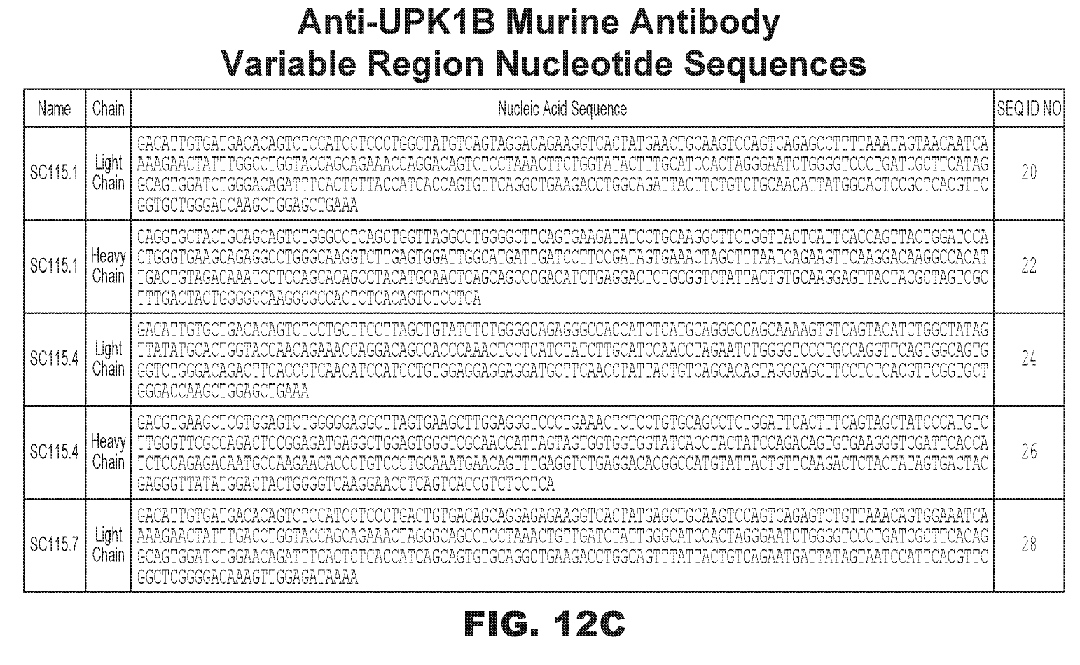

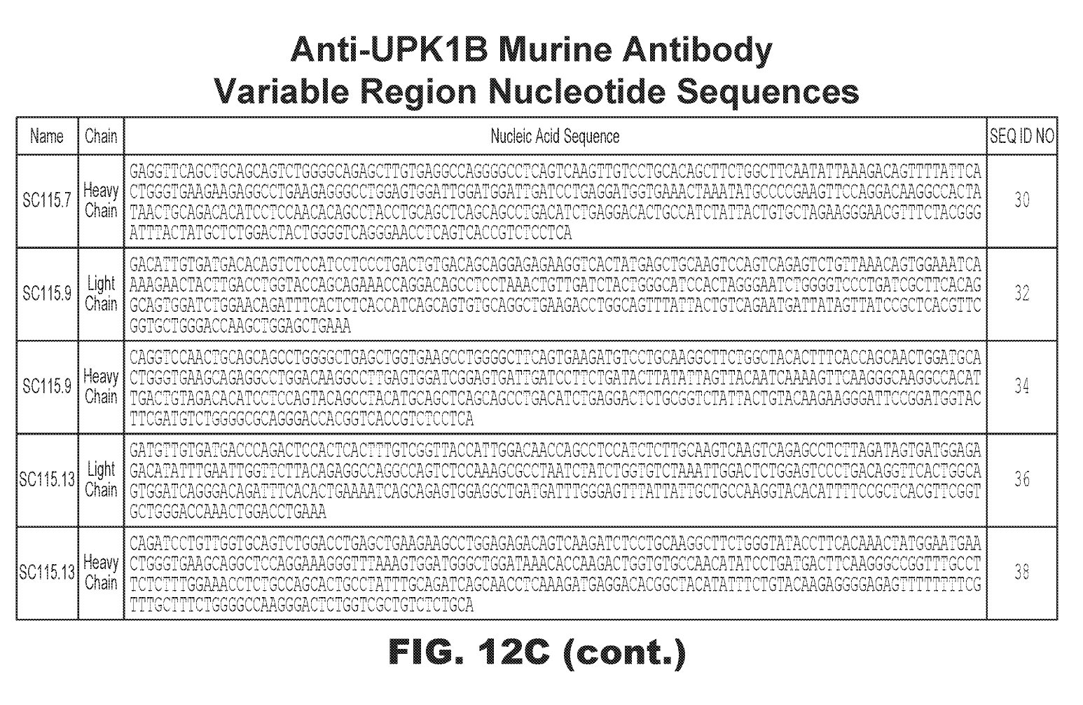

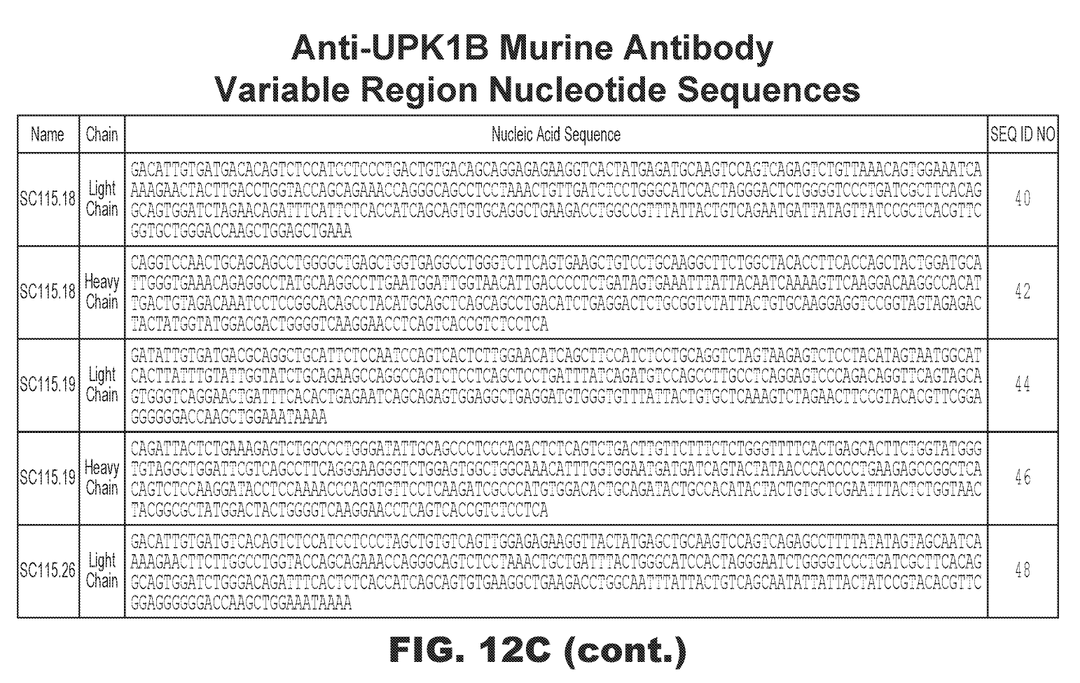

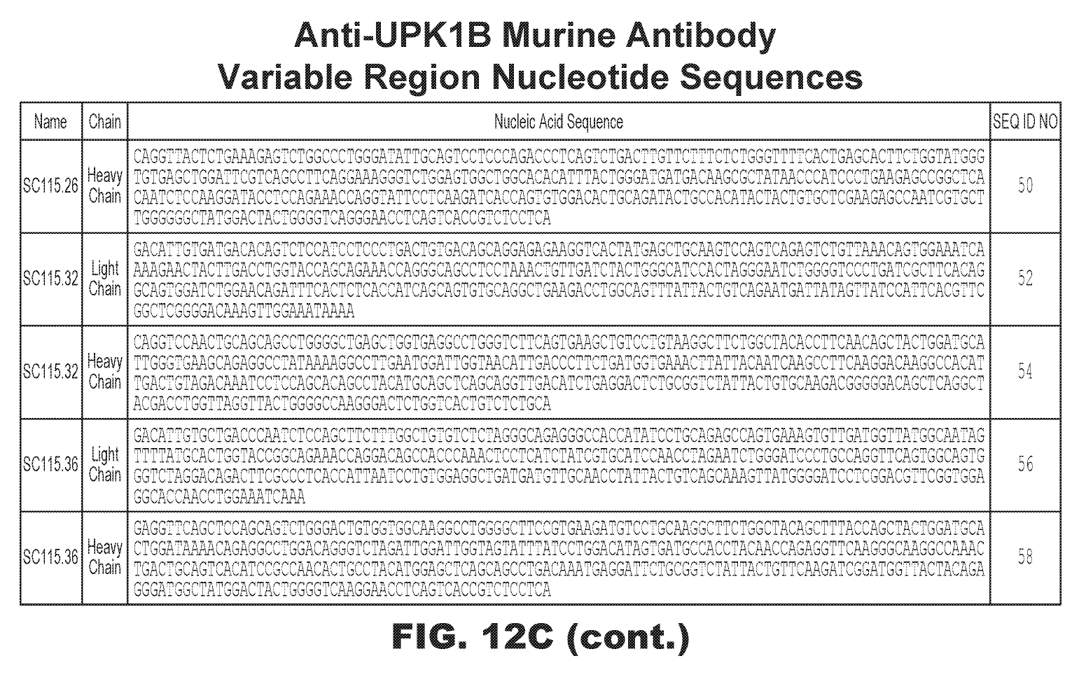

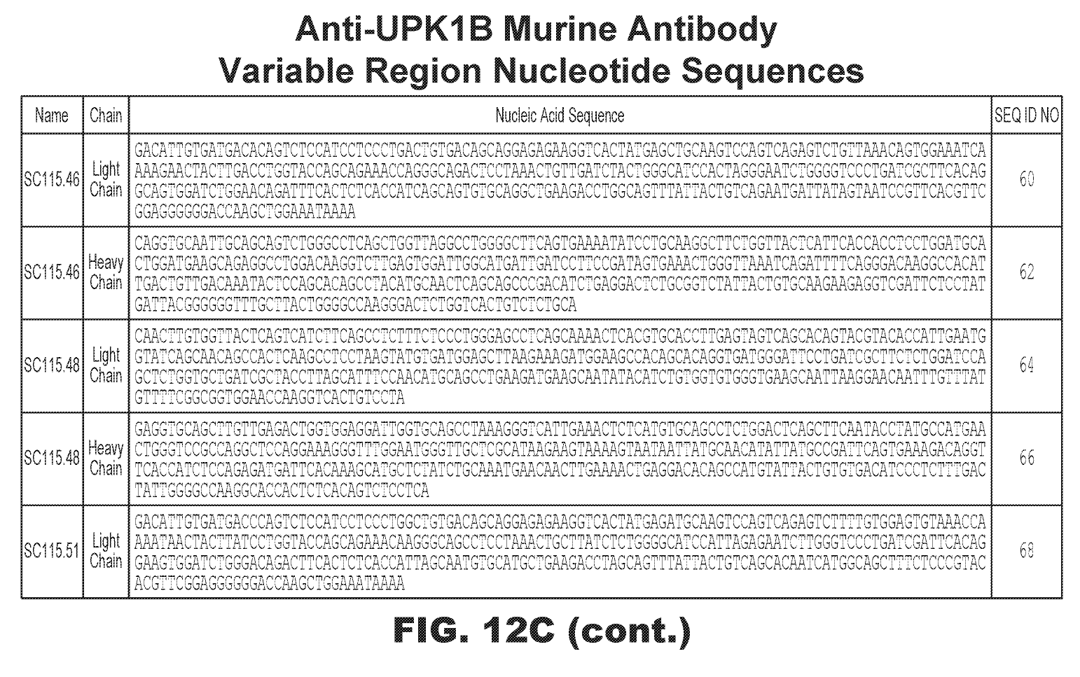

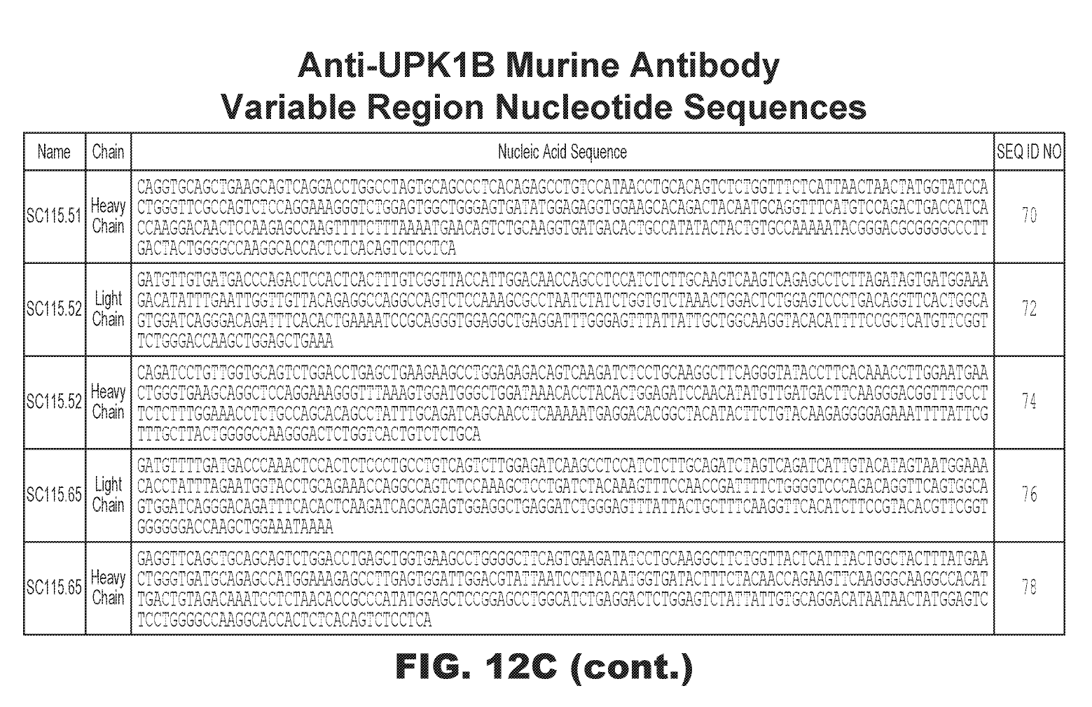

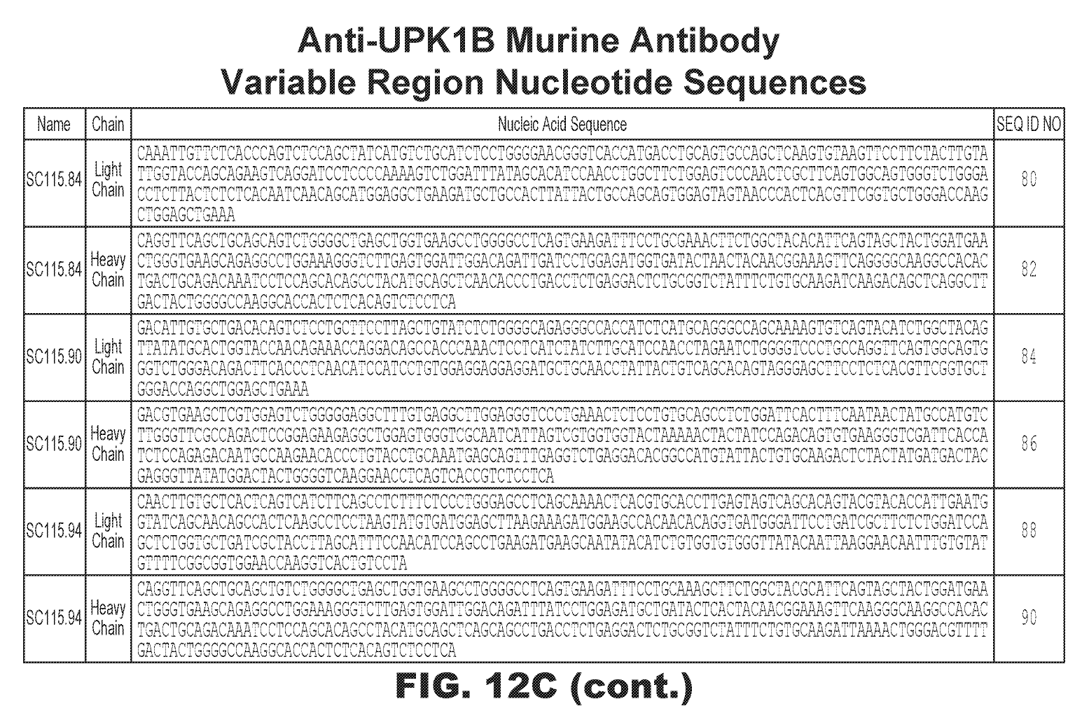

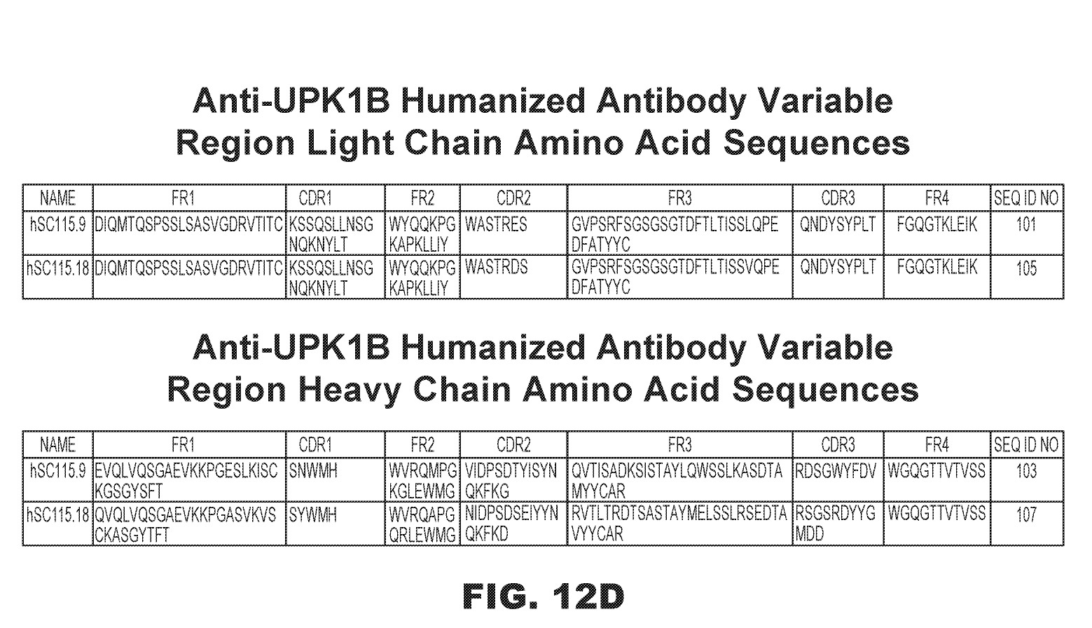

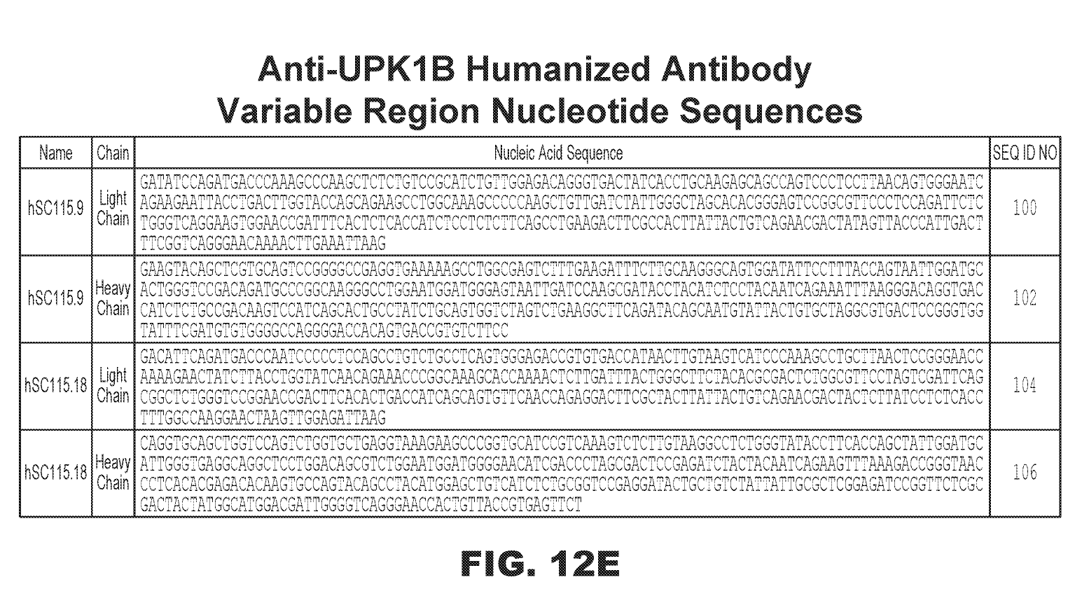

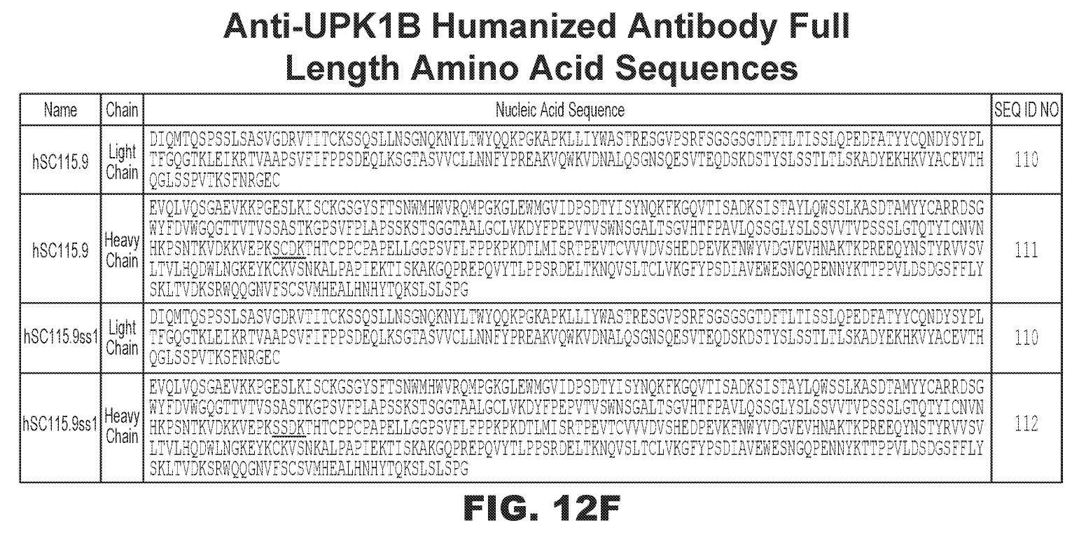

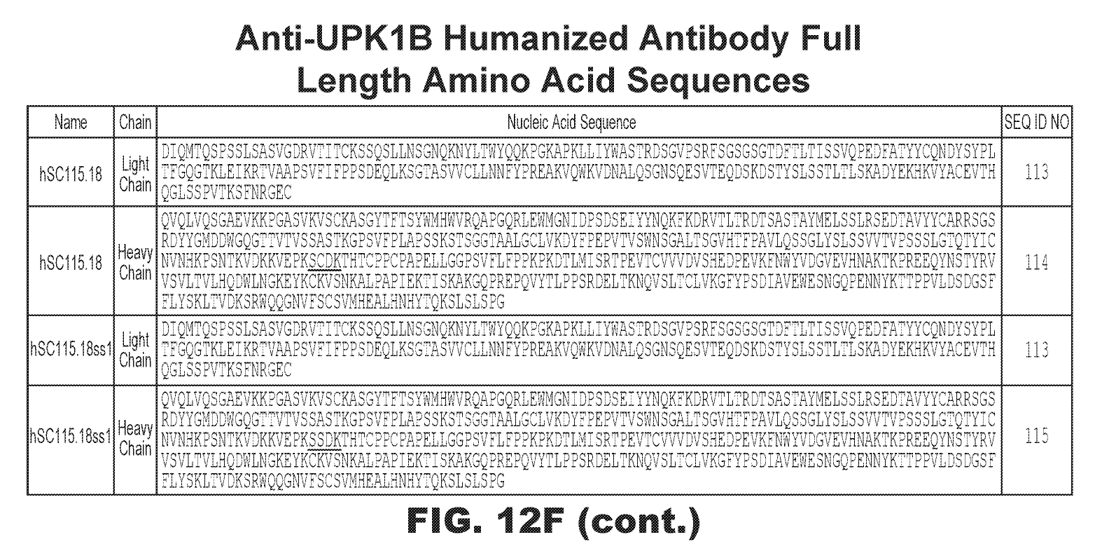

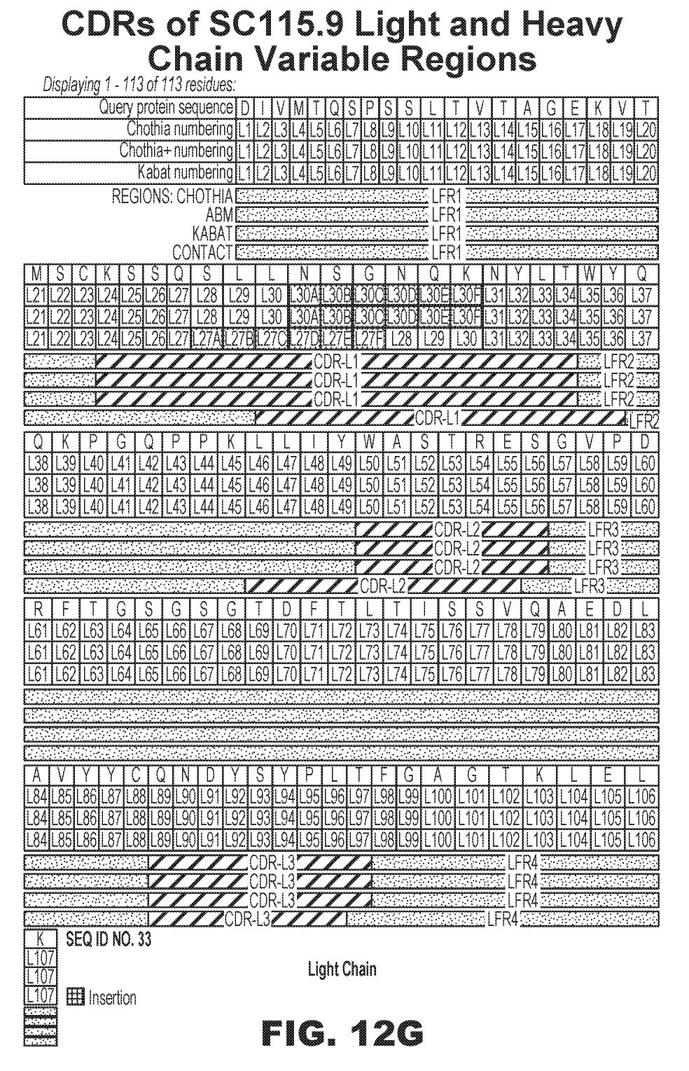

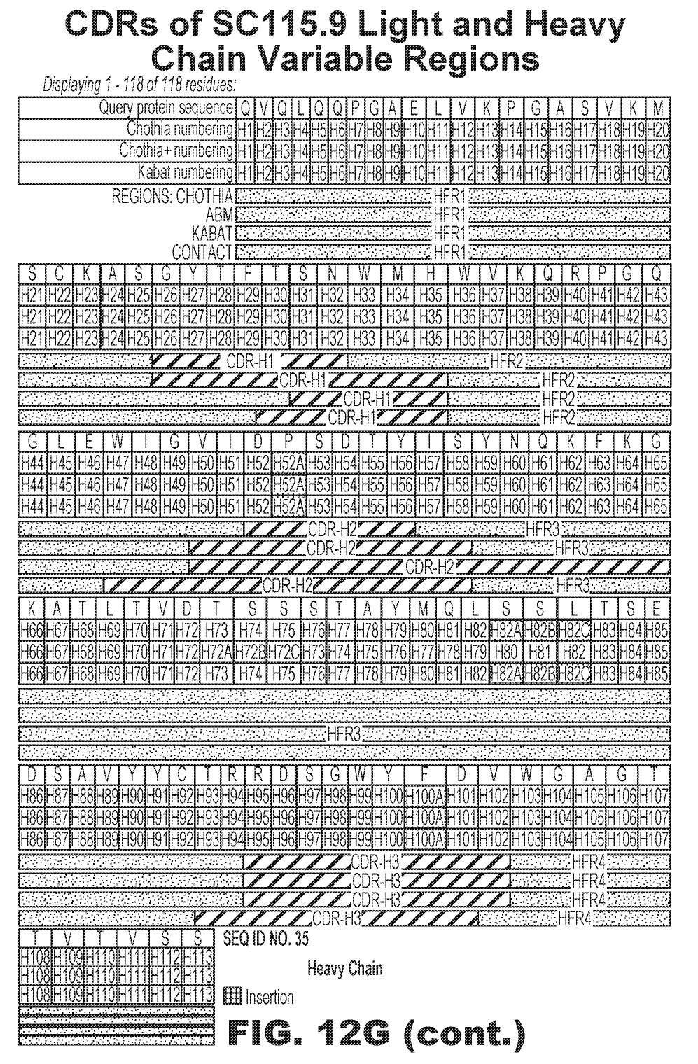

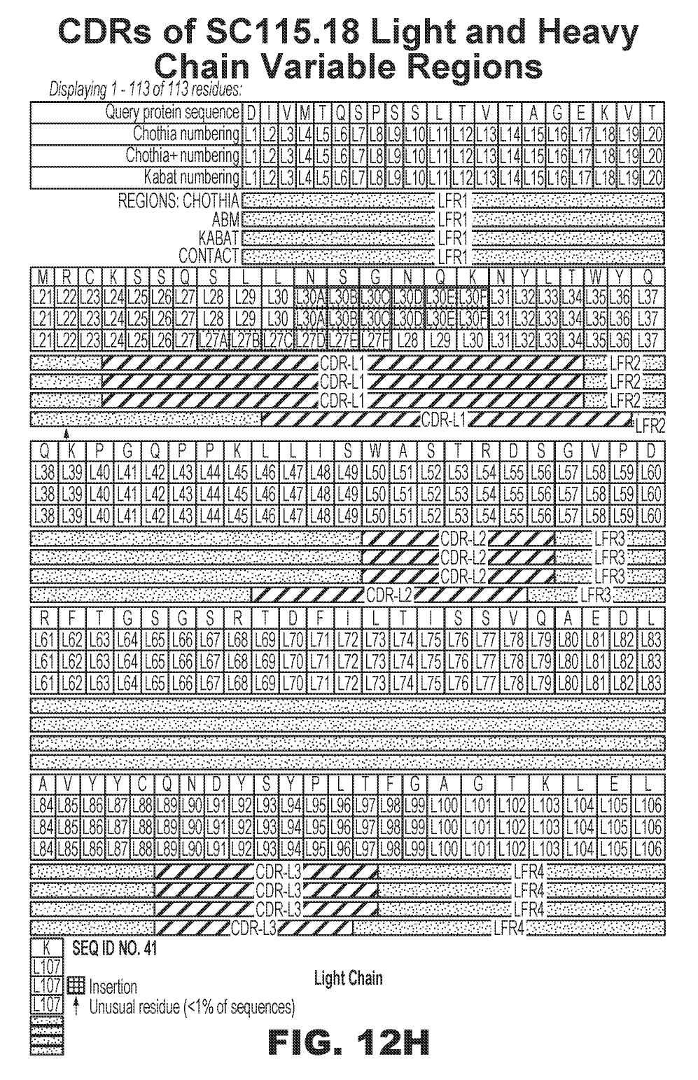

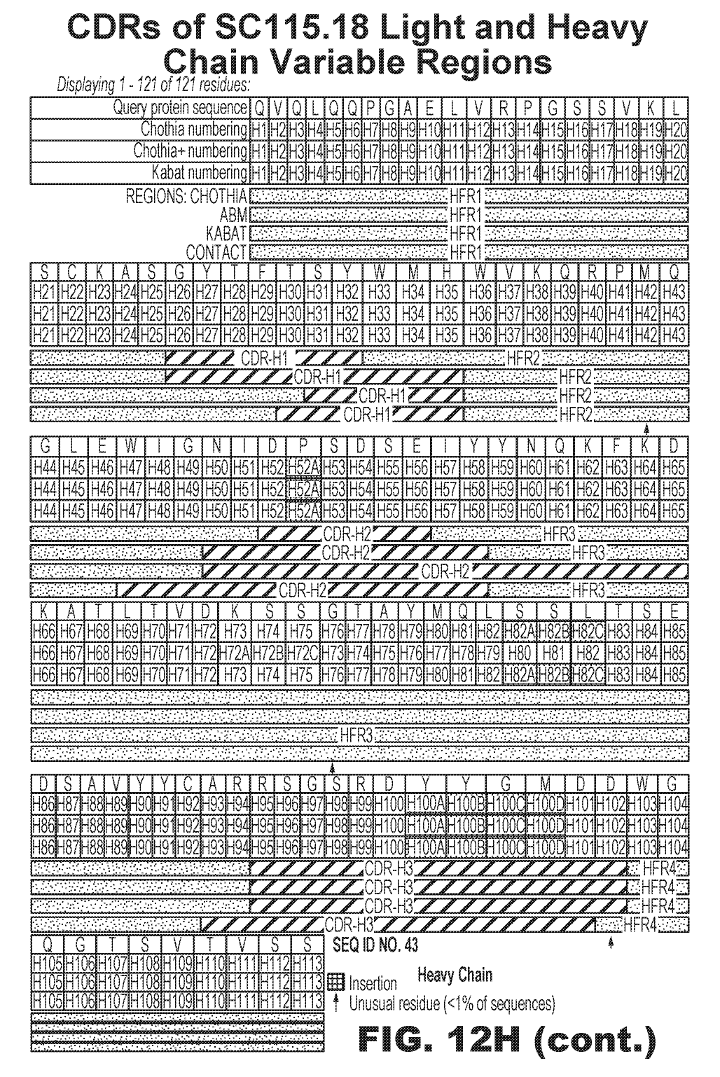

Preferably the sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. FIGS. 12G and 12H appended hereto show the results of such analysis in the annotation of exemplary heavy and light chain variable regions (VH and VL) for the SC115.9 and SC115.18 antibodies. Unless otherwise indicated, all CDRs set forth herein are derived according to the Abysis database website as per Kabat et al.

For heavy chain constant region amino acid positions discussed in the invention, numbering is according to the Eu index first described in Edelman et al., 1969, Proc. Natl. Acad. Sci. USA 63(1): 78-85 describing the amino acid sequence of the myeloma protein Eu, which reportedly was the first human IgG1 sequenced. The Eu index of Edelman is also set forth in Kabat et al., 1991 (supra.). Thus, the terms “Eu index as set forth in Kabat” or “Eu index of Kabat” or “Eu index” or “Eu numbering” in the context of the heavy chain refers to the residue numbering system based on the human IgG1 Eu antibody of Edelman et al. as set forth in Kabat et al., 1991 (supra.) The numbering system used for the light chain constant region amino acid sequence is similarly set forth in Kabat et al., (supra.) Exemplary kappa (SEQ ID NO: 5) and lambda (SEQ ID NO: 8) light chain constant region amino acid sequences compatible with the present invention is set forth immediately below:

(SEQ ID NO: 5)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.
(SEQ ID NO: 8)
QPKANPTVTLFPPSSEELQANKATLVCLISDFYPGAVTVAWKADGSPVKA
GVETTKPSKQSNNKYAASSYLSLTPEQWKSHRSYSCQVTHEGSTVEKTVA
PTECS.

Similarly, an exemplary IgG1 heavy chain constant region amino acid sequence compatible with the present invention is set forth immediately below:

(SEQ ID NO: 2)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG.

Those of skill in the art will appreciate that such heavy and light chain constant region sequences, either wild-type (e.g., see SEQ ID NOS: 2, 5 or 8) or engineered as disclosed herein to provide unpaired cysteines (e.g., see SEQ ID NOS: 3, 4, 6, 7, 9 or 10) may be operably associated with the disclosed heavy and light chain variable regions using standard molecular biology techniques to provide full-length antibodies that may be incorporated in the UPK1B antibody drug conjugates of the instant invention. Sequences of full-length heavy and light chains comprising selected antibodies of the instant invention (hSC115.9, hSC115.9ss1, hSC115.18 and hSC115.18ss1) are set forth in FIG. 12F appended hereto.

There are two types of disulfide bridges or bonds in immunoglobulin molecules: interchain and intrachain disulfide bonds. As is well known in the art the location and number of interchain disulfide bonds vary according to the immunoglobulin class and species. While the invention is not limited to any particular class or subclass of antibody, the IgG1 immunoglobulin shall be used throughout the instant disclosure for illustrative purposes. In wild-type IgG1 molecules there are twelve intrachain disulfide bonds (four on each heavy chain and two on each light chain) and four interchain disulfide bonds. Intrachain disulfide bonds are generally somewhat protected and relatively less susceptible to reduction than interchain bonds. Conversely, interchain disulfide bonds are located on the surface of the immunoglobulin, are accessible to solvent and are usually relatively easy to reduce. Two interchain disulfide bonds exist between the heavy chains and one from each heavy chain to its respective light chain. It has been demonstrated that interchain disulfide bonds are not essential for chain association. The IgG1 hinge region contain the cysteines in the heavy chain that form the interchain disulfide bonds, which provide structural support along with the flexibility that facilitates Fab movement. The heavy/heavy IgG1 interchain disulfide bonds are located at residues C226 and C229 (Eu numbering) while the IgG1 interchain disulfide bond between the light and heavy chain of IgG1 (heavy/light) are formed between C214 of the kappa or lambda light chain and C220 in the upper hinge region of the heavy chain.

B. Antibody Generation and Production

Antibodies of the invention can be produced using a variety of methods known in the art.

1. Generation of Polyclonal Antibodies in Host Animals

The production of polyclonal antibodies in various host animals is well known in the art (see for example, Harlow and Lane (Eds.) (1988) Antibodies: A Laboratory Manual, CSH Press; and Harlow et al. (1989) Antibodies, NY, Cold Spring Harbor Press). In order to generate polyclonal antibodies, an immunocompetent animal (e.g., mouse, rat, rabbit, goat, non-human primate, etc.) is immunized with an antigenic protein or cells or preparations comprising an antigenic protein. After a period of time, polyclonal antibody-containing serum is obtained by bleeding or sacrificing the animal. The serum may be used in the form obtained from the animal or the antibodies may be partially or fully purified to provide immunoglobulin fractions or isolated antibody preparations.

In this regard antibodies of the invention may be generated from any UPK1B determinant that induces an immune response in an immunocompetent animal. As used herein “determinant” or “target” means any detectable trait, property, marker or factor that is identifiably associated with, or specifically found in or on a particular cell, cell population or tissue. Determinants or targets may be morphological, functional or biochemical in nature and are preferably phenotypic. In preferred embodiments a determinant is a protein that is differentially expressed (over- or under-expressed) by specific cell types or by cells under certain conditions (e.g., during specific points of the cell cycle or cells in a particular niche). For the purposes of the instant invention a determinant preferably is differentially expressed on aberrant cancer cells and may comprise a UPK1B protein, or any of its splice variants, isoforms, homologs or family members, or specific domains, regions or epitopes thereof. An “antigen”, “immunogenic determinant”, “antigenic determinant” or “immunogen” means any UPK1B protein or any fragment, region or domain thereof that can stimulate an immune response when introduced into an immunocompetent animal and is recognized by the antibodies produced by the immune response. The presence or absence of the UPK1B determinants contemplated herein may be used to identify a cell, cell subpopulation or tissue (e.g., tumors, tumorigenic cells or CSCs).

Any form of antigen, or cells or preparations containing the antigen, can be used to generate an antibody that is specific for the UPK1B determinant. As set forth herein the term “antigen” is used in a broad sense and may comprise any immunogenic fragment or determinant of the selected target including a single epitope, multiple epitopes, single or multiple domains or the entire extracellular domain (ECD) or protein. The antigen may be an isolated full-length protein, a cell surface protein (e.g., immunizing with cells expressing at least a portion of the antigen on their surface), or a soluble protein (e.g., immunizing with only the ECD portion of the protein) or protein construct (e.g., Fc-antigen). The antigen may be produced in a genetically modified cell. Any of the aforementioned antigens may be used alone or in combination with one or more immunogenicity enhancing adjuvants known in the art. DNA encoding the antigen may be genomic or non-genomic (e.g., cDNA) and may encode at least a portion of the ECD, sufficient to elicit an immunogenic response. Any vectors may be employed to transform the cells in which the antigen is expressed, including but not limited to adenoviral vectors, lentiviral vectors, plasmids, and non-viral vectors, such as cationic lipids.

2. Monoclonal Antibodies

In selected embodiments, the invention contemplates use of monoclonal antibodies. As known in the art, the term “monoclonal antibody” or “mAb” refers to an antibody obtained from a population of substantially homogeneous antibodies, i.e., the individual antibodies comprising the population are identical except for possible mutations (e.g., naturally occurring mutations), that may be present in minor amounts.

Monoclonal antibodies can be prepared using a wide variety of techniques known in the art including hybridoma techniques, recombinant techniques, phage display technologies, transgenic animals (e.g., a XenoMouse®) or some combination thereof. For example, monoclonal antibodies can be produced using hybridoma and biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981). Following production of multiple monoclonal antibodies that bind specifically to a determinant, particularly effective antibodies may be selected through various screening processes, based on, for example, its affinity for the determinant or rate of internalization. Antibodies produced as described herein may be used as “source” antibodies and further modified to, for example, improve affinity for the target, improve its production in cell culture, reduce immunogenicity in vivo, create multispecific constructs, etc. A more detailed description of monoclonal antibody production and screening is set out below and in the appended Examples.

3. Human Antibodies

In another embodiment, the antibodies may comprise fully human antibodies. The term “human antibody” refers to an antibody which possesses an amino acid sequence that corresponds to that of an antibody produced by a human and/or has been made using any of the techniques for making human antibodies described below.

Human antibodies can be produced using various techniques known in the art. One technique is phage display in which a library of (preferably human) antibodies is synthesized on phages, the library is screened with the antigen of interest or an antibody-binding portion thereof, and the phage that binds the antigen is isolated, from which one may obtain the immunoreactive fragments. Methods for preparing and screening such libraries are well known in the art and kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; and Barbas et al., Proc. Natl. Acad. Sci. USA 88:7978-7982 (1991)).

In one embodiment, recombinant human antibodies may be isolated by screening a recombinant combinatorial antibody library prepared as above. In one embodiment, the library is a scFv phage display library, generated using human VL and VH cDNAs prepared from mRNA isolated from B-cells.

The antibodies produced by naive libraries (either natural or synthetic) can be of moderate affinity (Kₐ of about 10⁶ to 10⁷ M⁻¹), but affinity maturation can also be mimicked in vitro by constructing and reselecting from secondary libraries as described in the art. For example, mutation can be introduced at random in vitro by using error-prone polymerase (reported in Leung et al., Technique, 1: 11-15 (1989)). Additionally, affinity maturation can be performed by randomly mutating one or more CDRs, e.g. using PCR with primers carrying random sequence spanning the CDR of interest, in selected individual Fv clones and screening for higher-affinity clones. WO 9607754 described a method for inducing mutagenesis in a CDR of an immunoglobulin light chain to create a library of light chain genes. Another effective approach is to recombine the VH or VL domains selected by phage display with repertoires of naturally occurring V domain variants obtained from unimmunized donors and to screen for higher affinity in several rounds of chain reshuffling as described in Marks et al., Biotechnol., 10: 779-783 (1992). This technique allows the production of antibodies and antibody fragments with a dissociation constant K_{D} (k_{off}/kₒₙ) of about 10⁻⁹ M or less.

In other embodiments, similar procedures may be employed using libraries comprising eukaryotic cells (e.g., yeast) that express binding pairs on their surface. See, for example, U.S. Pat. No. 7,700,302 and U.S. Ser. No. 12/404,059. In one embodiment, the human antibody is selected from a phage library, where that phage library expresses human antibodies (Vaughan et al. Nature Biotechnology 14:309-314 (1996): Sheets et al. Proc. Natl. Acad. Sci. USA 95:6157-6162 (1998). In other embodiments, human binding pairs may be isolated from combinatorial antibody libraries generated in eukaryotic cells such as yeast. See e.g., U.S. Pat. No. 7,700,302. Such techniques advantageously allow for the screening of large numbers of candidate modulators and provide for relatively easy manipulation of candidate sequences (e.g., by affinity maturation or recombinant shuffling).

Human antibodies can also be made by introducing human immunoglobulin loci into transgenic animals, e.g., mice in which the endogenous immunoglobulin genes have been partially or completely inactivated and human immunoglobulin genes have been introduced. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and U.S. Pat. Nos. 6,075,181 and 6,150,584 regarding XenoMouse® technology; and Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual suffering from a neoplastic disorder or may have been immunized in vitro). See, e.g., Cole et al., Monoclonal Antibodies and Cancer Therapy, Alan R. Liss, p. 77 (1985); Boerner et al., J. Immunol, 147 (I):86-95 (1991); and U.S. Pat. No. 5,750,373.

Whatever the source it will be appreciated that the human antibody sequence may be fabricated using art-known molecular engineering techniques and introduced into expression systems and host cells as described herein. Such non-natural recombinantly produced human antibodies (and subject compositions) are entirely compatible with the teachings of this disclosure and are expressly held to be within the scope of the instant invention. In certain select aspects the UPK1B ADCs of the invention will comprise a recombinantly produced human antibody acting as a cell binding agent

4. Derived Antibodies:

Once source antibodies have been generated, selected and isolated as described above they may be further altered to provide anti-UPK1B antibodies having improved pharmaceutical characteristics. Preferably the source antibodies are modified or altered using known molecular engineering techniques to provide derived antibodies having the desired therapeutic properties.

4.1. Chimeric and Humanized Antibodies

Selected embodiments of the invention comprise murine monoclonal antibodies that immunospecifically bind to UPK1B and which can be considered “source” antibodies. In selected embodiments, antibodies of the invention can be derived from such “source” antibodies through optional modification of the constant region and/or the epitope-binding amino acid sequences of the source antibody. In certain embodiments an antibody is “derived” from a source antibody if selected amino acids in the source antibody are altered through deletion, mutation, substitution, integration or combination. In another embodiment, a “derived” antibody is one in which fragments of the source antibody (e.g., one or more CDRs or domains or the entire heavy and light chain variable regions) are combined with or incorporated into an acceptor antibody sequence to provide the derivative antibody (e.g. chimeric, CDR grafted or humanized antibodies). These “derived” antibodies can be generated using genetic material from the antibody producing cell and standard molecular biological techniques as described below, such as, for example, to improve affinity for the determinant; to improve antibody stability; to improve production and yield in cell culture; to reduce immunogenicity in vivo; to reduce toxicity; to facilitate conjugation of an active moiety; or to create a multispecific antibody. Such antibodies may also be derived from source antibodies through modification of the mature molecule (e.g., glycosylation patterns or pegylation) by chemical means or post-translational modification.

In one embodiment, the antibodies of the invention comprise chimeric antibodies that are derived from protein segments from at least two different species or class of antibodies that have been covalently joined. The term “chimeric” antibody is directed to constructs in which a portion of the heavy and/or light chain is identical or homologous to corresponding sequences in antibodies from a particular species or belonging to a particular antibody class or subclass, while the remainder of the chain(s) is identical or homologous to corresponding sequences in antibodies from another species or belonging to another antibody class or subclass, as well as fragments of such antibodies (U.S. Pat. No. 4,816,567). In some embodiments chimeric antibodies of the instant invention may comprise all or most of the selected murine heavy and light chain variable regions operably linked to human light and heavy chain constant regions. In other selected embodiments, anti-UPK1B antibodies may be “derived” from the mouse antibodies disclosed herein and comprise less than the entire heavy and light chain variable regions.

In other embodiments, chimeric antibodies of the invention are “CDR-grafted” antibodies, where the CDRs (as defined using Kabat, Chothia, McCallum, etc.) are derived from a particular species or belonging to a particular antibody class or subclass, while the remainder of the antibody is largely derived from an antibody from another species or belonging to another antibody class or subclass. For use in humans, one or more selected rodent CDRs (e.g., mouse CDRs) may be grafted into a human acceptor antibody, replacing one or more of the naturally occurring CDRs of the human antibody. These constructs generally have the advantages of providing full strength human antibody functions, e.g., complement dependent cytotoxicity (CDC) and antibody-dependent cell-mediated cytotoxicity (ADCC) while reducing unwanted immune responses to the antibody by the subject. In one embodiment the CDR grafted antibodies will comprise one or more CDRs obtained from a mouse incorporated in a human framework sequence.

Similar to the CDR-grafted antibody is a “humanized” antibody. As used herein, a “humanized” antibody is a human antibody (acceptor antibody) comprising one or more amino acid sequences (e.g. CDR sequences) derived from one or more non-human antibodies (donor or source antibody). In certain embodiments, “back mutations” can be introduced into the humanized antibody, in which residues in one or more FRs of the variable region of the recipient human antibody are replaced by corresponding residues from the non-human species donor antibody. Such back mutations may to help maintain the appropriate three-dimensional configuration of the grafted CDR(s) and thereby improve affinity and antibody stability. Antibodies from various donor species may be used including, without limitation, mouse, rat, rabbit, or non-human primate. Furthermore, humanized antibodies may comprise new residues that are not found in the recipient antibody or in the donor antibody to, for example, further refine antibody performance. CDR grafted and humanized antibodies compatible with the instant invention comprising murine components from source antibodies and human components from acceptor antibodies may be provided as set forth in the Examples below.

Various art-recognized techniques can be used to determine which human sequences to use as acceptor antibodies to provide humanized constructs in accordance with the instant invention. Compilations of compatible human germline sequences and methods of determining their suitability as acceptor sequences are disclosed, for example, in Dubel and Reichert (Eds.) (2014) Handbook of Therapeutic Antibodies, 2^{nd} Edition, Wiley-Blackwell GmbH; Tomlinson, I. A. et al. (1992) J. Mol. Biol. 227:776-798; Cook, G. P. et al. (1995) Immunol. Today 16: 237-242; Chothia, D. et al. (1992) J. Mol. Biol. 227:799-817; and Tomlinson et al. (1995) EMBO J 14:4628-4638). The V-BASE directory (VBASE2—Retter et al., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK) may also be used to identify compatible acceptor sequences. Additionally, consensus human framework sequences described, for example, in U.S. Pat. No. 6,300,064 may also prove to be compatible acceptor sequences are can be used in accordance with the instant teachings. In general, human framework acceptor sequences are selected based on homology with the murine source framework sequences along with an analysis of the CDR canonical structures of the source and acceptor antibodies. The derived sequences of the heavy and light chain variable regions of the derived antibody may then be synthesized using art recognized techniques.

By way of example CDR grafted and humanized antibodies, and associated methods, are described in U.S. Pat. Nos. 6,180,370 and 5,693,762. For further details, see, e.g., Jones et al., 1986, (PMID: 3713831); and U.S. Pat. Nos. 6,982,321 and 7,087,409.

The sequence identity or homology of the CDR grafted or humanized antibody variable region to the human acceptor variable region may be determined as discussed herein and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

It will be appreciated that the annotated CDRs and framework sequences as provided in the appended FIGS. 12A and 12B are defined as per Kabat et al. using a proprietary Abysis database. However, as discussed herein and shown in FIGS. 12G and 12H, one skilled in the art could readily identify CDRs in accordance with definitions provided by Chothia et al., ABM or MacCallum et al as well as Kabat et al. As such, anti-UPK1B humanized antibodies comprising one or more CDRs derived according to any of the aforementioned systems are explicitly held to be within the scope of the instant invention.

4.2. Site-Specific Antibodies

The antibodies of the instant invention may be engineered to facilitate conjugation to a cytotoxin or other anti-cancer agent (as discussed in more detail below). It is advantageous for the antibody drug conjugate (ADC) preparation to comprise a homogenous population of ADC molecules in terms of the position of the cytotoxin on the antibody and the drug to antibody ratio (DAR). Based on the instant disclosure one skilled in the art could readily fabricate site-specific engineered constructs as described herein. As used herein a “site-specific antibody” or “site-specific construct” means an antibody, or immunoreactive fragment thereof, wherein at least one amino acid in either the heavy or light chain is deleted, altered or substituted (preferably with another amino acid) to provide at least one free cysteine. Similarly, a “site-specific conjugate” shall be held to mean an ADC comprising a site-specific antibody and at least one cytotoxin or other compound (e.g., a reporter molecule) conjugated to the unpaired or free cysteine(s). In certain embodiments the unpaired cysteine residue will comprise an unpaired intrachain cysteine residue. In other embodiments the free cysteine residue will comprise an unpaired interchain cysteine residue. In still other embodiments the free cysteine may be engineered into the amino acid sequence of the antibody (e.g., in the CH3 domain). In any event the site-specific antibody can be of various isotypes, for example, IgG, IgE, IgA or IgD; and within those classes the antibody can be of various subclasses, for example, IgG1, IgG2, IgG3 or IgG4. For IgG constructs the light chain of the antibody can comprise either a kappa or lambda isotype each incorporating a C214 that, in selected embodiments, may be unpaired due to a lack of a C220 residue in the IgG1 heavy chain.

Thus, as used herein, the terms “free cysteine” or “unpaired cysteine” may be used interchangeably unless otherwise dictated by context and shall mean any cysteine (or thiol containing) constituent (e.g., a cysteine residue) of an antibody, whether naturally present or specifically incorporated in a selected residue position using molecular engineering techniques, that is not part of a naturally occurring (or “native”) disulfide bond under physiological conditions. In certain selected embodiments the free cysteine may comprise a naturally occurring cysteine whose native interchain or intrachain disulfide bridge partner has been substituted, eliminated or otherwise altered to disrupt the naturally occurring disulfide bridge under physiological conditions thereby rendering the unpaired cysteine suitable for site-specific conjugation. In other preferred embodiments the free or unpaired cysteine will comprise a cysteine residue that is selectively placed at a predetermined site within the antibody heavy or light chain amino acid sequences. It will be appreciated that, prior to conjugation, free or unpaired cysteines may be present as a thiol (reduced cysteine), as a capped cysteine (oxidized) or as part of a non-native intra- or intermolecular disulfide bond (oxidized) with another cysteine or thiol group on the same or different molecule depending on the oxidation state of the system. As discussed in more detail below, mild reduction of the appropriately engineered antibody construct will provide thiols available for site-specific conjugation. Accordingly, in particularly preferred embodiments the free or unpaired cysteines (whether naturally occurring or incorporated) will be subject to selective reduction and subsequent conjugation to provide homogenous DAR compositions.

It will be appreciated that the favorable properties exhibited by the disclosed engineered conjugate preparations is predicated, at least in part, on the ability to specifically direct the conjugation and largely limit the fabricated conjugates in terms of conjugation position and the absolute DAR value of the composition. Unlike most conventional ADC preparations the present invention need not rely entirely on partial or total reduction of the antibody to provide random conjugation sites and relatively uncontrolled generation of DAR species. Rather, in certain aspects the present invention preferably provides one or more predetermined unpaired (or free) cysteine sites by engineering the targeting antibody to disrupt one or more of the naturally occurring (i.e., “native”) interchain or intrachain disulfide bridges or to introduce a cysteine residue at any position. To this end it will be appreciated that, in selected embodiments, a cysteine residue may be incorporated anywhere along the antibody (or immunoreactive fragment thereof) heavy or light chain or appended thereto using standard molecular engineering techniques. In other preferred embodiments disruption of native disulfide bonds may be effected in combination with the introduction of a non-native cysteine (which will then comprise the free cysteine) that may then be used as a conjugation site.

In certain embodiments the engineered antibody comprises at least one amino acid deletion or substitution of an intrachain or interchain cysteine residue. As used herein “interchain cysteine residue” means a cysteine residue that is involved in a native disulfide bond either between the light and heavy chain of an antibody or between the two heavy chains of an antibody while an “intrachain cysteine residue” is one naturally paired with another cysteine in the same heavy or light chain. In one embodiment the deleted or substituted interchain cysteine residue is involved in the formation of a disulfide bond between the light and heavy chain. In another embodiment the deleted or substituted cysteine residue is involved in a disulfide bond between the two heavy chains. In a typical embodiment, due to the complementary structure of an antibody, in which the light chain is paired with the VH and CH1 domains of the heavy chain and wherein the CH2 and CH3 domains of one heavy chain are paired with the CH2 and CH3 domains of the complementary heavy chain, a mutation or deletion of a single cysteine in either the light chain or in the heavy chain would result in two unpaired cysteine residues in the engineered antibody.

In some embodiments an interchain cysteine residue is deleted. In other embodiments an interchain cysteine is substituted for another amino acid (e.g., a naturally occurring amino acid). For example, the amino acid substitution can result in the replacement of an interchain cysteine with a neutral (e.g. serine, threonine or glycine) or hydrophilic (e.g. methionine, alanine, valine, leucine or isoleucine) residue. In selected embodiments an interchain cysteine is replaced with a serine.

In some embodiments contemplated by the invention the deleted or substituted cysteine residue is on the light chain (either kappa or lambda) thereby leaving a free cysteine on the heavy chain. In other embodiments the deleted or substituted cysteine residue is on the heavy chain leaving the free cysteine on the light chain constant region. Upon assembly it will be appreciated that deletion or substitution of a single cysteine in either the light or heavy chain of an intact antibody results in a site-specific antibody having two unpaired cysteine residues.

In one embodiment the cysteine at position 214 (C214) of the IgG light chain (kappa or lambda) is deleted or substituted. In another embodiment the cysteine at position 220 (C220) on the IgG heavy chain is deleted or substituted. In further embodiments the cysteine at position 226 or position 229 on the heavy chain is deleted or substituted. In one embodiment C220 on the heavy chain is substituted with serine (C220S) to provide the desired free cysteine in the light chain. In another embodiment C214 in the light chain is substituted with serine (C214S) to provide the desired free cysteine in the heavy chain. Such site-specific constructs are described in more detail in the Examples below. A summary of compatible site-specific constructs is shown in Table 2 immediately below where numbering is generally according to the Eu index as set forth in Kabat, WT stands for “wild-type” or native constant region sequences without alterations and delta (A) designates the deletion of an amino acid residue (e.g., C214A indicates that the cysteine residue at position 214 has been deleted).

TABLE 2

| | Antibody | | |
|---|---|---|---|
| Designation | Component | Alteration | SEQ ID NOS: |
| ss1 | Heavy Chain | C220S | SEQ ID NO: 3 |
| | Light Chain | WT | SEQ ID NOS: 5, 8 |
| ss2 | Heavy Chain | C220Δ | SEQ ID NO: 4 |
| | Light Chain | WT | SEQ ID NOS: 5, 8 |
| ss3 | Heavy Chain | WT | SEQ ID NO: 2 |
| | Light Chain | C214Δ | SEQ ID NOS: 7, 10 |
| ss4 | Heavy Chain | WT | SEQ ID NO: 2 |
| | Light Chain | C214S | SEQ ID NOS: 6, 9 |

Exemplary engineered light and heavy chain constant regions compatible with site-specific constructs of the instant invention are set forth immediately below where SEQ ID NOS: 3 and 4 comprise, respectively, C220S IgG1 and C220A IgG1 heavy chain constant regions, SEQ ID NOS: 6 and 7 comprise, respectively, C214S and C214A kappa light chain constant regions and SEQ ID NOS: 9 and 10 comprise, respectively, exemplary C214S and C214A lambda light chain constant regions. In each case the site of the altered or deleted amino acid (along with the flanking residues) is underlined.

(SEQ ID NO: 3)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG
(SEQ ID NO: 4)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPG
(SEQ ID NO: 6)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGES
(SEQ ID NO: 7)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGE
(SEQ ID NO: 9)
QPKANPTVTLFPPSSEELQANKATLVCLISDFYPGAVTVAWKADGSPVKA
GVETTKPSKQSNNKYAASSYLSLTPEQWKSHRSYSCQVTHEGSTVEKTVA
PTESS
(SEQ ID NO: 10)
QPKANPTVTLFPPSSEELQANKATLVCLISDFYPGAVTVAWKADGSPVKA
GVETTKPSKQSNNKYAASSYLSLTPEQWKSHRSYSCQVTHEGSTVEKTVA
PTES

As discussed above each of the heavy and light chain variants may be operably associated with the disclosed heavy and light chain variable regions (or derivatives thereof such as humanized or CDR grafted constructs) to provide site-specific anti-UPK1B antibodies as disclosed herein. Such engineered antibodies are particularly compatible for use in the disclosed ADCs.

With regard to the introduction or addition of a cysteine residue or residues to provide a free cysteine (as opposed to disrupting a native disulfide bond) compatible position(s) on the antibody or antibody fragment may readily be discerned by one skilled in the art. Accordingly, in selected embodiments the cysteine(s) may be introduced in the CH1 domain, the CH2 domain or the CH3 domain or any combination thereof depending on the desired DAR, the antibody construct, the selected payload and the antibody target. In other preferred embodiments the cysteines may be introduced into a kappa or lambda CL domain and, in particularly preferred embodiments, in the c-terminal region of the CL domain. In each case other amino acid residues proximal to the site of cysteine insertion may be altered, removed or substituted to facilitate molecular stability, conjugation efficiency or provide a protective environment for the payload once it is attached. In particular embodiments, the substituted residues occur at any accessible sites of the antibody. By substituting such surface residues with cysteine, reactive thiol groups are thereby positioned at readily accessible sites on the antibody and may be selectively reduced as described further herein. In particular embodiments, the substituted residues occur at accessible sites of the antibody. By substituting those residues with cysteine, reactive thiol groups are thereby positioned at accessible sites of the antibody and may be used to selectively conjugate the antibody. In certain embodiments, any one or more of the following residues may be substituted with cysteine: V205 (Kabat numbering) of the light chain; A118 (Eu numbering) of the heavy chain; and S400 (Eu numbering) of the heavy chain Fc region. Additional substitution positions and methods of fabricating compatible site-specific antibodies are set forth in U.S. Pat. No. 7,521,541 which is incorporated herein in its entirety.

The strategy for generating antibody drug conjugates with defined sites and stoichiometries of drug loading, as disclosed herein, is broadly applicable to all anti-UPK1B antibodies as it primarily involves engineering of the conserved constant domains of the antibody. As the amino acid sequences and native disulfide bridges of each class and subclass of antibody are well documented, one skilled in the art could readily fabricate engineered constructs of various antibodies without undue experimentation and, accordingly, such constructs are expressly contemplated as being within the scope of the instant invention.

4.3. Constant Region Modifications and Altered Glycosylation

Selected embodiments of the present invention may also comprise substitutions or modifications of the constant region (i.e. the Fc region), including without limitation, amino acid residue substitutions, mutations and/or modifications, which result in a compound with characteristics including, but not limited to: altered pharmacokinetics, increased serum half-life, increase binding affinity, reduced immunogenicity, increased production, altered Fc ligand binding to an Fc receptor (FcR), enhanced or reduced ADCC or CDC, altered glycosylation and/or disulfide bonds and modified binding specificity.

Compounds with improved Fc effector functions can be generated, for example, through changes in amino acid residues involved in the interaction between the Fc domain and an Fc receptor (e.g., FcγRI, FcγRIIA and B, FcγRIII and FcRn), which may lead to increased cytotoxicity and/or altered pharmacokinetics, such as increased serum half-life (see, for example, Ravetch and Kinet, Annu. Rev. Immunol 9:457-92 (1991); Capel et al., Immunomethods 4:25-34 (1994); and de Haas et al., J. Lab. Clin. Med. 126:330-41 (1995).

In selected embodiments, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S.P.N. 2003/0190311). With regard to such embodiments, Fc variants may provide half-lives in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-life results in a higher serum titer which thus reduces the frequency of the administration of the antibodies and/or reduces the concentration of the antibodies to be administered. Binding to human FcRn in vivo and serum half-life of human FcRn high affinity binding polypeptides can be assayed, e.g., in transgenic mice or transfected human cell lines expressing human FcRn, or in primates to which the polypeptides with a variant Fc region are administered. WO 2000/42072 describes antibody variants with improved or diminished binding to FcRns. See also, e.g., Shields et al. J. Biol. Chem. 9(2):6591-6604 (2001).

In other embodiments, Fc alterations may lead to enhanced or reduced ADCC or CDC activity. As in known in the art, CDC refers to the lysing of a target cell in the presence of complement, and ADCC refers to a form of cytotoxicity in which secreted Ig bound onto FcRs present on certain cytotoxic cells (e.g., Natural Killer cells, neutrophils, and macrophages) enables these cytotoxic effector cells to bind specifically to an antigen-bearing target cell and subsequently kill the target cell with cytotoxins. In the context of the instant invention antibody variants are provided with “altered” FcR binding affinity, which is either enhanced or diminished binding as compared to a parent or unmodified antibody or to an antibody comprising a native sequence FcR. Such variants which display decreased binding may possess little or no appreciable binding, e.g., 0-20% binding to the FcR compared to a native sequence, e.g. as determined by techniques well known in the art. In other embodiments the variant will exhibit enhanced binding as compared to the native immunoglobulin Fc domain. It will be appreciated that these types of Fc variants may advantageously be used to enhance the effective anti-neoplastic properties of the disclosed antibodies. In yet other embodiments, such alterations lead to increased binding affinity, reduced immunogenicity, increased production, altered glycosylation and/or disulfide bonds (e.g., for conjugation sites), modified binding specificity, increased phagocytosis; and/or down regulation of cell surface receptors (e.g. B cell receptor; BCR), etc.

Still other embodiments comprise one or more engineered glycoforms, e.g., a site-specific antibody comprising an altered glycosylation pattern or altered carbohydrate composition that is covalently attached to the protein (e.g., in the Fc domain). See, for example, Shields, R. L. et al. (2002) J. Biol. Chem. 277:26733-26740. Engineered glycoforms may be useful for a variety of purposes, including but not limited to enhancing or reducing effector function, increasing the affinity of the antibody for a target or facilitating production of the antibody. In certain embodiments where reduced effector function is desired, the molecule may be engineered to express an aglycosylated form. Substitutions that may result in elimination of one or more variable region framework glycosylation sites to thereby eliminate glycosylation at that site are well known (see e.g. U.S. Pat. Nos. 5,714,350 and 6,350,861). Conversely, enhanced effector functions or improved binding may be imparted to the Fc containing molecule by engineering in one or more additional glycosylation sites.

Other embodiments include an Fc variant that has an altered glycosylation composition, such as a hypofucosylated antibody having reduced amounts of fucosyl residues or an antibody having increased bisecting GlcNAc structures. Such altered glycosylation patterns have been demonstrated to increase the ADCC ability of antibodies. Engineered glycoforms may be generated by any method known to one skilled in the art, for example by using engineered or variant expression strains, by co-expression with one or more enzymes (for example N-acetylglucosaminyltransferase III (GnTIII)), by expressing a molecule comprising an Fc region in various organisms or cell lines from various organisms or by modifying carbohydrate(s) after the molecule comprising Fc region has been expressed (see, for example, WO 2012/117002).

4.4. Fragments

Regardless of which form of antibody (e.g. chimeric, humanized, etc.) is selected to practice the invention it will be appreciated that immunoreactive fragments, either by themselves or as part of an antibody drug conjugate, of the same may be used in accordance with the teachings herein. An “antibody fragment” comprises at least a portion of an intact antibody. As used herein, the term “fragment” of an antibody molecule includes antigen-binding fragments of antibodies, and the term “antigen-binding fragment” refers to a polypeptide fragment of an immunoglobulin or antibody that immunospecifically binds or reacts with a selected antigen or immunogenic determinant thereof or competes with the intact antibody from which the fragments were derived for specific antigen binding.

Exemplary immunoreactive fragments include: variable light chain fragments (VL), variable heavy chain fragments (VH), scFvs, F(ab′)2 fragment, Fab fragment, Fd fragment, Fv fragment, single domain antibody fragments, diabodies, linear antibodies, single-chain antibody molecules and multispecific antibodies formed from antibody fragments. In addition, an active site-specific fragment comprises a portion of the antibody that retains its ability to interact with the antigen/substrates or receptors and modify them in a manner similar to that of an intact antibody (though maybe with somewhat less efficiency). Such antibody fragments may further be engineered to comprise one or more free cysteines as described herein.

In particularly preferred embodiments the UPK1B binding domain will comprise a scFv construct. As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain and light chain fragments are linked via a spacer sequence. Various methods for preparing an scFv are known, and include methods described in U.S. Pat. No. 4,694,778.

In other embodiments, an antibody fragment is one that comprises the Fc region and that retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fc sequence comprising at least one free cysteine capable of conferring in vivo stability to the fragment.

As would be well recognized by those skilled in the art, fragments can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragment.

In selected embodiments antibody fragments of the invention will comprise ScFv constructs which may be used in various configurations. For example such anti-UPK1B ScFv constructs may be used in adoptive immunity gene therapy to treat tumors. In certain embodiments the antibodies of the invention (e.g. ScFv fragments) may be used to generate a chimeric antigen receptors (CAR) that immunoselectively react with UPK1B. In accordance with the instant disclosure an anti-UPK1B CAR is a fused protein comprising the anti-UPK1B antibodies of the invention or immunoreactive fragments thereof (e.g. ScFv fragments), a transmembrane domain, and at least one intracellular domain. In certain embodiments, T-cells, natural killer cells or dendritic cells that have been genetically engineered to express an anti-UPK1B CAR can be introduced into a subject suffering from cancer in order to stimulate the immune system of the subject to specifically target tumor cells expressing UPK1B. In some embodiments the CARs of the invention will comprise an intracellular domain that initiates a primary cytoplasmic signaling sequence, that is, a sequence for initiating antigen-dependent primary activation via a T-cell receptor complex, for example, intracellular domains derived from CD3ζ, FcRγ, FcRβ, CD3γ, CD3δ, CD3ε, CD5, CD22, CD79a, CD79b, and CD66d. In other embodiments, the CARs of the invention will comprise an intracellular domain that initiates a secondary or co-stimulating signal, for example, intracellular domains derived from CD2, CD4, CD5, CD8α, CD8β, CD28, CD134, CD137, ICOS, CD154, 4-1BB and glucocorticoid-induced tumor necrosis factor receptor (see U.S.P.N. US/2014/0242701).

4.5. Multivalent Constructs

In other embodiments, the antibodies and conjugates of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105.

In one embodiment, the antibodies are bispecific antibodies in which the two chains have different specificities, as described in Millstein et al., 1983, Nature, 305:537-539. Other embodiments include antibodies with additional specificities such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

Multivalent antibodies may immunospecifically bind to different epitopes of the desired target molecule or may immunospecifically bind to both the target molecule as well as a heterologous epitope, such as a heterologous polypeptide or solid support material. While selected embodiments may only bind two antigens (i.e. bispecific antibodies), antibodies with additional specificities such as trispecific antibodies are also encompassed by the instant invention. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

5. Recombinant Production of Antibodies

Antibodies and fragments thereof may be produced or modified using genetic material obtained from antibody producing cells and recombinant technology (see, for example; Dubel and Reichert (Eds.) (2014) Handbook of Therapeutic Antibodies, 2^{nd} Edition, Wiley-Blackwell GmbH; Sambrook and Russell (Eds.) (2000) Molecular Cloning: A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc.; and U.S. Pat. No. 7,709,611).

Another aspect of the invention pertains to nucleic acid molecules that encode the antibodies of the invention. The nucleic acids may be present in whole cells, in a cell lysate, or in a partially purified or substantially pure form. A nucleic acid is “isolated” or rendered substantially pure when separated from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the invention can be, for example, DNA (e.g. genomic DNA, cDNA), RNA and artificial variants thereof (e.g., peptide nucleic acids), whether single-stranded or double-stranded or RNA, RNA and may or may not contain introns. In selected embodiments the nucleic acid is a cDNA molecule.

Nucleic acids of the invention can be obtained using standard molecular biology techniques. For antibodies expressed by hybridomas (e.g., hybridomas prepared as described in the Examples below), cDNAs encoding the light and heavy chains of the antibody can be obtained by standard PCR amplification or cDNA cloning techniques. For antibodies obtained from an immunoglobulin gene library (e.g., using phage display techniques), nucleic acid molecules encoding the antibody can be recovered from the library.

DNA fragments encoding VH and VL segments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, means that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3 in the case of IgG1). The sequences of human heavy chain constant region genes are known in the art (see e.g., Kabat, et al. (1991) (supra)) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. An exemplary IgG1 constant region is set forth in SEQ ID NO: 2. For a Fab fragment heavy chain gene, the VH-encoding DNA can be operatively linked to another DNA molecule encoding only the heavy chain CH1 constant region.

Isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, et al. (1991) (supra)) and DNA fragments encompassing these regions can be obtained by standard PCR amplification. The light chain constant region can be a kappa or lambda constant region, but most preferably is a kappa constant region. An exemplary compatible kappa light chain constant region is set forth in SEQ ID NO: 5 while an exemplary compatible lambda light chain constant region is set forth in SEQ ID NO: 8.

In each case the VH or VL domains may be operatively linked to their respective constant regions (CH or CL) where the constant regions are site-specific constant regions and provide site-specific antibodies. In selected embodiments the resulting site-specific antibodies will comprise two unpaired cysteines on the heavy chains while in other embodiments the site-specific antibodies will comprise two unpaired cysteines in the CL domain.

Contemplated herein are certain polypeptides (e.g. antigens or antibodies) that exhibit “sequence identity”, sequence similarity” or “sequence homology” to the polypeptides of the invention. For example, a derived humanized antibody VH or VL domain may exhibit a sequence similarity with the source (e.g., murine) or acceptor (e.g., human) VH or VL domain. A “homologous” polypeptide may exhibit 65%, 70%, 75%, 80%, 85%, or 90% sequence identity. In other embodiments a “homologous” polypeptides may exhibit 93%, 95% or 98% sequence identity. As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting Examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When using BLAST and Gapped BLAST programs, the default parameters of the respective programs (e.g., XBLAST and NBLAST) can be used.

Residue positions which are not identical may differ by conservative amino acid substitutions or by non-conservative amino acid substitutions. A “conservative amino acid substitution” is one in which an amino acid residue is substituted by another amino acid residue having a side chain with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. In cases where there is a substitution with a non-conservative amino acid, in embodiments the polypeptide exhibiting sequence identity will retain the desired function or activity of the polypeptide of the invention (e.g., antibody.)

Also contemplated herein are nucleic acids that that exhibit “sequence identity”, sequence similarity” or “sequence homology” to the nucleic acids of the invention. A “homologous sequence” means a sequence of nucleic acid molecules exhibiting at least about 65%, 70%, 75%, 80%, 85%, or 90% sequence identity. In other embodiments, a “homologous sequence” of nucleic acids may exhibit 93%, 95% or 98% sequence identity to the reference nucleic acid.

The instant invention also provides vectors comprising such nucleic acids described above, which may be operably linked to a promoter (see, e.g., WO 86/05807; WO 89/01036; and U.S. Pat. No. 5,122,464); and other transcriptional regulatory and processing control elements of the eukaryotic secretory pathway. The invention also provides host cells harboring those vectors and host-expression systems.

As used herein, the term “host-expression system” includes any kind of cellular system that can be engineered to generate either the nucleic acids or the polypeptides and antibodies of the invention. Such host-expression systems include, but are not limited to microorganisms (e.g., E. coli or B. subtilis) transformed or transfected with recombinant bacteriophage DNA or plasmid DNA; yeast (e.g., Saccharomyces) transfected with recombinant yeast expression vectors; or mammalian cells (e.g., COS, CHO-S, HEK293T, 3T3 cells) harboring recombinant expression constructs containing promoters derived from the genome of mammalian cells or viruses (e.g., the adenovirus late promoter). The host cell may be co-transfected with two expression vectors, for example, the first vector encoding a heavy chain derived polypeptide and the second vector encoding a light chain derived polypeptide.

Methods of transforming mammalian cells are well known in the art. See, for example, U.S. Pat. Nos. 4,399,216, 4,912,040, 4,740,461, and 4,959,455. The host cell may also be engineered to allow the production of an antigen binding molecule with various characteristics (e.g. modified glycoforms or proteins having GnTIII activity).

For long-term, high-yield production of recombinant proteins stable expression is preferred. Accordingly, cell lines that stably express the selected antibody may be engineered using standard art recognized techniques and form part of the invention. Rather than using expression vectors that contain viral origins of replication, host cells can be transformed with DNA controlled by appropriate expression control elements (e.g., promoter or enhancer sequences, transcription terminators, polyadenylation sites, etc.), and a selectable marker. Any of the selection systems well known in the art may be used, including the glutamine synthetase gene expression system (the GS system) which provides an efficient approach for enhancing expression under selected conditions. The GS system is discussed in whole or part in connection with EP 0 216 846, EP 0 256 055, EP 0 323 997 and EP 0 338 841 and U.S. Pat. Nos. 5,591,639 and 5,879,936. Another compatible expression system for the development of stable cell lines is the Freedom™ CHO-S Kit (Life Technologies).

Once an antibody of the invention has been produced by recombinant expression or any other of the disclosed techniques, it may be purified or isolated by methods known in the art in that it is identified and separated and/or recovered from its natural environment and separated from contaminants that would interfere with diagnostic or therapeutic uses for the antibody or related ADC. Isolated antibodies include antibodies in situ within recombinant cells.

These isolated preparations may be purified using various art-recognized techniques, such as, for example, ion exchange and size exclusion chromatography, dialysis, diafiltration, and affinity chromatography, particularly Protein A or Protein G affinity chromatography. Compatible methods are discussed more fully in the Examples below.

6. Post-Production Selection

No matter how obtained, antibody producing cells (e.g., hybridomas, yeast colonies, etc.) may be selected, cloned and further screened for desirable characteristics including, for example, robust growth, high antibody production and desirable antibody characteristics such as high affinity for the antigen of interest. Hybridomas can be expanded in vitro in cell culture or in vivo in syngeneic immunocompromised animals. Methods of selecting, cloning and expanding hybridomas and/or colonies are well known to those of ordinary skill in the art. Once the desired antibodies are identified the relevant genetic material may be isolated, manipulated and expressed using common, art-recognized molecular biology and biochemical techniques.

The antibodies produced by naïve libraries (either natural or synthetic) may be of moderate affinity (Kₐ of about 10⁶ to 10⁷ M⁻¹). To enhance affinity, affinity maturation may be mimicked in vitro by constructing antibody libraries (e.g., by introducing random mutations in vitro by using error-prone polymerase) and reselecting antibodies with high affinity for the antigen from those secondary libraries (e.g. by using phage or yeast display). WO 9607754 describes a method for inducing mutagenesis in a CDR of an immunoglobulin light chain to create a library of light chain genes.

Various techniques can be used to select antibodies, including but not limited to, phage or yeast display in which a library of human combinatorial antibodies or scFv fragments is synthesized on phages or yeast, the library is screened with the antigen of interest or an antibody-binding portion thereof, and the phage or yeast that binds the antigen is isolated, from which one may obtain the antibodies or immunoreactive fragments (Vaughan et al., 1996, PMID: 9630891; Sheets et al., 1998, PMID: 9600934; Boder et al., 1997, PMID: 9181578; Pepper et al., 2008, PMID: 18336206). Kits for generating phage or yeast display libraries are commercially available. There also are other methods and reagents that can be used in generating and screening antibody display libraries (see U.S. Pat. No. 5,223,409; WO 92/18619, WO 91/17271, WO 92/20791, WO 92/15679, WO 93/01288, WO 92/01047, WO 92/09690; and Barbas et al., 1991, PMID: 1896445). Such techniques advantageously allow for the screening of large numbers of candidate antibodies and provide for relatively easy manipulation of sequences (e.g., by recombinant shuffling).

### IV. Characteristics of Antibodies

In certain embodiments, antibody-producing cells (e.g., hybridomas or yeast colonies) may be selected, cloned and further screened for favorable properties including, for example, robust growth, high antibody production and, as discussed in more detail below, desirable site-specific antibody characteristics. In other cases characteristics of the antibody may be imparted by selecting a particular antigen (e.g., a specific UPK1B isoform) or immunoreactive fragment of the target antigen for inoculation of the animal. In still other embodiments the selected antibodies may be engineered as described above to enhance or refine immunochemical characteristics such as affinity or pharmacokinetics.

A. Neutralizing Antibodies

In selected embodiments the antibodies of the invention may be “antagonists” or “neutralizing” antibodies, meaning that the antibody may associate with a determinant and block or inhibit the activities of said determinant either directly or by preventing association of the determinant with a binding partner such as a ligand or a receptor, thereby interrupting the biological response that otherwise would result from the interaction of the molecules. A neutralizing or antagonist antibody will substantially inhibit binding of the determinant to its ligand or substrate when an excess of antibody reduces the quantity of binding partner bound to the determinant by at least about 20%, 30%, 40%, 50%, 60%, 70%, 80%, 85%, 90%, 95%, 97%, 99% or more as measured, for example, by target molecule activity or in an in vitro competitive binding assay. It will be appreciated that the modified activity may be measured directly using art recognized techniques or may be measured by the impact the altered activity has downstream (e.g., oncogenesis or cell survival).

B. Internalizing Antibodies

In certain embodiments the antibodies may comprise internalizing antibodies such that the antibody will bind to a determinant and will be internalized (along with any conjugated pharmaceutically active moiety) into a selected target cell including tumorigenic cells. The number of antibody molecules internalized may be sufficient to kill an antigen-expressing cell, especially an antigen-expressing tumorigenic cell. Depending on the potency of the antibody or, in some instances, antibody drug conjugate, the uptake of a single antibody molecule into the cell may be sufficient to kill the target cell to which the antibody binds. With regard to the instant invention there is evidence that a substantial portion of expressed UPK1B protein remains associated with the tumorigenic cell surface, thereby allowing for localization and internalization of the disclosed antibodies or ADCs. In selected embodiments such antibodies will be associated with, or conjugated to, one or more drugs that kill the cell upon internalization. In some embodiments the ADCs of the instant invention will comprise an internalizing site-specific ADC.

As used herein, an antibody that “internalizes” is one that is taken up (along with any conjugated cytotoxin) by a target cell upon binding to an associated determinant. The number of such ADCs internalized will preferably be sufficient to kill the determinant-expressing cell, especially a determinant expressing cancer stem cell. Depending on the potency of the cytotoxin or ADC as a whole, in some instances the uptake of a few antibody molecules into the cell is sufficient to kill the target cell to which the antibody binds. For example, certain drugs such as PBDs or calicheamicin are so potent that the internalization of a few molecules of the toxin conjugated to the antibody is sufficient to kill the target cell. Whether an antibody internalizes upon binding to a mammalian cell can be determined by various art-recognized assays (e.g., saporin assays such as Mab-Zap and Fab-Zap; Advanced Targeting Systems) including those described in the Examples below. Methods of detecting whether an antibody internalizes into a cell are also described in U.S. Pat. No. 7,619,068.

C. Depleting Antibodies

In other embodiments the antibodies of the invention are depleting antibodies. The term “depleting” antibody refers to an antibody that preferably binds to an antigen on or near the cell surface and induces, promotes or causes the death of the cell (e.g., by CDC, ADCC or introduction of a cytotoxic agent). In embodiments, the selected depleting antibodies will be conjugated to a cytotoxin.

Preferably a depleting antibody will be able to kill at least 20%, 30%, 40%, 50%, 60%, 70%, 80%, 85%, 90%, 95%, 97%, or 99% of UPK1B-expressing cells in a defined cell population. The term “apparent IC50”, as used herein, refers to the concentration at which a primary antibody linked to a toxin kills 50 percent of the cells expressing the antigen(s) recognized by the primary antibody. The toxin can be directly conjugated to the primary antibody, or can be associated with the primary antibody via a secondary antibody or antibody fragment that recognizes the primary antibody, and which secondary antibody or antibody fragment is directly conjugated to a toxin. Preferably a depleting antibody will have an IC50 of less than 5 μM, less than 1 μM, less than 100 nM, less than 50 nM, less than 30 nM, less than 20 nM, less than 10 nM, less than 5 nM, less than 2 nM or less than 1 nM. In some embodiments the cell population may comprise enriched, sectioned, purified or isolated tumorigenic cells, including cancer stem cells. In other embodiments the cell population may comprise whole tumor samples or heterogeneous tumor extracts that comprise cancer stem cells. Standard biochemical techniques may be used to monitor and quantify the depletion of tumorigenic cells in accordance with the teachings herein.

D. Binding Affinity

Disclosed herein are antibodies that have a high binding affinity for a specific determinant e.g. UPK1B. The term “K_{D}” refers to the dissociation constant or apparent affinity of a particular antibody-antigen interaction. An antibody of the invention can immunospecifically bind its target antigen when the dissociation constant K_{D} (k_{off}/kₒₙ) is ≤10⁻⁷ M. The antibody specifically binds antigen with high affinity when the K_{D} is ≤5×10⁻⁷ M, and with very high affinity when the K_{D} is ≤5×10⁻¹⁰ M. In one embodiment of the invention, the antibody has a K_{D} of ≤10⁻⁹ M and an off-rate of about 1×10⁻⁴/sec. In one embodiment of the invention, the off-rate is <1×10⁻⁵/sec. In other embodiments of the invention, the antibodies will bind to a determinant with a K_{D} of between about 10⁻⁷ M and 10⁻¹⁰ M, and in yet another embodiment it will bind with a K_{D}≤2×10⁻¹⁰ M. Still other selected embodiments of the invention comprise antibodies that have a K_{D} (k_{off}/kₒₙ) of less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M or less than 5×10⁻¹⁵ M.

In certain embodiments, an antibody of the invention that immunospecifically binds to a determinant e.g. UPK1B may have an association rate constant or kₒₙ (or kₐ) rate (antibody+antigen (Ag)^{k}ₒₙ←antibody-Ag) of at least 10⁵ M^{-l}s^{-l}, at least 2×10⁵ M^{-l}s^{-l}, at least 5×10⁵ M^{-l}s^{-l}, at least 10⁶ M^{-l}s^{-l}, at least 5×10⁶ M^{-l}s^{-l}, at least 10⁷ M^{-l}s^{-l}, at least 5×10⁷ M^{-l}s^{-l}, or at least 10⁸ M^{-l}s^{-l}.

In another embodiment, an antibody of the invention that immunospecifically binds to a determinant e.g. UPK1B may have a disassociation rate constant or k_{off} (or k_{d}) rate (antibody+antigen (Ag)^{k}_{off}←antibody-Ag) of less than l0^{-l} s^{-l}, less than 5×l0^{-l} s^{-l}, less than l0⁻² s^{-l}, less than 5×10⁻² s^{-l}, less than l0⁻³ s^{-l}, less than 5×l0⁻⁴ s^{-l}, less than l0⁻⁴ s^{-l}, less than 5×l0⁻⁴ s^{-l}, less than l0⁻⁵ s^{-l}, less than 5×l0⁻⁵ s^{-l}, less than l0⁻⁶ s^{-l}, less than 5×l0⁻⁴ s^{-l} less than l0⁻⁷ s^{-l}, less than 5×l0⁻⁷ s^{-l}, less than l0⁻⁸ s^{-l}, less than 5×l0⁻⁸ s^{-l}, less than l0⁻⁹ s^{-l}, less than 5×l0⁻⁹ s^{-l} or less than l0⁻¹⁰ s^{-l}.

Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry.

E. Binning and Epitope Mapping

Antibodies disclosed herein may be characterized in terms of the discrete epitope with which they associate. An “epitope” is the portion(s) of a determinant to which the antibody or immunoreactive fragment specifically binds. Immunospecific binding can be confirmed and defined based on binding affinity, as described above, or by the preferential recognition by the antibody of its target antigen in a complex mixture of proteins and/or macromolecules (e.g. in competition assays). A “linear epitope”, is formed by contiguous amino acids in the antigen that allow for immunospecific binding of the antibody. The ability to preferentially bind linear epitopes is typically maintained even when the antigen is denatured. Conversely, a “conformational epitope”, usually comprises non-contiguous amino acids in the antigen's amino acid sequence but, in the context of the antigen's secondary, tertiary or quaternary structure, are sufficiently proximate to be bound concomitantly by a single antibody. When antigens with conformational epitopes are denatured, the antibody will typically no longer recognize the antigen. An epitope (contiguous or non-contiguous) typically includes at least 3, and more usually, at least 5 or 8-10 or 12-20 amino acids in a unique spatial conformation.

It is also possible to characterize the antibodies of the invention in terms of the group or “bin” to which they belong. “Binning” refers to the use of competitive antibody binding assays to identify pairs of antibodies that are incapable of binding an immunogenic determinant simultaneously, thereby identifying antibodies that “compete” for binding. Competing antibodies may be determined by an assay in which the antibody or immunologically functional fragment being tested prevents or inhibits specific binding of a reference antibody to a common antigen. Typically, such an assay involves the use of purified antigen (e.g., UPK1B or a domain or fragment thereof) bound to a solid surface or cells, an unlabeled test antibody and a labeled reference antibody. Competitive inhibition is measured by determining the amount of label bound to the solid surface or cells in the presence of the test antibody. Additional details regarding methods for determining competitive binding are provided in the Examples herein. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more. Conversely, when the reference antibody is bound it will preferably inhibit binding of a subsequently added test antibody (i.e., a UPK1B antibody) by at least 30%, 40%, 45%, 50%, 55%, 60%, 65%, 70% or 75%. In some instance, binding of the test antibody is inhibited by at least 80%, 85%, 90%, 95%, or 97% or more.

Generally binning or competitive binding may be determined using various art-recognized techniques, such as, for example, immunoassays such as western blots, radioimmunoassays, enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such immunoassays are routine and well known in the art (see, Ausubel et al, eds, (1994) Current Protocols in Molecular Biology, Vol. 1, John Wiley & Sons, Inc., New York). Additionally, cross-blocking assays may be used (see, for example, WO 2003/48731; and Harlow et al. (1988) Antibodies, A Laboratory Manual, Cold Spring Harbor Laboratory, Ed Harlow and David Lane).

Other technologies used to determine competitive inhibition (and hence “bins”), include: surface plasmon resonance using, for example, the BIAcore™ 2000 system (GE Healthcare); bio-layer interferometry using, for example, a ForteBio® Octet RED (ForteBio); or flow cytometry bead arrays using, for example, a FACSCanto II (BD Biosciences) or a multiplex LUMINEX™ detection assay (Luminex).

Luminex is a bead-based immunoassay platform that enables large scale multiplexed antibody pairing. The assay compares the simultaneous binding patterns of antibody pairs to the target antigen. One antibody of the pair (capture mAb) is bound to Luminex beads, wherein each capture mAb is bound to a bead of a different color. The other antibody (detector mAb) is bound to a fluorescent signal (e.g. phycoerythrin (PE)). The assay analyzes the simultaneous binding (pairing) of antibodies to an antigen and groups together antibodies with similar pairing profiles. Similar profiles of a detector mAb and a capture mAb indicates that the two antibodies bind to the same or closely related epitopes. In one embodiment, pairing profiles can be determined using Pearson correlation coefficients to identify the antibodies which most closely correlate to any particular antibody on the panel of antibodies that are tested. In embodiments a test/detector mAb will be determined to be in the same bin as a reference/capture mAb if the Pearson's correlation coefficient of the antibody pair is at least 0.9. In other embodiments the Pearson's correlation coefficient is at least 0.8, 0.85, 0.87 or 0.89. In further embodiments, the Pearson's correlation coefficient is at least 0.91, 0.92, 0.93, 0.94, 0.95, 0.96, 0.97, 0.98, 0.99 or 1. Other methods of analyzing the data obtained from the Luminex assay are described in U.S. Pat. No. 8,568,992. The ability of Luminex to analyze 100 different types of beads (or more) simultaneously provides almost unlimited antigen and/or antibody surfaces, resulting in improved throughput and resolution in antibody epitope profiling over a biosensor assay (Miller, et al., 2011, PMID: 21223970).

Similarly binning techniques comprising surface plasmon resonance are compatible with the instant invention. As used herein “surface plasmon resonance,” refers to an optical phenomenon that allows for the analysis of real-time specific interactions by detection of alterations in protein concentrations within a biosensor matrix. Using commercially available equipment such as the BIAcore™ 2000 system it may readily be determined if selected antibodies compete with each other for binding to a defined antigen.

In other embodiments, a technique that can be used to determine whether a test antibody “competes” for binding with a reference antibody is “bio-layer interferometry”, an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. Such biolayer interferometry assays may be conducted using a ForteBio Octet RED machine as follows. A reference antibody (Ab1) is captured onto an anti-mouse capture chip, a high concentration of non-binding antibody is then used to block the chip and a baseline is collected. Monomeric, recombinant target protein is then captured by the specific antibody (Ab1) and the tip is dipped into a well with either the same antibody (Ab1) as a control or into a well with a different test antibody (Ab2). If no further binding occurs, as determined by comparing binding levels with the control Ab1, then Ab1 and Ab2 are determined to be “competing” antibodies. If additional binding is observed with Ab2, then Ab1 and Ab2 are determined not to compete with each other. This process can be expanded to screen large libraries of unique antibodies using a full row of antibodies in a 96-well plate representing unique bins. In embodiments a test antibody will compete with a reference antibody if the reference antibody inhibits specific binding of the test antibody to a common antigen by at least 40%, 45%, 50%, 55%, 60%, 65%, 70% or 75%. In other embodiments, binding is inhibited by at least 80%, 85%, 90%, 95%, or 97% or more.

Once a bin, encompassing a group of competing antibodies, has been defined further characterization can be carried out to determine the specific domain or epitope on the antigen to which that group of antibodies binds. Domain-level epitope mapping may be performed using a modification of the protocol described by Cochran et al., 2004, PMID: 15099763. Fine epitope mapping is the process of determining the specific amino acids on the antigen that comprise the epitope of a determinant to which the antibody binds.

In certain embodiments fine epitope mapping can be performed using phage or yeast display. Other compatible epitope mapping techniques include alanine scanning mutants, peptide blots (Reineke, 2004, PMID: 14970513), or peptide cleavage analysis. In addition, methods such as epitope excision, epitope extraction and chemical modification of antigens can be employed (Tomer, 2000, PMID: 10752610) using enzymes such as proteolytic enzymes (e.g., trypsin, endoproteinase Glu-C, endoproteinase Asp-N, chymotrypsin, etc.); chemical agents such as succinimidyl esters and their derivatives, primary amine-containing compounds, hydrazines and carbohydrazines, free amino acids, etc. In another embodiment Modification-Assisted Profiling, also known as Antigen Structure-based Antibody Profiling (ASAP) can be used to categorize large numbers of monoclonal antibodies directed against the same antigen according to the similarities of the binding profile of each antibody to chemically or enzymatically modified antigen surfaces (U.S.P.N. 2004/0101920).

Once a desired epitope on an antigen is determined, it is possible to generate additional antibodies to that epitope, e.g., by immunizing with a peptide comprising the selected epitope using techniques described herein.

### V. Antibody Conjugates

In some embodiments the antibodies of the invention may be conjugated with pharmaceutically active or diagnostic moieties to form an “antibody drug conjugate” (ADC) or “antibody conjugate”. The term “conjugate” is used broadly and means the covalent or non-covalent association of any pharmaceutically active or diagnostic moiety with an antibody of the instant invention regardless of the method of association. In certain embodiments the association is effected through a lysine or cysteine residue of the antibody. In some embodiments the pharmaceutically active or diagnostic moieties may be conjugated to the antibody via one or more site-specific free cysteine(s). The disclosed ADCs may be used for therapeutic and diagnostic purposes.

The ADCs of the instant invention may be used to deliver cytotoxins or other payloads to the target location (e.g., tumorigenic cells and/or cells expressing UPK1B). As set forth herein the terms “drug” or “warhead” may be used interchangeably and will mean a biologically active or detectable molecule or drug, including anti-cancer agents or cytotoxins as described below. A “payload” may comprise a “drug” or “warhead” in combination with an optional linker compound. The warhead on the conjugate may comprise peptides, proteins or prodrugs which are metabolized to an active agent in vivo, polymers, nucleic acid molecules, small molecules, binding agents, mimetic agents, synthetic drugs, inorganic molecules, organic molecules and radioisotopes. In a preferred embodiment, the disclosed ADCs will direct the bound payload to the target site in a relatively unreactive, non-toxic state before releasing and activating the warhead (e.g., PBDS 1-5 as disclosed herein). This targeted release of the warhead is preferably achieved through stable conjugation of the payloads (e.g., via one or more cysteines on the antibody) and the relatively homogeneous composition of the ADC preparations which minimize over-conjugated toxic ADC species. Coupled with drug linkers that are designed to largely release the warhead once it has been delivered to the tumor site, the conjugates of the instant invention can substantially reduce undesirable non-specific toxicity. This advantageously provides for relatively high levels of the active cytotoxin at the tumor site while minimizing exposure of non-targeted cells and tissue thereby providing an enhanced therapeutic index.

It will be appreciated that, while some embodiments of the invention comprise payloads incorporating therapeutic moieties (e.g., cytotoxins), other payloads incorporating diagnostic agents and biocompatible modifiers may benefit from the targeted release provided by the disclosed conjugates. Accordingly, any disclosure directed to exemplary therapeutic payloads is also applicable to payloads comprising diagnostic agents or biocompatible modifiers as discussed herein unless otherwise dictated by context. The selected payload may be covalently or non-covalently linked to, the antibody and exhibit various stoichiometric molar ratios depending, at least in part, on the method used to effect the conjugation.

Conjugates of the instant invention may be generally represented by the formula:

Ab-[L-D]n or a pharmaceutically acceptable salt thereof wherein:
a) Ab comprises an anti-UPK1B antibody;
b) L comprises an optional linker;
c) D comprises a drug; and
d) n is an integer from about 1 to about 20.

Those of skill in the art will appreciate that conjugates according to the aforementioned formula may be fabricated using a number of different linkers and drugs and that conjugation methodology will vary depending on the selection of components. As such, any drug or drug linker compound that associates with a reactive residue (e.g., cysteine or lysine) of the disclosed antibodies are compatible with the teachings herein. Similarly, any reaction conditions that allow for conjugation (including site-specific conjugation) of the selected drug to an antibody are within the scope of the present invention. Notwithstanding the foregoing, some preferred embodiments of the instant invention comprise selective conjugation of the drug or drug linker to free cysteines using stabilization agents in combination with mild reducing agents as described herein. Such reaction conditions tend to provide more homogeneous preparations with less non-specific conjugation and contaminants and correspondingly less toxicity.

A. Warheads

1. Therapeutic Agents

The antibodies of the invention may be conjugated, linked or fused to or otherwise associated with a pharmaceutically active moiety which is a therapeutic moiety or a drug such as an anti-cancer agent including, but not limited to, cytotoxic agents, cytostatic agents, anti-angiogenic agents, debulking agents, chemotherapeutic agents, radiotherapeutic agents, targeted anti-cancer agents, biological response modifiers, cancer vaccines, cytokines, hormone therapies, anti-metastatic agents and immunotherapeutic agents.

Exemplary anti-cancer agents (including homologs and derivatives thereof) comprise 1-dehydrotestosterone, anthramycins, actinomycin D, bleomycin, calicheamicin, colchicin, cyclophosphamide, cytochalasin B, dactinomycin (formerly actinomycin), dihydroxy anthracin, dione, duocarmycin, emetine, epirubicin, ethidium bromide, etoposide, glucocorticoids, gramicidin D, lidocaine, maytansinoids such as DM-1 and DM-4 (Immunogen), mithramycin, mitomycin, mitoxantrone, paclitaxel, procaine, propranolol, puromycin, tenoposide, tetracaine and pharmaceutically acceptable salts or solvates, acids or derivatives of any of the above.

Additional compatible cytotoxins comprise dolastatins and auristatins, including monomethyl auristatin E (MMAE) and monomethyl auristatin F (MMAF) (Seattle Genetics), amanitins such as alpha-amanitin, beta-amanitin, gamma-amanitin or epsilon-amanitin (Heidelberg Pharma), DNA minor groove binding agents such as duocarmycin derivatives (Syntarga), alkylating agents such as modified or dimeric pyrrolobenzodiazepines (PBD), mechlorethamine, thioepa, chlorambucil, melphalan, carmustine (BCNU), lomustine (CCNU), cyclothosphamide, busulfan, dibromomannitol, streptozotocin, mitomycin C and cisdichlorodiamine platinum (II) (DDP) cisplatin, splicing inhibitors such as meayamycin analogs or derivatives (e.g., FR901464 as set forth in U.S. Pat. No. 7,825,267), tubular binding agents such as epothilone analogs and tubulysins, paclitaxel and DNA damaging agents such as calicheamicins and esperamicins, antimetabolites such as methotrexate, 6-mercaptopurine, 6-thioguanine, cytarabine, and 5-fluorouracil decarbazine, anti-mitotic agents such as vinblastine and vincristine and anthracyclines such as daunorubicin (formerly daunomycin) and doxorubicin and pharmaceutically acceptable salts or solvates, acids or derivatives of any of the above.

In certain aspects the ADCs of the instant invention will comprise a dolastatin warhead. Compatible dolastatins comprise both dolastatin 10 and dolastatin 15 each of which may be in the form of a monomethyl analog (e.g., monomethyl dolastatin 10). Dolastatin 10 and dolastatin 15 are marine natural products isolated from the Indian Ocean sea hare Dollabella auricularia. Being small linear peptide molecules, dolastatin 10 and 15 are considered promising anti-cancer drugs having shown activity against various tumors. The dolastatins are mitotic inhibitors interfering with microtubule assembly and thereby resulting in the formation of tubulin aggregates and inhibition of mitosis. The agents also induce tumor cell apoptosis through a mechanism involving bcl-2, an oncoprotein that is overexpressed in some cancers. Structures of compatible warheads monomethyl dolastatin 10 and dolastatin 15 are shown immediately below:

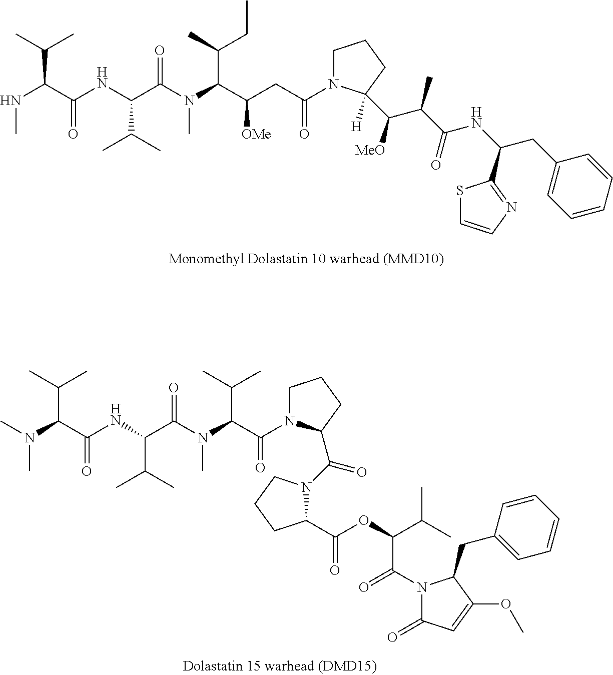 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H](NC)C(C)C)C(C)C ; COC1=CC(=O)N(C(=O)[C@@H](OC(=O)[C@@H]2CCCN2C(=O)[C@@H]2CCCN2C(=O)[C@H](C(C)C)N(C)C(=O)[C@@H](NC(=O)[C@H](C(C)C)N(C)C)C(C)C)C(C)C)[C@H]1Cc1ccccc1]

It will be appreciated that both dimethyl and monomethyl dolastatin warheads are compatible with the disclosed ADCs and are expressly contemplated as being within the scope of the instant invention (e.g., monomethyl dolastatin 10, monomethyl dolastatin 15, dimethyl dolastatin 10 and dimethyl dolastatin 15).

In addition to the dolastatins it will further be appreciated that warheads compatible with the teachings herein may comprise auristatins. As is well known in the art the dolastatins have been structurally modified to provide closely related auristatins which, in certain cases are equipotent derivatives suitable for clinical development. These synthetic agents interact with the Vinca alkaloid binding site on α-tubulin and block its polymerization and prevent the formation of the mitotic apparatus. Particularly compatible auristatins comprise monomethyl auristatin E (MMAE) and monomethyl auristatin F (MMAF) whose structures are shown immediately below:

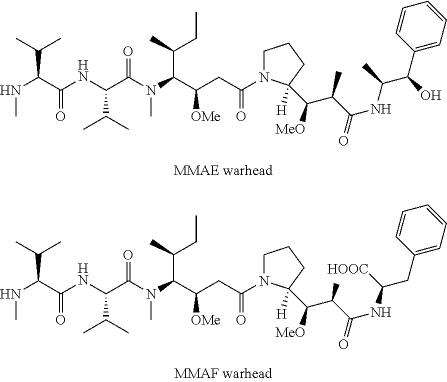 [SMILES: CC[C@H](C)[C@@H]([C@@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@@H](NC)C(C)C)C(C)C ; CC[C@H](C)[C@@H]([C@@H](CC(=O)N1CCC[C@H]1[C@H](OC)[C@@H](C)C(=O)N[C@H](Cc1ccccc1)C(=O)O)OC)N(C)C(=O)[C@@H](NC(=O)[C@@H](NC)C(C)C)C(C)C]

As with the dolastatins It will be appreciated that both dimethyl and monomethyl auristatin warheads are compatible with the disclosed ADCs and are expressly contemplated as being within the scope of the instant invention (e.g., monomethyl auristatin E, monomethyl auristatin F, dimethyl auristatin E and dimethyl auristatin F).

It will be appreciated that each of the aforementioned dolastatin and auristatin warheads will preferably be released upon internalization by the target cell and destruction of the linker. As described in more detail below, certain linkers will comprise cleavable linkers which may incorporate a self-immolation moiety that allows release of the active warhead (e.g., MMD10) without retention of any part of the linker.

In another embodiment the antibodies of the instant invention may be associated with anti-CD3 binding molecules to recruit cytotoxic T-cells and have them target tumorigenic cells (BITE technology; see e.g., Fuhrmann et. al. (2010) Annual Meeting of AACR Abstract No. 5625).

In further embodiments ADCs of the invention may comprise therapeutic radioisotopes conjugated using appropriate linkers. Exemplary radioisotopes that may be compatible with such embodiments include, but are not limited to, iodine (¹³¹I, ¹²⁵I, ¹²³I, ¹²¹I,), carbon (¹⁴C), copper (⁶²Cu, ⁶⁴Cu, ⁶⁷Cu), sulfur (³⁵S), radium (²²³Ra), tritium (³H), indium (¹¹⁵In, ¹¹³n, ¹¹²In, ¹¹¹In,), bismuth (²¹²Bi, ²¹³Bi), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se ¹¹³Sn, ¹¹⁷Sn ²²⁵Ac, ⁷⁶Br, ²¹¹At and ²²⁵Ac. Other radionuclides are also available as diagnostic and therapeutic agents, especially those in the energy range of 60 to 4,000 keV.

In certain some embodiments, the ADCs of the invention may comprise PBDs, and pharmaceutically acceptable salts or solvates, acids or derivatives thereof, as warheads. PBDs are alkylating agents that exert antitumor activity by covalently binding to DNA in the minor groove and inhibiting nucleic acid synthesis. PBDs have been shown to have potent antitumor properties while exhibiting minimal bone marrow depression. PBDs compatible with the invention may be linked to an antibody using several types of linkers (e.g., a peptidyl linker comprising a maleimido moiety with a free sulfhydryl), and in certain embodiments are dimeric in form (i.e., PBD dimers). Compatible PBDs (and optional linkers) that may be conjugated to the disclosed antibodies are described, for example, in U.S. Pat. Nos. 6,362,331, 7,049,311, 7,189,710, 7,429,658, 7,407,951, 7,741,319, 7,557,099, 8,034,808, 8,163,736, 2011/0256157 and PCT filings WO2011/130613, WO2011/128650, WO2011/130616, WO2014/057073 and WO2014/057074.

In other selected embodiments the ADCs of the instant invention will be conjugated to a cytotoxic benzodiazepine derivative warhead. Compatible benzodiazepine derivatives (and optional linkers) that may be conjugated to the disclosed antibodies are described, for example, in U.S. Pat. No. 8,426,402 and PCT filings WO2012/128868 and WO2014/031566. As with the aforementioned PBDs, compatible benzodiazepine derivatives are believed to bind in the minor grove of DNA and inhibit nucleic acid synthesis. Such compounds reportedly have potent antitumor properties and, as such, are particularly suitable for use in the ADCs of the instant invention.

In addition to the aforementioned agents the antibodies of the present invention may also be conjugated to biological response modifiers. For example, in some embodiments the drug moiety can be a polypeptide possessing a desired biological activity. Such proteins may include, for example, a toxin such as abrin, ricin A, Onconase (or another cytotoxic RNase), pseudomonas exotoxin, cholera toxin, diphtheria toxin; an apoptotic agent such as tumor necrosis factor e.g. TNF-α or TNF-β, α-interferon, β-interferon, nerve growth factor, platelet derived growth factor, tissue plasminogen activator, AIM I (WO 97/33899), AIM II (WO 97/34911), Fas Ligand (Takahashi et al., 1994, PMID: 7826947), and VEGI (WO 99/23105), a thrombotic agent, an anti-angiogenic agent, e.g., angiostatin or endostatin, a lymphokine, for example, interleukin-1 (IL-1), interleukin-2 (IL-2), interleukin-6 (IL-6), granulocyte macrophage colony stimulating factor (GM-CSF), and granulocyte colony stimulating factor (G-CSF), or a growth factor e.g., growth hormone (GH).

2. Diagnostic or Detection Agents

In other embodiments, the antibodies of the invention, or fragments or derivatives thereof, are conjugated to a diagnostic or detectable agent, marker or reporter which may be, for example, a biological molecule (e.g., a peptide or nucleotide), a small molecule, fluorophore, or radioisotope. Labeled antibodies can be useful for monitoring the development or progression of a hyperproliferative disorder or as part of a clinical testing procedure to determine the efficacy of a particular therapy including the disclosed antibodies (i.e. theragnostics) or to determine a future course of treatment. Such markers or reporters may also be useful in purifying the selected antibody, for use in antibody analytics (e.g., epitope binding or antibody binning), separating or isolating tumorigenic cells or in preclinical procedures or toxicology studies.

Such diagnosis, analysis and/or detection can be accomplished by coupling the antibody to detectable substances including, but not limited to, various enzymes comprising for example horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I,), carbon (¹⁴C), sulfur (³⁵S), tritium (³H), indium (¹¹⁵In, ¹¹³In, ¹²¹In, ¹¹¹In), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ⁸⁹Zr, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, non-radioactive paramagnetic metal ions, and molecules that are radiolabeled or conjugated to specific radioisotopes. In such embodiments appropriate detection methodology is well known in the art and readily available from numerous commercial sources.

In other embodiments the antibodies or fragments thereof can be fused or conjugated to marker sequences or compounds, such as a peptide or fluorophore to facilitate purification or diagnostic or analytic procedures such as immunohistochemistry, bio-layer interferometry, surface plasmon resonance, flow cytometry, competitive ELISA, FACs, etc. In some embodiments, the marker comprises a histidine tag such as that provided by the pQE vector (Qiagen), among others, many of which are commercially available. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag (U.S. Pat. No. 4,703,004).

3. Biocompatible Modifiers

In selected embodiments the antibodies of the invention may be conjugated with biocompatible modifiers that may be used to adjust, alter, improve or moderate antibody characteristics as desired. For example, antibodies or fusion constructs with increased in vivo half-lives can be generated by attaching relatively high molecular weight polymer molecules such as commercially available polyethylene glycol (PEG) or similar biocompatible polymers. Those skilled in the art will appreciate that PEG may be obtained in many different molecular weights and molecular configurations that can be selected to impart specific properties to the antibody (e.g. the half-life may be tailored). PEG can be attached to antibodies or antibody fragments or derivatives with or without a multifunctional linker either through conjugation of the PEG to the N- or C-terminus of said antibodies or antibody fragments or via epsilon-amino groups present on lysine residues. Linear or branched polymer derivatization that results in minimal loss of biological activity may be used. The degree of conjugation can be closely monitored by SDS-PAGE and mass spectrometry to ensure optimal conjugation of PEG molecules to antibody molecules. Unreacted PEG can be separated from antibody-PEG conjugates by, e.g., size exclusion or ion-exchange chromatography. In a similar manner, the disclosed antibodies can be conjugated to albumin in order to make the antibody or antibody fragment more stable in vivo or have a longer half-life in vivo. The techniques are well known in the art, see e.g., WO 93/15199, WO 93/15200, and WO 01/77137; and EP 0 413, 622. Other biocompatible conjugates are evident to those of ordinary skill and may readily be identified in accordance with the teachings herein.

B. Linker Compounds

As indicated above payloads compatible with the instant invention comprise one or more warheads and, optionally, a linker associating the warheads with the antibody targeting agent. Numerous linker compounds can be used to conjugate the antibodies of the invention to the relevant warhead. The linkers merely need to covalently bind with the reactive residue on the antibody (preferably a cysteine or lysine) and the selected drug compound. Accordingly, any linker that reacts with the selected antibody residue and may be used to provide the relatively stable conjugates (site-specific or otherwise) of the instant invention is compatible with the teachings herein.

Compatible linkers can advantageously bind to reduced cysteines and lysines, which are nucleophilic. Conjugation reactions involving reduced cysteines and lysines include, but are not limited to, thiol-maleimide, thiol-halogeno (acyl halide), thiol-ene, thiol-yne, thiol-vinylsulfone, thiol-bisulfone, thiol-thiosulfonate, thiol-pyridyl disulfide and thiol-parafluoro reactions. As further discussed herein, thiol-maleimide bioconjugation is one of the most widely used approaches due to its fast reaction rates and mild conjugation conditions. One issue with this approach is the possibility of the retro-Michael reaction and loss or transfer of the maleimido-linked payload from the antibody to other proteins in the plasma, such as, for example, human serum albumin. However, in some embodiments the use of selective reduction and site-specific antibodies as set forth herein in the Examples below may be used to stabilize the conjugate and reduce this undesired transfer. Thiol-acyl halide reactions provide bioconjugates that cannot undergo retro-Michael reaction and therefore are more stable. However, the thiol-halide reactions in general have slower reaction rates compared to maleimide-based conjugations and are thus not as efficient in providing undesired drug to antibody ratios. Thiol-pyridyl disulfide reaction is another popular bioconjugation route. The pyridyl disulfide undergoes fast exchange with free thiol resulting in the mixed disulfide and release of pyridine-2-thione. Mixed disulfides can be cleaved in the reductive cell environment releasing the payload. Other approaches gaining more attention in bioconjugation are thiol-vinylsulfone and thiol-bisulfone reactions, each of which are compatible with the teachings herein and expressly included within the scope of the invention.

In selected embodiments compatible linkers will confer stability on the ADCs in the extracellular environment, prevent aggregation of the ADC molecules and keep the ADC freely soluble in aqueous media and in a monomeric state. Before transport or delivery into a cell, the ADC is preferably stable and remains intact, i.e. the antibody remains linked to the drug moiety. While the linkers are stable outside the target cell they may be designed to be cleaved or degraded at some efficacious rate inside the cell. Accordingly an effective linker will: (i) maintain the specific binding properties of the antibody; (ii) allow intracellular delivery of the conjugate or drug moiety; (iii) remain stable and intact, i.e. not cleaved or degraded, until the conjugate has been delivered or transported to its targeted site; and (iv) maintain a cytotoxic, cell-killing effect or a cytostatic effect of the drug moiety (including, in some cases, any bystander effects). The stability of the ADC may be measured by standard analytical techniques such as HPLC/UPLC, mass spectroscopy, HPLC, and the separation/analysis techniques LC/MS and LC/MS/MS. As set forth above covalent attachment of the antibody and the drug moiety requires the linker to have two reactive functional groups, i.e. bivalency in a reactive sense. Bivalent linker reagents that are useful to attach two or more functional or biologically active moieties, such as MMAE and antibodies are known, and methods have been described to provide resulting conjugates compatible with the teachings herein.

Linkers compatible with the present invention may broadly be classified as cleavable and non-cleavable linkers. Cleavable linkers, which may include acid-labile linkers (e.g., oximes and hydrozones), protease cleavable linkers and disulfide linkers, are internalized into the target cell and are cleaved in the endosomal-lysosomal pathway inside the cell. Release and activation of the cytotoxin relies on endosome/lysosome acidic compartments that facilitate cleavage of acid-labile chemical linkages such as hydrazone or oxime. If a lysosomal-specific protease cleavage site is engineered into the linker the cytotoxins will be released in proximity to their intracellular targets. Alternatively, linkers containing mixed disulfides provide an approach by which cytotoxic payloads are released intracellularly as they are selectively cleaved in the reducing environment of the cell, but not in the oxygen-rich environment in the bloodstream. By way of contrast, compatible non-cleavable linkers containing amide linked polyethylene glycol or alkyl spacers liberate toxic payloads during lysosomal degradation of the ADC within the target cell. In some respects the selection of linker will depend on the particular drug used in the conjugate, the particular indication and the antibody target.

Accordingly, certain embodiments of the invention comprise a linker that is cleavable by a cleaving agent that is present in the intracellular environment (e.g., within a lysosome or endosome or caveolae). The linker can be, for example, a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, each of which is known to hydrolyze dipeptide drug derivatives resulting in the release of active drug inside target cells. Exemplary peptidyl linkers that are cleavable by the thiol-dependent protease cathepsin-B are peptides comprising Phe-Leu since cathepsin-B has been found to be highly expressed in cancerous tissue. Other examples of such linkers are described, for example, in U.S. Pat. No. 6,214,345. In specific embodiments, the peptidyl linker cleavable by an intracellular protease is a Val-Cit linker, a Val-Ala linker or a Phe-Lys linker. One advantage of using intracellular proteolytic release of the therapeutic agent is that the agent is typically attenuated when conjugated and the serum stabilities of the conjugates are relatively high.

In other embodiments, the cleavable linker is pH-sensitive. Typically, the pH-sensitive linker will be hydrolyzable under acidic conditions. For example, an acid-labile linker that is hydrolyzable in the lysosome (e.g., a hydrazone, oxime, semicarbazone, thiosemicarbazone, cis-aconitic amide, orthoester, acetal, ketal, or the like) can be used (See, e.g., U.S. Pat. Nos. 5,122,368; 5,824,805; 5,622,929). Such linkers are relatively stable under neutral pH conditions, such as those in the blood, but are unstable (e.g., cleavable) at below pH 5.5 or 5.0 which is the approximate pH of the lysosome.

In yet other embodiments, the linker is cleavable under reducing conditions (e.g., a disulfide linker). A variety of disulfide linkers are known in the art, including, for example, those that can be formed using SATA (N-succinimidyl-S-acetytthioacetate), SPDP (N-succinimidyl-3-(2-pyridyldithio)propionate), SPDB (N-succinimidyl-3-(2-pyridyldithio) butyrate) and SMPT (N-succinimidyl-oxycarbonyl-alpha-methyl-alpha-(2-pyndyl-dithio)toluene). In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In certain aspects of the invention the selected linker will comprise a compound of the formula:

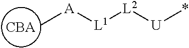

wherein the asterisk indicates the point of attachment to the drug, CBA (i.e. cell binding agent) comprises the anti-UPK1B antibody, L¹ comprises a linker unit and optionally a cleavable linker unit, A is a connecting group (optionally comprising a spacer) connecting L¹ to a reactive residue on the antibody, L² is preferably a covalent bond and U, which may or may not be present, can comprise all or part of a self-immolative unit that facilitates a clean separation of the linker from the warhead at the tumor site.

In some embodiments (such as those set forth in U.S.P.N. 2011/0256157) compatible linkers may comprise:

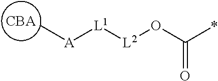

where the asterisk indicates the point of attachment to the drug, CBA (i.e. cell binding agent) comprises the anti-UPK1B antibody, L¹ comprises a linker and optionally a cleavable linker, A is a connecting group (optionally comprising a spacer) connecting L¹ to a reactive residue on the antibody and L² is a covalent bond or together with —OC(═O)— forms a self-immolative moiety.

It will be appreciated that the nature of L¹ and L², where present, can vary widely. These groups are chosen on the basis of their cleavage characteristics, which may be dictated by the conditions at the site to which the conjugate is delivered. Those linkers that are cleaved by the action of enzymes are preferred, although linkers that are cleavable by changes in pH (e.g. acid or base labile), temperature or upon irradiation (e.g. photolabile) may also be used. Linkers that are cleavable under reducing or oxidizing conditions may also find use in the present invention.

In certain embodiments L¹ may comprise a contiguous sequence of amino acids. The amino acid sequence may be the target substrate for enzymatic cleavage, thereby allowing release of the drug.

In one embodiment, L¹ is cleavable by the action of an enzyme. In one embodiment, the enzyme is an esterase or a peptidase.

In another embodiment L¹ is as a cathepsin labile linker.

In one embodiment, L¹ comprises a dipeptide. The dipeptide may be represented as —NH—X₁—X₂—CO—, where —NH— and —CO— represent the N- and C-terminals of the amino acid groups X₁ and X₂ respectively. The amino acids in the dipeptide may be any combination of natural amino acids. Where the linker is a cathepsin labile linker, the dipeptide may be the site of action for cathepsin-mediated cleavage.

Additionally, for those amino acids groups having carboxyl or amino side chain functionality, for example Glu and Lys respectively, CO and NH may represent that side chain functionality.

In one embodiment, the group —X₁—X₂— in dipeptide, —NH—X₁—X₂CO—, is selected from: -Phe-Lys-, -Val-Ala-, -Val-Lys-, -Ala-Lys-, -Val-Cit-, -Phe-Cit-, -Leu-Cit-, -Ile-Cit-, -Phe-Arg- and -Trp-Cit- where Cit is citrulline.

Preferably, the group —X₁—X₂— in dipeptide, —NH—X₁—X₂—CO—, is selected from: -Phe-Lys-, -Val-Ala-, -Val-Lys-, -Ala-Lys-, and -Val-Cit-.

Most preferably, the group —X₁—X₂— in dipeptide, —NH—X₁—X₂—CO—, is -Phe-Lys- or -Val-Ala- or Val-Cit. In certain selected embodiments the dipeptide will comprise -Val-Ala-. In certain other embodiments the dipeptide will comprise -Val-Cit-.

In one embodiment, L2 is present in the form of a covalent bond.

In one embodiment, L2 is present and together with —C(═O)O— forms a self-immolative linker.

In one embodiment, L2 is a substrate for enzymatic activity, thereby allowing release of the warhead.

In one embodiment, where L¹ is cleavable by the action of an enzyme and L² is present, the enzyme cleaves the bond between L¹ and L².

L¹ and L2, where present, may be connected by a bond selected from: —C(═O)NH—, —C(═O)O—, —NHC(═O)—, —OC(═O)—, —OC(═O)O—, —NHC(═O)O—, —OC(═O)NH—, and —NHC(═O)NH—.

An amino group of L¹ that connects to L² may be the N-terminus of an amino acid or may be derived from an amino group of an amino acid side chain, for example a lysine amino acid side chain.

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as omithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴C, ¹⁵N), protected forms, and racemic mixtures thereof.

In one embodiment, —C(═O)O— and L² together form the group:

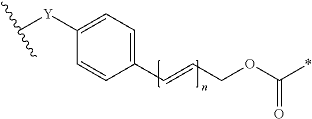

where the asterisk indicates the point of attachment to the drug or cytotoxic agent position, the wavy line indicates the point of attachment to the linker L¹, Y is —N(H)—, —O—, —C(═O)N(H)— or —C(═O)O—, and n is 0 to 3. The phenylene ring is optionally substituted with one, two or three substituents. In one embodiment, the phenylene group is optionally substituted with halo, NO₂, alkyl or hydroxyalkyl.

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

In other embodiments the linker may include a self-immolative linker and the dipeptide together form the group —NH-Val-Cit-CO—NH-PABC-. In other selected embodiments the linker may comprise the group —NH-Val-Ala-CO—NH-PABC-, which is illustrated below:

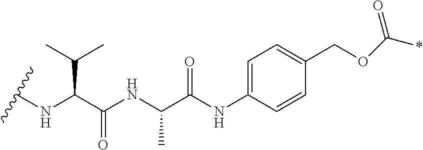 [SMILES: *N[C@H](C(=O)N[C@@H](C)C(=O)Nc1ccc(COC(*)=O)cc1)C(C)C]

where the asterisk indicates the point of attachment to the selected cytotoxic moiety, and the wavy line indicates the point of attachment to the remaining portion of the linker (e.g., the spacer-antibody binding segments) which may be conjugated to the antibody. Upon enzymatic cleavage of the dipeptide, the self-immolative linker will allow for clean release of the protected compound (i.e., the cytotoxin) when a remote site is activated, proceeding along the lines shown below:

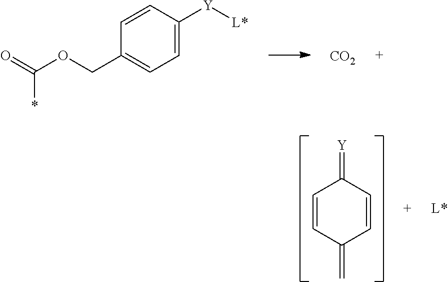

where the asterisk indicates the point of attachment to the selected cytotoxic moiety and where L* is the activated form of the remaining portion of the linker comprising the now cleaved peptidyl unit. The clean release of the warhead ensures it will maintain the desired toxic activity.

In one embodiment, A is a covalent bond. Thus, L¹ and the antibody are directly connected. For example, where L¹ comprises a contiguous amino acid sequence, the N-terminus of the sequence may connect directly to the antibody residue.

In another embodiment, A is a spacer group. Thus, L¹ and the antibody are indirectly connected.

In certain embodiments L¹ and A may be connected by a bond selected from: —C(═O)NH—, —C(═O)O—, —NHC(═O)—, —OC(═O)—, —OC(═O)O—, —NHC(═O)O—, —OC(═O)NH—, and —NHC(═O)NH—.

As will be discussed in more detail below the drug linkers of the instant invention will preferably be linked to reactive thiol nucleophiles on cysteines, including free cysteines. To this end the cysteines of the antibodies may be made reactive for conjugation with linker reagents by treatment with various reducing agent such as DTT or TCEP or mild reducing agents as set forth herein. In other embodiments the drug linkers of the instant invention will preferably be linked to a lysine.

Preferably, the linker contains an electrophilic functional group for reaction with a nucleophilic functional group on the antibody. Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) maleimide groups (ii) activated disulfides, (iii) active esters such as NHS (N-hydroxysuccinimide) esters, HOBt (N-hydroxybenzotriazole) esters, haloformates, and acid halides; (iv) alkyl and benzyl halides such as haloacetamides; and (v) aldehydes, ketones and carboxyl groups.

Exemplary functional groups compatible with the invention are illustrated immediately below:

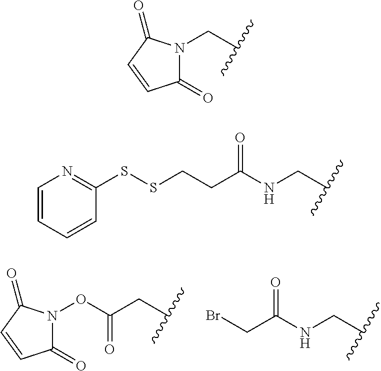 [SMILES: *CN1C(=O)C=CC1=O ; *CNC(=O)CCSSc1ccccn1 ; *CC(=O)ON1C(=O)C=CC1=O ; *CNC(=O)CBr]

In some embodiments the connection between a cysteine (including a free cysteine of a site-specific antibody) and the drug-linker moiety is through a thiol residue and a terminal maleimide group of present on the linker. In such embodiments, the connection between the antibody and the drug-linker may be:

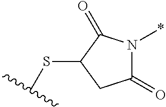 [SMILES: *SC1CC(=O)N(*)C1=O]

where the asterisk indicates the point of attachment to the remaining portion of drug-linker and the wavy line indicates the point of attachment to the remaining portion of the antibody. In such embodiments, the S atom may preferably be derived from a site-specific free cysteine.

With regard to other compatible linkers the binding moiety may comprise a terminal bromo or iodoacetamide that may be reacted with activated residues on the antibody to provide the desired conjugate. In any event one skilled in the art could readily conjugate each of the disclosed drug-linker compounds with a compatible anti-UPK1B antibody (including site-specific antibodies) in view of the instant disclosure.

In accordance with the instant disclosure the invention provides methods of making compatible antibody drug conjugates comprising conjugating an anti-UPK1B antibody with a drug-linker compound (i.e., the [L-D] in the disclosed formula Ab-[L-D]n) selected from the group consisting of:

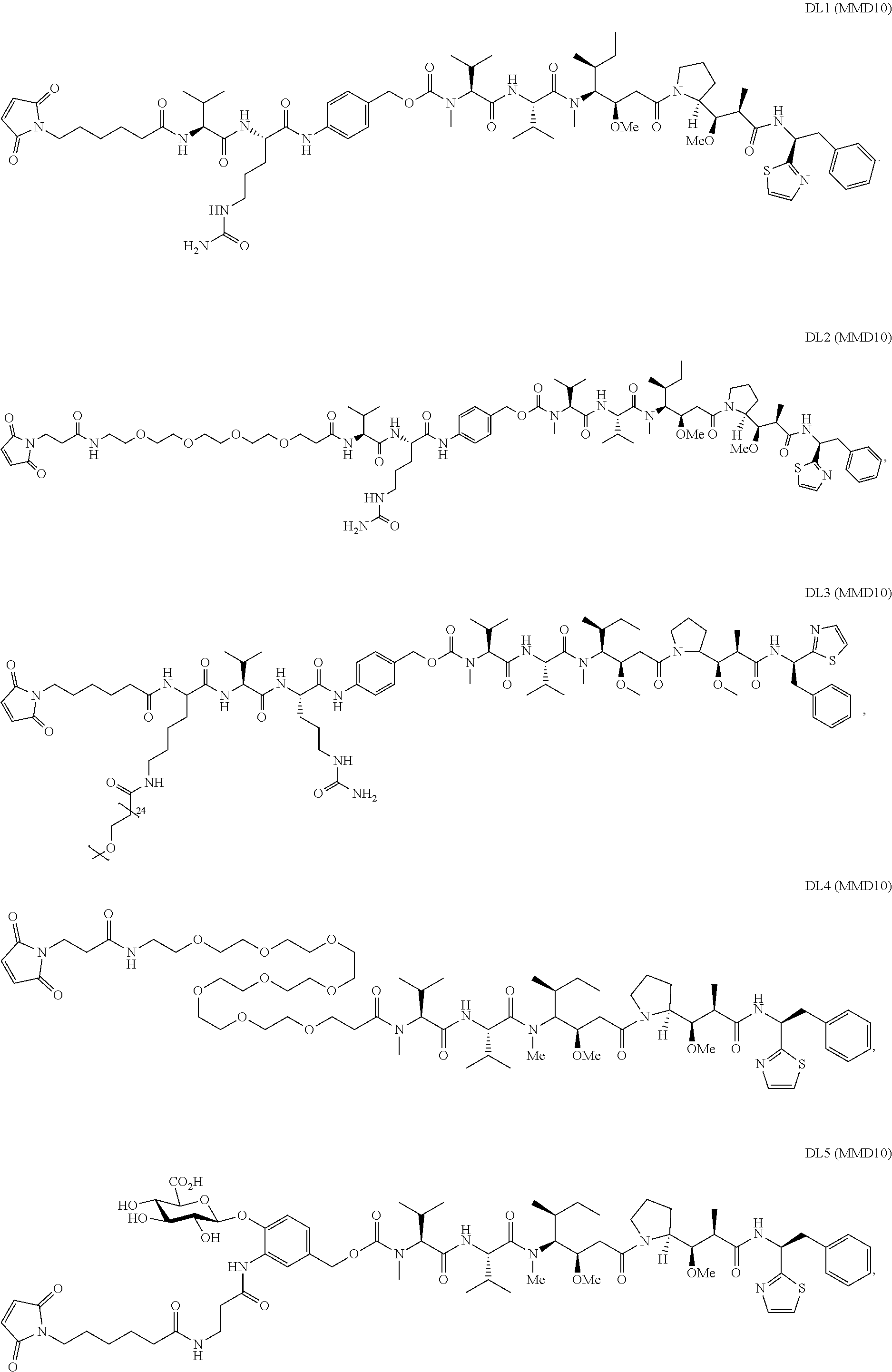

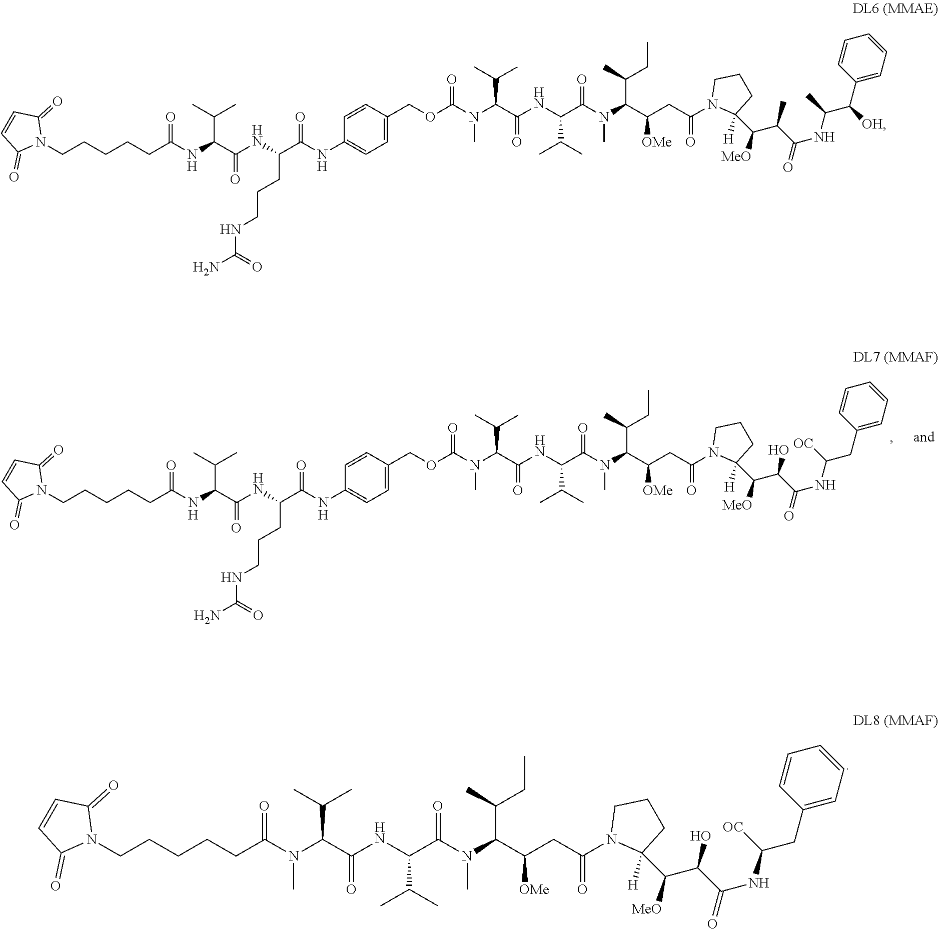

For the purposes of then instant application DL will be used as an abbreviation for “drug-linker” (or “linker-drug in the formula Ab-[L-D]n) and will comprise drug linkers 1-8 (i.e., DL1, DL2, DL3, DL4 DL5, DL6, DL7 and DL8) as set forth above. Note that DL1 to DL5 comprise the same warhead (MMD10) which will be released upon release from the linker. The same pattern also applies for DL7 and DL8 where MMAF is released in each case.

It will be appreciated that the linker appended terminal maleimido moiety may be conjugated to free sulfhydryl(s) on the selected UPK1B antibody using art-recognized techniques. Synthetic routes for the aforementioned compounds are well known in the art while specific methods of conjugating such drug linker combinations are set forth in the Examples below.

Thus, in selected aspects the present invention relates to UPK1B antibodies conjugated to the disclosed DL moieties (DL1-DL8) to provide UPK1B immunoconjugates of the formula Ab-[L-D]n substantially as set forth in ADCs 1-8 immediately below. Accordingly, in certain aspects the invention is directed to an ADC of the formula Ab-[L-D]n comprising a structure selected from the group consisting of:

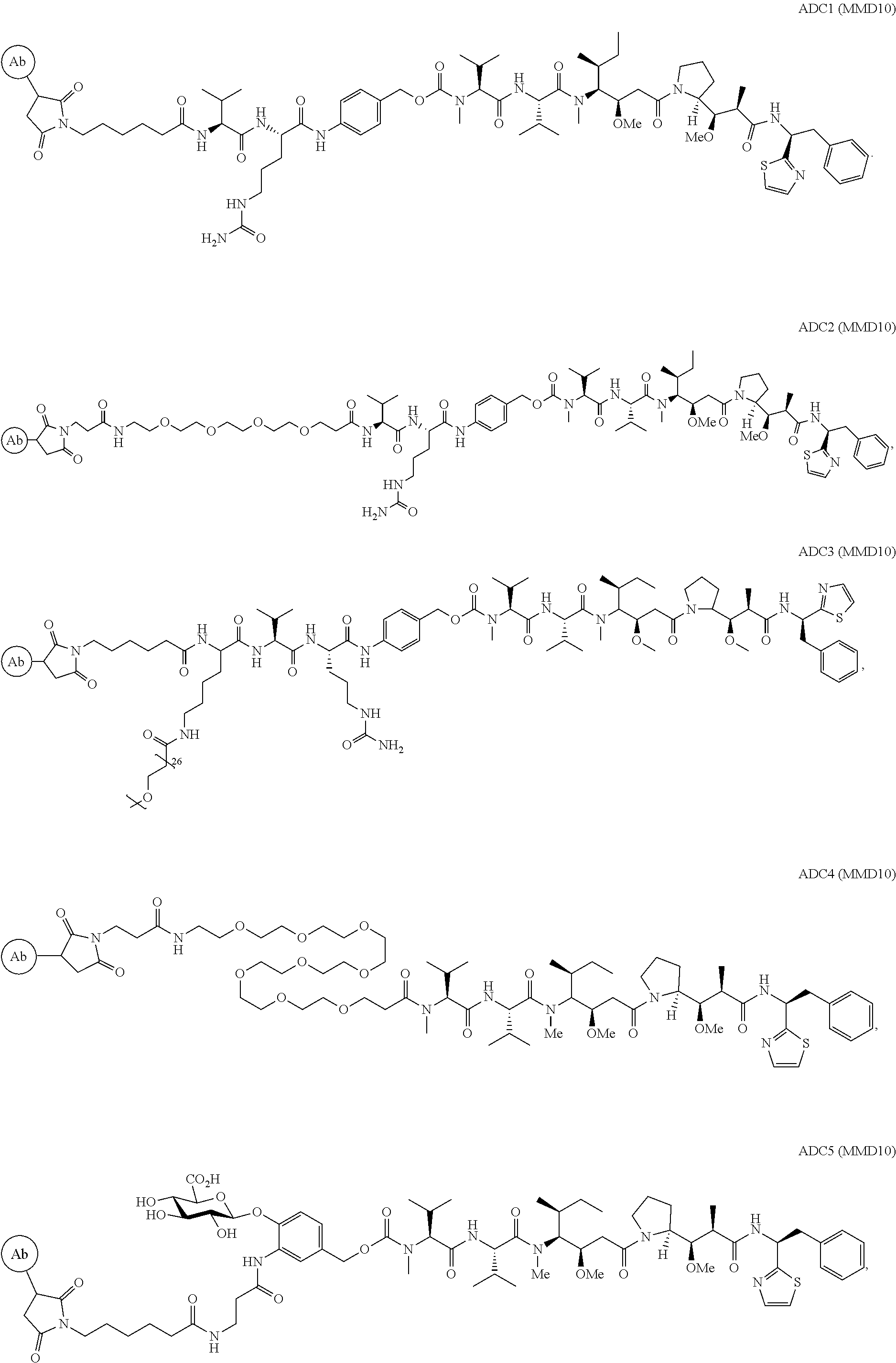

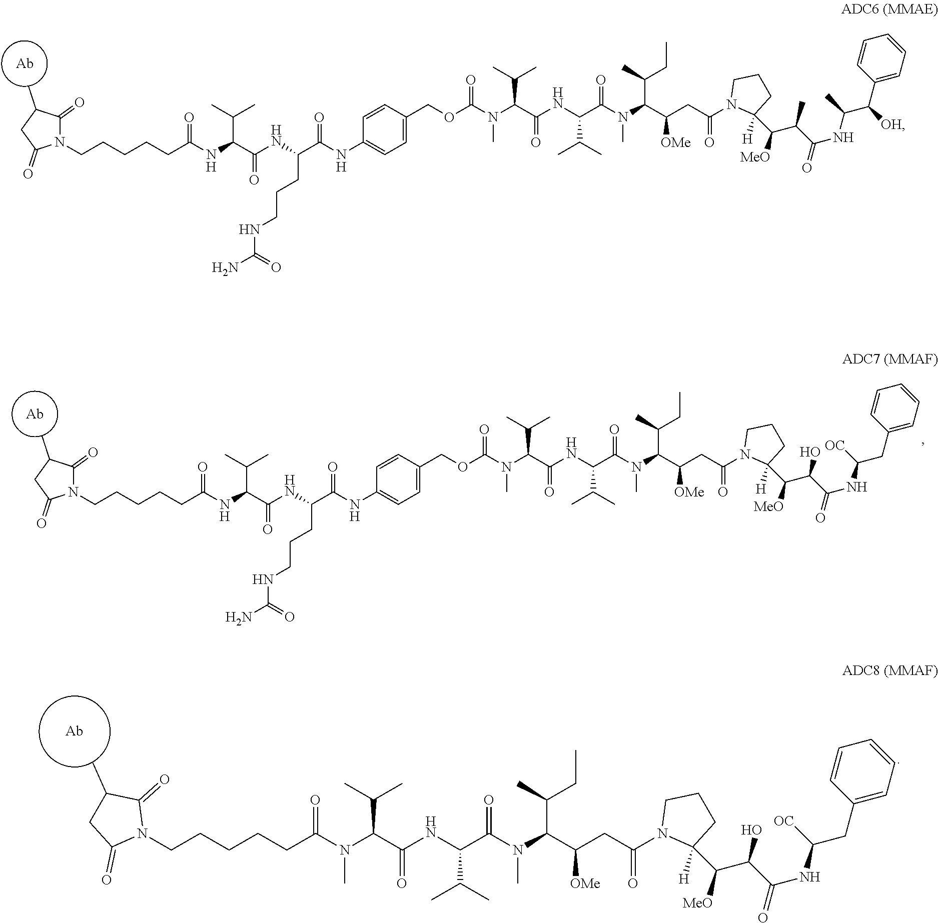

wherein Ab comprises an anti-UPK1B antibody or immunoreactive fragment thereof and n is an integer from about 1 to about 20.

Those of skill in the art will appreciate that the aforementioned structures are defined by the formula Ab-[L-D]n and more than one drug-linker molecule as depicted therein may be covalently conjugated to the UPK1B antibody (e.g., n may be an integer from about 1 to about 20). More particularly, as discussed in more detail below it will be appreciated that more than one payload may be conjugated to each antibody and that the schematic representations above must be construed as such. By way of example ADC1 may comprise an UPK1B antibody conjugated to 1, 2, 3, 4, 5, 6, 7 or 8 or more payloads and that compositions of such ADCs will generally comprise a mixture of drug to antibody ratio (DAR) species.

In certain aspects the UPK1B ADCs of the invention, such as those depicted immediately above, will comprise an anti-UPK1B antibody as set forth in the appended Examples or an immunoreactive fragment thereof. In a particular embodiment ADC1 will comprise hSC115.9ss1 (e.g., hSC115.9ss1 MMD10). In other aspects the UPK1B ADCs of the invention will comprise hSC115.18ss1 (e.g., hSC115.18ss1 MMD10).

C. Conjugation

It will be appreciated that a number of well-known reactions may be used to attach the drug moiety and/or linker to the selected antibody. For example, various reactions exploiting sulfhydryl groups of cysteines may be employed to conjugate the desired moiety. Some embodiments will comprise conjugation of antibodies comprising one or more free cysteines as discussed in detail below. In other embodiments ADCs of the instant invention may be generated through conjugation of drugs to solvent-exposed amino groups of lysine residues present in the selected antibody. Still other embodiments comprise activation of N-terminal threonine and serine residues which may then be used to attach the disclosed payloads to the antibody. The selected conjugation methodology will preferably be tailored to optimize the number of drugs attached to the antibody and provide a relatively high therapeutic index.

Various methods are known in the art for conjugating a therapeutic compound to a cysteine residue and will be apparent to the skilled artisan. Under basic conditions the cysteine residues will be deprotonated to generate a thiolate nucleophile which may be reacted with soft electrophiles such as maleimides and iodoacetamides. Generally reagents for such conjugations may react directly with a cysteine thiol to form the conjugated protein or with a linker-drug to form a linker-drug intermediate. In the case of a linker, several routes, employing organic chemistry reactions, conditions, and reagents are known to those skilled in the art, including: (1) reaction of a cysteine group of the protein of the invention with a linker reagent, to form a protein-linker intermediate, via a covalent bond, followed by reaction with an activated compound; and (2) reaction of a nucleophilic group of a compound with a linker reagent, to form a drug-linker intermediate, via a covalent bond, followed by reaction with a cysteine group of a protein of the invention. As will be apparent to the skilled artisan from the foregoing, bifunctional (or bivalent) linkers are useful in the present invention. For example, the bifunctional linker may comprise a thiol modification group for covalent linkage to the cysteine residue(s) and at least one attachment moiety (e.g., a second thiol modification moiety) for covalent or non-covalent linkage to the compound.

Prior to conjugation, antibodies may be made reactive for conjugation with linker reagents by treatment with a reducing agent such as dithiothreitol (DTT) or (tris(2-carboxyethyl)phosphine (TCEP). In other embodiments additional nucleophilic groups can be introduced into antibodies through the reaction of lysines with reagents, including but not limited to, 2-iminothiolane (Traut's reagent), SATA, SATP or SAT(PEG)4, resulting in conversion of an amine into a thiol.

With regard to such conjugations cysteine thiol or lysine amino groups are nucleophilic and capable of reacting to form covalent bonds with electrophilic groups on linker reagents or compound-linker intermediates or drugs including: (i) active esters such as NHS esters, HOBt esters, haloformates, and acid halides; (ii) alkyl and benzyl halides, such as haloacetamides; (iii) aldehydes, ketones, carboxyl, and maleimide groups; and (iv) disulfides, including pyridyl disulfides, via sulfide exchange. Nucleophilic groups on a compound or linker include, but are not limited to amine, thiol, hydroxyl, hydrazide, oxime, hydrazine, thiosemicarbazone, hydrazine carboxylate, and aryihydrazide groups capable of reacting to form covalent bonds with electrophilic groups on linker moieties and linker reagents.

Conjugation reagents commonly include maleimide, haloacetyl, iodoacetamide succinimidyl ester, isothiocyanate, sulfonyl chloride, 2,6-dichlorotriazinyl, pentafluorophenyl ester, and phosphoramidite, although other functional groups can also be used. In certain embodiments methods include, for example, the use of maleimides, iodoacetimides or haloacetyl/alkyl halides, aziridne, acryloyl derivatives to react with the thiol of a cysteine to produce a thioether that is reactive with a compound. Disulphide exchange of a free thiol with an activated piridyldisulphide is also useful for producing a conjugate (e.g., use of 5-thio-2-nitrobenzoic (TNB) acid). Preferably, a maleimide is used.

As indicated above, lysine may also be used as a reactive residue to effect conjugation as set forth herein. The nucleophilic lysine residue is commonly targeted through amine-reactive succinimidylesters. To obtain an optimal number of deprotonated lysine residues, the pH of the aqueous solution must be below the pKa of the lysine ammonium group, which is around 10.5, so the typical pH of the reaction is about 8 and 9. The common reagent for the coupling reaction is NHS-ester which reacts with nucleophilic lysine through a lysine acylation mechanism. Other compatible reagents that undergo similar reactions comprise isocyanates and isothiocyanates which also may be used in conjunction with the teachings herein to provide ADCs. Once the lysines have been activated, many of the aforementioned linking groups may be used to covalently bind the warhead to the antibody.

Methods are also known in the art for conjugating a compound to a threonine or serine residue (preferably a N-terminal residue). For example methods have been described in which carbonyl precursors are derived from the 1,2-aminoalcohols of serine or threonine, which can be selectively and rapidly converted to aldehyde form by periodate oxidation. Reaction of the aldehyde with a 1,2-aminothiol of cysteine in a compound to be attached to a protein of the invention forms a stable thiazolidine product. This method is particularly useful for labeling proteins at N-terminal serine or threonine residues.

In some embodiments reactive thiol groups may be introduced into the selected antibody (or fragment thereof) by introducing one, two, three, four, or more free cysteine residues (e.g., preparing antibodies comprising one or more free non-native cysteine amino acid residues). Such site-specific antibodies or engineered antibodies allow for conjugate preparations that exhibit enhanced stability and substantial homogeneity due, at least in part, to the provision of engineered free cysteine site(s) and/or the novel conjugation procedures set forth herein. Unlike conventional conjugation methodology that fully or partially reduces each of the intrachain or interchain antibody disulfide bonds to provide conjugation sites (and is fully compatible with the instant invention), the present invention additionally provides for the selective reduction of certain prepared free cysteine sites and attachment of the drug-linker to the same.

In this regard it will be appreciated that the conjugation specificity promoted by the engineered sites and the selective reduction allows for a high percentage of site directed conjugation at the desired positions. Significantly some of these conjugation sites, such as those present in the terminal region of the light chain constant region, are typically difficult to conjugate effectively as they tend to cross-react with other free cysteines. However, through molecular engineering and selective reduction of the resulting free cysteines, efficient conjugation rates may be obtained which considerably reduces unwanted high-DAR contaminants and non-specific toxicity. More generally the engineered constructs and disclosed novel conjugation methods comprising selective reduction provide ADC preparations having improved pharmacokinetics and/or pharmacodynamics and, potentially, an improved therapeutic index.

In certain embodiments site-specific constructs present free cysteine(s) which, when reduced, comprise thiol groups that are nucleophilic and capable of reacting to form covalent bonds with electrophilic groups on linker moieties such as those disclosed above. As discussed above antibodies of the instant invention may have reducible unpaired interchain or intrachain cysteines or introduced non-native cysteines, i.e. cysteines providing such nucleophilic groups. Thus, in certain embodiments the reaction of free sulfhydryl groups of the reduced free cysteines and the terminal maleimido or haloacetamide groups of the disclosed drug-linkers will provide the desired conjugation. In such cases free cysteines of the antibodies may be made reactive for conjugation with linker reagents by treatment with a reducing agent such as dithiothreitol (DTT) or (tris (2-carboxyethyl)phosphine (TCEP). Each free cysteine will thus present, theoretically, a reactive thiol nucleophile. While such reagents are particularly compatible with the instant invention it will be appreciated that conjugation of site-specific antibodies may be effected using various reactions, conditions and reagents generally known to those skilled in the art.

In addition it has been found that the free cysteines of engineered antibodies may be selectively reduced to provide enhanced site-directed conjugation and a reduction in unwanted, potentially toxic contaminants. More specifically “stabilizing agents” such as arginine have been found to modulate intra- and inter-molecular interactions in proteins and may be used, in conjunction with selected reducing agents (preferably relatively mild), to selectively reduce the free cysteines and to facilitate site-specific conjugation as set forth herein. As used herein the terms “selective reduction” or “selectively reducing” may be used interchangeably and shall mean the reduction of free cysteine(s) without substantially disrupting native disulfide bonds present in the engineered antibody. In selected embodiments this selective reduction may be effected by the use of certain reducing agents or certain reducing agent concentrations. In other embodiments selective reduction of an engineered construct will comprise the use of stabilization agents in combination with reducing agents (including mild reducing agents). It will be appreciated that the term “selective conjugation” shall mean the conjugation of an engineered antibody that has been selectively reduced in the presence of a cytotoxin as described herein. In this respect the use of such stabilizing agents (e.g., arginine) in combination with selected reducing agents can markedly improve the efficiency of site-specific conjugation as determined by extent of conjugation on the heavy and light antibody chains and DAR distribution of the preparation. Compatible antibody constructs and selective conjugation techniques and reagents are extensively disclosed in WO2015/031698 which is incorporated herein specifically as to such methodology and constructs.

While not wishing to be bound by any particular theory, such stabilizing agents may act to modulate the electrostatic microenvironment and/or modulate conformational changes at the desired conjugation site, thereby allowing relatively mild reducing agents (which do not materially reduce intact native disulfide bonds) to facilitate conjugation at the desired free cysteine site(s). Such agents (e.g., certain amino acids) are known to form salt bridges (via hydrogen bonding and electrostatic interactions) and can modulate protein-protein interactions in such a way as to impart a stabilizing effect that may cause favorable conformational changes and/or reduce unfavorable protein-protein interactions. Moreover, such agents may act to inhibit the formation of undesired intramolecular (and intermolecular) cysteine-cysteine bonds after reduction thus facilitating the desired conjugation reaction wherein the engineered site-specific cysteine is bound to the drug (preferably via a linker). Since selective reduction conditions do not provide for the significant reduction of intact native disulfide bonds, the subsequent conjugation reaction is naturally driven to the relatively few reactive thiols on the free cysteines (e.g., preferably 2 free thiols per antibody). As previously alluded to, such techniques may be used to considerably reduce levels of non-specific conjugation and corresponding unwanted DAR species in conjugate preparations fabricated in accordance with the instant disclosure.

In selected embodiments stabilizing agents compatible with the present invention will generally comprise compounds with at least one moiety having a basic pKa. In certain embodiments the moiety will comprise a primary amine while in other embodiments the amine moiety will comprise a secondary amine. In still other embodiments the amine moiety will comprise a tertiary amine or a guanidinium group. In other selected embodiments the amine moiety will comprise an amino acid while in other compatible embodiments the amine moiety will comprise an amino acid side chain. In yet other embodiments the amine moiety will comprise a proteinogenic amino acid. In still other embodiments the amine moiety comprises a non-proteinogenic amino acid. In some embodiments, compatible stabilizing agents may comprise arginine, lysine, proline and cysteine. In certain preferred embodiments the stabilizing agent will comprise arginine. In addition compatible stabilizing agents may include guanidine and nitrogen containing heterocycles with basic pKa.

In certain embodiments compatible stabilizing agents comprise compounds with at least one amine moiety having a pKa of greater than about 7.5, in other embodiments the subject amine moiety will have a pKa of greater than about 8.0, in yet other embodiments the amine moiety will have a pKa greater than about 8.5 and in still other embodiments the stabilizing agent will comprise an amine moiety having a pKa of greater than about 9.0. Other embodiments will comprise stabilizing agents where the amine moiety will have a pKa of greater than about 9.5 while certain other embodiments will comprise stabilizing agents exhibiting at least one amine moiety having a pKa of greater than about 10.0. In still other embodiments the stabilizing agent will comprise a compound having the amine moiety with a pKa of greater than about 10.5, in other embodiments the stabilizing agent will comprise a compound having a amine moiety with a pKa greater than about 11.0, while in still other embodiments the stabilizing agent will comprise a amine moiety with a pKa greater than about 11.5. In yet other embodiments the stabilizing agent will comprise a compound having an amine moiety with a pKa greater than about 12.0, while in still other embodiments the stabilizing agent will comprise an amine moiety with a pKa greater than about 12.5. Those of skill in the art will understand that relevant pKa's may readily be calculated or determined using standard techniques and used to determine the applicability of using a selected compound as a stabilizing agent.

The disclosed stabilizing agents are shown to be particularly effective at targeting conjugation to free site-specific cysteines when combined with certain reducing agents. For the purposes of the instant invention, compatible reducing agents may include any compound that produces a reduced free site-specific cysteine for conjugation without significantly disrupting the native disulfide bonds of the engineered antibody. Under such conditions, preferably provided by the combination of selected stabilizing and reducing agents, the activated drug linker is largely limited to binding to the desired free site-specific cysteine site(s). Relatively mild reducing agents or reducing agents used at relatively low concentrations to provide mild conditions are particularly preferred. As used herein the terms “mild reducing agent” or “mild reducing conditions” shall be held to mean any agent or state brought about by a reducing agent (optionally in the presence of stabilizing agents) that provides thiols at the free cysteine site(s) without substantially disrupting native disulfide bonds present in the engineered antibody. That is, mild reducing agents or conditions (preferably in combination with a stabilizing agent) are able to effectively reduce free cysteine(s) (provide a thiol) without significantly disrupting the protein's native disulfide bonds. The desired reducing conditions may be provided by a number of sulfhydryl-based compounds that establish the appropriate environment for selective conjugation. In embodiments mild reducing agents may comprise compounds having one or more free thiols while in some embodiments mild reducing agents will comprise compounds having a single free thiol. Non-limiting examples of reducing agents compatible with the selective reduction techniques of the instant invention comprise glutathione, n-acetyl cysteine, cysteine, 2-aminoethane-1-thiol and 2-hydroxyethane-1-thiol.

It will be appreciated that selective reduction process set forth above is particularly effective at targeted conjugation to the free cysteine. In this respect the extent of conjugation to the desired target site (defined here as “conjugation efficiency”) in site-specific antibodies may be determined by various art-accepted techniques. The efficiency of the site-specific conjugation of a drug to an antibody may be determined by assessing the percentage of conjugation on the target conjugation site(s) (e.g. free cysteines on the c-terminus of each light chain) relative to all other conjugated sites. In certain embodiments, the method herein provides for efficiently conjugating a drug to an antibody comprising free cysteines. In some embodiments, the conjugation efficiency is at least 5%, at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 98% or more as measured by the percentage of target conjugation relative to all other conjugation sites.

It will further be appreciated that engineered antibodies capable of conjugation may contain free cysteine residues that comprise sulfhydryl groups that are blocked or capped as the antibody is produced or stored. Such caps include small molecules, proteins, peptides, ions and other materials that interact with the sulfhydryl group and prevent or inhibit conjugate formation. In some cases the unconjugated engineered antibody may comprise free cysteines that bind other free cysteines on the same or different antibodies. As discussed herein such cross-reactivity may lead to various contaminants during the fabrication procedure. In some embodiments, the engineered antibodies may require uncapping prior to a conjugation reaction. In specific embodiments, antibodies herein are uncapped and display a free sulfhydryl group capable of conjugation. In specific embodiments, antibodies herein are subjected to an uncapping reaction that does not disturb or rearrange the naturally occurring disulfide bonds. It will be appreciated that in most cases the uncapping reactions will occur during the normal reduction reactions (reduction or selective reduction).

D. DAR Distribution and Purification

In selected embodiments conjugation and purification methodology compatible with the present invention advantageously provides the ability to generate relatively homogeneous ADC preparations comprising a narrow DAR distribution. In this regard the disclosed constructs (e.g., site-specific constructs) and/or selective conjugation provides for homogeneity of the ADC species within a sample in terms of the stoichiometric ratio between the drug and the engineered antibody and with respect to the toxin location. As briefly discussed above the term “drug to antibody ratio” or “DAR” refers to the molar ratio of drug to antibody. In certain embodiments a conjugate preparation may be substantially homogeneous with respect to its DAR distribution, meaning that within the ADC preparation is a predominant species of site-specific ADC with a particular DAR (e.g., a DAR of 2 or 4) that is also uniform with respect to the site of loading (i.e., on the free cysteines). In other certain embodiments of the invention it is possible to achieve the desired homogeneity through the use of site-specific antibodies and/or selective reduction and conjugation. In other embodiments the desired homogeneity may be achieved through the use of site-specific constructs in combination with selective reduction. In yet other embodiments compatible preparations may be purified using analytical or preparative chromatography techniques to provide the desired homogeneity. In each of these embodiments the homogeneity of the ADC sample can be analyzed using various techniques known in the art including but not limited to mass spectrometry, HPLC (e.g. size exclusion HPLC, RP-HPLC, HIC-HPLC etc.) or capillary electrophoresis.

With regard to the purification of ADC preparations it will be appreciated that standard pharmaceutical preparative methods may be employed to obtain the desired purity. As discussed herein liquid chromatography methods such as reverse phase (RP) and hydrophobic interaction chromatography (HIC) may separate compounds in the mixture by drug loading value. In some cases, ion-exchange (IEC) or mixed-mode chromatography (MMC) may also be used to isolate species with a specific drug load.

The disclosed ADCs and preparations thereof may comprise drug and antibody moieties in various stoichiometric molar ratios depending on the configuration of the antibody and, at least in part, on the method used to effect conjugation. In certain embodiments the drug loading per ADC may comprise from 1-20 payloads or warheads (i.e., n is 1-20). Other selected embodiments may comprise ADCs with a drug loading of from 1 to 15 warheads. In still other embodiments the ADCs may comprise from 1-12 warheads or, more preferably, from 1-10 warheads. In some embodiments the ADCs will comprise from 1 to 8 warheads.

While theoretical drug loading may be relatively high, practical limitations such as free cysteine cross reactivity and warhead hydrophobicity tend to limit the generation of homogeneous preparations comprising such DAR due to aggregates and other contaminants. That is, higher drug loading, e.g. >8 or 10, may cause aggregation, insolubility, toxicity, or loss of cellular permeability of certain antibody-drug conjugates depending on the payload. In view of such concerns drug loading provided by the instant invention preferably ranges from 1 to 8 drugs per conjugate, i.e. where 1, 2, 3, 4, 5, 6, 7, or 8 drugs are covalently attached to each antibody (e.g., for IgG1, other antibodies may have different loading capacity depending the number of disulfide bonds). Preferably the DAR of compositions of the instant invention will be approximately 2, 4 or 6 and in some embodiments the DAR will comprise approximately 2.

Despite the relatively high level of homogeneity provided by the instant invention the disclosed compositions actually comprise a mixture of conjugates with a range of drugs compounds (potentially from 1 to 8 in the case of an IgG1). As such, the disclosed ADC compositions include mixtures of conjugates where most of the constituent antibodies are covalently linked to one or more drug moieties and (despite the relative conjugate specificity provided by engineered constructs and selective reduction) where the drug moieties may be attached to the antibody by various thiol groups. That is, following conjugation ADC compositions of the invention will comprise a mixture of conjugates with different drug loads (e.g., from 1 to 8 drugs per IgG1 antibody) at various concentrations (along with certain reaction contaminants primarily caused by free cysteine cross reactivity). However using selective reduction and post-fabrication purification the conjugate compositions may be driven to the point where they largely contain a single predominant desired ADC species (e.g., with a drug loading of 2) with relatively low levels of other ADC species (e.g., with a drug loading of 1, 4, 6, etc.). The average DAR value represents the weighted average of drug loading for the composition as a whole (i.e., all the ADC species taken together). Due to inherent uncertainty in the quantification methodology employed and the difficulty in completely removing the non-predominant ADC species in a commercial setting, acceptable DAR values or specifications are often presented as an average, a range or distribution (i.e., an average DAR of 2+/−0.5). Preferably compositions comprising a measured average DAR within the range (i.e., 1.5 to 2.5) would be used in a pharmaceutical setting.

Thus, in some embodiments the present invention will comprise compositions having an average DAR of 1, 2, 3, 4, 5, 6, 7 or 8 each +/−0.5. In other embodiments the present invention will comprise an average DAR of 2, 4, 6 or 8+/−0.5. Finally, in selected embodiments the present invention will comprise an average DAR of 2+/−0.5 or 4+/−0.5. It will be appreciated that the range or deviation may be less than 0.4 in some embodiments. Thus, in other embodiments the compositions will comprise an average DAR of 1, 2, 3, 4, 5, 6, 7 or 8 each +/−0.3, an average DAR of 2, 4, 6 or 8+/−0.3, even more preferably an average DAR of 2 or 4+/−0.3 or even an average DAR of 2+/−0.3. In other embodiments IgG1 conjugate compositions will preferably comprise a composition with an average DAR of 1, 2, 3, 4, 5, 6, 7 or 8 each +/−0.4 and relatively low levels (i.e., less than 30%) of non-predominant ADC species. In other embodiments the ADC composition will comprise an average DAR of 2, 4, 6 or 8 each +/−0.4 with relatively low levels (<30%) of non-predominant ADC species. In some embodiments the ADC composition will comprise an average DAR of 2+/−0.4 with relatively low levels (<30%) of non-predominant ADC species. In yet other embodiments the predominant ADC species (e.g., DAR of 2 or DAR of 4) will be present at a concentration of greater than 50%, at a concentration of greater than 55%, at a concentration of greater than 60%, at a concentration of greater than 65%, at a concentration of greater than 70%, at a concentration of greater than 75%, at a concentration of greater that 80%, at a concentration of greater than 85%, at a concentration of greater than 90%, at a concentration of greater than 93%, at a concentration of greater than 95% or even at a concentration of greater than 97% when measured against all other DAR species present in the composition.

As detailed in the Examples below the distribution of drugs per antibody in preparations of ADC from conjugation reactions may be characterized by conventional means such as UV-Vis spectrophotometry, reverse phase HPLC, HIC, mass spectroscopy, ELISA, and electrophoresis. The quantitative distribution of ADC in terms of drugs per antibody may also be determined. By ELISA, the averaged value of the drugs per antibody in a particular preparation of ADC may be determined. However, the distribution of drug per antibody values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues.

### VI. Diagnostics and Screening

A. Diagnostics

The invention provides in vitro and in vivo methods for detecting, diagnosing or monitoring proliferative disorders and methods of screening cells from a patient to identify tumor cells including tumorigenic cells. Such methods include identifying an individual having cancer for treatment or monitoring progression of a cancer, comprising contacting the patient or a sample obtained from a patient (either in vivo or in vitro) with a detection agent (e.g., an antibody or nucleic acid probe) capable of specifically recognizing and associating with a UPK1B determinant and detecting the presence or absence, or level of association of the detection agent in the sample. In selected embodiments the detection agent will comprise an antibody associated with a detectable label or reporter molecule as described herein. In certain other embodiments the UPK1B antibody will be administered and detected using a secondary labelled antibody (e.g., an anti-murine antibody). In yet other embodiments (e.g., In situ hybridization or ISH) a nucleic acid probe that reacts with a genomic UPK1B determinant will be used in the detection, diagnosis or monitoring of the proliferative disorder.

More generally the presence and/or levels of UPK1B determinants may be measured using any of a number of techniques available to the person of ordinary skill in the art for protein or nucleic acid analysis, e.g., direct physical measurements (e.g., mass spectrometry), binding assays (e.g., immunoassays, agglutination assays, and immunochromatographic assays), Polymerase Chain Reaction (PCR, RT-PCR; RT-qPCR) technology, branched oligonucleotide technology, Northern blot technology, oligonucleotide hybridization technology and in situ hybridization technology. The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

In some embodiments, the association of the detection agent with particular cells or cellular components in the sample indicates that the sample may contain tumorigenic cells, thereby denoting that the individual having cancer may be effectively treated with an antibody or ADC as described herein.

In certain preferred embodiments the assays may comprise immunohistochemistry (IHC) assays or variants thereof (e.g., fluorescent, chromogenic, standard ABC, standard LSAB, etc.), immunocytochemistry or variants thereof (e.g., direct, indirect, fluorescent, chromogenic, etc.) or In situ hybridization (ISH) or variants thereof (e.g., chromogenic in situ hybridization (CISH) or fluorescence in situ hybridization (DNA-FISH or RNA-FISH]))

In this regard certain aspects of the instant invention comprise the use of labeled UPK1B for immunohistochemistry (IHC). More particularly UPK1B IHC may be used as a diagnostic tool to aid in the diagnosis of various proliferative disorders and to monitor the potential response to treatments including UPK1B antibody therapy. In certain embodiments the UPK1B will be conjugated to one or more reporter molecules. In other embodiments the UPK1B antibody (e.g. SC115.7) will be unlabeled and will be detected with a separate agent (e.g., an anti-murine antibody) associated with one or more reporter molecules. As discussed herein and shown in the Examples below compatible diagnostic assays may be performed on tissues that have been chemically fixed (including but not limited to: formaldehyde, gluteraldehyde, osmium tetroxide, potassium dichromate, acetic acid, alcohols, zinc salts, mercuric chloride, chromium tetroxide and picric acid) and embedded (including but not limited to: glycol methacrylate, paraffin and resins) or preserved via freezing. Such assays can be used to guide treatment decisions and determine dosing regimens and timing.

Other particularly compatible aspects of the invention involve the use of in situ hybridization to detect or monitor UPK1B determinants. In situ hybridization technology or ISH is well known to those of skill in the art. Briefly, cells are fixed and detectable probes which contain a specific nucleotide sequence are added to the fixed cells. If the cells contain complementary nucleotide sequences, the probes, which can be detected, will hybridize to them. Using the sequence information set forth herein, probes can be designed to identify cells that express genotypic UPK1B determinants. Probes preferably hybridize to a nucleotide sequence that corresponds to such determinants. Hybridization conditions can be routinely optimized to minimize background signal by non-fully complementary hybridization though preferably the probes are preferably fully complementary to the selected UPK1B determinant. In selected embodiments the probes are labeled with fluorescent dye attached to the probes that is readily detectable by standard fluorescent methodology.

Compatible in vivo theragnostics or diagnostic assays may comprise art-recognized imaging or monitoring techniques such as magnetic resonance imaging, computerized tomography (e.g. CAT scan), positron tomography (e.g., PET scan) radiography, ultrasound, etc., as would be known by those skilled in the art.

In certain embodiments the antibodies of the instant invention may be used to detect and quantify levels of a particular determinant (e.g., UPK1B protein) in a patient sample (e.g., plasma or blood) which may, in turn, be used to detect, diagnose or monitor proliferative disorders that are associated with the relevant determinant. For example, blood and bone marrow samples may be used in conjunction with flow cytometry to detect and measure UPK1B expression (or another co-expressed marker) and monitor the progression of the disease and/or response to treatment. In related embodiments the antibodies of the instant invention may be used to detect, monitor and/or quantify circulating tumor cells either in vivo or in vitro (WO 2012/0128801). In still other embodiments the circulating tumor cells may comprise tumorigenic cells.

In certain embodiments of the invention, the tumorigenic cells in a subject or a sample from a subject may be assessed or characterized using the disclosed antibodies prior to therapy or regimen to establish a baseline. In other examples, the tumorigenic cells can be assessed from a sample that is derived from a subject that was treated.

In another embodiment, the invention provides a method of analyzing cancer progression and/or pathogenesis in vivo. In another embodiment, analysis of cancer progression and/or pathogenesis in vivo comprises determining the extent of tumor progression. In another embodiment, analysis comprises the identification of the tumor. In another embodiment, analysis of tumor progression is performed on the primary tumor. In another embodiment, analysis is performed over time depending on the type of cancer as known to one skilled in the art. In another embodiment, further analysis of secondary tumors originating from metastasizing cells of the primary tumor is conducted in vivo. In another embodiment, the size and shape of secondary tumors are analyzed. In some embodiments, further ex vivo analysis is performed.

In another embodiment, the invention provides a method of analyzing cancer progression and/or pathogenesis in vivo including determining cell metastasis or detecting and quantifying the level of circulating tumor cells. In yet another embodiment, analysis of cell metastasis comprises determination of progressive growth of cells at a site that is discontinuous from the primary tumor. In some embodiments, procedures may be undertaken to monitor tumor cells that disperse via blood vasculature, lymphatics, within body cavities or combinations thereof. In another embodiment, cell metastasis analysis is performed in view of cell migration, dissemination, extravasation, proliferation or combinations thereof.

In certain examples, the tumorigenic cells in a subject or a sample from a subject may be assessed or characterized using the disclosed antibodies prior to therapy to establish a baseline. In other examples the sample is derived from a subject that was treated. In some examples the sample is taken from the subject at least about 1, 2, 4, 6, 7, 8, 10, 12, 14, 15, 16, 18, 20, 30, 60, 90 days, 6 months, 9 months, 12 months, or >12 months after the subject begins or terminates treatment. In certain examples, the tumorigenic cells are assessed or characterized after a certain number of doses (e.g., after 2, 5, 10, 20, 30 or more doses of a therapy). In other examples, the tumorigenic cells are characterized or assessed after 1 week, 2 weeks, 1 month, 2 months, 1 year, 2 years, 3 years, 4 years or more after receiving one or more therapies.

B. Screening

In certain embodiments, antibodies of the instant invention can be used to screen samples in order to identify compounds or agents (e.g., antibodies or ADCs) that alter a function or activity of tumor cells by interacting with a determinant. In one embodiment, tumor cells are put in contact with an antibody or ADC and the antibody or ADC can be used to screen the tumor for cells expressing a certain target (e.g. UPK1B) in order to identify such cells for purposes, including but not limited to, diagnostic purposes, to monitor such cells to determine treatment efficacy or to enrich a cell population for such target-expressing cells.

In yet another embodiment, a method includes contacting, directly or indirectly, tumor cells with a test agent or compound and determining if the test agent or compound modulates an activity or function of the determinant-associated tumor cells for example, changes in cell morphology or viability, expression of a marker, differentiation or de-differentiation, cell respiration, mitochondrial activity, membrane integrity, maturation, proliferation, viability, apoptosis or cell death. One example of a direct interaction is physical interaction, while an indirect interaction includes, for example, the action of a composition upon an intermediary molecule that, in turn, acts upon the referenced entity (e.g., cell or cell culture).

Screening methods include high throughput screening, which can include arrays of cells (e.g., microarrays) positioned or placed, optionally at pre-determined locations, for example, on a culture dish, tube, flask, roller bottle or plate. High-throughput robotic or manual handling methods can probe chemical interactions and determine levels of expression of many genes in a short period of time. Techniques have been developed that utilize molecular signals, for example via fluorophores or microarrays (Mocellin and Rossi, 2007, PMID: 17265713) and automated analyses that process information at a very rapid rate (see, e.g., Pinhasov et al., 2004, PMID: 15032660). Libraries that can be screened include, for example, small molecule libraries, phage display libraries, fully human antibody yeast display libraries (Adimab), siRNA libraries, and adenoviral transfection vectors.

### VII. Pharmaceutical Preparations and Therapeutic Uses

A. Formulations and Routes of Administration

The antibodies or ADCs of the invention can be formulated in various ways using art recognized techniques. In some embodiments, the therapeutic compositions of the invention can be administered neat or with a minimum of additional components while others may optionally be formulated to contain suitable pharmaceutically acceptable carriers. As used herein, “pharmaceutically acceptable carriers” comprise excipients, vehicles, adjuvants and diluents that are well known in the art and can be available from commercial sources for use in pharmaceutical preparation (see, e.g., Gennaro (2003) Remington: The Science and Practice of Pharmacy with Facts and Comparisons: Drugfacts Plus, 20th ed., Mack Publishing; Ansel et al. (2004) Pharmaceutical Dosage Forms and Drug Delivery Systems, 7^{th} ed., Lippencott Williams and Wilkins; Kibbe et al. (2000) Handbook of Pharmaceutical Excipients, 3^{rd} ed., Pharmaceutical Press.)

Suitable pharmaceutically acceptable carriers comprise substances that are relatively inert and can facilitate administration of the antibody or ADC or can aid processing of the active compounds into preparations that are pharmaceutically optimized for delivery to the site of action.

Such pharmaceutically acceptable carriers include agents that can alter the form, consistency, viscosity, pH, tonicity, stability, osmolarity, pharmacokinetics, protein aggregation or solubility of the formulation and include buffering agents, wetting agents, emulsifying agents, diluents, encapsulating agents and skin penetration enhancers. Certain non-limiting examples of carriers include saline, buffered saline, dextrose, arginine, sucrose, water, glycerol, ethanol, sorbitol, dextran, sodium carboxymethyl cellulose and combinations thereof. Antibodies for systemic administration may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington: The Science and Practice of Pharmacy (2000) 20th Ed. Mack Publishing.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection) include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additionally contain other pharmaceutically acceptable carriers, such as anti-oxidants, buffers, preservatives, stabilizers, bacteriostats, suspending agents, thickening agents, and solutes that render the formulation isotonic with the blood (or other relevant bodily fluid) of the intended recipient. Examples of excipients include, for example, water, alcohols, polyols, glycerol, vegetable oils, and the like. Examples of suitable isotonic pharmaceutically acceptable carriers for use in such formulations include Sodium Chloride Injection, Ringer's Solution, or Lactated Ringer's Injection.

In particularly preferred embodiments formulated compositions of the present invention may be lyophilized to provide a powdered form of the antibody or ADC that may then be reconstituted prior to administration. Sterile powders for the preparation of injectable solutions may be generated by lyophilizing a solution comprising the disclosed antibodies or ADCs to yield a powder comprising the active ingredient along with any optional co-solubilized biocompatible ingredients. Generally, dispersions or solutions are prepared by incorporating the active compound into a sterile vehicle that contains a basic dispersion medium or solvent (e.g., a diluent) and, optionally, other biocompatible ingredients. A compatible diluent is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

In certain preferred embodiments the anti-UPK1B antibodies or ADCs will be lyophilized in combination with a pharmaceutically acceptable sugar. A “pharmaceutically acceptable sugar” is a molecule which, when combined with a protein of interest, significantly prevents or reduces chemical and/or physical instability of the protein upon storage. When the formulation is intended to be lyophilized and then reconstituted. As used herein pharmaceutically acceptable sugars may also be referred to as a “lyoprotectant”. Exemplary sugars and their corresponding sugar alcohols include: an amino acid such as monosodium glutamate or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; PLURONICS®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred pharmaceutically-acceptable sugars are the non-reducing sugars trehalose or sucrose. Pharmaceutically acceptable sugars are added to the formulation in a “protecting amount” (e.g. pre-lyophilization) which means that the protein essentially retains its physical and chemical stability and integrity during storage (e.g., after reconstitution and storage).

Whether reconstituted from a lyophilized powder or a native solution, compatible formulations of the disclosed antibodies or ADCs for parenteral administration (e.g., intravenous injection) may comprise ADC or antibody concentrations of from about 10 μg/mL to about 100 mg/mL. In certain selected embodiments antibody or ADC concentrations will comprise 20 μg/mL, 40 μg/mL, 60 μg/mL, 80 μg/mL, 100 μg/mL, 200 μg/mL, 300, pg/mL, 400 μg/mL, 500 μg/mL, 600 μg/mL, 700 μg/mL, 800 μg/mL, 900 μg/mL or 1 mg/mL. In other embodiments ADC concentrations will comprise 2 mg/mL, 3 mg/mL, 4 mg/mL, 5 mg/mL, 6 mg/mL, 8 mg/mL, 10 mg/mL, 12 mg/mL, 14 mg/mL, 16 mg/mL, 18 mg/mL, 20 mg/mL, 25 mg/mL, 30 mg/mL, 35 mg/mL, 40 mg/mL, 45 mg/mL, 50 mg/mL, 60 mg/mL, 70 mg/mL, 80 mg/mL, 90 mg/mL or 100 mg/mL.

In certain preferred aspects compositions of the present invention will comprise a liquid formulation comprising 10 mg/ml UPK1B ADC, 20 mM histidine hydrochloride, 0.175M sucrose, 0.4 mg/mL polysorbate 20 at pH 6.0. In one aspect compositions of the instant invention comprise 10 mg/ml UPK1B ADC, 20 mM histidine hydrochloride, 0.175M sucrose, 0.4 mg/mL polysorbate 20 at pH 6.0. In another aspect compositions of the instant invention comprise 10 mg/ml UPK1B ADC, 20 mM histidine hydrochloride, 0.175M sucrose, 0.4 mg/mL polysorbate 20 at pH 6.0. As discussed herein such liquid formulations may be lyophilized to provide powdered compositions that may be reconstituted with a pharmaceutically compatible (e.g., aqueous) carrier prior to use. When in a liquid solution such compositions should preferably be stored at −70° C. and protected from light. When lyophilized the UPK1B ADC powdered formulations should preferably be stored at 2-8° C. and protected from light. Each of the aforementioned solutions or powders is preferably contained in a sterile glass vial (e.g., USP Type I 10 ml) associated with a label indicating the appropriate storage conditions and may be configured to consistently provide a set volume (e.g., 3 or 5 mL) of 10 mg/mL UPK1B ADC (in a native or reconstituted solution).

Whether reconstituted from lyophilized powder or not, the liquid UPK1B ADC formulations (e.g., as set forth immediately above) may be further diluted (preferably in an aqueous carrier) prior to administration. For example the aforementioned liquid formulations may further be diluted into an infusion bag containing 0.9% Sodium Chloride Injection, USP, or equivalent (mutatis mutandis), to achieve the desired dose level for administration. In certain aspects the fully diluted UPK1B ADC solution will be administered via intravenous infusion using an IV apparatus. Preferably the administered UPK1B ADC drug solution (whether by intravenous (IV) infusion or injection) is clear, colorless and free from visible particulates.

The compounds and compositions of the invention may be administered in vivo, to a subject in need thereof, by various routes, including, but not limited to, oral, intravenous, intra-arterial, subcutaneous, parenteral, intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

B. Dosages and Dosing Regimens

The particular dosage regimen, i.e., dose, timing and repetition, will depend on the particular individual, as well as empirical considerations such as pharmacokinetics (e.g., half-life, clearance rate, etc.). Determination of the frequency of administration may be made by persons skilled in the art, such as an attending physician based on considerations of the condition and severity of the condition being treated, age and general state of health of the subject being treated and the like. Frequency of administration may be adjusted over the course of therapy based on assessment of the efficacy of the selected composition and the dosing regimen. Such assessment can be made on the basis of markers of the specific disease, disorder or condition. In embodiments where the individual has cancer, these include direct measurements of tumor size via palpation or visual observation; indirect measurement of tumor size by x-ray or other imaging techniques; an improvement as assessed by direct tumor biopsy and microscopic examination of a tumor sample; the measurement of an indirect tumor marker (e.g., PSA for prostate cancer) or an antigen identified according to the methods described herein; reduction in the number of proliferative or tumorigenic cells, maintenance of the reduction of such neoplastic cells; reduction of the proliferation of neoplastic cells; or delay in the development of metastasis.

The UPK1B antibodies or ADCs of the invention may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In selected embodiments the UPK1B antibodies or ADCs will be administered (preferably intravenously) at approximately 10, 20, 30, 40, 50, 60, 70, 80, 90 or 100 μg/kg body weight per dose. Other embodiments may comprise the administration of antibodies or ADCs at about 200, 300, 400, 500, 600, 700, 800, 900, 1000, 1100, 1200, 1300, 1400, 1500, 1600, 1700, 1800, 1900 or 2000 μg/kg body weight per dose. In other embodiments the disclosed conjugates will be administered at 2.5, 3, 3.5, 4, 4.5, 5, 5.5, 6, 6.5, 7, 7.5, 8, 9 or 10 mg/kg. In still other embodiments the conjugates may be administered at 12, 14, 16, 18 or 20 mg/kg body weight per dose. In yet other embodiments the conjugates may be administered at 25, 30, 35, 40, 45, 50, 55, 60, 65, 70, 75, 80, 90 or 100 mg/kg body weight per dose. With the teachings herein one of skill in the art could readily determine appropriate dosages for various UPK1B antibodies or ADCs based on preclinical animal studies, clinical observations and standard medical and biochemical techniques and measurements.

Other dosing regimens may be predicated on Body Surface Area (BSA) calculations as disclosed in U.S. Pat. No. 7,744,877. As is well known, the BSA is calculated using the patient's height and weight and provides a measure of a subject's size as represented by the surface area of his or her body. In certain embodiments, the conjugates may be administered in dosages from 1 mg/m² to 800 mg/m², from 50 mg/m² to 500 mg/m² and at dosages of 100 mg/m², 150 mg/m², 200 mg/m², 250 mg/m², 300 mg/m², 350 mg/m², 400 mg/m² or 450 mg/m². It will also be appreciated that art recognized and empirical techniques may be used to determine appropriate dosage.

Anti-UPK1B antibodies or ADCs may be administered on a specific schedule. Generally, an effective dose of the UPK1B conjugate is administered to a subject one or more times. More particularly, an effective dose of the ADC is administered to the subject once a month, more than once a month, or less than once a month. In certain embodiments, the effective dose of the UPK1B antibody or ADC may be administered multiple times, including for periods of at least a month, at least six months, at least a year, at least two years or a period of several years. In yet other embodiments, several days (2, 3, 4, 5, 6 or 7), several weeks (1, 2, 3, 4, 5, 6, 7 or 8) or several months (1, 2, 3, 4, 5, 6, 7 or 8) or even a year or several years may lapse between administration of the disclosed antibodies or ADCs.

In some embodiments the course of treatment involving conjugated antibodies will comprise multiple doses of the selected drug product over a period of weeks or months. More specifically, antibodies or ADCs of the instant invention may administered once every day, every two days, every four days, every week, every ten days, every two weeks, every three weeks, every month, every six weeks, every two months, every ten weeks or every three months. In this regard it will be appreciated that the dosages may be altered or the interval may be adjusted based on patient response and clinical practices. The invention also contemplates discontinuous administration or daily doses divided into several partial administrations. The compositions of the instant invention and anti-cancer agent may be administered interchangeably, on alternate days or weeks; or a sequence of antibody treatments may be given, followed by one or more treatments of anti-cancer agent therapy. In any event, as will be understood by those of ordinary skill in the art, the appropriate doses of chemotherapeutic agents will be generally around those already employed in clinical therapies wherein the chemotherapeutics are administered alone or in combination with other chemotherapeutics.

In another embodiment the UPK1B antibodies or ADCs of the instant invention may be used in maintenance therapy to reduce or eliminate the chance of tumor recurrence following the initial presentation of the disease. Preferably the disorder will have been treated and the initial tumor mass eliminated, reduced or otherwise ameliorated so the patient is asymptomatic or in remission. At such time the subject may be administered pharmaceutically effective amounts of the disclosed antibodies one or more times even though there is little or no indication of disease using standard diagnostic procedures.

In another preferred embodiment the modulators of the present invention may be used to prophylactically or as an adjuvant therapy to prevent or reduce the possibility of tumor metastasis following a debulking procedure. As used in the instant disclosure a “debulking procedure” means any procedure, technique or method that reduces the tumor mass or ameliorates the tumor burden or tumor proliferation. Exemplary debulking procedures include, but are not limited to, surgery, radiation treatments (i.e., beam radiation), chemotherapy, immunotherapy or ablation. At appropriate times readily determined by one skilled in the art in view of the instant disclosure the disclosed ADCs may be administered as suggested by clinical, diagnostic or theragnostic procedures to reduce tumor metastasis.

Yet other embodiments of the invention comprise administering the disclosed antibodies or ADCs to subjects that are asymptomatic but at risk of developing cancer. That is, the antibodies or ADCs of the instant invention may be used in a truly preventative sense and given to patients that have been examined or tested and have one or more noted risk factors (e.g., genomic indications, family history, in vivo or in vitro test results, etc.) but have not developed neoplasia.

Dosages and regimens may also be determined empirically for the disclosed therapeutic compositions in individuals who have been given one or more administration(s). For example, individuals may be given incremental dosages of a therapeutic composition produced as described herein. In selected embodiments the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

C. Combination Therapies

Combination As alluded to above combination therapies may be particularly useful in decreasing or inhibiting unwanted neoplastic cell proliferation, decreasing the occurrence of cancer, decreasing or preventing the recurrence of cancer, or decreasing or preventing the spread or metastasis of cancer. In such cases the antibodies or ADCs of the instant invention may function as sensitizing or chemosensitizing agents by removing CSCs that would otherwise prop up and perpetuate the tumor mass and thereby allow for more effective use of current standard of care debulking or anti-cancer agents. That is, the disclosed antibodies or ADCs may, in certain embodiments, provide an enhanced effect (e.g., additive or synergistic in nature) that potentiates the mode of action of another administered therapeutic agent. In the context of the instant invention “combination therapy” shall be interpreted broadly and merely refers to the administration of an anti-UPK1B antibody or ADC and one or more anti-cancer agents that include, but are not limited to, cytotoxic agents, cytostatic agents, anti-angiogenic agents, debulking agents, chemotherapeutic agents, radiotherapy and radiotherapeutic agents, targeted anti-cancer agents (including both monoclonal antibodies and small molecule entities), BRMs, therapeutic antibodies, cancer vaccines, cytokines, hormone therapies, radiation therapy and anti-metastatic agents and immunotherapeutic agents, including both specific and non-specific approaches.

There is no requirement for the combined results to be additive of the effects observed when each treatment (e.g., antibody and anti-cancer agent) is conducted separately. Although at least additive effects are generally desirable, any increased anti-tumor effect above one of the single therapies is beneficial. Furthermore, the invention does not require the combined treatment to exhibit synergistic effects. However, those skilled in the art will appreciate that with certain selected combinations that comprise preferred embodiments, synergism may be observed.

As such, in certain aspects the combination therapy has therapeutic synergy or improves the measurable therapeutic effects in the treatment of cancer over (i) the anti-UPK1B antibody or ADC used alone, or (ii) the therapeutic moiety used alone, or (iii) the use of the therapeutic moiety in combination with another therapeutic moiety without the addition of an anti-UPK1B antibody or ADC. The term “therapeutic synergy”, as used herein, means the combination of an anti-UPK1B antibody or ADC and one or more therapeutic moiety(ies) having a therapeutic effect greater than the additive effect of the combination of the anti-UPK1B antibody or ADC and the one or more therapeutic moiety(ies).

Desired outcomes of the disclosed combinations are quantified by comparison to a control or baseline measurement. As used herein, relative terms such as “improve,” “increase,” or “reduce” indicate values relative to a control, such as a measurement in the same individual prior to initiation of treatment described herein, or a measurement in a control individual (or multiple control individuals) in the absence of the anti-UPK1B antibodies or ADCs described herein but in the presence of other therapeutic moiety(ies) such as standard of care treatment. A representative control individual is an individual afflicted with the same form of cancer as the individual being treated, who is about the same age as the individual being treated (to ensure that the stages of the disease in the treated individual and the control individual are comparable).

Changes or improvements in response to therapy are generally statistically significant. As used herein, the term “significance” or “significant” relates to a statistical analysis of the probability that there is a non-random association between two or more entities. To determine whether or not a relationship is “significant” or has “significance,” a “p-value” can be calculated. P-values that fall below a user-defined cut-off point are regarded as significant. A p-value less than or equal to 0.1, less than 0.05, less than 0.01, less than 0.005, or less than 0.001 may be regarded as significant.

A synergistic therapeutic effect may be an effect of at least about two-fold greater than the therapeutic effect elicited by a single therapeutic moiety or anti-UPK1B antibody or ADC, or the sum of the therapeutic effects elicited by the anti-UPK1B antibody or ADC or the single therapeutic moiety(ies) of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single therapeutic moiety or anti-UPK1B antibody or ADC, or the sum of the therapeutic effects elicited by the anti-UPK1B antibody or ADC or the single therapeutic moiety(ies) of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

In practicing combination therapy, the anti-UPK1B antibody or ADC and therapeutic moiety(ies) may be administered to the subject simultaneously, either in a single composition, or as two or more distinct compositions using the same or different administration routes. Alternatively, treatment with the anti-UPK1B antibody or ADC may precede or follow the therapeutic moiety treatment by, e.g., intervals ranging from minutes to weeks. In one embodiment, both the therapeutic moiety and the antibody or ADC are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the antibody and the therapeutic moiety.

The combination therapy can be administered until the condition is treated, palliated or cured on various schedules such as once, twice or three times daily, once every two days, once every three days, once weekly, once every two weeks, once every month, once every two months, once every three months, once every six months, or may be administered continuously. The antibody and therapeutic moiety(ies) may be administered on alternate days or weeks; or a sequence of anti-UPK1B antibody or ADC treatments may be given, followed by one or more treatments with the additional therapeutic moiety. In one embodiment an anti-UPK1B antibody or ADC is administered in combination with one or more therapeutic moiety(ies) for short treatment cycles. In other embodiments the combination treatment is administered for long treatment cycles. The combination therapy can be administered via any route.

In selected embodiments the compounds and compositions of the present invention may be used in conjunction with checkpoint inhibitors such as PD-1 inhibitors or PD-L1 inhibitors. PD-1, together with its ligand PD-L1, are negative regulators of the antitumor T lymphocyte response. In one embodiment the combination therapy may comprise the administration of anti-UPK1B antibodies or ADCs together with an anti-PD-1 antibody (e.g. pembrolizumab, nivolumab, pidilizumab) and optionally one or more other therapeutic moiety(ies). In another embodiment the combination therapy may comprise the administration of anti-UPK1B antibodies or ADCs together with an anti-PD-L1 antibody (e.g. avelumab, atezolizumab, durvalumab) and optionally one or more other therapeutic moiety(ies). In yet another embodiment, the combination therapy may comprise the administration of anti-UPK1B antibodies or ADCs together with an anti PD-1 antibody or anti-PD-L1 administered to patients who continue progress following treatments with checkpoint inhibitors and/or targeted BRAF combination therapies (e.g. vemurafenib or dabrafinib).

In some embodiments the anti-UPK1B antibodies or ADCs may be used in combination with various first line cancer treatments. Thus, in selected embodiments the combination therapy comprises the use of an anti-UPK1B antibody or ADC and a cytotoxic agent such as ifosfamide, mytomycin C, vindesine, vinblastine, etoposide, ironitecan, gemcitabine, taxanes, vinorelbine, methotrexate, and pemetrexed) and optionally one or more other therapeutic moiety(ies). In certain neoplastic indications (e.g., hematological indications such as AML or multiple myeloma) the disclosed ADCs may be used in combination with cytotoxic agents such as cytarabine (AraC) plus an anthracycyline (aclarubicin, amsacrine, doxorubicin, daunorubicin, idarubixcin, etc.) or mitoxantrone, fludarabine; hydroxyurea, clofarabine, cloretazine. In other embodiments the ADCs of the invention may be administered in combination with G-CSF or GM-CSF priming, demethylating agents such as azacitidine or decitabine, FLT3-selective tyrosine kinase inhibitors (eg, midostaurin, lestaurtinib and sunitinib), all-trans retinoic acid (ATRA) and arsenic trioxide (where the last two combinations may be particularly effective for acute promyelocytic leukemia (APL)).

In another embodiment the combination therapy comprises the use of an anti-UPK1B antibody or ADC and a platinum-based drug (e.g. carboplatin or cisplatin) and optionally one or more other therapeutic moiety(ies) (e.g. vinorelbine; gemcitabine; a taxane such as, for example, docetaxel or paclitaxel; irinotican; or pemetrexed).

In certain embodiments, for example in the treatment of BR-ERPR, BR-ER or BR-PR cancer, the combination therapy comprises the use of an anti-UPK1B antibody or ADC and one or more therapeutic moieties described as “hormone therapy”. “Hormone therapy” as used herein, refers to, e.g., tamoxifen; gonadotropin or luteinizing releasing hormone (GnRH or LHRH); everolimus and exemestane; toremifene; or aromatase inhibitors (e.g. anastrozole, letrozole, exemestane or fulvestrant).

In another embodiment, for example, in the treatment of BR-HER2, the combination therapy comprises the use of an anti-UPK1B antibody or ADC and trastuzumab or ado-trastuzumab emtansine (Kadcyla) and optionally one or more other therapeutic moiety(ies) (e.g. pertuzumab and/or docetaxel).

In some embodiments, for example, in the treatment of metastatic breast cancer, the combination therapy comprises the use of an anti-UPK1B antibody or ADC and a taxane (e.g. docetaxel or paclitaxel) and optionally an additional therapeutic moiety(ies), for example, an anthracycline (e.g. doxorubicin or epirubicin) and/or eribulin.

In another embodiment, for example, in the treatment of metastatic or recurrent breast cancer or BRCA-mutant breast cancer, the combination therapy comprises the use of an anti-UPK1B antibody or ADC and megestrol and optionally an additional therapeutic moiety(ies).

In further embodiments, for example, in the treatment of BR-TNBC, the combination therapy comprises the use of an anti-UPK1B antibody or ADC and a poly ADP ribose polymerase (PARP) inhibitor (e.g. BMN-673, olaparib, rucaparib and veliparib) and optionally an additional therapeutic moiety(ies).

In another embodiment the combination therapy comprises the use of an anti-UPK1B antibody or ADC and a PARP inhibitor and optionally one or more other therapeutic moiety(ies).

In another embodiment, for example, in the treatment of breast cancer, the combination therapy comprises the use of an anti-UPK1B antibody or ADC and cyclophosphamide and optionally an additional therapeutic moiety(ies) (e.g. doxorubicin, a taxane, epirubicin, 5-FU and/or methotrexate.

In another embodiment combination therapy for the treatment of EGFR-positive NSCLC comprises the use of an anti-UPK1B antibody or ADC and afatinib and optionally one or more other therapeutic moiety(ies) (e.g. erlotinib and/or bevacizumab).

In another embodiment combination therapy for the treatment of EGFR-positive NSCLC comprises the use of an anti-UPK1B antibody or ADC and erlotinib and optionally one or more other therapeutic moiety(ies) (e.g. bevacizumab).

In another embodiment combination therapy for the treatment of ALK-positive NSCLC comprises the use of an anti-UPK1B antibody or ADC and ceritinib and optionally one or more other therapeutic moiety(ies).

In another embodiment combination therapy for the treatment of ALK-positive NSCLC comprises the use of an anti-UPK1B antibody or ADC and crizotinib and optionally one or more other therapeutic moiety(ies).

In another embodiment the combination therapy comprises the use of an anti-UPK1B antibody or ADC and bevacizumab and optionally one or more other therapeutic moiety(ies) (e.g. a taxane such as, for example, docetaxel or paclitaxel; and/or a platinum analog).

In another embodiment the combination therapy comprises the use of an anti-UPK1B antibody or ADC and bevacizumab and optionally one or more other therapeutic moiety(ies) (e.g. gemcitabine and/or a platinum analog).

In one embodiment the combination therapy comprises the use of an anti-UPK1B antibody or ADC and a platinum-based drug (e.g. carboplatin or cisplatin) analog and optionally one or more other therapeutic moiety(ies) (e.g. a taxane such as, for example, docetaxel and paclitaxel).

In one embodiment the combination therapy comprises the use of an anti-UPK1B antibody or ADC and platinum-based drug (e.g. carboplatin or cisplatin) analog and optionally one or more other therapeutic moiety(ies) (e.g. a taxane such, for example, docetaxel and paclitaxel and/or gemcitabine and/or doxorubicin).

In a particular embodiment the combination therapy for the treatment of platinum-resistant tumors comprises the use of an anti-UPK1B antibody or ADC and doxorubicin and/or etoposide and/or gemcitabine and/or vinorelbine and/or ifosfamide and/or leucovorin-modulated 5-fluoroucil and/or bevacizumab and/or tamoxifen; and optionally one or more other therapeutic moiety(ies).

In another embodiment the combination therapy comprises the use of an anti-UPK1B antibody or ADC and a PARP inhibitor and optionally one or more other therapeutic moiety(ies).

In another embodiment the combination therapy comprises the use of an anti-UPK1B antibody or ADC and bevacizumab and optionally cyclophosphamide.

The combination therapy may comprise an anti-UPK1B antibody or ADC and a chemotherapeutic moiety that is effective on a tumor (e.g. melanoma) comprising a mutated or aberrantly expressed gene or protein (e.g. BRAF V600E).

T lymphocytes (e.g., cytotoxic lymphocytes (CTL)) play an important role in host defense against malignant tumors. CTL are activated by the presentation of tumor associated antigens on antigen presenting cells. Active specific immunotherapy is a method that can be used to augment the T lymphocyte response to cancer by vaccinating a patient with peptides derived from known cancer associated antigens. In one embodiment the combination therapy may comprise an anti-UPK1B antibody or ADC and a vaccine to a cancer associated antigen (e.g. WT1.) In other embodiments the combination therapy may comprise administration of an anti-UPK1B antibody or ADC together with in vitro expansion, activation, and adoptive reintroduction of autologous CTLs or natural killer cells. CTL activation may also be promoted by strategies that enhance tumor antigen presentation by antigen presenting cells. Granulocyte macrophage colony stimulating factor (GM-CSF) promotes the recruitment of dendritic cells and activation of dendritic cell cross-priming. In one embodiment the combination therapy may comprise the isolation of antigen presenting cells, activation of such cells with stimulatory cytokines (e.g. GM-CSF), priming with tumor-associated antigens, and then adoptive reintroduction of the antigen presenting cells into patients in combination with the use of anti-UPK1B antibodies or ADCs and optionally one or more different therapeutic moiety(ies).

In some embodiments the anti-UPK1B antibodies or ADCs may be used in combination with various first line melanoma treatments. In one embodiment the combination therapy comprises the use of an anti-UPK1B antibody or ADC and dacarbazine and optionally one or more other therapeutic moiety(ies). In further embodiments the combination therapy comprises the use of an anti-UPK1B antibody or ADC and temozolamide and optionally one or more other therapeutic moiety(ies). In another embodiment the combination therapy comprises the use of an anti-UPK1B antibody or ADC and a platinum-based therapeutic moiety (e.g. carboplatin or cisplatin) and optionally one or more other therapeutic moiety(ies). In some embodiments the combination therapy comprises the use of an anti-UPK1B antibody or ADC and a vinca alkaloid therapeutic moiety (e.g. vinblastine, vinorelbine, vincristine, or vindesine) and optionally one or more other therapeutic moiety(ies). In one embodiment the combination therapy comprises the use of an anti-UPK1B antibody or ADC and interleukin-2 and optionally one or more other therapeutic moiety(ies). In another embodiment the combination therapy comprises the use of an anti-UPK1B antibody or ADC and interferon-alpha and optionally one or more other therapeutic moiety(ies).

In other embodiments, the anti-UPK1B antibodies or ADCs may be used in combination with adjuvant melanoma treatments and/or a surgical procedure (e.g. tumor resection.) In one embodiment the combination therapy comprises the use of an anti-UPK1B antibody or ADC and interferon-alpha and optionally one or more other therapeutic moiety(ies).

The invention also provides for the combination of anti-UPK1B antibodies or ADCs with radiotherapy. The term “radiotherapy”, as used herein, means, any mechanism for inducing DNA damage locally within tumor cells such as gamma-irradiation, X-rays, UV-irradiation, microwaves, electronic emissions and the like. Combination therapy using the directed delivery of radioisotopes to tumor cells is also contemplated, and may be used in combination or as a conjugate of the anti-UPK1B antibodies disclosed herein. Typically, radiation therapy is administered in pulses over a period of time from about 1 to about 2 weeks. Optionally, the radiation therapy may be administered as a single dose or as multiple, sequential doses.

In other embodiments an anti-UPK1B antibody or ADC may be used in combination with one or more of the chemotherapeutic agents described below.

D. Anti-Cancer Agents

The term “anti-cancer agent” as used herein is one subset of “therapeutic moieties”, which in turn is a subset of the agents described as “pharmaceutically active moieties”. More particularly “anti-cancer agent” means any agent (or a pharmaceutically acceptable salt thereof) that can be used to treat a cell proliferative disorder such as cancer, and includes, but is not limited to, cytotoxic agents, cytostatic agents, anti-angiogenic agents, debulking agents, chemotherapeutic agents, radiotherapeutic agents, targeted anti-cancer agents, biological response modifiers, therapeutic antibodies, cancer vaccines, cytokines, hormone therapy, anti-metastatic agents and immunotherapeutic agents. Note that the foregoing classifications of anti-cancer agents are not exclusive of each other and that selected agents may fall into one or more categories. For example, a compatible anti-cancer agent may be classified as a cytotoxic agent and a chemotherapeutic agent. Accordingly, each of the foregoing terms should be construed in view of the instant disclosure and then in accordance with their use in the medical arts.

In preferred embodiments an anti-cancer agent can include any chemical agent (e.g., a chemotherapeutic agent) that inhibits or eliminates, or is designed to inhibit or eliminate, a cancerous cell or a cell likely to become cancerous or generate tumorigenic progeny (e.g., tumorigenic cells). In this regard selected chemical agents (cell-cycle dependent agents) are often directed to intracellular processes necessary for cell growth or division, and are thus particularly effective against cancerous cells, which generally grow and divide rapidly. For example, vincristine depolymerizes microtubules and thus inhibits rapidly dividing tumor cells from entering mitosis. In other cases the selected chemical agents are cell-cycle independent agents that interfere with cell survival at any point of its lifecycle and may be effective in directed therapeutics (e.g., ADCs). By way of example certain pyrrolobenzodiazepines bind to the minor groove of cellular DNA and inhibit transcription upon delivery to the nucleus. With regard to combination therapy or selection of an ADC component it will be appreciated that one skilled in the art could readily identify compatible cell-cycle dependent agents and cell-cycle independent agents in view of the instant disclosure.

In any event, and as alluded to above, it will be appreciated that the selected anti-cancer agents may be administered in combination with each other (e.g., CHOP therapy) in addition to the disclosed anti-UPK1B antibodies and ADCs disclosed herein. Moreover, it will further be appreciated that in selected embodiments such anti-cancer agents may comprise conjugates and may be associated with antibodies prior to administration. In certain embodiments the disclosed anti-cancer agent will be linked to an anti-UPK1B antibody to provide an ADC as disclosed herein.

As used herein the term “cytotoxic agent” (or cytotoxin) generally means a substance that is toxic to cells in that it decreases or inhibits cellular function and/or causes the destruction of tumor cells. In certain embodiments the substance is a naturally occurring molecule derived from a living organism or an analog thereof (purified from natural sources or synthetically prepared). Examples of cytotoxic agents include, but are not limited to, small molecule toxins or enzymatically active toxins of bacteria (e.g., calicheamicin, Diptheria toxin, Pseudomonas endotoxin and exotoxin, Staphylococcal enterotoxin A), fungal (e.g., a-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins [PAPI, PAPII, and PAP-S], Momordica charantia inhibitor, curcin, crotin, saponaria officinalis inhibitor, mitegellin, restrictocin, phenomycin, neomycin, and the tricothecenes) or animals, (e.g., cytotoxic RNases, such as extracellular pancreatic RNases; DNase I, including fragments and/or variants thereof). Additional compatible cytotoxic agents including certain radioisotopes, maytansinoids, auristatins, dolastatins, duocarmycins, amanitins and pyrrolobenzodiazepines are set forth herein.

More generally examples of cytotoxic agents or anti-cancer agents that may be used in combination with (or conjugated to) the antibodies of the invention include, but are not limited to, alkylating agents, alkyl sulfonates, anastrozole, amanitins, aziridines, ethylenimines and methylamelamines, acetogenins, a camptothecin, BEZ-235, bortezomib, bryostatin, callystatin, CC-1065, ceritinib, crizotinib, cryptophycins, dolastatin, duocarmycin, eleutherobin, erlotinib, pancratistatin, a sarcodictyin, spongistatin, nitrogen mustards, antibiotics, enediyne dynemicin, bisphosphonates, esperamicin, chromoprotein enediyne antiobiotic chromophores, aclacinomysins, actinomycin, authramycin, azaserine, bleomycins, cactinomycin, canfosfamide, carabicin, carminomycin, carzinophilin, chromomycinis, cyclosphosphamide, dactinomycin, daunorubicin, detorubicin, 6-diazo-5-oxo-L-norleucine, doxorubicin, epirubicin, esorubicin, exemestane, fluorouracil, fulvestrant, gefitinib, idarubicin, lapatinib, letrozole, lonafamib, marcellomycin, megestrol acetate, mitomycins, mycophenolic acid, nogalamycin, olivomycins, pazopanib, peplomycin, potfiromycin, puromycin, quelamycin, rapamycin, rodorubicin, sorafenib, streptonigrin, streptozocin, tamoxifen, tamoxifen citrate, temozolomide, tepodina, tipifamib, tubercidin, ubenimex, vandetanib, vorozole, XL-147, zinostatin, zorubicin; anti-metabolites, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, polysaccharide complex, razoxane; rhizoxin; SF-1126, sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, chloranbucil; gemcitabine; 6-thioguanine; mercaptopurine; methotrexate; platinum analogs, vinblastine; platinum; etoposide; ifosfamide; mitoxantrone; vincristine; vinorelbine; novantrone; teniposide; edatrexate; daunomycin; aminopterin; xeloda; ibandronate; irinotecan, topoisomerase inhibitor RFS 2000; difluorometlhylomithine; retinoids; capecitabine; combretastatin; leucovorin; oxaliplatin; XL518, inhibitors of PKC-alpha, Raf, H-Ras, EGFR and VEGF-A that reduce cell proliferation and pharmaceutically acceptable salts or solvates, acids or derivatives of any of the above. Also included in this definition are anti-hormonal agents that act to regulate or inhibit hormone action on tumors such as anti-estrogens and selective estrogen receptor antibodies, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts or solvates, acids or derivatives of any of the above.

Compatible cytotoxic agents or anti-cancer agents may also comprise commercially or clinically available compounds such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®). Additional commercially or clinically available anti-cancer agents comprise ibrutinib (IMBRUVICA®, AbbVie) oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafamib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifamib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rlNN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASE® (megestrol acetate), AROMASIN (exemestane; Pfizer), formestanie, fadrozole, RIVISOR® (vorozole), FEMARA® (letrozole; Novartis), and ARIMIDEX® (anastrozole; AstraZeneca).

The term “pharmaceutically acceptable salt” or “salt” means organic or inorganic salts of a molecule or macromolecule. Acid addition salts can be formed with amino groups. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′ methylene bis-(2-hydroxy 3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Where multiple charged atoms are part of the pharmaceutically acceptable salt, the salt can have multiple counter ions. Hence, a pharmaceutically acceptable salt can have one or more charged atoms and/or one or more counterion.

Similarly a “pharmaceutically acceptable solvate” or “solvate” refers to an association of one or more solvent molecules and a molecule or macromolecule. Examples of solvents that form pharmaceutically acceptable solvates include, but are not limited to, water, isopropanol, ethanol, methanol, DMSO, ethyl acetate, acetic acid, and ethanolamine.

In other embodiments the antibodies or ADCs of the instant invention may be used in combination with any one of a number of antibodies (or immunotherapeutic agents) presently in clinical trials or commercially available. The disclosed antibodies may be used in combination with an antibody selected from the group consisting of abagovomab, adecatumumab, afutuzumab, alemtuzumab, altumomab, amatuximab, anatumomab, arcitumomab, atezolizumab, avelumab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, dacetuzumab, dalotuzumab, daratumumab, detumomab, drozitumab, duligotumab, durvalumab, dusigitumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lambrolizumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nivolumab, nofetumomabn, obinutuzumab, ocaratuzumab, ofatumumab, olaratumab, olaparib, onartuzumab, oportuzumab, oregovomab, panitumumab, parsatuzumab, patritumab, pembrolizumab pemtumomab, pertuzumab, pidilizumab, pintumomab, pritumumab, racotumomab, radretumab, ramucirumab, rilotumumab, rituximab, robatumumab, satumomab, selumetinib, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8, MEDIO0680, MDX-1105 and combinations thereof.

Other embodiments comprise the use of antibodies approved for cancer therapy including, but not limited to, rituximab, gemtuzumab ozogamcin, alemtuzumab, ibritumomab tiuxetan, tositumomab, bevacizumab, cetuximab, patitumumab, ofatumumab, ipilimumab and brentuximab vedotin. Those skilled in the art will be able to readily identify additional anti-cancer agents that are compatible with the teachings herein.

E. Radiotherapy

The present invention also provides for the combination of antibodies or ADCs with radiotherapy (i.e., any mechanism for inducing DNA damage locally within tumor cells such as gamma-irradiation, X-rays, UV-irradiation, microwaves, electronic emissions and the like).

Combination therapy using the directed delivery of radioisotopes to tumor cells is also contemplated, and the disclosed antibodies or ADCs may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### VIII. Indications

The invention provides for the use of antibodies and ADCs of the invention for the diagnosis, theragnosis, treatment and/or prophylaxis of various disorders including neoplastic, inflammatory, angiogenic and immunologic disorders and disorders caused by pathogens. In certain embodiments the diseases to be treated comprise neoplastic conditions comprising solid tumors. In other embodiments the diseases to be treated comprise hematologic malignancies. In certain embodiments the antibodies or ADCs of the invention will be used to treat tumors or tumorigenic cells expressing an UPK1B determinant. Preferably the “subject” or “patient” to be treated will be human although, as used herein, the terms are expressly held to comprise any mammalian species.

Neoplastic conditions subject to treatment in accordance with the instant invention may be benign or malignant; solid tumors or hematologic malignancies; and may be selected from the group including, but not limited to: adrenal gland tumors, AIDS-associated cancers, alveolar soft part sarcoma, astrocytic tumors, autonomic ganglia tumors, bladder cancer (squamous cell carcinoma and transitional cell carcinoma), blastocoelic disorders, bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, epithelial disorders, Ewing's tumors, extraskeletal myxoid chondrosarcoma, fibrogenesis imperfecta ossium, fibrous dysplasia of the bone, gallbladder and bile duct cancers, gastric cancer, gastrointestinal, gestational trophoblastic disease, germ cell tumors, glandular disorders, head and neck cancers, hypothalamic, intestinal cancer, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lymphomas (Hodgkin's and Non-Hodgkin's lymphoma), lung cancers (small cell carcinoma, adenocarcinoma, squamous cell carcinoma, large cell carcinoma etc.), macrophagal disorders, medulloblastoma, melanoma, meningiomas, multiple endocrine neoplasia, multiple myeloma including plasmacytoma, localized myeloma, and extramedullary myeloma), myelodysplastic syndrome, myeloproliferative diseases (including myelofibrosis, polycythemia vera, and essential thrombocytopenia) neuroblastoma, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, stromal disorders, synovial sarcoma, testicular cancer, thymic carcinoma, thymoma, thyroid metastatic cancer, and uterine cancers (carcinoma of the cervix, endometrial carcinoma, and leiomyoma).

It will be appreciated that the compounds and compositions of the instant invention may be used to treat subjects at various stages of disease and at different points in their treatment cycle. Accordingly, in certain embodiments the antibodies and ADCs of the instant invention will be used as a front line therapy and administered to subjects who have not previously been treated for the cancerous condition. In other embodiments the antibodies and ADCs of the invention will be used to treat second and third line patients (i.e., those subjects that have previously been treated for the same condition one or two times respectively). Still other embodiments will comprise the treatment of fourth line or higher patients (e.g., gastric or colorectal cancer patients) that have been treated for the same or related condition three or more times with the disclosed UPK1B ADCs or with different therapeutic agents. In other embodiments the compounds and compositions of the present invention will be used to treat subjects that have previously been treated (with antibodies or ADCs of the present invention or with other anti-cancer agents) and have relapsed or are determined to be refractory to the previous treatment. In selected embodiments the compounds and compositions of the instant invention may be used to treat subjects that have recurrent tumors.

In certain embodiments the compounds and compositions of the instant invention will be used as a front line or induction therapy either as a single agent or in combination and administered to subjects who have not previously been treated for the cancerous condition. In other embodiments the compounds and compositions of the present invention will be used during consolidation or maintenance therapy as either a single agent or in combination. In other embodiments the compounds and compositions of the present invention will be used to treat subjects that have previously been treated (with antibodies or ADCs of the present invention or with other anti-cancer agents) and have relapsed or determined to be refractory to the previous treatment. In selected embodiments the compounds and compositions of the instant invention may be used to treat subjects that have recurrent tumors. In other embodiments the compounds and compositions of the present invention will be used as part of a conditioning regimen in preparation of receiving either an autologous or allogeneic hematopoietic stem cell transplant with bone marrow, cord blood or mobilized peripheral blood as the stem cell source.

With regard to hematologic malignancies it will be further be appreciated that the compounds and methods of the present invention may be particularly effective in treating a variety of leukemias including acute myeloid leukemia (AML, cognizant of its various subtypes based on the FAB nomenclature (M0-M7), WHO classification, molecular marker/mutations, karyotype, morphology, and other characteristics), lineage acute lymphoblastic leukemia (ALL), chronic myeloid leukemia (CML), chronic lymphocytic leukemia (CLL), hairy cell leukemia (HCL), chronic myelomonocytic leukemia (CMML), juvenile myelomonocytic leukemia (JMML) and large granular lymphocytic leukemia (LGL) as well as B-cell lymphomas, including Hodgkin's lymphoma (classic Hodgkin's lymphoma and nodular lymphocyte-predominant Hodgkin lymphoma), Non-Hodgkin's lymphoma including diffuse large B-cell lymphoma (DLBCL), follicular lymphoma (FL), low grade/NHL follicular cell lymphoma (FCC), small lymphocytic lymphoma (SLL), mucosa-associated lymphatic tissue (MALT) lymphoma, mantle cell lymphoma (MCL), and Burkitt lymphoma (BL); intermediate grade/follicular NHL, intermediate grade diffuse NHL, high grade immunoblastic NHL, high grade lymphoblastic NHL, high grade small non-cleaved cell NHL, bulky disease NHL, Waldenstrom's Macroglobulinemia, lymphoplasmacytoid lymphoma (LPL), AIDS-related lymphomas, monocytic B cell lymphoma, angioimmunoblastic lymphoadenopathy, diffuse small cleaved cell, large cell immunoblastic lymphoblastoma, small, non-cleaved, Burkitt's and non-Burkitts, follicular, predominantly large cell; follicular, predominantly small cleaved cell; and follicular, mixed small cleaved and large cell lymphomas. See, Gaidono et al., “Lymphomas”, IN CANCER: PRINCIPLES & PRACTICE OF ONCOLOGY, Vol. 2: 2131-2145 (DeVita et al., eds., 5.sup.th ed. 1997). It should be clear to those of skill in the art that these lymphomas will often have different names due to changing systems of classification, and that patients having lymphomas classified under different names may also benefit from the combined therapeutic regimens of the present invention.

In other preferred embodiments the proliferative disorder will comprise a solid tumor including, but not limited to, adrenal, liver, kidney, bladder, breast, gastric, ovarian, cervical, uterine, esophageal, colorectal, prostate, pancreatic, lung (both small cell and non-small cell), thyroid, carcinomas, sarcomas, glioblastomas and various head and neck tumors. In certain selected aspects, and as shown in the Examples below, the disclosed ADCs are especially effective at treating pancreatic cancer. In one embodiment, the pancreatic cancer is refractory, relapsed or resistant to a cytotoxic agent (e.g., irinotecan, gemcitabine, paclitaxeland/or a combination of these agents (e.g. leucovorin, fluorourocil, irinotecan and oxaliplatin).

As indicated the disclosed antibodies and ADCs are especially effective at treating pancreatic cancer. In selected embodiments the antibodies and ADCs can be administered to patients exhibiting limited stage disease or extensive stage disease. In other embodiments the disclosed conjugated antibodies will be administered to refractory patients (i.e., those whose disease recurs during or shortly after completing a course of initial therapy); sensitive patients (i.e., those whose relapse is longer than 2-3 months after primary therapy); or patients exhibiting resistance to a cytotoxic agent (e.g. irinotecan, gemcitabine, paclitaxel) and/or a combination of these agents (e.g. leucovorin, fluorourocil, irinotecan and oxaliplatin). In certain preferred embodiments the UPK1B ADCs of the instant invention may be administered to frontline patients. In other embodiments the UPK1B ADCs of the instant invention may be administered to second line patients. In still other embodiments the UPK1B ADCs of the instant invention may be administered to third line patients.

In particularly preferred embodiments the disclosed ADCs may be used to treat bladder cancer. With regard to such embodiments the conjugated modulators may be administered to patients exhibiting limited stage disease. In other embodiments the disclosed ADCs will be administered to patients exhibiting extensive stage disease. In other preferred embodiments the disclosed ADCs will be administered to refractory patients (i.e., those who recur during or shortly after completing a course of initial therapy) or recurrent bladder cancer patients. Still other embodiments comprise the administration of the disclosed ADCs to sensitive patients (i.e., those whose relapse is longer than 2-3 months after primary therapy. In each case it will be appreciated that compatible ADCs may be used in combination with other anti-cancer agents depending the selected dosing regimen and the clinical diagnosis.

### IX. Articles of Manufacture

The invention includes pharmaceutical packs and kits comprising one or more containers or receptacles, wherein a container can comprise one or more doses of an antibody or ADC of the invention. Such kits or packs may be diagnostic or therapeutic in nature. In certain embodiments, the pack or kit contains a unit dosage, meaning a predetermined amount of a composition comprising, for example, an antibody or ADC of the invention, with or without one or more additional agents and optionally, one or more anti-cancer agents. In certain other embodiments, the pack or kit contains a detectable amount of an anti-UPK1B antibody or ADC, with or without an associated reporter molecule and optionally one or more additional agents for the detection, quantitation and/or visualization of cancerous cells.

In any event kits of the invention will generally comprise an antibody or ADC of the invention in a suitable container or receptacle a pharmaceutically acceptable formulation and, optionally, one or more anti-cancer agents in the same or different containers. The kits may also contain other pharmaceutically acceptable formulations or devices, either for diagnosis or combination therapy. Examples of diagnostic devices or instruments include those that can be used to detect, monitor, quantify or profile cells or markers associated with proliferative disorders (for a full list of such markers, see above). In some embodiments the devices may be used to detect, monitor and/or quantify circulating tumor cells either in vivo or in vitro (see, for example, WO 2012/0128801). In still other embodiments the circulating tumor cells may comprise tumorigenic cells. The kits contemplated by the invention can also contain appropriate reagents to combine the antibody or ADC of the invention with an anti-cancer agent or diagnostic agent (e.g., see U.S. Pat. No. 7,422,739).

When the components of the kit are provided in one or more liquid solutions, the liquid solution can be non-aqueous, though typically an aqueous solution is preferred, with a sterile aqueous solution being particularly preferred. The formulation in the kit can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, phosphate-buffered saline, Ringer's solution or dextrose solution. Where the kit comprises the antibody or ADC of the invention in combination with additional therapeutics or agents, the solution may be pre-mixed, either in a molar equivalent combination, or with one component in excess of the other. Alternatively, the antibody or ADC of the invention and any optional anti-cancer agent or other agent (e.g., steroids) can be maintained separately within distinct containers prior to administration to a patient.

In certain preferred embodiments the aforementioned kits comprising compositions of the invention will comprise a label, marker, package insert, bar code and/or reader indicating that the kit contents may be used for the treatment, prevention and/or diagnosis of cancer. In other preferred embodiments the kit may comprise a label, marker, package insert, bar code and/or reader indicating that the kit contents may be administered in accordance with a certain dosage or dosing regimen to treat a subject suffering from cancer. In a particularly preferred aspect the label, marker, package insert, bar code and/or reader indicates that the kit contents may be used for the treatment, prevention and/or diagnosis of a hematologic malignancy (e.g., AML) or provide dosages or a dosing regimen for treatment of the same. In other particularly preferred aspects the label, marker, package insert, bar code and/or reader indicates that the kit contents may be used for the treatment, prevention and/or diagnosis of lung cancer (e.g., adenocarcinoma) or a dosing regimen for treatment of the same.

Suitable containers or receptacles include, for example, bottles, vials, syringes, infusion bags (i.v. bags), etc. The containers can be formed from a variety of materials such as glass or pharmaceutically compatible plastics. In certain embodiments the receptacle(s) can comprise a sterile access port. For example, the container may be an intravenous solution bag or a vial having a stopper that can be pierced by a hypodermic injection needle.

In some embodiments the kit can contain a means by which to administer the antibody and any optional components to a patient, e.g., one or more needles or syringes (pre-filled or empty), an eye dropper, pipette, or other such like apparatus, from which the formulation may be injected or introduced into the subject or applied to a diseased area of the body. The kits of the invention will also typically include a means for containing the vials, or such like, and other components in close confinement for commercial sale, such as, e.g., blow-molded plastic containers into which the desired vials and other apparatus are placed and retained.

### X. Miscellaneous

Unless otherwise defined herein, scientific and technical terms used in connection with the invention shall have the meanings that are commonly understood by those of ordinary skill in the art. Further, unless otherwise required by context, singular terms shall include pluralities and plural terms shall include the singular. In addition, ranges provided in the specification and appended claims include both end points and all points between the end points. Therefore, a range of 2.0 to 3.0 includes 2.0, 3.0, and all points between 2.0 and 3.0.

Generally, techniques of cell and tissue culture, molecular biology, immunology, microbiology, genetics and chemistry described herein are those well-known and commonly used in the art. The nomenclature used herein, in association with such techniques, is also commonly used in the art. The methods and techniques of the invention are generally performed according to conventional methods well known in the art and as described in various references that are cited throughout the present specification unless otherwise indicated.

### XI. References

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PBD, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described.

### EXAMPLES

The invention, generally described above, will be understood more readily by reference to the following examples, which are provided by way of illustration and are not intended to be limiting of the instant invention. The examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

### Sequence Listing Summary

TABLE 3 provides a summary of amino acid and nucleic acid sequences included herein.

TABLE 3

| SEQ ID NO | Description |
|---|---|
| 1 | Amino acid sequence of UPK1B. |
| 2 | IgG1 heavy chain constant region protein |
| 3 | C220S IgG1 heavy constant region protein |
| 4 | C220Δ IgG1 heavy constant region protein |
| 5 | kappa light chain constant region protein |
| 6 | C214S kappa light chain constant region protein |
| 7 | C214Δ kappa light chain constant region protein |
| 8 | lambda light chain constant region protein |
| 9 | C214S lambda light chain constant region protein |
| 10 | C214Δ lambda light chain constant region protein |
| 11-19 | Reserved |
| 20 | SC115.1 VL DNA |
| 21 | SC115.1 VL protein |
| 22 | SC115.1 VH DNA |
| 23 | SC115.1 VH protein |
| 24-91 | Additional murine clones in the same order as |
| | SEQ ID NOS 20-23 |
| 92-99 | Reserved |
| 100 | hSC115.9 VL DNA |
| 101 | hSC115.9 VL protein |
| 102 | hSC115.9 VH DNA |
| 103 | hSC115.9 VH protein |
| 104 | hSC115.18 VL DNA |
| 105 | hSC115.18 VL protein |
| 106 | hSC115.18 VH DNA |
| 107 | hSC115.18 VH protein |
| 108-109 | Reserved |
| 110 | hSC115.9 full length light chain protein |
| 111 | hSC115.9 full length heavy chain protein |
| 112 | hSC115.9ss1 full length heavy chain protein |
| 113 | hSC115.18 full length light chain protein |
| 114 | hSC115.18 full length heavy chain protein |
| 115 | hSC115.18ss1 full length heavy chain protein |

### Tumor Cell Line Summary

PDX tumor cell types are denoted by an abbreviation followed by a number, which indicates the particular tumor cell line. The passage number of the tested sample is indicated by p0-p# appended to the sample designation where p0 is indicative of an unpassaged sample obtained directly from a patient tumor and p# is indicative of the number of times the tumor has been passaged through a mouse prior to testing. As used herein, the abbreviations of the tumor types and subtypes are shown in TABLE 4 as follows:

TABLE 4

| Tumor Type | Abbreviation | Tumor subtype | Abbreviation |
|---|---|---|---|
| Acute | AML | | |
| myelogenous | | | |
| leukemia | | | |
| Bladder | BL | | |
| Breast | BR | | |
| | | basal-like | BR-Basal-Like |
| | | estrogen receptor | BR-ERPR |
| | | positive and/or | |
| | | progesterone | |
| | | receptor | |
| | | positive | |
| | | ERBB2/Neu | BR-ERBB2/Neu |
| | | positive | |
| | | HER2 positive | BR-HER2 |
| | | triple-negative | TNBC |
| | | luminal A | BR-LumA |
| | | luminal B | BR-LumB |
| | | claudin subtype of | TNBC-CL |
| | | triple-negative | |
| | | claudin low | BR-CLDN-Low |
| | | normal-like | BR-NL |
| Cervical | CER | | |
| Colorectal | CR | | |
| | | rectum adeno- | RE-Ad |
| | | carcinoma | |
| Endometrial | EM | | |
| Esophageal | ES | | |
| Gastric | GA | | |
| | | diffuse adeno- | GA-Ad-Dif/Muc |
| | | carcinoma | |
| | | intestinal adeno- | GA-Ad-Int |
| | | carcinoma | |
| | | stromal tumors | GA-GIST |
| Glioblastoma | GB | | |
| Head and neck | HN | head and neck | HNSC |
| | | squamous cell | |
| | | carcinoma | |
| Kidney | KDY | | |
| | | clear renal cell | KDY-CC |
| | | carcinoma | |
| | | papillary renal | KDY-PAP |
| | | cell carcinoma | |
| | | transitional cell | KDY-URO |
| | | or urothelial | |
| | | carcinoma | |
| | | unknown | KDY-UNK |
| Liver | LIV | | |
| | | hepatocellular | LIV-HCC |
| | | carcinoma | |
| | | cholangio- | LIV-CHOL |
| | | carcinoma | |
| Lymphoma | LYM | | |
| | DLBC | diffuse large B-cell | |
| Lung | LU | | |
| | | adenocarcinoma | LU-Ad |
| | | carcinoid | LU-CAR |
| | | large cell neuro- | LU-LCC |
| | | endocrine | |
| | | non-small cell | NSCLC |
| | | squamous cell | LU-SCC |
| | | small cell | SCLC |
| | | spindle cell | LU-SPC |
| | | mesothelioma | MESO |
| Multiple Myeloma | MM | | |
| Ovarian | OV | | |
| | | clear cell | OV-CC |
| | | endometroid | OV-END |
| | | mixed subtype | OV-MIX |
| | | malignant mixed | OV-MMMT |
| | | mesodermal | |
| | | mucinous | OV-MUC |
| | | neuroendocrine | OV-NET |
| | | papillary serous | OV-PS |
| | | serous | OV-S |
| | | small cell | OV-SC |
| | | transitional cell | OV-TCC |
| | | carcinoma | |
| Pancreatic | PA | | |
| | | acinar cell | PA-ACC |
| | | carcinoma | |
| | | duodenal | PA-DC |
| | | carcinoma | |
| | | mucinous adeno- | PA-MAD |
| | | carcinoma | |
| | | neuroendocrine | PA-NET |
| | | adenocarcinoma | PA-PAC |
| | | adenocarcinoma | PA-PACe |
| | | exocrine type | |
| | | ductal adeno- | PA-PDAC |
| | | carcinoma | |
| | | ampullary adeno- | PA-AAC |
| | | carcinoma | |
| Prostate | PR | | |
| Skin | SK | | |
| | | melanoma | MEL |
| | | squamous cell | SK-SCC |
| | | carcinomas | |
| | | uveal melanoma | UVM |
| Testicular | TES | | |
| Thyroid | THY | | |
| | | medullary thyroid | MTC |
| | | carcinoma | |
| Uterine | UCEC | uterine corpus endo- | |
| | | metrial carcinoma | |

### Example 1

### Identification of UPK1B Expression Using Whole Transcriptome Sequencing

To characterize the cellular heterogeneity of solid tumors as they exist in cancer patients and identify clinically relevant therapeutic targets, a large PDX tumor bank was developed and maintained using art recognized techniques. The PDX tumor bank, comprising a large number of discrete tumor cell lines, was propagated in immunocompromised mice through multiple passages of tumor cells originally obtained from cancer patients afflicted by a variety of solid tumor malignancies. Low passage PDX tumors are representative of tumors in their native environments, providing clinically relevant insight into underlying mechanisms driving tumor growth and resistance to current therapies.

As previously alluded to tumor cells may be divided broadly into two types of cell subpopulations: non-tumorigenic cells (NTG) and tumor initiating cells (TICs). TICs have the ability to form tumors when implanted into immunocompromised mice. Cancer stem cells (CSCs) are a subset of TICs that are able to self-replicate indefinitely while maintaining the capacity for multilineage differentiation. NTGs, while sometimes able to grow in vivo, will not form tumors that recapitulate the heterogeneity of the original tumor when implanted.

In order to perform whole transcriptome analysis, PDX tumors were resected from mice after they reached 800-2,000 mm³ or for AML after the leukemia was established in the bone marrow (<5% of bone marrow cellularity of human origin). Resected PDX tumors were dissociated into single cell suspensions using art-recognized enzymatic digestion techniques (see, for example, U.S.P.N. 2007/0292414). Dissociated bulk tumor cells were incubated with 4′,6-diamidino-2-phenylindole (DAPI) to detect dead cells, anti-mouse CD45 and H-2K^{d} antibodies to identify mouse cells and anti-human EPCAM antibody to identify human cells. In addition the tumor cells were incubated with fluorescently conjugated anti-human CD46 and/or CD324 antibodies to identify CD46^{hi}CD324⁺ CSCs or CD46^{lo/-}CD324⁻ NTG cells and were then sorted using a FACSAria cell sorter (BD Biosciences) (see U.S.P.Ns 2013/0260385, 2013/0061340 and 2013/0061342).

RNA was extracted from tumor cells by lysing the cells in RLTplus RNA lysis buffer (Qiagen) supplemented with 1% 2-mercaptoethanol, freezing the lysates at −80° C. and then thawing the lysates for RNA extraction using an RNeasy isolation kit (Qiagen). RNA was quantified using a Nanodrop spectrophotometer (Thermo Scientific) and/or a Bioanalyzer 2100 (Agilent Technologies). Normal tissue RNA was purchased from various sources (Life Technology, Agilent, ScienCell, BioChain, and Clontech). The resulting total RNA preparations were assessed by genetic sequencing and gene expression analyses.

More particularly whole transcriptome sequencing of high quality RNA was performed using two different systems. Some samples were analyzed using Applied Biosystems (ABI) Sequencing by Oligo Ligation/Detection (SOLiD) 4.5 or SOLID 5500×1 next generation sequencing system (Life Technologies). Other samples were analyzed using Illumina HiSeq 2000 or 2500 next generation sequencing system (Illumina).

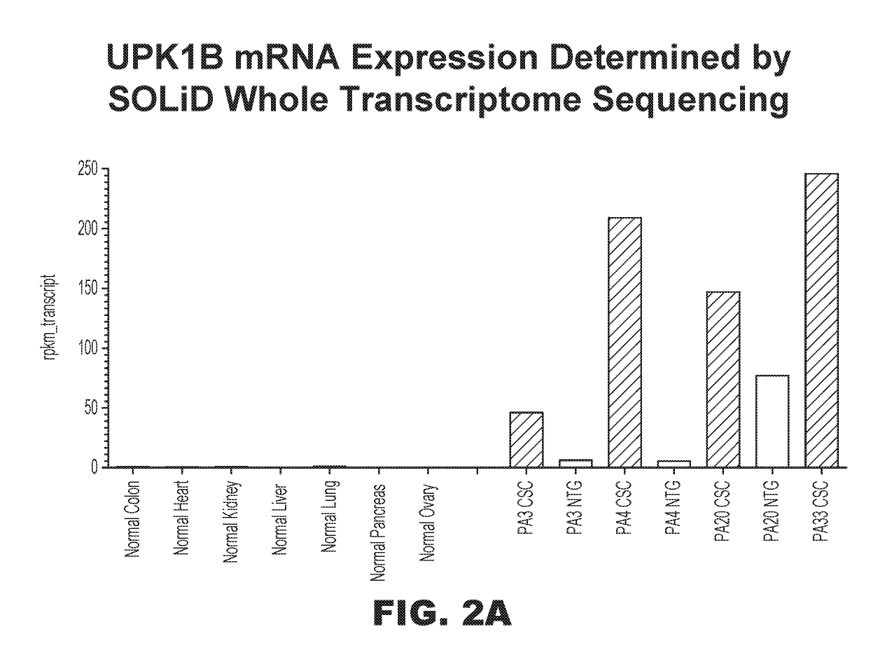

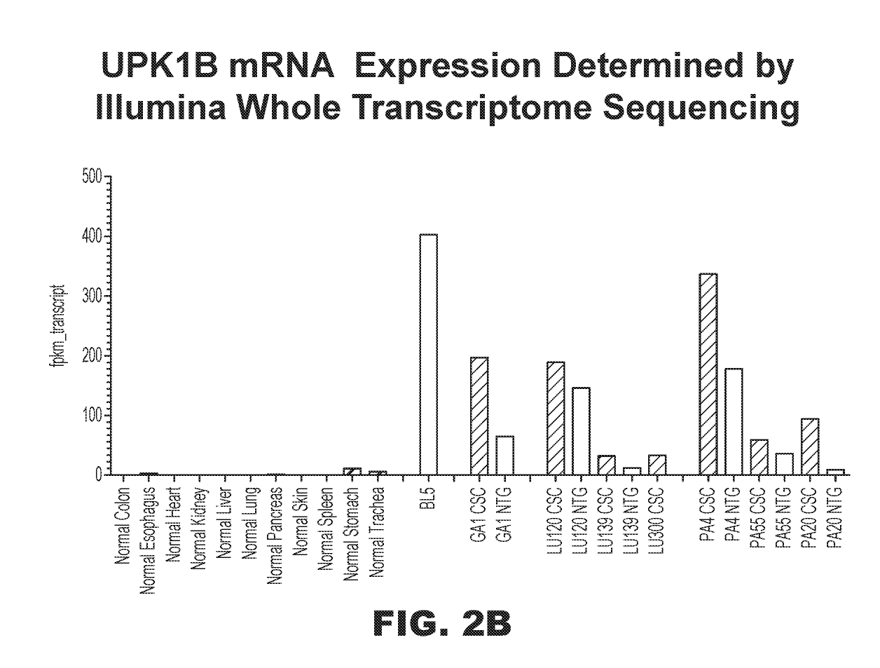

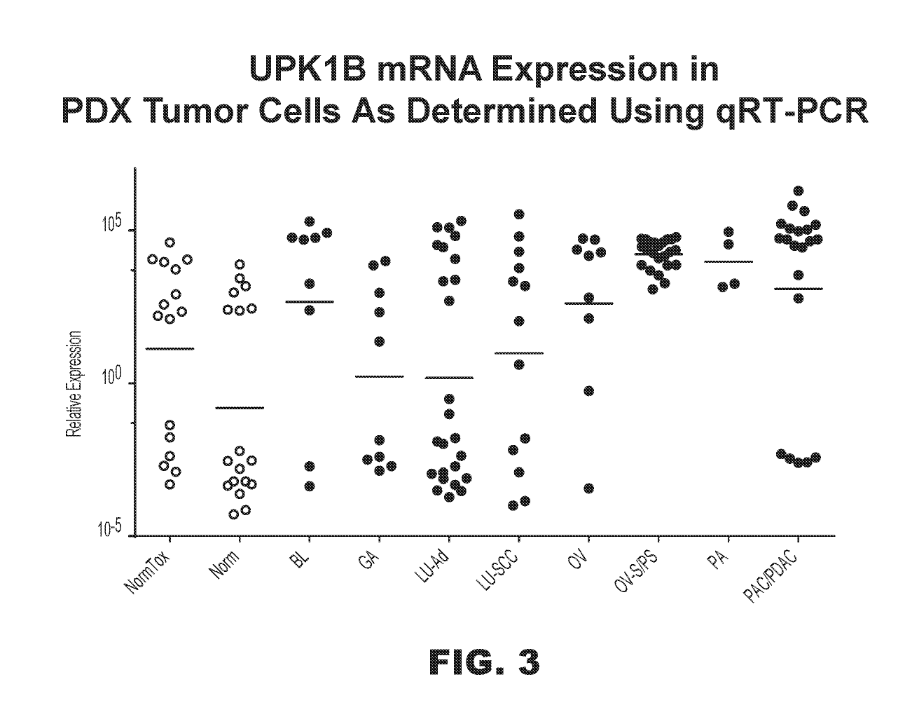

SOLID whole transcriptome analysis was performed with cDNA that was generated from 1 ng total RNA from bulk tumor samples using either a modified whole transcriptome protocol from ABI designed for low input total RNA or the Ovation RNA-Seq System V2™ (NuGEN Technologies). The resulting cDNA library was fragmented, and barcode adapters were added to allow pooling of fragment libraries from different samples during sequencing runs. Data generated by the SOUD platform mapped to 34,609 genes as annotated by RefSeq version 47 using NCBI version hg19.2 of the published human genome and provided verifiable measurements of RNA levels in most samples. Sequencing data from the SOLID platform is nominally represented as a transcript expression value using the metrics RPM (reads per million) or RPKM (read per kilobase per million) mapped to exon regions of genes, enabling basic gene expression analysis to be normalized and enumerated as RPM_Transcript or RPKM_Transcript UPK1B mRNA was elevated in PA CSC populations (black bars) when compared to corresponding NTG samples (empty bars) and normal tissues (gray bars) (FIG. 2A)

Illumina whole transcriptome analysis was performed with cDNA that was generated using 5 ng total RNA extracted from either NTG or CSC tumor subpopulations that were isolated as described above. The library was created using the TruSeq RNA Sample Preparation Kit v2 (Illumina, Inc.). The resulting cDNA library was fragmented and barcoded. Sequencing data from the Illumina platform is nominally represented as a fragment expression value using the metric FPKM (fragment per kilobase per million) mapped to exon regions of genes, enabling basic gene expression analysis to be normalized and enumerated as FPKM transcript. As shown in FIG. 2B UPK1B mRNA expression in the PA, LU, and GA CSC cancer stem cell subpopulation (black bars) was generally higher than expression in both normal cells (grey bars) and the NTG cell populations (white bars). In addition, BL5 bulk tumor showed high expression of UPK1B mRNA (FIG. 2B).

The identification of elevated UPK1B mRNA expression in LU, PA, and GA tumor CSC populations indicates that UPK1B merits further evaluation as a potential diagnostic and immunotherapeutic target. Furthermore, increased expression of UPK1B in CSC compared to NTG in LU, PA and GA PDX tumors indicates that UPK1B is a good marker of tumorigenic cells in these tumor types.

### Example 2

### Expression of UPK1B mRNA in Tumors Using qRT-PCR

To confirm UPK1B RNA expression in tumor cells, qRT-PCR was performed on various PDX cell lines using the Fluidigm BioMark™ HD System according to industry standard protocols. RNA was extracted from bulk PDX tumor cells or sorted CSC and NTG subpopulations as described in Example 1. 1.0 ng of RNA was converted to cDNA using the High Capacity cDNA Archive kit (Life Technologies) according to the manufacturer's instructions. cDNA material, pre-amplified using an UPK1B probe specific Taqman assay, was then used for subsequent qRT-PCR experiments.

UPK1B expression in normal tissues (NormTox or Norm) was compared to expression in BL, GA, LU-Ad, LU-SCC, OV, and PAC/PDAC PDX tumor cell lines (FIG. 3; each dot represents the average relative expression of each individual tissue or PDX cell line, with the small horizontal line representing the geometric mean). “NormTox” represents samples of various normal tissues as follows: adrenal, colon (whole organ, sorted epithelial, stromal fibroblasts, vascular endothelial and blood cells), dorsal root ganglion, endothelial cells (artery, vein), esophagus (whole organ and sorted epithelial, smooth muscle, basal and transient amplifying cells), heart, kidney (whole organ and sorted epithelial cells, distal and proximal tubule, and progenitors), liver, lung (whole organ and sorted epithelial and blood cells), pancreas, skeletal muscle, skin (whole organ and sorted epithelial cells, differentiated epithelial cells, transient amplifying cells, fibroblasts, and keratinocytes), small intestine, spleen, stomach, and trachea (whole organ and sorted epithelial cells). Another set of normal tissues designated “Norm” represents the following samples of normal tissue with a presumed lower risk for toxicity in relation to ADC-type drugs: peripheral blood mononuclear cells and various sorted subpopulations (B cells, monocytes, NK cells, neutrophils, T cells), adipose, bladder, brain, breast, cervix, melanocytes, normal bone marrow and various sorted subpopulations, ovary, prostate, salivary gland, testes, and thymus.

A review of FIG. 3 shows that, on average, UPK1B expression was elevated in PA and OV-S/PS and in subsets of BL, GA, LU-Ad, LU-SCC, and OV, with the geometric mean being lower overall in GA, LU-Ad and LU-SCC. This data supports the earlier finding of elevated expression of UPK1B in PA, OV-S/PS and in selected BL, GA, LU-Ad, LU-SCC, and other OV PDX compared to most normal tissues.

### Example 3

### Determination of Expression of UPK1B mRNA in Tumors Using Microarray Analysis

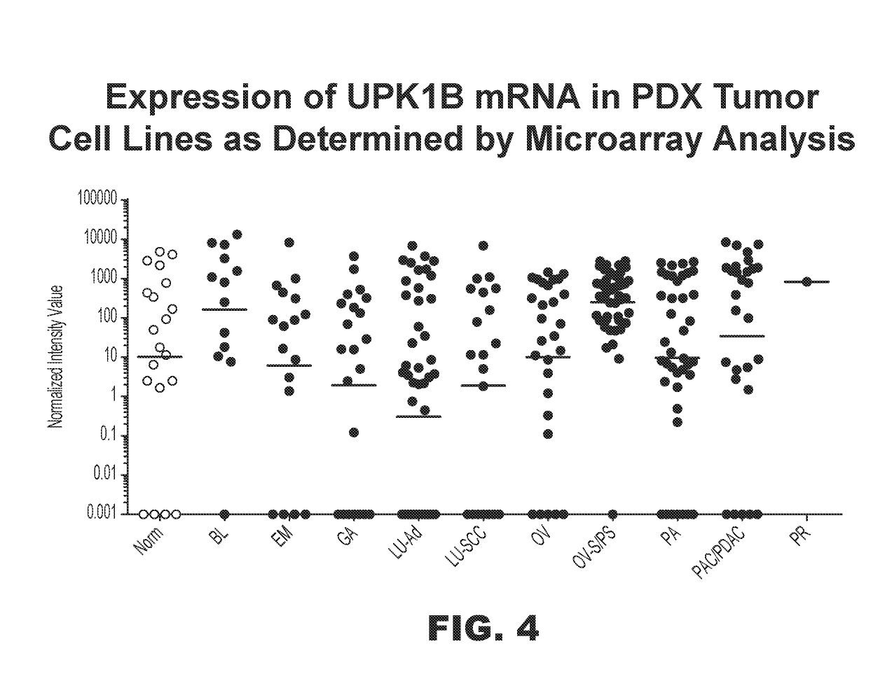

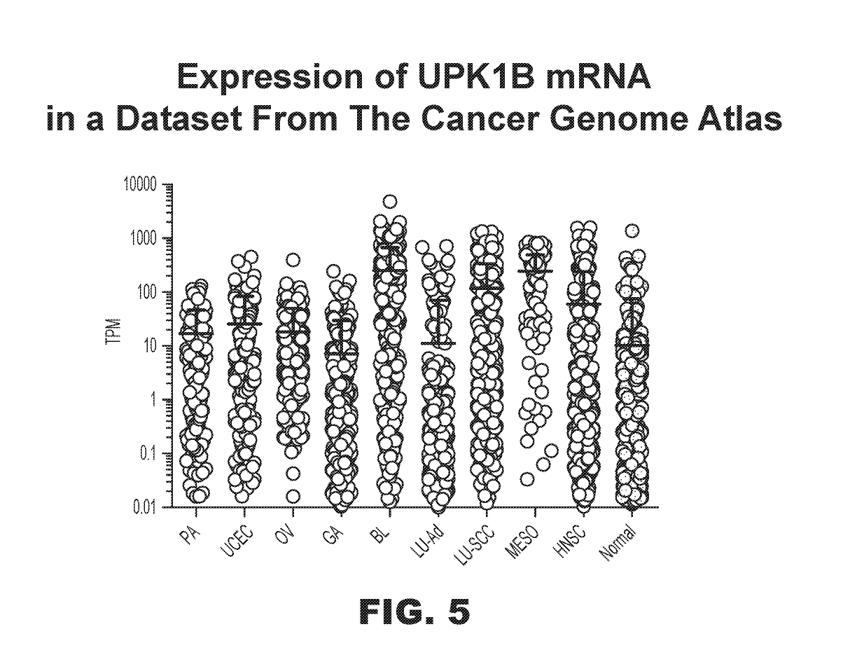

Microarray experiments to determine the expression levels of UPK1B in various tumor cell lines were conducted and data was analyzed as follows. 1-2 μg of whole tumor total RNA was extracted, substantially as described in Example 1, from BL, EM, GA, LU-Ad, LU-SCC, OV, PAC/PDAC, and PR cell lines. Additionally, RNA was extracted from samples of normal tissues (e.g., bladder, breast, colon, heart, kidney, liver, lung, ovary, pancreas, skin, spleen, PBMC, and stomach). The RNA samples were analyzed using the Agilent SurePrint GE Human 8×60 v2 microarray platform, which contains 50,599 biological probes designed against 27,958 genes and 7,419 IncRNAs in the human genome. Standard industry practices were used to normalize and transform the intensity values to quantify gene expression for each sample. The normalized intensity of UPK1B expression in each sample is plotted in FIG. 4 and the geometric mean derived for each tumor type is indicated by the horizontal bar.

A closer review of FIG. 4 shows that UPK1B expression is upregulated in most PA, BL, and OV tumor cell lines and a substantial subset of tumor samples of LU-Ad, LU-SCC, GA, EM and PR compared to normal tissues. The observation of elevated UPK1B expression in the aforementioned tumor types confirms the results of the previous Examples. In particular PA and OV tumor samples analyzed on all three platforms show substantially elevated UPK1B expression. More generally these data demonstrate that UPK1B is expressed in a large fraction of a number of tumor subtypes including LU-Ad, LU-SCC, BL, and GA, and may be a good target for the development of an antibody-based therapeutic in these indications.

### Example 4

### UPK1B Expression in Tumors Using the Cancer Genome Atlas

Overexpression of hUPK1B mRNA in various tumors was confirmed using a large, publically available dataset of primary tumors and normal samples known as The Cancer Genome Atlas (TCGA).

More specifically hUPK1B expression data from the IlluminaHiSeq_RNASeqV2 platform was downloaded from the Genomic Data Commons (GDC) Legacy Archive (https://gdc-portal.nci.nih.gov/legacy-archivel and the scaled_estimate from RSEM was multiplied by 1,000,000 to yield transcripts per million (TPM) [Li and Dewey, BMC Bioinformatics 2011]. FIG. 5 shows that UPK1B expression is elevated in PA, LU-Ad, LU-SCC, OV, BL, MESO, HNSC, and GA primary patient samples compared to normal tissue. These data further confirm that elevated levels of UPK1B mRNA may be found in various tumor types, indicating that anti-UPK1B antibodies and ADCs may be useful therapeutics for these tumors.

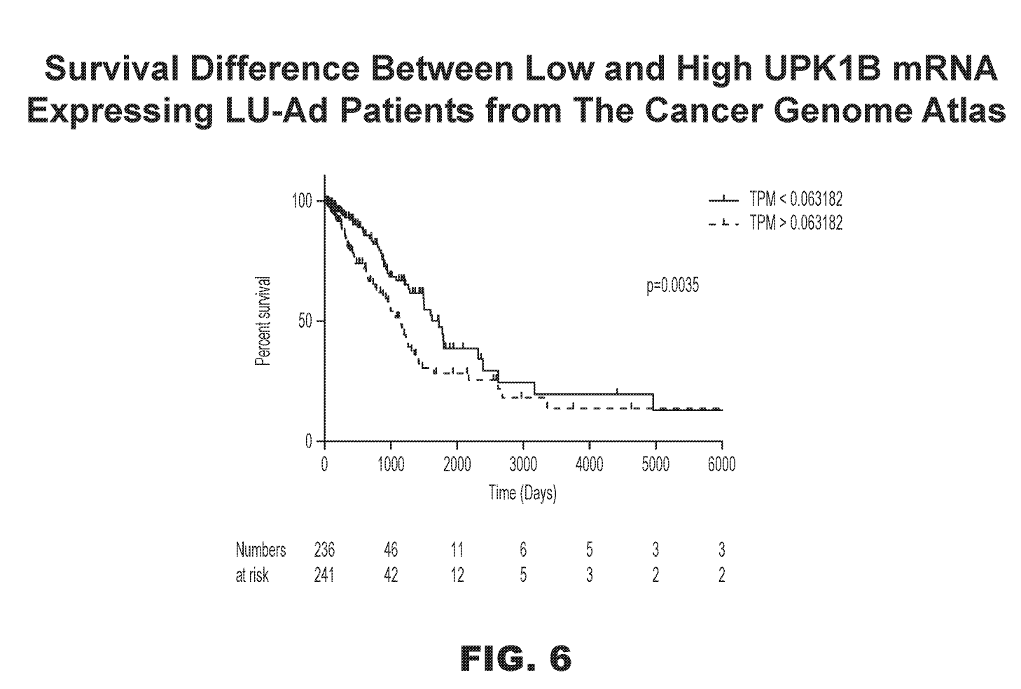

FIG. 6 shows Kaplan Meier survival curves for a subset of LU-Ad TCGA tumors where patient survival data was available. Patients were stratified based on high expression of UPK1B mRNA i.e. expression over the threshold index value or low expression of UPK1B mRNA i.e. expression under the threshold index value in LU-Ad tumors. The threshold index value was calculated as the median of the TPM values, which was calculated to be 0.063.

The “numbers at risk” listed below the plot shows the number of surviving patients remaining in the dataset every 1000 days after the day at which each patient was first diagnosed (day 0). The two survival curves are significantly different (p=0.0035) by the Log-rank (Mantel-Cox) test or p=0.0014 by the Gehan-Breslow-Wilcoxon test. These data show that patients with LU-Ad tumors exhibiting high expression of UPK1B have a shorter survival time compared to patients with LU-Ad tumors exhibiting low expression of UPK1B. This suggests the usefulness of anti-UPK1B therapies to treat LU-Ad, and the usefulness of UPK1B expression as a prognostic biomarker on the basis of which treatment decisions can be made.

### Example 5

### Cloning and Expression of Recombinant UPK1B Proteins and Engineering of Cell Lines Overexpressing Cell Surface UPK1B Proteins

Human UPK1B (hUPK1B) Lentiviral DNA Constructs

To generate cell lines overexpressing full length hUPK1B protein, lentiviral vectors containing an open reading frame encoding the hUPK1B protein (derived from NCBI accession NM_006952) were constructed by subcloning a codon-optimized, synthetic DNA fragment (GeneArt) into the multiple cloning site of the lentiviral vector pCDH-CMV-MCS-EF1-copGFP (System Biosciences), to yield pLMEGPA-hUPK1B. This dual promoter construct employs a CMV promoter to drive expression of hUPK1B independent of a downstream EF1 promoter that drives expression of the copGFP T2A Puro reporter and selectable marker. The T2A sequence promotes ribosomal skipping of a peptide bond condensation, resulting in expression of two independent proteins: high level expression of the reporter copGFP upstream of the T2A peptide, with co-expression of the Puro selectable marker protein downstream of the T2A peptide to allow selection of transduced cells in the presence of puromycin.

DNA Constructs Encoding hUPK1B Extracellular Domain Fusion Proteins.

To produce immunogens that may be used to generate immunoreactive antibodies to the ECD of the hUPK1B protein, chimeric fusion genes encoding the second extracellular domain of the hUPK1B protein (e.g., amino acids T108-H229 from the NCBI reference sequence NP_008883) were created as follows. A PCR product encoding the indicated UPK1B amino acid residues was amplified from the pLMEGPA-hUPK1B template, and the resultant DNA fragment subcloned into a CMV-driven expression vector in-frame and downstream of an immunoglobulin kappa (IgK) signal peptide sequence and upstream and in-frame with DNA encoding either a 9×-Histidine tag (yielding phUPK1B(108-229)-His) or a human IgG2 Fc protein (yielding phUPK1B(108-229)-Fc), using standard molecular techniques. These CMV-driven expression vectors permit high level transient expression in HEK293T and/or CHO-S cells.

Cynomolgus UPK1B (cUPK1B) and Rat UPK1B (rUPK1B) DNA Constructs

To generate cell lines overexpressing full length cUPK1B or rUPK1B protein, lentiviral vectors containing an open reading frame encoding either the cUPK1B or rUPK1B protein were constructed by subcloning codon-optimized, synthetic DNA fragments (GeneArt) of cUPK1B (derived from NCBI accession XM_00548075) or rUPK1B (derived from NCBI accession NM_001024253) into the multiple cloning site of the lentiviral vector pCDH-CMV-MCS-EF1-copGFP (System Biosciences), to yield pLMEGPA-cUPK1B or pLMEGPA-rUPK1B, respectively.

To generate soluble, recombinant proteins pertaining to the second extracellular domain of the cUPK1B (e.g., T108-H229) or rUPK1B (e.g., T108-H229) proteins, gBlock DNA fragments (IDT) encoding these residues were synthesized and subcloned directly into a CMV driven expression vector in-frame and downstream of an IgK signal peptide sequence and upstream of either a 9×-Histidine tag or a human IgG2 Fc cDNA. The resulting constructs were called pcUPK1B-His, pcUPK1B-Fc, prUPK1B-His, or prUPK1B-Fc respectively.

UPK1B Fusion Protein Production

Suspension or adherent cultures of HEK293T cells, or suspension CHO-S cells were transfected with an expression construct selected from one of the following: phUPK1B(108-229)-His, phUPK1B(108-229)-Fc, pcUPK1B-His, pcUPK1B-Fc, prUPK1B-His, or prUPK1B-Fc, using polyethylenimine polymer as the transfecting reagent. Three to five days after transfection, the His or Fc fusion proteins were purified from clarified cell-supematants using either Nickel-EDTA (Qiagen) or MabSelect SuRe™ Protein A (GE Healthcare Life Sciences) columns as appropriate to the tag, per manufacturer's instructions.

Cell Line Engineering

Three lentiviral vectors—pLMEGPA-hUPK1B, pLMEGPA-cUPK1B, or pLMEGPA-rUPK1B-were used to create stable HEK293T-based cell lines overexpressing hUPK1B cUPK1B, or rUPK1B proteins, respectively, using standard lentiviral transduction techniques well known to those skilled in the art. Transduced cells were selected using puromycin, followed by fluorescent activated cell sorting (FACS) of high-expressing HEK293T subclones (e.g., cells that were strongly positive for GFP).

### Example 6

### Generation of Anti-UPK1B Antibodies

Anti-UPK1B mouse antibodies were produced by inoculating two BALB/c mice, two CD-1 mice, and two FVB mice with 10 μg hUPK1B protein, emulsified with an equal volume of TiterMax^{e}Gold Adjuvant (Sigma Aldrich #H4 T2684-1ML, lot#MKBT701V). Following the initial inoculation, the mice were injected at weekly intervals, 9 times with 10 μg hUPK1b protein emulsified with an equal volume of Imject® Alum (ThermoScientific #77161) plus “CpG” (InvivoGen ODN1826 #tlr1-1826-1). The final injection prior to the fusion was with 10 μg hUPK1B in PBS.

Mice were sacrificed, and draining lymph nodes (popliteal, inguinal, and medial iliac) were dissected and used as a source for antibody producing cells. A single-cell suspension of B cells was produced and (300×10⁶ cells) were fused with non-secreting SP2/0-Ag14 myeloma cells (ATCC # CRL-1581) at a ratio of 1:1 by electro cell fusion using a model BTX Hybrimmune System (BTX Harvard Apparatus). Cells were re-suspended in hybridoma selection medium consisting of DMEM medium supplemented with azaserine, 15% fetal clone I serum (Thermo #SH30080-03), 10% BM condimed (Roche #10663573001, lot#10557500), 1 mM nonessential amino acids (Corning #25-025-CI) 1 mM HEPES Corning #25-060-CI), 100 IU penicillin-streptomycin (Corning #30-002-CI), 100 IU L-glutamine (Corning #25-005-CI) and were cultured in three T225 flasks containing 100 mL selection medium. The flasks were placed in a humidified 37° C. incubator containing 7% CO₂ and 95% air for 6 days.

On days 6 and 7 after the fusion, the Hybridoma cells were sorted from the flask and plated at one cell per well (using a BD FACSAria cell sorter) in 90 pL of supplemented hybridoma selection medium (as described above) into 12 Falcon 384-well plates. Remaining unused hybridoma library cells were frozen in liquid nitrogen for future library testing and screening.

Sorted clonal hybridomas were cultured for 8 days and the supematants were collected, re-arrayed onto 384-well plates, and screened for antibodies specific to hUPK1B, cUPK1B, and rUPK1B expressed on the surface of transduced HEK/293T cells (ATCC CRL-11268) using flow cytometry, as follows. A mixture of the 293T cells stably transduced with hUPK1B, cUPK1B, and rUPK1B in each well were incubated for 30 minutes with 25 pL hybridoma supernatant and then washed with PBS/2% FCS. Cells were incubated for 15 minutes with 25 pL per sample Alexa Fluor® 647 AffiniPure F(ab′)2 Fragment Goat Anti-Mouse IgG, Fcγ Fragment Specific secondary antibody diluted in PBS/2% FCS, washed twice and re-suspended with PBS/2% FCS. The cells were then analyzed by flow cytometry (BD FACSCanto II).

A number of hUPK1B/cUPK1B/rUPK1b immunospecific antibodies were identified.

### Example 7

### Characteristics of Anti-UPK1B Antibodies

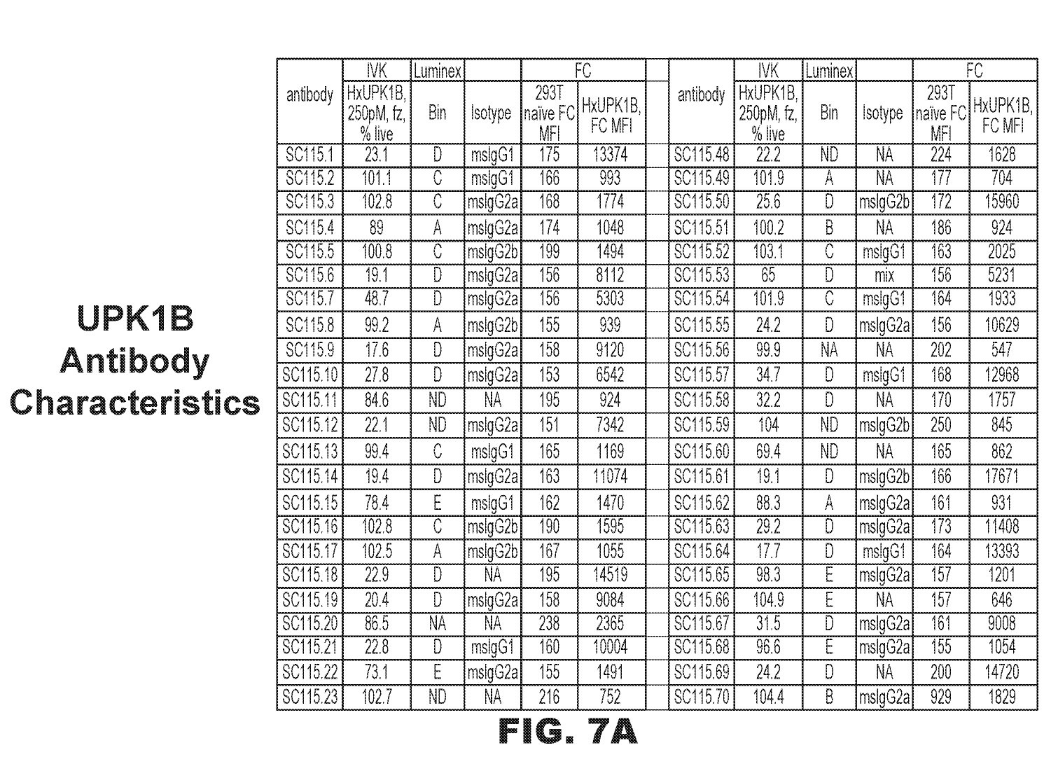

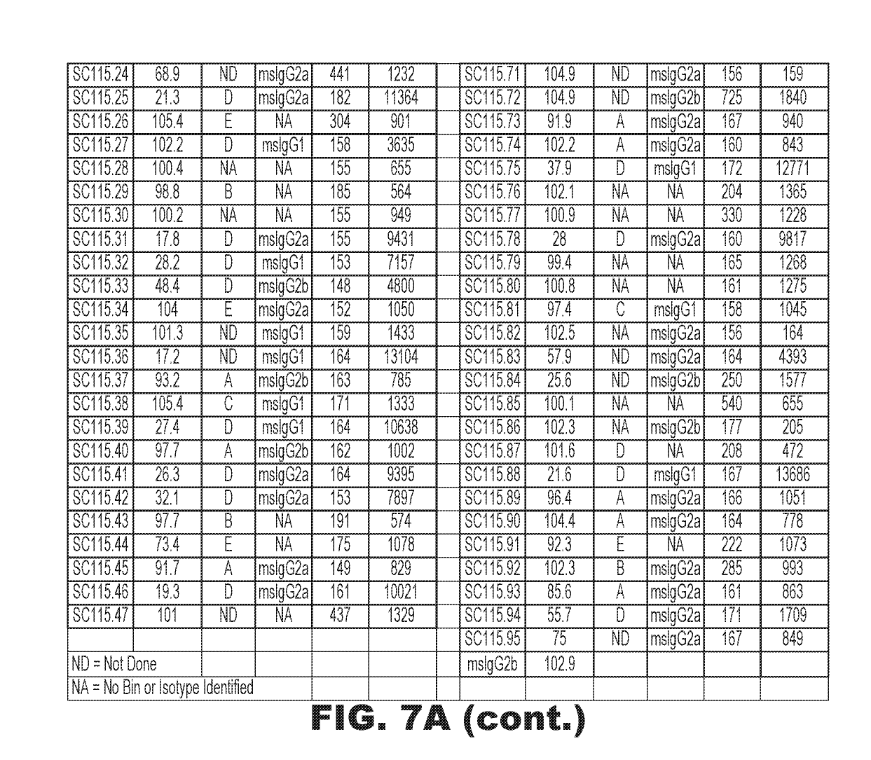

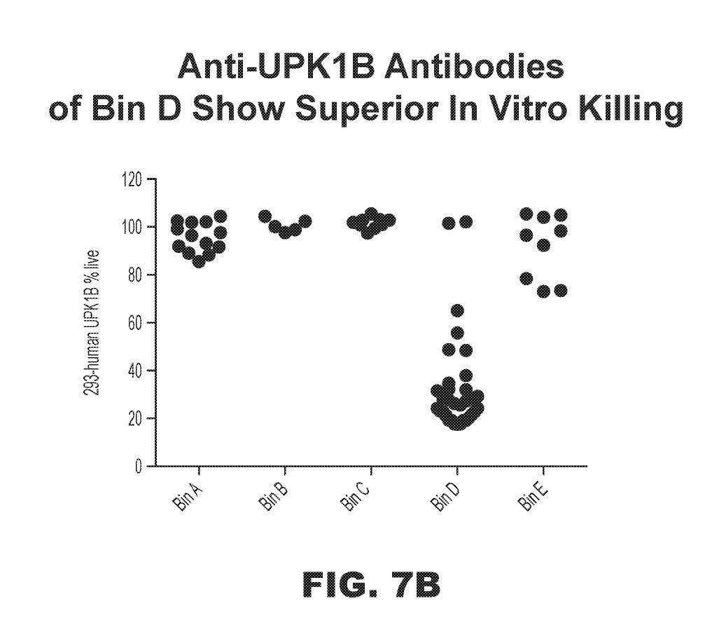

Various methods were used to characterize the anti-UPK1B mouse antibodies generated in Example 6 in terms of isotype, epitope binning, and the ability to recognize and kill cells expressing human UPK1B. FIG. 7A provides a table summarizing the aforementioned characteristics for a number of exemplary murine antibodies. In FIG. 7A a blank cell or “N/A” indicates that the data was not generated in that instance.

The isotype of a representative number of antibodies was determined using the Milliplex mouse immunoglobulin isotyping kit (Millipore) according to the manufacturer's protocols. Results for the exemplary UPK1B-specific antibodies are set forth in the column headed “isotype” in FIG. 7A.

Antibodies were grouped into bins using a multiplexed competition immunoassay (Luminex). 100 μl of each unique anti-UPK1B antibody (capture mAb) at a concentration of 10 μg/mL was incubated for 1 hour with magnetic beads (Luminex) that had been conjugated to an anti-mouse kappa antibody (Miller et al., 2011, PMID: 21223970). The capture mAb/conjugated bead complexes were washed with PBSTA buffer (1% BSA in PBS with 0.05% Tween20) and then pooled. Following removal of residual wash buffer the beads were incubated for 1 hour with 2 μg/mL hUPK1B-His protein, washed and then resuspended in PBSTA. The pooled bead mixture was distributed into a 96 well plate, each well containing a unique anti-UPK1B antibody (detector mAb) and incubated for 1 hour with shaking. Following a wash step, anti-mouse kappa antibody (the same as that used above), conjugated to PE, was added at a concentration of 5 μg/ml to the wells and incubated for 1 hour. Beads were washed again and resuspended in PBSTA. Mean fluorescence intensity (MFI) values were measured with a Luminex MAGPIX instrument Antibody pairing was visualized as a dendrogram of a distance matrix computed from the Pearson correlation coefficients of the antibody pairs. Binning was determined on the basis of the dendrogram and analysis of the MFI values of antibody pairs. Antibodies that had low affinity binding for UPK1B and could not be placed in a specific Bin are denoted with “NA” or “ND”. The data is presented in the column headed “bin” where FIG. 7A shows that the anti-UPK1B antibodies that were screened can be grouped into at least five unique bins (A-E) on the hUPK1B protein.

The exemplary antibodies were also tested using flow cytometry for their ability to associate with hUPK1B on the surface of cells. To this end engineered HEK293T cells overexpressing hUPK1B (prepared as per Example 5) along with naïve control cells were incubated for 30 minutes with the denoted antibodies and analyzed for hUPK1B expression by flow cytometry using a BD FACS Canto II flow cytometer according to the manufacturer's instructions. Antigen expression is quantified as the change in geometric mean fluorescence intensity (AMFI) observed on the surface of the engineered cells which have been stained with an anti-UPK1B antibody compared to the same cells that have been stained with an isotype control antibody. A change in geometric mean fluorescence intensity (ΔMFI) was also observed between engineered cells and those that had not been engineered. Results of the assay in terms of mean fluorescence intensity are set forth in FIG. 7A in the columns labelled FC. A review of the data shows that several of the disclosed antibodies bind hUPK1B on the surface of cells.

To determine whether anti-UPK1B antibodies of the invention were able to internalize in order to mediate the delivery of cytotoxic agents to live tumor cells, an in vitro cell killing assay was performed using exemplary anti-UPK1B antibodies and a secondary anti-mouse antibody FAB fragment linked to saporin. Saporin is a plant toxin that deactivates ribosomes, thereby inhibiting protein synthesis and resulting in the death of the cell. Saporin is only cytotoxic inside the cell where it has access to ribosomes, but is unable to internalize independently. Therefore, saporin-mediated cellular cytotoxicity in these assays is indicative of the ability of the anti-mouse FAB-saporin construct to internalize upon binding and internalization of the associated anti-UPK1B mouse antibodies into the target cells. Single cell suspensions of HEK293T cells overexpressing hUPK1B (prepared as per Example 5) were plated at 500 cells per well into BD Tissue Culture plates (BD Biosciences). One day later, various concentrations of the purified anti-UPK1B antibodies were added to the culture together with a fixed concentration of 2 nM anti-mouse IgG FAB-saporin constructs (Advanced Targeting Systems). After incubation for 96 hours viable cells were enumerated using CellTiter-Glo® (Promega) as per the manufacturer's instructions. Raw luminescence counts using cultures containing cells incubated only with the secondary FAB-saporin conjugate were set as 100% reference values and all other counts were calculated as a percentage of the reference value. The results, shown in FIG. 7A in the column labeled IVK are presented as the percentage of surviving cells.

These data demonstrate that a subset of anti-UPK1B antibody-saporin conjugates at a concentration of 250 pM effectively killed HEK293T cells overexpressing hUPK1B with varying efficacy (FIG. 7A).

In order to determine whether epitope position plays a role in the ability of an antibody to mediate cell killing, the killing data set forth in FIG. 7A for 293 cells expressing hUPK1B was plotted by bin to provide FIG. 7B. A review of FIG. 7B shows that those antibodies mapped to bin D exhibit higher cell killing activity when used in conjunction with saporin as set forth above. These data indicate that antibodies in bin D may be particularly effective when used as a component of an antibody drug conjugate as disclosed herein.

### Example 8

### UPK1B Protein is Expressed in PDX Tumor Cell Lines

Given the elevated UPK1B mRNA transcript levels associated with various tumors described in Examples 1-3, work was undertaken to test whether UPK1B protein expression was also elevated in PDX tumors. To detect and quantify UPK1B protein expression, an electrochemiluminscence UPK1B sandwich ELISA assay was developed using the MSD Discovery Platform (Meso Scale Discovery).

PDX tumors were excised from mice and flash frozen on dry ice/ethanol. Protein Extraction Buffer (Biochain Institute) was added to the thawed tumor pieces and tumors were pulverized using a TissueLyser system (Qiagen). Lysates were cleared by centrifugation (20,000 g, 20 min., 4° C.) and the total protein concentration in each lysate was quantified using bicinchoninic acid. The protein lysates were then normalized to 5 mg/mL and stored at −80° C. until used. Normal tissues were purchased from a commercial source.

The ELISA sandwich antibody pair used in the MSD assay consisted of SC115.22 capture and SC115.18 detection. This pair should still be specific to hUPK1B because the capture is UPK1B specific and should pull down only UPK1B protein. UPK1B protein concentrations from the lysate samples were determined by interpolating the values from a standard protein concentration curve that was generated using purified recombinant hUPK1B-Fc protein, generated as described in Example 5. The UPK1B protein standard curve and protein quantification assay were conducted as follows:

MSD standard plates were coated overnight at 4° C. with 15 μL of SC115.22 capture antibody at 2 μg/mL in PBS. Plates were washed in PBST and blocked in 35 μL MSD 3% Blocker A solution for one hour while shaking. Plates were again washed in PBST. 10 μL of 10× diluted lysate (or serially diluted recombinant UPK1B standard) in MSD 1% Blocker A containing 10% Protein Extraction Buffer was also added to the wells and incubated for two hours while shaking. Plates were again washed in PBST. The SC115.18 detection antibody was then sulfo-tagged using an MSD® SULFO-TAG NHS Ester according to the manufacturer's protocol. 10 μL of the tagged SC115.18 antibody was added to the washed plates at 0.5 μg/mL in MSD 1% Blocker A for 1 hour at room temperature while shaking. Plates were washed in PBST. MSD Read Buffer T with surfactant was diluted to 1× in water and 35 μL was added to each well. Plates were read on an MSD Sector Imager 2400 using an integrated software analysis program to derive UPK1B concentrations in PDX samples via interpolation from the standard curve. Values were then divided by total protein concentration to yield nanograms of UPK1B per milligram of total lysate protein.

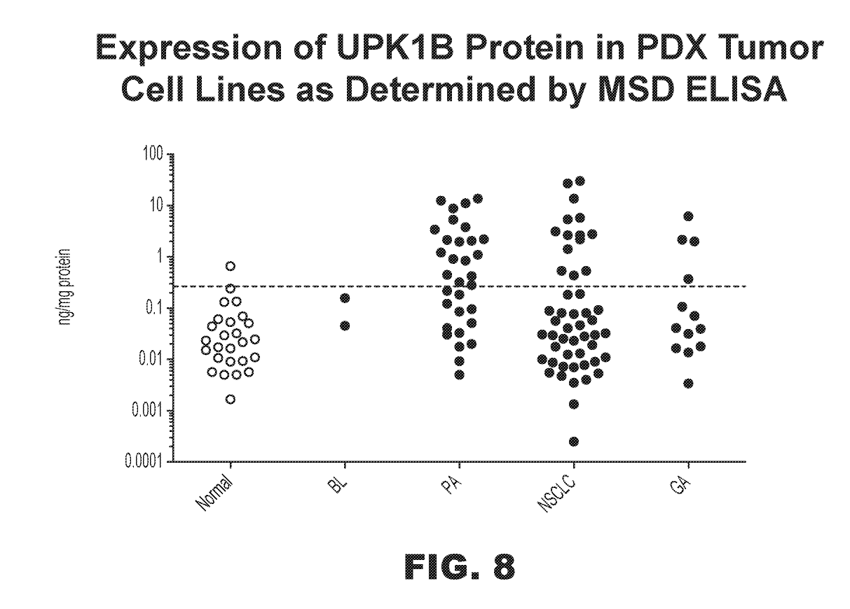

The resulting concentrations are set forth in FIG. 8 wherein each spot represents UPK1B protein concentrations derived from a single PDX tumor line. While each spot is derived from a single PDX line, in most cases multiple biological samples were tested from the same PDX line and values were averaged to provide the data point.

FIG. 8 shows that representative samples of BL, PA, NSCLC, and GA tumor samples exhibited high UPK1B protein expression. The levels of UPK1B protein expression for each sample are given in ng/mg total protein and the median derived for each tumor type is indicated by the horizontal bar. Normal tissues that were tested include adrenal gland, artery, colon, esophagus, gall bladder, heart, kidney, liver, lung, peripheral and sciatic nerve, pancreas, skeletal muscle, skin, small intestine, spleen, stomach, trachea, red and white blood cells and platelets, bladder, brain, breast, eye, lymph node, ovary, pituitary gland, prostate and spinal cord. Only one normal tissue (trachea) was detected at levels above the lower limit of quantitation of the assay in any of the normal tissue samples. These data, combined with the mRNA transcription data for UPK1B expression set forth above strongly reinforce the proposition that UPK1B is an attractive target for antibody-based therapeutic intervention.

### Example 9

### Immunohistochemistry of UPK1B Protein Expression in Tumors

Immunohistochemistry (IHC) was performed on PDX tumors and patient biopsies to assess the expression and location of UPK1B in tumor cells. IHC was also performed on UPK1B+engineered cells as a control.

In order to identify an IHC-compatible anti-UPK1B antibody, IHC was performed on HEK-293T parental cell pellets or UPK1B-expressing HEK-293T cell pellets using numerous anti-UPK1B antibodies of the invention. IHC was performed, as described below, on HEK-293T cells pellets that were formalin fixed and paraffin embedded (FFPE) as is standard in the art.

Planar sections of cell pellet blocks were cut and mounted on glass microscope slides. After xylene de-paraffinization 5 μm sections were pre-treated with Antigen Retrieval Solution (Dako) for 20 minutes at 99° C., cooled to 75° C. and then treated with 3% hydrogen peroxide in PBS followed by Avidin/Biotin Blocking Solution (Vector Laboratories). FFPE slides were then blocked with 10% horse serum in 3% BSA in PBS buffer and incubated with a primary anti-UPK1B antibody of the invention, diluted to 7.5 μg/ml for human tissues and 10 μg/ml for PDX lines, in 3% BSA/PBS, for 30 minutes at room temperature. FFPE slides were incubated with biotin-conjugated horse anti-mouse antibody (Vector Laboratories), diluted to 2.5 μg/ml in 3% BSA/PBS, for 30 minutes at room temperature followed by incubation in streptavidin-HRP (ABC Elite Kit; Vector Laboratories). Chromogenic detection was developed with 3,3′-diaminobenzidine (Thermo Scientific) for 5 minutes at room temperature and tissues were counterstained with Meyer's hematoxylin (IHC World), dehydrated with alcohol and immersed in xylene. Stained slides were analyzed by light microscopy. An H-score was utilized to quantify staining. The H-score is a method of assessing the extent of staining by utilizing the following formula: 3× percentage of tumor cells staining at 3+intensity+2× percentage of tumor cells staining at 2+intensity+1× percentage of tumor cells staining at 1+intensity, giving a range from 0 to 300.

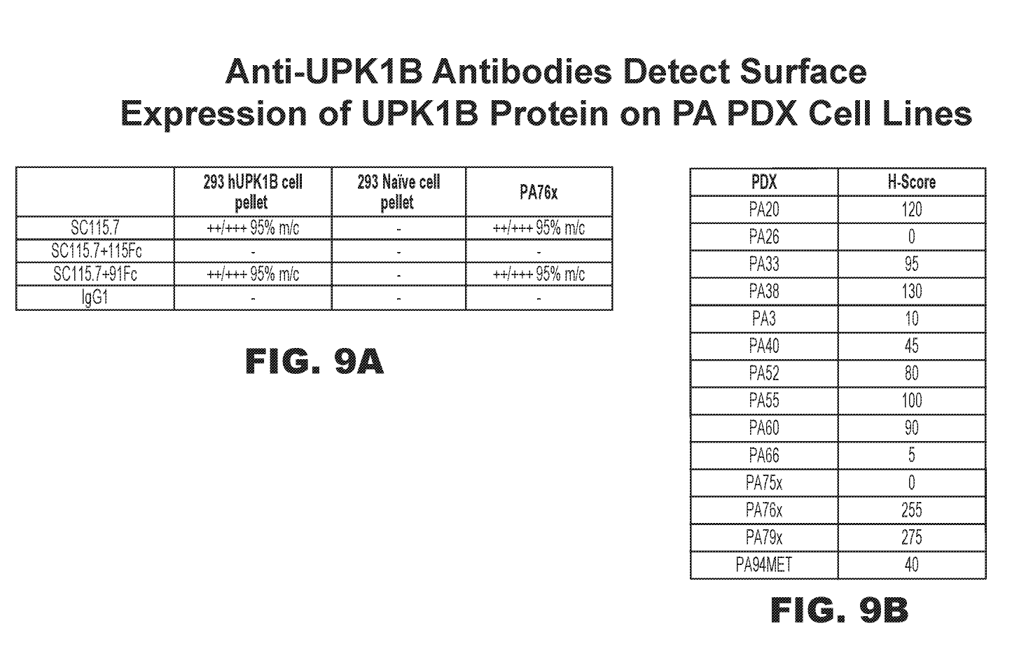

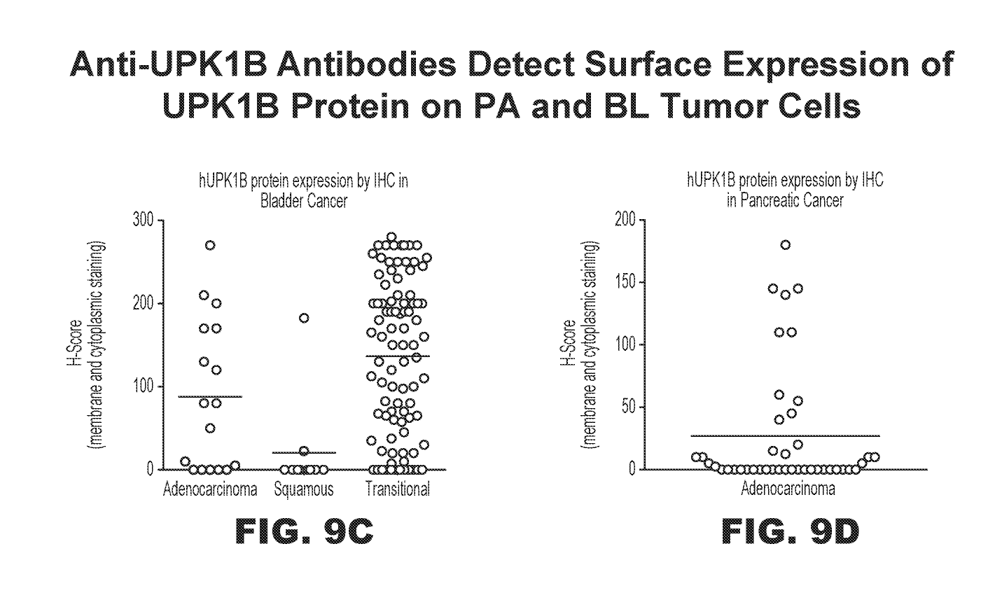

An anti-UPK1B antibody (SCI15.7) was able to specifically detect UPK1B-overexpressing HEK-293T cell pellets more effectively than other anti-UPK1B antibodies of the invention that were tested (data not shown). The ability of these antibodies to specifically detect UPK1B was confirmed by a competition experiment in which the relevant anti-UPK1B antibody was mixed with a 5× molar ratio excess of hUPK1B-Fc or irrelevant protein (SCRx91-Fc) and then incubated with UPK1B-expressing HEK293T formalin fixed and paraffin embedded (FFPE) sections. The absence of positive staining demonstrated that the hUPK1B-Fc protein interfered with the binding of the anti-UPK1B antibody to the UPK1B-overexpressing HEK293T cells (FIG. 9A).

Using substantially the same methodology the anti-UPK1B antibody SC115.7 was then used to determine whether hUPK1B was expressed in various PDX models. FIG. 9B shows that UPK1B is positive in 12/14 (86%) pancreatic PDX lines.

Immunohistochemistry was also performed on primary patient cancer biopsy samples where FIG. 9C shows that UPK1B is expressed in 12/17 (70%) of bladder adenocarcinoma, 2/10 (20%) of bladder squamous cell carcinoma and 78/90 (87%) of bladder transitional cell carcinoma. Finally, using similar techniques to provide the data, FIG. 9D shows that UPK1B is expressed in 20/42 (48%) of pancreatic adenocarcinoma.

This positive staining on a number of various PDX and primary patient tumors confirms expression of the marker and strongly suggests the viability of using the UPK1B as a diagnostic and therapeutic target.

### Example 10

### Flow Cytometry Detection of UPK1B Protein Expression in Tumors

Flow cytometry was used to assess the ability of the anti-UPK1B antibodies of the invention to specifically detect the presence of human UPK1B protein on the surface of pancreatic and bladder PDX tumor cell lines. In addition, expression of UPK1B on the surface of PA and BL CSCs was also determined. The PDX tumors were harvested and dissociated using art-recognized enzymatic tissue digestion techniques to obtain single cell suspensions of PDX tumor cells (see, for example, U.S.P.N. 2007/0292424). PDX tumor single cell suspensions were incubated with 4′6-diamidino-2-phenylindole (DAPI) to detect dead cells, anti-mouse CD45 and H-2K^{d} antibodies to identify mouse cells and anti-human EPCAM antibodies to identify human carcinoma cells. The resulting single cell suspensions comprised a bulk sample of tumor cells including both NTG cells and CSCs. In order to partition bulk tumor cell populations into NTG and CSC subpopulations, PDX tumor cells were incubated with anti-human CD46 and or CD324 and ESA antibodies (see U.S.P.N.s. 2013/0260385, 2013/0061340 and 2013/0061342). Bulk or sorted tumor cells were analyzed for hUPK1B expression by flow cytometry using a BD FACS Canto II flow cytometer with SC115.46, an anti-UPK1B antibody.

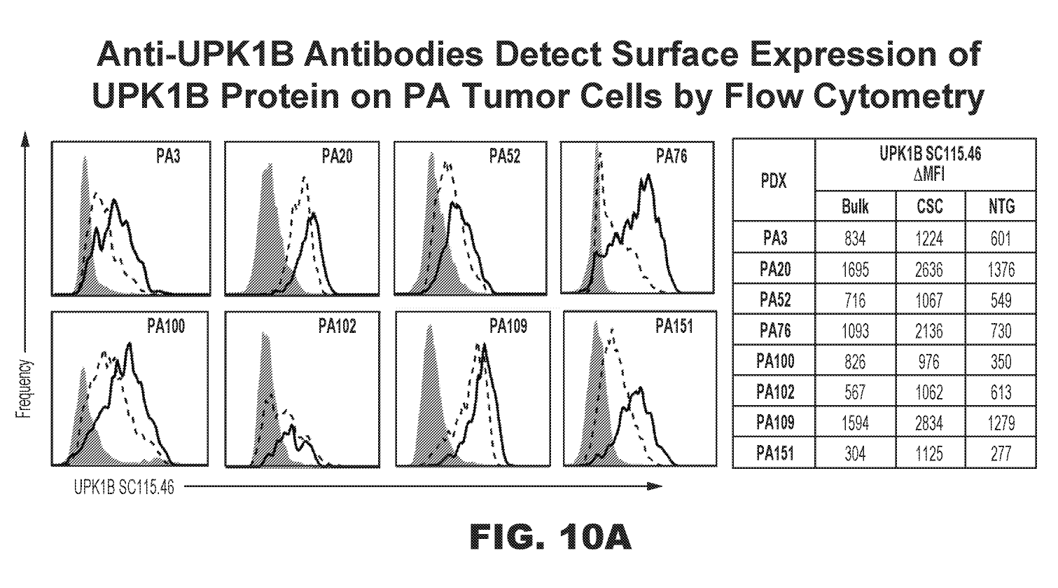

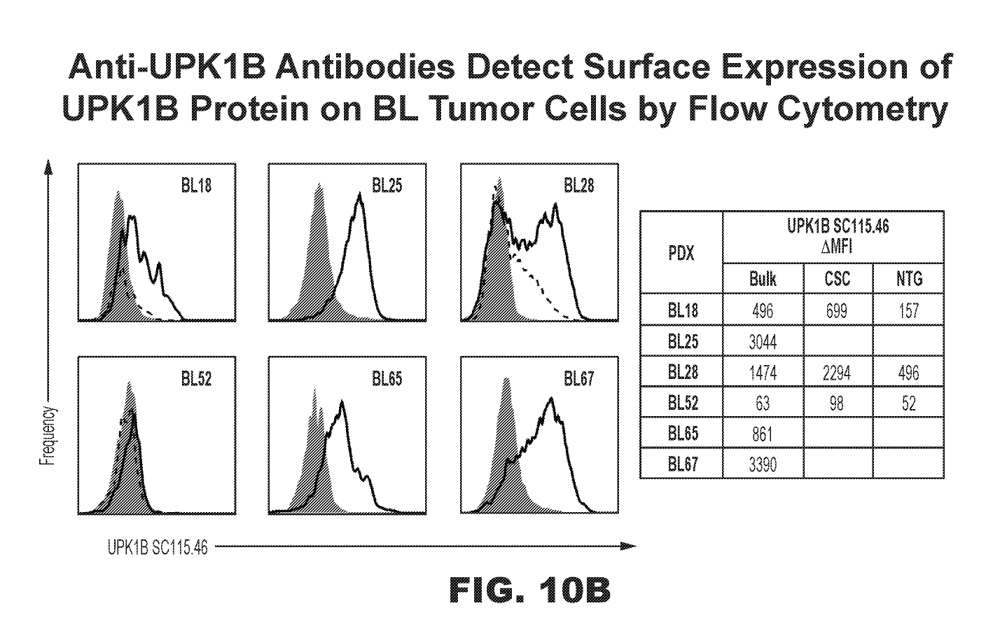

FIG. 10A shows that the anti-hUPK1B antibody SC115.46 detected expression of hUPK1B on the surface of bulk PA PDX tumor cells. In all samples, the anti-UPK1B antibody (black line) detected increased UPK1B expression compared to the IgG isotype control antibody (gray-filled). PDX tumor samples PA3, PA76, PA109, and PA151 showed increased hUPK1B expression on CSC (solid black line) and NTG subpopulations PA PDX tumor cells (dashed line) compared to the IgG isotype control antibody (gray-filled). This demonstrates that UPK1B is expressed on CSC in a number of PA tumor subtypes. Further, expression can be quantified as the change in geometric mean fluorescence intensity (ΔMFI) observed on the surface of tumor cells which have been stained with an anti-UPK1B antibody compared to the same tumor that has been stained with an isotype control antibody. A table summarizing the ΔMFI of for each of the tumor cell lines that were analyzed is shown as an insert in FIG. 10A. This data confirms the IHC results in FIG. 9B, in which pancreatic cancer PDX lines PA20, PA52, and PA76 also show positive staining by IHC.

FIG. 10B shows that the anti-UPK1B antibody SC115.46 detected expression of hUPK1B on the surface of bulk BL PDX tumor cells. In all samples, except BL52, the anti-UPK1B antibody (black line) detected increased UPK1B expression compared to the IgG isotype control antibody (grey-filled). PDX tumor samples BL18 and BL28 showed increased hUPK1B expression on CSC (solid black line) and NTG subpopulations PA PDX tumor cells (dashed line) compared to the IgG isotype control antibody (gray-filled). This demonstrates that UPK1B is expressed on CSC in a number of BL tumor subtypes. Further, expression can be quantified as the change in geometric mean fluorescence intensity (ΔMFI) observed on the surface of tumor cells which have been stained with an anti-UPK1B antibody compared to the same tumor that has been stained with an isotype control antibody. A table summarizing the ΔMFI of for each of the tumor cell lines that were analyzed is shown as an insert in FIG. 10B.

Collectively, this data suggests that UPK1Bis expressed in PA and BL PDX tumor cells making this a good indication for targeted therapy with an anti-UPK1B antibody drug conjugate.

### Example 11

### UPK1B Expression Status and Somatic Mutations

The mutational status of various genes in PA patient derived xenograft (PDX) lines may be determined by performing targeted re-sequencing of genomic DNA (gDNA). In some embodiments, the mutational status of pancreatic cancer-related genes can be used as a surrogate biomarker (as described in more detail below) to determine whether there is a correlation between various genetic mutations and the expression of UPK1B. In other embodiments the mutational status of the pancreatic cancer-related genes can be used to determine whether there is a correlation between genetic mutations and the response to treatment with the anti-UPK1B antibodies or ADCs of the invention. In further embodiments the mutational status of the pancreatic cancer-relate genes can be used to determine effective combination therapies.

To determine mutations that may predict expression of UPK1B, gDNA from PA PDX tumors was analyzed by targeted re-sequencing of major cancer driver genes using Ion Ampliseq and Ion Torrent PGM technologies. Briefly, gDNA from these tumors was harvested using standard molecular techniques and the Ion AmpliSeq Library Kit 2.0 was used to prepare libraries from a custom panel of AmpliSeq primers (Life Technologies) encompassing over 3000 amplicons of up to 250 bp, covering coding and non-coding regions of several hundred major cancer driver genes. Each PDX-derived library sample was then ligated to a unique Ion Xpress Barcode Adapter (Life Technologies) to allow pooling of multiple library samples within each sequencing run. Sequencing was then performed on an Ion Torrent PGM machine according to manufacturer's instructions.

PA tumors with a range of expression of UPK1B, as determined by microarray (Example 3 above) or flow cytometry (Example 10 above) were examined for correlations between mutation data and UPK1B expression. A mutation is defined by any non-synonymous alteration occurring in the protein-coding region of the sequenced gene, including missense non-synonymous, insertions or deletions of codons, amplicon deletions or amplicon amplifications, nonsense non-synonymous, frameshift, and mutations that lead to altered splice-site variants of the gene sequenced.

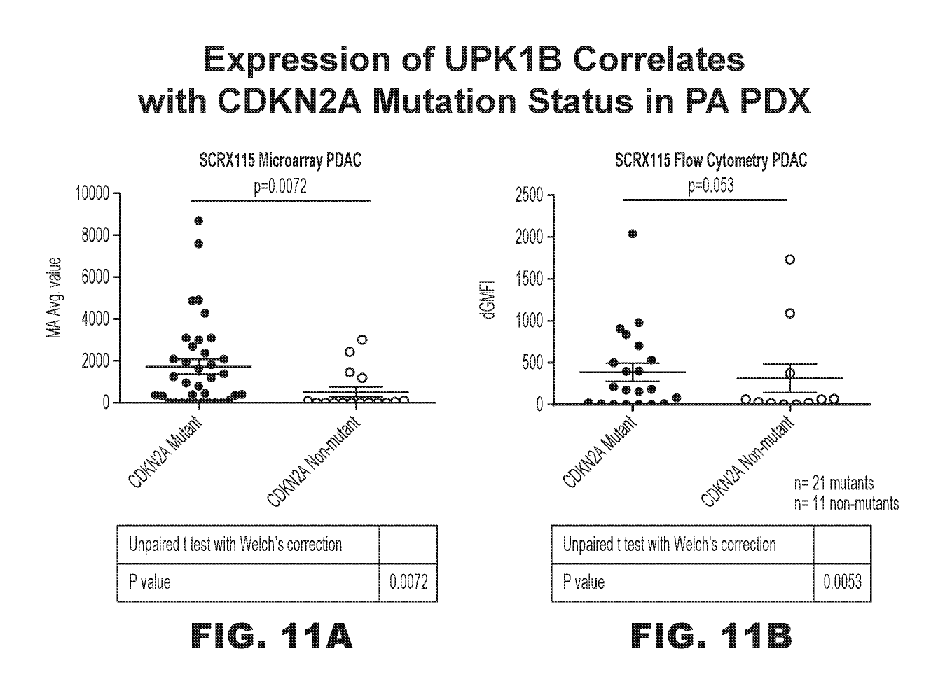

It was observed that PA PDX tumors carrying a mutation in the CDKN2A gene demonstrated significantly higher expression (p<0.05, Welch's T-test) of UPK1B compared with PDX tumors not carrying mutations in either of these genes, where UPK1B expression was determined by either microarray (FIG. 11A) or flow cytometry (FIG. 11B). These data suggest that mutations detected in this gene correlates with expression or absence of expression of UPK1B. This mutation can be used as a biomarker to predict expression of UPK1B in patient populations and more accurately guide treatment for these subsets of tumors

### Example 12

### Sequencing of UPK1B Antibodies

The anti-UPK1B mouse antibodies that were generated in Example 6 were sequenced as described below. Total RNA was purified from selected hybridoma cells using the RNeasy Miniprep Kit (Qiagen) according to the manufacturer's instructions. Between 10⁴ and 10⁵ cells were used per sample. Isolated RNA samples were stored at −80° C. until used.

The variable region of the Ig heavy chain of each hybridoma was amplified using two 5′ primer mixes comprising eighty-six mouse specific leader sequence primers designed to target the complete mouse VH repertoire in combination with a 3′ mouse Cγ primer specific for all mouse Ig isotypes. Similarly, two primer mixes containing sixty-four 5′ Vκ leader sequences designed to amplify each of the Vκ mouse families was used in combination with a single reverse primer specific to the mouse kappa constant region in order to amplify and sequence the kappa light chain. The VH and VL transcripts were amplified from 100 ng total RNA using the Qiagen One Step RT-PCR kit as follows. A total of four RT-PCR reactions were run for each hybridoma, two for the Vκ light chain and two for the VH heavy chain. PCR reaction mixtures included 1.5 μL of RNA, 0.4 μL of 100 μM of either heavy chain or kappa light chain primers (custom synthesized by Integrated DNA Technologies), 5 μL of 5×RT-PCR buffer, 1 μL dNTPs, and 0.6 μL of enzyme mix containing reverse transcriptase and DNA polymerase. The thermal cycler program was RT step 50° C. for 60 min., 95° C. for 15 min. followed by 35 cycles of (94.5° C. for 30 seconds, 57° C. for 30 seconds, 72° C. for 1 min.). There was then a final incubation at 72° C. for 10 min.

The extracted PCR products were sequenced using the same specific variable region primers as described above for the amplification of the variable regions. PCR products were sent to an extemal sequencing vendor (MCLAB) for PCR purification and sequencing services. Nucleotide sequences were analyzed using the IMGT sequence analysis tool (http://www.imgt.org/IMGTmedical/sequence_analysis.html) to identify germline V, D and J gene members with the highest sequence homology. The derived sequences were compared to known germline DNA sequences of the Ig V- and J-regions by alignment of VH and VL genes to the mouse germline database using a proprietary antibody sequence database.

FIG. 12A depicts the contiguous amino acid sequences of several novel murine light chain variable regions from anti-UPK1B antibodies while FIG. 12B depicts the contiguous amino acid sequences of novel murine heavy chain variable regions from the same anti-UPK1B antibodies. Taken together murine light and heavy chain variable region amino acid sequences are provided in SEQ ID NOS: 21-91 odd numbers.

More particularly FIGS. 12A and 12B provide the annotated sequences of several mouse anti-UPK1B antibodies, termed SC115.1, having a VL of SEQ ID NO: 21 and VH of SEQ ID NO: 23; SC115.4, having a VL of SEQ ID NO: 25 and a VH of SEQ ID NO: 27; SC115.7, having a VL of SEQ ID NO: 29 and a VH of SEQ ID NO: 31; SC115.9, having a VL of SEQ ID NO: 33 and a VH of SEQ ID NO: 35; SC115.13, having a VL of SEQ ID NO: 37 and a VH of SEQ ID NO: 39; SC115.18 having a VL of SEQ ID NO: 41 and a VH of SEQ ID NO: 43; SC115.19, having a VL of SEQ ID NO: 45 and a VH of SEQ ID NO: 47; SC115.26, having a VL of SEQ ID NO: 49 and a VH of SEQ ID NO: 51; SC115.32, having a VL of SEQ ID NO: 53 and a VH of SEQ ID NO: 55; SC115.36, having a VL of SEQ ID NO: 57 and a VH of SEQ ID NO: 59; SC115.46, having a VL of SEQ ID NO: 61 and a VH of SEQ ID NO: 63; SC115.48, having a VL of SEQ ID NO: 65 and a VH of SEQ ID NO: 67; SC115.51, having a VL of SEQ ID NO: 69 and a VH of SEQ ID NO: 71; SC115.52, having a VL of SEQ ID NO: 73 and a VH of SEQ ID NO: 75; SC115.65, having a VL of SEQ ID NO: 77 and a VH of SEQ ID NO: 79; SC115.84, having a VL of SEQ ID NO: 81 and a VH of SEQ ID NO: 83; SC115.90, having a VL of SEQ ID NO: 85 and a VH of SEQ ID NO: 87 and SC115.94, having a VL of SEQ ID NO: 89 and a VH of SEQ ID NO: 91.

A summary of the disclosed antibodies (or clones producing them), along with their respective variable region nucleic acid or amino acid SEQ ID NOS (see FIGS. 12A-12C) are shown immediately below in Table 5.

TABLE 5

| | VL | VH |
|---|---|---|
| | SEQ ID NO: | SEQ ID NO: |
| Clone | NA/AA | NA/AA |
| 115.1 | 20/21 | 22/23 |
| 115.4 | 24/25 | 26/27 |
| 115.7 | 28/29 | 30/31 |
| 115.9 | 32/33 | 34/35 |
| 115.13 | 36/37 | 38/39 |
| 115.18 | 40/41 | 42/43 |
| 115.19 | 44/45 | 46/47 |
| 115.26 | 48/49 | 50/51 |
| 115.32 | 52/53 | 54/55 |
| 115.36 | 56/57 | 58/59 |
| 115.46 | 60/61 | 62/63 |
| 115.48 | 64/65 | 66/67 |
| 115.51 | 68/69 | 70/71 |
| 115.52 | 72/73 | 74/75 |
| 115.65 | 76/77 | 78/79 |
| 115.84 | 80/81 | 82/83 |
| 115.90 | 84/85 | 86/87 |
| 115.94 | 88/89 | 90/91 |

The VL and VH amino acid sequences in FIGS. 12A and 12B are annotated to identify the framework regions (i.e. FR1-FR4) and the complementarity determining regions (i.e., CDRL1-CDRL3 in FIG. 12A or CDRH1-CDRH3 in FIG. 12B), defined as per Kabat et al. The variable region sequences were analyzed using a proprietary version of the Abysis database to provide the CDR and FR designations. Though the CDRs are defined as per Kabat et al., those skilled in the art will appreciate that the CDR and FR designations can also be defined according to Chothia, McCallum or any other accepted nomenclature system. In addition FIG. 12C provides the nucleic acid sequences (SEQ ID NOS: 20-90 even numbers) encoding the amino acid sequences set forth in FIGS. 12A and 12B.

As seen in FIGS. 12A and 12B and Table 5 the SEQ ID NOS. of the heavy and light chain variable region amino acid sequences for each particular murine antibody are sequential odd numbers. Thus the monoclonal anti-UPK1B antibody, SC115.1, comprises amino acid SEQ ID NOS: 21 and 23 for the light and heavy chain variable regions respectively; SC115.4 comprises SEQ ID NOS: 25 and 27; SC115.7 comprises SEQ ID NOS: 29 and 31, and so on. In addition the corresponding nucleic acid sequence encoding the murine antibody amino acid sequence (set forth in FIG. 12C) has a SEQ ID NO. immediately preceding the corresponding amino acid SEQ ID NO. Thus, for example, the SEQ ID NOS. of the nucleic acid sequences of the VL and VH of the SC115.1 antibody are SEQ ID NOS: 20 and 22, respectively.

In addition to the annotated sequences in FIGS. 12A-12C, FIGS. 12G and 12H provide CDR designations for the light and heavy chain variable regions of SC115.9 and SC115.18 as determined using Kabat, Chothia, ABM and Contact methodology. The CDR designations depicted in FIGS. 12G and 12H were derived using a proprietary version of the Abysis database as discussed above. As shown in subsequent Examples those of skill in the art will appreciate that the disclosed murine CDRs may be grafted into human framework sequences to provide CDR grafted or humanized anti-UPK1B antibodies in accordance with the instant invention. Moreover, in view of the instant disclosure one could easily determine the CDRs of any anti-UPK1B antibody made and sequenced in accordance with the teachings herein and use the derived CDR sequences to provide CDR grafted or humanized anti-UPK1B antibodies of the instant invention. This is particularly true of the antibodies with the heavy and light chain variable region sequences set forth in FIGS. 12A-12B.

### Example 13

### Generation of Chimeric and Humanized of UPK1B Antibodies

Chimeric anti-UPK1B antibodies were generated using art-recognized techniques as follows. Total RNA was extracted from the anti-UPK1B antibody-producing hybridomas using the method described in Example 1 and the RNA was PCR amplified. Data regarding V, D and J gene segments of the VH and VL chains of the mouse antibodies were obtained from the nucleic acid sequences (FIG. 12C) of the anti-UPK1B antibodies of the invention. Primer sets specific to the framework sequence of the VH and VL chain of the antibodies were designed using the following restriction sites: AgeI and XhoI for the VH fragments, and XmaI and DraIII for the VL fragments. PCR products were purified with a Qiaquick PCR purification kit (Qiagen), followed by digestion with restriction enzymes AgeI and XhoI for the VH fragments and XmaI and DraIII for the VL fragments. The VH and VL digested PCR products were purified and ligated into IgH or IgK expression vectors, respectively. Ligation reactions were performed in a total volume of 10 μL with 200U T4-DNA Ligase (New England Biolabs), 7.5 μL of digested and purified gene-specific PCR product and 25 ng linearized vector DNA. Competent E. coli DH10B bacteria (Life Technologies) were transformed via heat shock at 42° C. with 3 μL ligation product and plated onto ampicillin plates at a concentration of 100 μg/mL. Following purification and digestion of the amplified ligation products, the VH fragment was cloned into the AgeI-XhoI restriction sites of the pEE6.4 expression vector (Lonza) comprising HulgG1 (pEE6.4HulgG1) and the VL fragment was cloned into the XmaI-DraIII restriction sites of the pEE12.4 expression vector (Lonza) comprising a human kappa light constant region (pEE12.4Hu-Kappa).

Chimeric antibodies were expressed by co-transfection of CHO-S cells with pEE6.4HulgG1 and pEE12.4Hu-Kappa expression vectors. 2.5 μg each of pEE6.4HulgG1 and pEE12.4Hu-Kappa vector DNA were added to 15 μg PEI transfection reagent in 400 μL Opti-MEM. The mix was incubated for 10 min. at room temperature and added to cells. Supernatants were harvested three to six days after transfection. Culture supematants containing recombinant chimeric antibodies were cleared from cell debris by centrifugation at 800×g for 10 min. and stored at 4° C. Recombinant chimeric antibodies were purified with Protein A beads.

In addition, selected murine anti-UPK1B antibodies (SC115.9 and SC115.18) were humanized with the aid of a proprietary analytical program (Abysis Database, UCL Business) and standard molecular engineering techniques as follows. Human framework regions of the variable regions were selected/designed based on the highest homology between the framework sequences and CDR canonical structures of human germline antibody sequences and the framework sequences and CDRs of the relevant mouse antibodies. For the purpose of the analysis the assignment of amino acids to each of the CDR domains was done in accordance with Kabat et al. numbering. Once the variable regions were selected, they were generated from synthetic gene segments (Integrated DNA Technologies). Humanized antibodies were cloned and expressed using the molecular methods described above for chimeric antibodies.

The VL and VH amino acid and nucleic acid sequences of the humanized antibody hSC115.9 (FIGS. 12D and 12E; SEQ ID NOS: 101 and 103, AA and SEQ ID NOS: 100 and 102, NA) were derived from the VL and VH sequences of the corresponding murine antibody SC115.9 (SEQ ID NOS: 33 and 35) while the VL and VH amino acid sequences of the humanized antibody hSC115.18 (FIGS. 12D and 12E; SEQ ID NOS: 105 and 107, AA and SEQ ID NOS: 104 and 106, NA) were derived from the VL and VH sequences of the corresponding murine antibody SC115.118 (SEQ ID NOS: 41 and 43). TABLE 6 below shows that framework residue changes were made in the hSC115.18 constructs to maintain the binding affinity of the humanized antibody. More specifically changes were made at position 69 in the heavy chain and 78 in the light chain (Kabat numbering) to preserve the favorable characteristics of the molecule.

TABLE 6

| | | | | VH FR | VH CDR | | | VK FR | VK CDR |
|---|---|---|---|---|---|---|---|---|---|
| mAb | Isotype | human VH | human JH | changes | changes | human VK | human JK | changes | changes |
| hSC115.9 | IgG1/κ | IGHV5-51*01 | JH6 | None | None | IGKV1-39*01 | JK2 | None | None |
| hSC115.9ss1 | IgG1 | IGHV5-51*01 | JH6 | None | None | IGKV1-39*01 | JK2 | None | None |
| | C220S/κ | | | | | | | | |
| hSC115.18 | IgG1/κ | IGHV1-3*01 | JH6 | I69L | None | IGKV1-39*01 | JK2 | L78V | None |
| hSC115.18ss1 | IgG1 | IGHV1-3*01 | JH6 | I69L | None | IGKV1-39*01 | JK2 | L78V | None |
| | C220S/κ | | | | | | | | |

As discussed in Example 16 below, Table 6 also shows the composition of the exemplary site-specific antibodies (hSC115.9ss1 and hSC115.18ss1) fabricated as described herein.

### Example 14

### Humanized UPK1B Antibody Characteristics

The kinetic characteristics and affinities of the anti-UPK1B antibodies for human UPK1B protein was determined by surface plasmon resonance using a Biacore T200 (GE Healthcare). An anti-human antibody capture kit was used to immobilize anti-human antibodies on a CM5 biosensor chip. Then, in independent flow cells, CHO expressed Fc-fusion human UPK1B protein was immobilized. Prior to each anti-UPK1B antibody Fab fragment injection cycle, the human Fc-fusion protein was captured at a concentration of 1 μg/mL on the surface with a contact time of 12 seconds and a flow rate of 20 μL/min. The captured human UPK1B Fc-fusion protein loading from baseline was on average 166 response units (range 146-186 response units) on the BiacoreT200 Following human UPK1B Fc-fusion protein capture, papain-digested anti-UPK1B antibody Fab fragment was flowed over the surface at concentrations of 200 nM on the BiacoreT200 during the association phase followed by a 180 second dissociation phase at a flow rate of 40 μL/min. On the Biacore T200 following capture of the human UPK1B Fc-fusion protein, anti-UPK1B antibody Fab fragment was successively injected 4 times at increasing concentrations (12.5 nM, 25 nM, 50 nM, 100 nM) using the high performance injection (single concentration) method followed by a 180 second dissociation phase. The CM5 anti-human chip surface was regenerated with 1 min. contact time of 10 mM Glycine, pH 1.7 at 10 μL/min. following each cycle.

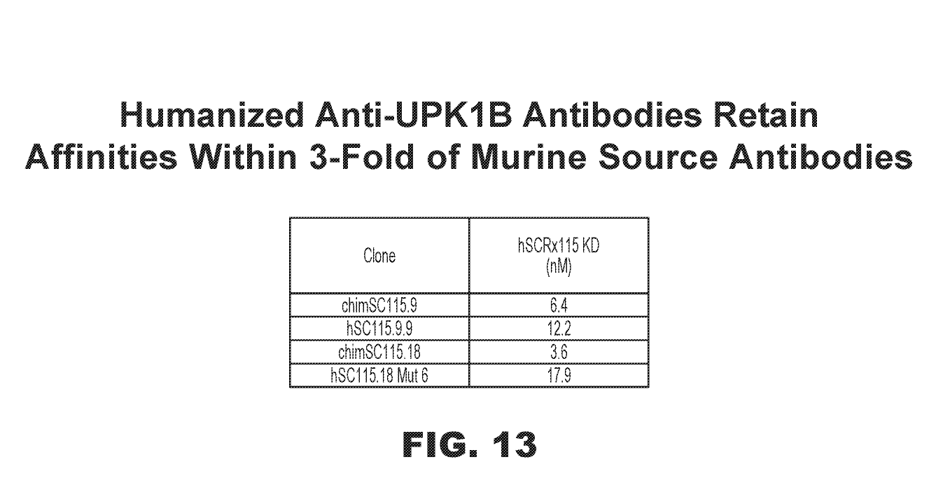

The data was processed by subtracting a control irrelevant human Fc-fusion protein surface response from the specific human UPK1B Fc-fusion protein surface response and data was truncated to the association and dissociation phase. The resulting response curves were used to evaluate the kinetic characteristics of the antibodies for experiments done on the Biacore T200. Association and dissociation data was fit with a 1:1 langmuir binding model using the Biacore T200 Evaluation Software (GE Healthcare). As shown in FIG. 13 the affinities for the humanized anti-UPK1B clones were within 3-fold of the parental chimeric antibody.

### Example 15

### Humanized UPK1B Antibodies Mediate Cell Killing In Vitro

To determine whether humanized anti-UPK1B antibodies of the invention were able to internalize in order to mediate the delivery of cytotoxic agents to live tumor cells, an in vitro cell killing assay was performed using the two humanized anti-UPK1B (hSC115.9 and hSC115.18) antibodies and a secondary anti-human antibody FAB fragment linked to saporin. Saporin is a plant toxin that deactivates ribosomes, thereby inhibiting protein synthesis and resulting in the death of the cell. Saporin is only cytotoxic inside the cell where it has access to ribosomes, but is unable to internalize independently. Therefore, saporin-mediated cellular cytotoxicity in these assays is indicative of the ability of the anti-human FAB-saporin construct to internalize upon binding and internalization of the associated anti-UPK1B humanized antibodies into the target cells.

Single cell suspensions of HEK293T cells overexpressing hUPK1B were plated at 500 cells per well into BD Tissue Culture plates (BD Biosciences). One day later, various concentrations of purified anti-UPK1B antibodies were added to the culture together with a fixed concentration of 2 nM anti-human IgG FAB-saporin constructs (Advanced Targeting Systems). After incubation for 96 hours viable cells were enumerated using CellTiter-Glo® (Promega) as per the manufacturer's instructions. Raw luminescence counts using cultures containing cells incubated only with the secondary FAB-saporin conjugate were set as 100% reference values and all other counts were calculated as a percentage of the reference value.

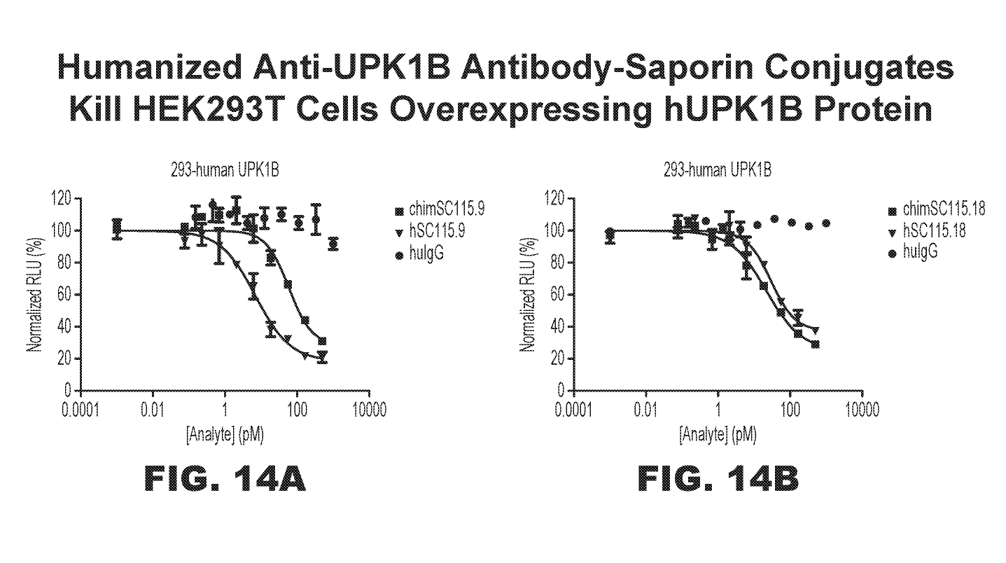

Both humanized anti-UPK1B antibody-saporin conjugates at a concentration of 100 μM effectively killed HEK293T cells overexpressing hUPK1B with varying efficacy (FIG. 14A, hSC115.9 and FIG. 14B, hSC115.18), whereas the human IgG1 isotype control antibody at the same concentration did not. The humanized antibodies showed comparable efficacy to the chimeric antibodies from which they were derived. The above results demonstrate the ability of anti-UPK1B antibodies to mediate internalization of a conjugated cytotoxic payload, supporting the hypothesis that anti-UPK1B antibodies may have therapeutic utility as the targeting moiety for an ADC

### Example 16

### Generation of Site-Specific UPK1B Antibodies

In addition to the native humanized IgG1 anti-UPK1B hSC115.9 and hSC115.18 antibodies engineered human IgG1/kappa anti-UPK1B site-specific antibodies were constructed comprising native light chain (LC) constant regions and heavy chain (HC) constant regions mutated to provide an unpaired cysteine in the light chains. In this respect cysteine 220 (C220) in the upper hinge region of the HC, which usually forms an interchain disulfide bond with cysteine 214 (C214) in the LC in native IgG1 antibodies, was substituted with serine (C220S). When assembled, the HCs and LCs form an antibody comprising two free cysteines at the c-terminal ends of the light chain constant regions that are suitable for conjugation to a therapeutic agent. Unless otherwise noted all numbering of constant region residues is in accordance with the EU numbering scheme as set forth in Kabat et al.

To generate humanized native IgG1 antibodies and site-specific constructs a VH nucleic acid was cloned onto an expression vector containing a HC constant region (e.g., SEQ ID NO: 2) or a C220S mutation of the same (e.g., SEQ ID NO: 3). Vectors encoding the native hSC115.9 HC (FIG. 12F, SEQ ID NO: 111) or hSC115.18 HC (FIG. 12F, SEQ ID NO: 114) and mutant C220S HCs of hSC115.9ss1 (FIG. 12F, SEQ ID NO: 112) or hSC115.18ss1 (FIG. 12F, SEQ ID NO: 115) were co-transfected in CHO-S cells with a vector encoding the selected VL (hSC115.9, SEQ ID NO: 101 or hSC115.18, SEQ ID NO: 105) operably associated with a wild-type IgG1 kappa LC (SEQ ID NO: 5) to provide the hSC115.9 LC (FIG. 12F, SEQ ID NO: 110) or the hSC115.18 LC (FIG. 12F, SEQ ID NO: 113). The transfected CHO cells were then used to provide the antibodies which were expressed using a mammalian transient expression system. The resulting anti-UPK1B site-specific antibodies containing the C220S mutant HC were termed hSC115.9ss1 and hSC115.18ss1 while the native versions were termed hSC115.9 and hSC115.18. In this regard the amino acid sequences of the full-length hSC115.9 site-specific antibody heavy and light chains are shown in FIG. 12F (along with native humanized antibody hSC115.9) where hSC115.9ss1 comprises an LC and HC of SEQ ID NOS: 110 and 112 respectively and hSC115.9 comprises an LC and HC of SEQ ID NOS: 110 and 111 respectively. Similarly, the amino acid sequences of the full-length hSC115.18 site-specific antibody heavy and light chains are shown in FIG. 12F (along with native humanized antibody hSC115.18) where hSC115.18ss1 comprises an LC and HC of SEQ ID NOS: 113 and 115 respectively and hSC115.18 comprises an LC and HC of SEQ ID NOS: 113 and 114 respectively.

The engineered anti-UPK1B site-specific antibodies were characterized by SDS-PAGE to confirm that the correct mutants had been generated. SDS-PAGE was conducted on a pre-cast 10% Tris-Glycine mini gel from Life Technologies in the presence and absence of a reducing agent such as DTT (dithiothreitol). Following electrophoresis, the gels were stained with a colloidal coomassie solution (data not shown). Under reducing conditions, two bands corresponding to the free LCs and free HCs, were observed. This pattern is typical of IgG molecules in reducing conditions. Under non-reducing conditions, the band patterns were different from native IgG molecules, indicative of the absence of a disulfide bond between the HC and LC. A band around 98 kD corresponding to the HC-HC dimer was observed. In addition, a faint band corresponding to the free LC and a predominant band around 48 kD that corresponded to a LC-LC dimer was observed. The formation of some amount of LC-LC species is expected due to the free cysteines on the c-terminus of each LC.

As discussed herein the ability to fabricate site-specific UPK1B antibodies allows for the preparation of more homogeneous compositions and may provide an improved therapeutic index when compared with standard prior art ADC compositions.

### Example 17

### Preparation of UPK1B Antibody-Drug Conjugates

Various chimeric antibodies with murine variable regions and humanized anti-UPK1B antibodies (including site-specific constructs of hSC115.9ss1 and hSC115.18ss1) were conjugated to a PBD or MMD10 (DL1) via a terminal maleimido moiety with a free sulfhydryl group to create antibody drug conjugates (ADCs) termed hSC115.9-PBD, hSC115.9ss1-PBD, hSC115.9-MMD10, hSC115.9ss1-MMD10, hSC115.18-PBD, hSC115.18ss1-PBD, hSC115.18-MMD10, hSC115.18ss1-MMD10 and hSC115.9ss1-MMAE. These conjugates will be used in the subsequent Examples along with appropriate conjugated and unconjugated controls.

The native anti-UPK1B ADCs were prepared as follows. The cysteine bonds of anti-UPK1B antibodies were partially reduced with a pre-determined molar addition of mol tris(2-carboxyethyl)-phosphine (TCEP) per mol antibody for 90 min. at room temperature in phosphate buffered saline (PBS) with 5 mM EDTA. The resulting partially reduced preparations were then conjugated to the drug linker via a maleimide linker for a minimum of 30 mins. at room temperature. The reaction was then quenched with the addition of excess N-acetyl cysteine (NAC) compared to linker-drug using a 10 mM stock solution prepared in water. After a minimum quench time of 20 mins, the pH was adjusted to 6.0 with the addition of 0.5 M acetic acid. Preparations of the ADCs were buffer exchanged into diafiltration buffer by diafiltration using a 30 kDa membrane. The dialfiltered anti-UPK1B ADCs were then formulated with sucrose and polysorbate-20 to the target final concentration. The resulting anti-UPK1B ADCs were analyzed for protein concentration (by measuring UV), aggregation (SEC), drug to antibody ratio (DAR) by reverse-phase HPLC (RP-HPLC) and activity (in vitro cytotoxicity).

The exemplary site-specific humanized anti-UPK1B ADCs were conjugated using a modified partial reduction process. The desired product is an ADC that is maximally conjugated on the unpaired cysteine (C214 in ss1 constructs) on each LC constant region and that minimizes ADCs having a drug to antibody ratio (DAR) which is greater than 2 (DAR>2) while maximizing ADCs having a DAR of 2 (DAR=2). In order to further improve the specificity of the conjugation, the antibodies were selectively reduced using a process comprising a stabilizing agent (e.g. L-arginine) and a mild reducing agent (e.g. glutathione) prior to conjugation with the linker-drug, followed by a diafiltration and formulation step.

A preparation of each site-specific antibody were selectively reduced in a buffer containing 1M L-arginine/5 mM EDTA with a pre-determined concentration of reduced glutathione (GSH), pH 8.0 for a minimum of two hours at room temperature. All preparations were then buffer exchanged into a 20 mM Tris/3.2 mM EDTA, pH 7.0 buffer using a 30 kDa membrane (Millipore Amicon Ultra) to remove the reducing buffer. The resulting selectively reduced preparations were then conjugated to the drug linker via a maleimide linker for a minimum of 30 mins. at room temperature. The reaction was then quenched with the addition of excess NAC compared to linker-drug using a 10 mM stock solution prepared in water. After a minimum quench time of 20 mins. the pH was adjusted to 6.0 with the addition of 0.5 M acetic acid. The resulting site-specific preparations of ADCs were buffer exchanged into diafiltration buffer by diafiltration using a 30 kDa membrane. The dialfiltered anti-UPK1B ADC was then formulated with sucrose and polysorbate-20 to the target final concentration. The resulting site-specific anti-UPK1B ADCs were analyzed for protein concentration (by measuring UV), aggregation (SEC), drug to antibody ratio (DAR) by reverse-phase HPLC (RP-HPLC) and activity (in vitro cytotoxicity).

The resulting conjugates were stored until use.

### Example 18

### UPK1B Antibody Drug Conjugates Facilitate Delivery of Cytotoxic Agents In Vitro

To determine whether anti-UPK1B ADCs of the invention were able to internalize in order to mediate the delivery of cytotoxic agents to live tumor cells, an in vitro cell killing assay was performed using the anti-UPK1B ADCs, hSC115.9ss1-PBD, hSC115.18ss1-PBD, hSC115.9ss1-MMD10 (ADC1), and hSC115.9ss1-MMAE (ADC6) each produced as described in Example 18 above.

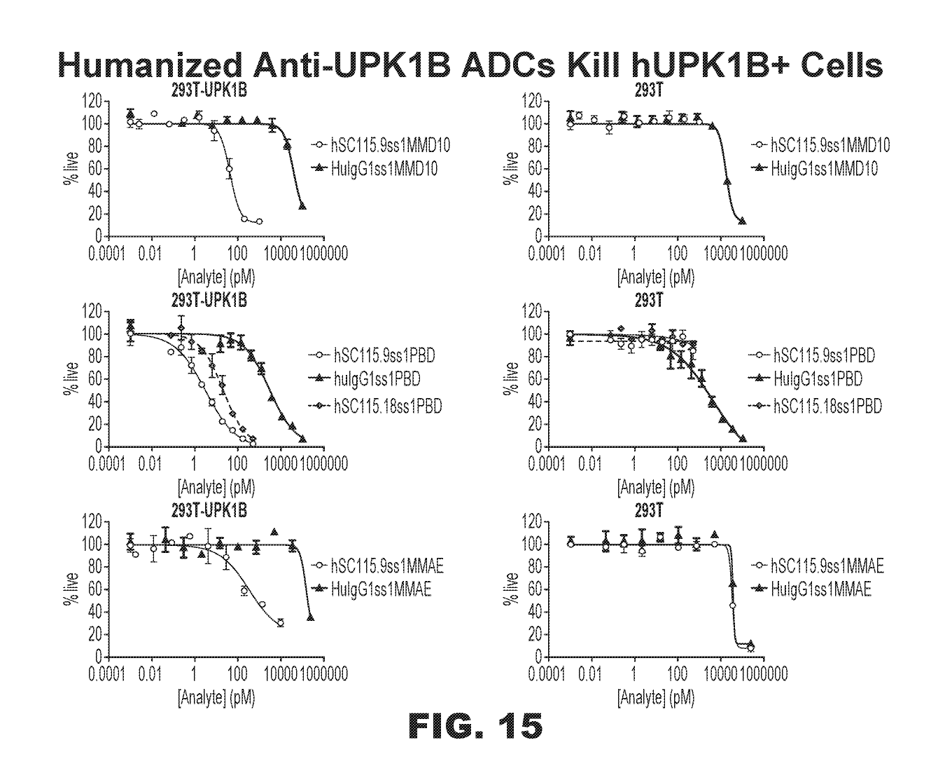

Single cell suspensions of HEK293T cells overexpressing human UPK1B or naïve HEK293T cells were plated at 500 cells per well into BD Tissue Culture plates (BD Biosciences). One day later, various concentrations of purified ADC or human IgG1 control antibody conjugated to a PBD, dolastatin10 or MMAE were added to the cultures. The cells were incubated for 96 hours at 37 C/5% CO2. After the incubation viable cells were enumerated using CellTiter-Glo® (Promega) as per the manufacturer's instructions. Raw luminescence counts using cultures containing non-treated cells were set as 100% reference values and all other counts were calculated as a percentage of the reference value. FIG. 15 shows that cells were much more sensitive to the anti-UPK1B ADCs compared to the human IgG1 control antibody. Furthermore, the UPK1B ADCs had very little effect on naive HEK293T cells that did not overexpress UPK1B compared to the HEK293T cells overexpressing UPK1B, demonstrating the specificity of the ADCs to the UPK1B antigen (FIG. 15).

The above results demonstrate the ability of anti-UPK1B ADCs to specifically mediate internalization and delivery of cytotoxic payloads (PBD, auristatin and dolastatin) to cells expressing UPK1B.

### Example 19

### UPK1B Antibody-Drug Conjugates Suppress Tumor Growth In Vivo

Based on the aforementioned results work was undertaken to demonstrate that conjugated UPK1B modulators of the instant invention shrink and suppress growth of UPK1B expressing human tumors in vivo. In this regard a selected murine antibody modulator (SC115.9) was covalently associated with a PBD cytotoxic agent and the resulting ADCs were tested to demonstrate their ability to suppress human PDX tumor growth in immunodeficient mice.

To this end patient-derived xenograft (PDX) tumors were grown subcutaneously in the flanks of female NOD/SCID recipient mice using art-recognized techniques. Tumor volumes and mouse weights were monitored twice weekly. When tumor volumes reached 150-250 mm³, mice were randomly assigned to treatment groups and injected with indicated doses of UPK1B ADC or an anti-hapten control IgG1-PBD (each produced substantially as described in Example 18) via intraperitoneal injection. Mice were given a single injection. Following treatment, tumor volumes and mouse weights were monitored until tumors exceeded 800 mm³ or mice became sick. For all tests, treated mice exhibited no adverse health effects beyond those typically seen in immunodeficient tumor-bearing NOD/SCID mice.

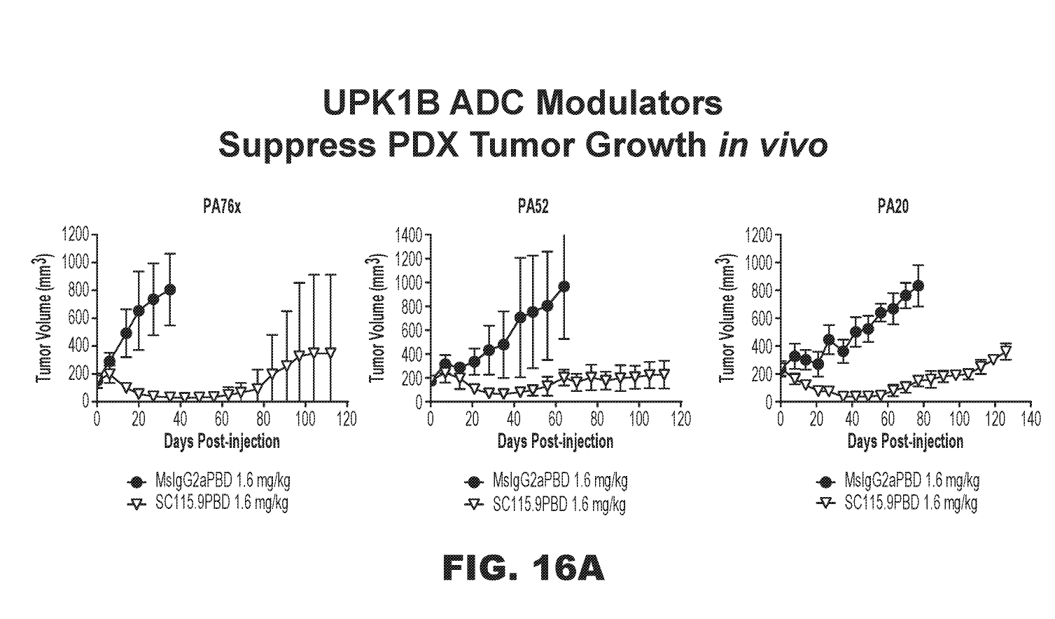

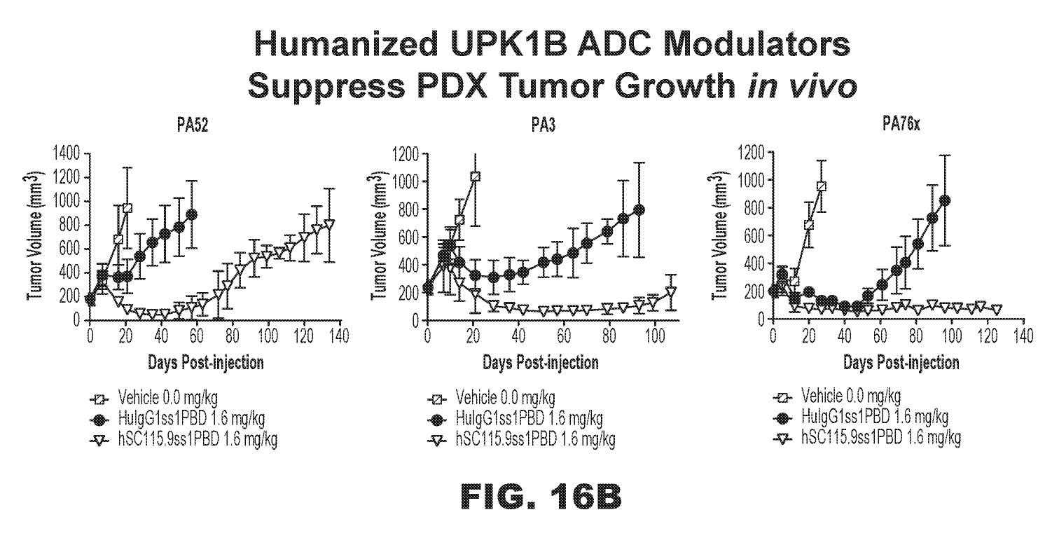

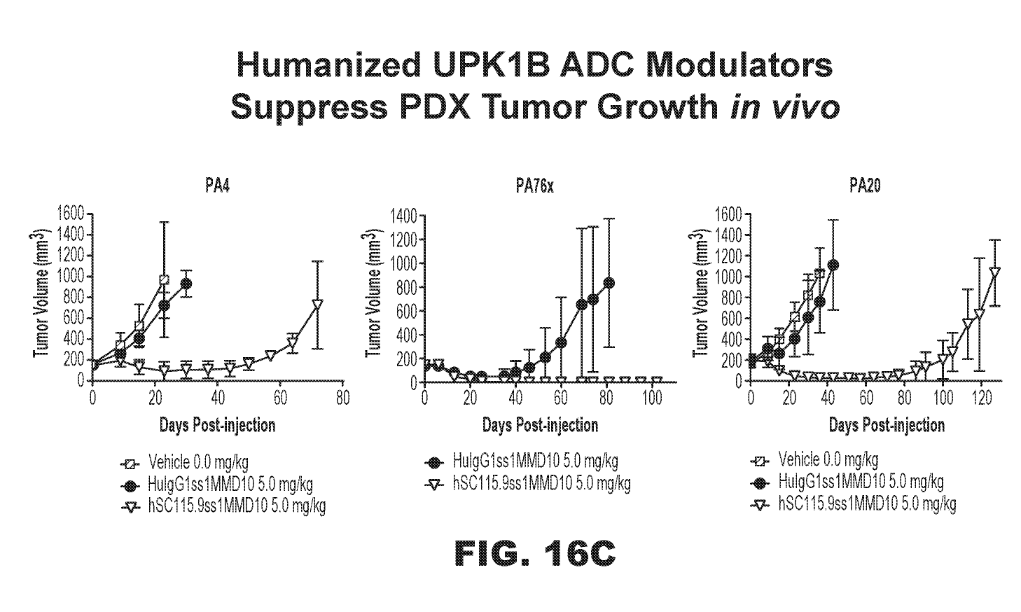

FIG. 16A shows the impact of the disclosed ADCs on tumor growth in mice bearing different pancreatic tumors exhibiting UPK1B expression. In this respect treatment of PA76, a pancreatic ductal adenocarcinoma, with exemplary UPK1B antibody SC115.9 conjugated to a PBD resulted in tumor shrinkage lasting as long as 40 days before tumor regrowth began. Treatment of a PA20 tumor retarded tumor growth for a period of approximately 60 days. Finally treatment of PA52, a pancreatic ductal adenocarcinoma, with exemplary antibody SC115.9-PBD produced tumor shrinkage and inhibited tumor regrowth for over 100 days (FIG. 14A).

Given the impressive results provided by UPK1B ADCs in previous Examples, additional experiments were performed to demonstrate the efficacy of exemplary humanized ADC modulators in treating pancreatic tumors in vivo. Specifically, selected humanized anti-UPK1B antibody (hSC115.9) produced as set forth in Example 13 above) were conjugated to a PBD and MMD10 as described herein and, with controls, administered to PDX tumor implanted immunodeficient mice as set forth above. In tests of humanized antibodies conjugated to MMD10, mice were injected with unconjugated anti-hapten control hIgG1 30 minutes prior to ADC injection to block sites of non-specific antibody binding. In each study, tumor volumes and mouse weights of the control animals were monitored until tumors exceeded 800 mm³ or mice became sick. The results of these experiments are presented in FIGS. 16B and 16C.

A review of FIGS. 16B and 16C show that tumor volume reduction was achieved following treatment with 1.6 mg/kg hSC115.9ss1-PBD and 5 mg/kg hSC115.9ss1-MMD10. For example, in PA76x, a pancreatic ductal adenocarcinoma, treatment with hSC115.9ss1-PBD or hSC115.9ss1-MMD10 produced tumor shrinkage and durable remission beyond 100 days. Treatment of PA3 with hSC115.9ss1-PBD produced tumor shrinkage lasting beyond 80 days post treatment (FIG. 16B). Treatment of PA52 with hSC115.9ss1-PBD resulted in tumor shrinkage and suppressed growth lasting 40 days (FIG. 16B). Treatment of hSC115.9ss1-MMD10 in PA4 and PA20, both pancreatic ductal adenocarcinomas, resulted in tumor shrinkage and suppressed tumor growth for 50 and 80 days post treatment respectively (FIG. 16C).

The surprising ability of a variety of conjugated modulators to dramatically shrink tumor volumes in vivo for extended periods further validates the use of anti-UPK1B antibodies as a therapeutic target for the treatment of proliferative disorders.

### Example 20

### UPK1B Expression Correlates with PDX Tumor Growth Suppression by UPK1B ADCs

To determine if the expression level of hUPK1B could be used to predict response to treatment with anti-UPK1B ADCs the RNA and protein levels of hUPK1B in PA PDX were plotted against the time to tumor progression (TTP) observed when these PDX models were treated with anti-hUPK1B ADCs in vivo. The expression level of hUPK1B was determined by microarray (as outlined in Example 3 above) or MSD (as outlined in Example 8 above). Delta time to tumor progression (dTTP) was calculated for each PA PDX dosed with either the murine or humanized anti-hUPK1B ADCs.

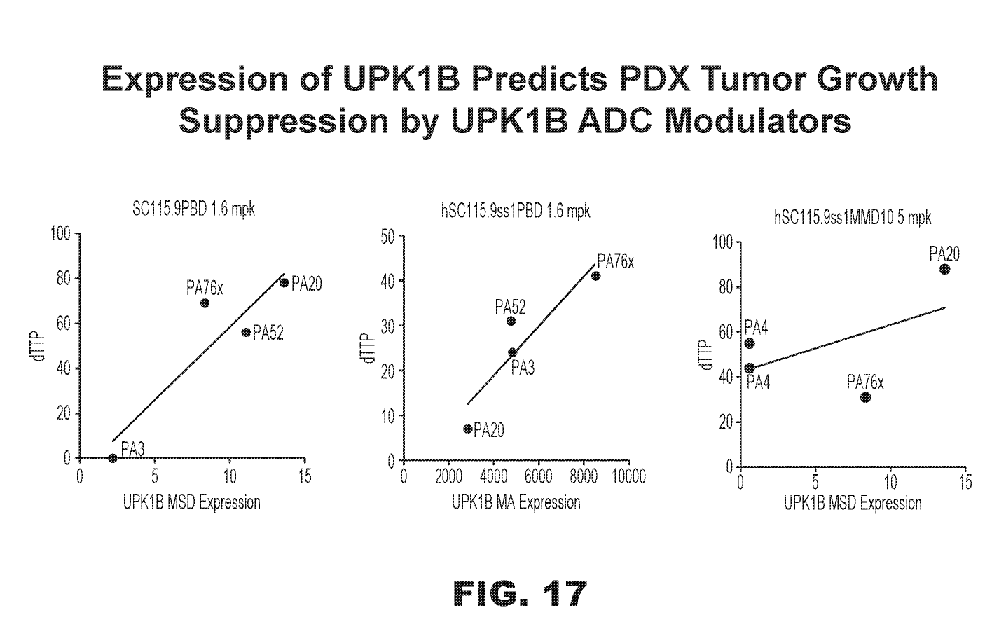

As shown in FIG. 17, there is a positive association between the amount of tumor growth inhibition and the expression level of hUPK1B.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. An isolated antibody that binds to tumor initiating cells expressing UPK1B.

2. An isolated antibody that binds to human UPK1B comprising SEQ ID NO: 1.

3. An isolated antibody that binds to UPK1B and comprises or competes for binding with an antibody comprising:
a light chain variable region (VL) of SEQ ID NO: 21 and a heavy chain variable region (VH) of SEQ ID NO: 23; or
a VL of SEQ ID NO: 25 and a VH of SEQ ID NO: 27; or
a VL of SEQ ID NO: 29 and a VH of SEQ ID NO: 31; or
a VL of SEQ ID NO: 33 and a VH of SEQ ID NO: 35; or
a VL of SEQ ID NO: 37 and a VH of SEQ ID NO: 39; or
a VL of SEQ ID NO: 41 and a VH of SEQ ID NO: 43; or
a VL of SEQ ID NO: 45 and a VH of SEQ ID NO: 47; or
a VL of SEQ ID NO: 49 and a VH of SEQ ID NO: 51; or
a VL of SEQ ID NO: 53 and a VH of SEQ ID NO: 55; or
a VL of SEQ ID NO: 57 and a VH of SEQ ID NO: 59; or
a VL of SEQ ID NO: 61 and a VH of SEQ ID NO: 63; or
a VL of SEQ ID NO: 65 and a VH of SEQ ID NO: 67; or
a VL of SEQ ID NO: 69 and a VH of SEQ ID NO: 71; or
a VL of SEQ ID NO: 73 and a VH of SEQ ID NO: 75; or
a VL of SEQ ID NO: 77 and a VH of SEQ ID NO: 79; or
a VL of SEQ ID NO: 81 and a VH of SEQ ID NO: 83; or
a VL of SEQ ID NO: 85 and a VH of SEQ ID NO: 87; or
a VL of SEQ ID NO: 89 and a VH of SEQ ID NO: 91.

4. An isolated antibody of any of claims 1-3, which is an internalizing antibody.

5. An isolated antibody of any of claims 1-4, which is a chimeric, CDR grafted, humanized or human antibody, or an immunoreactive fragment thereof.

6. An isolated antibody of any of claims 1-5 wherein the antibody comprises a VL of SEQ ID NO: 101 and a VH of SEQ ID NO: 103.

7. An isolated antibody of any of claims 1-6 wherein the antibody comprises a VL of SEQ ID NO: 105 and a VH of SEQ ID NO: 107.

8. An isolated antibody of any of claims 1-7 wherein the antibody comprises a site-specific antibody.

9. The antibody of any one of claims 1-8, wherein the antibody is conjugated to a payload.

10. A pharmaceutical composition comprising an antibody of any one of claims 1-8.

11. A nucleic acid encoding all or part of an antibody of any one of claims 1-8.

12. A vector comprising the nucleic acid of claim 11.

13. A host cell comprising the nucleic acid of claim 11 or the vector of claim 12.

14. An ADC of the formula Ab-[L-D]n or a pharmaceutically acceptable salt thereof wherein:
a) Ab comprises an anti-UPK1B antibody;
b) L comprises an optional linker;
c) D comprises a drug; and
d) n is an integer from about 1 to about 20.

15. The ADC of claim 14 where the anti-UPK1B antibody comprises a chimeric, CDR grafted, humanized or human antibody or an immunoreactive fragment thereof.

16. The ADC of claim 14 where Ab is an anti-UPK1B antibody of any one of claims 1-8.

17. The ADC of claim 14 where n comprises an integer of from about 2 to about 8.

18. The ADC of claim 14 wherein D comprises a compound selected from the group consisting of dolastatins, auristatins, maytansinoids, pyrrolobenzodiazepines (PBDs), benzodiazepine derivatives, calicheamicin and amanitins.

19. A pharmaceutical composition comprising an ADC of any one of claims 14 to 18.

20. A method of treating cancer comprising administering a pharmaceutical composition of claim 10 or claim 19 to a subject in need thereof.

21. The method of claim 20 wherein the cancer comprises a hematologic malignancy.

22. The method of claim 21 wherein the hematologic malignancy comprises leukemia or lymphoma.

23. The method of claim 20 wherein the cancer comprises a solid tumor.

24. The method of claim 23 wherein the cancer is selected from the group consisting of adrenal cancer, liver cancer, kidney cancer, bladder cancer, breast cancer, gastric cancer, ovarian cancer, cervical cancer, uterine cancer, esophageal cancer, colorectal cancer, prostate cancer, melanoma, pancreatic cancer, lung cancer (both small cell and non-small cell), thyroid cancer and glioblastoma.

25. The method of claim 24, wherein the cancer comprises pancreatic cancer.

26. The method of claim 24, wherein the cancer comprises bladder cancer.

27. The method of claim 20, further comprising administering to the subject at least one additional therapeutic moiety.

28. A method of reducing tumor initiating cells in a tumor cell population, wherein the method comprises contacting a tumor cell population comprising tumor initiating cells and tumor cells other than tumor initiating cells, with an ADC of claims 14-18 whereby the frequency of tumor initiating cells is reduced.

29. The method of claim 28, wherein the contacting is performed in vivo.

30. The method of claim 28, wherein the contacting is performed in vitro.

31. A method of delivering a cytotoxin to a cell comprising contacting the cell with an ADC of any one of claims 14 to 18.

32. A method of detecting, diagnosing, or monitoring cancer in a subject, the method comprising the steps of (a) contacting tumor cells with an antibody of any one of claims 1-9; and (b) detecting the antibody on the tumor cells.

33. The method of claim 32, wherein the contacting is performed in vitro.

34. The method of claim 32 wherein the contacting is performed in vivo

35. A method of producing an ADC of claim 14 comprising the step of conjugating an anti-UPK1B antibody (Ab) with a drug (D).

36. The method of claim 35 wherein the antibody comprises a site-specific antibody.

37. A kit comprising:
(a) one or more containers containing a pharmaceutical composition of claim 19; and
(b) a label or package insert associated with the one or more containers indicating that the composition is for treating a subject having cancer.

38. A kit comprising:
(a) one or more containers containing a pharmaceutical composition of claim 19; and
(b) a label or package insert associated with one or more containers indicating a dosage regimen for a subject having cancer.

39. The kits of claim 37 or claim 38 wherein the cancer is pancreatic cancer.

40. An ADC of the formula Ab-[L-D]n comprising a structure selected from the group consisting of: 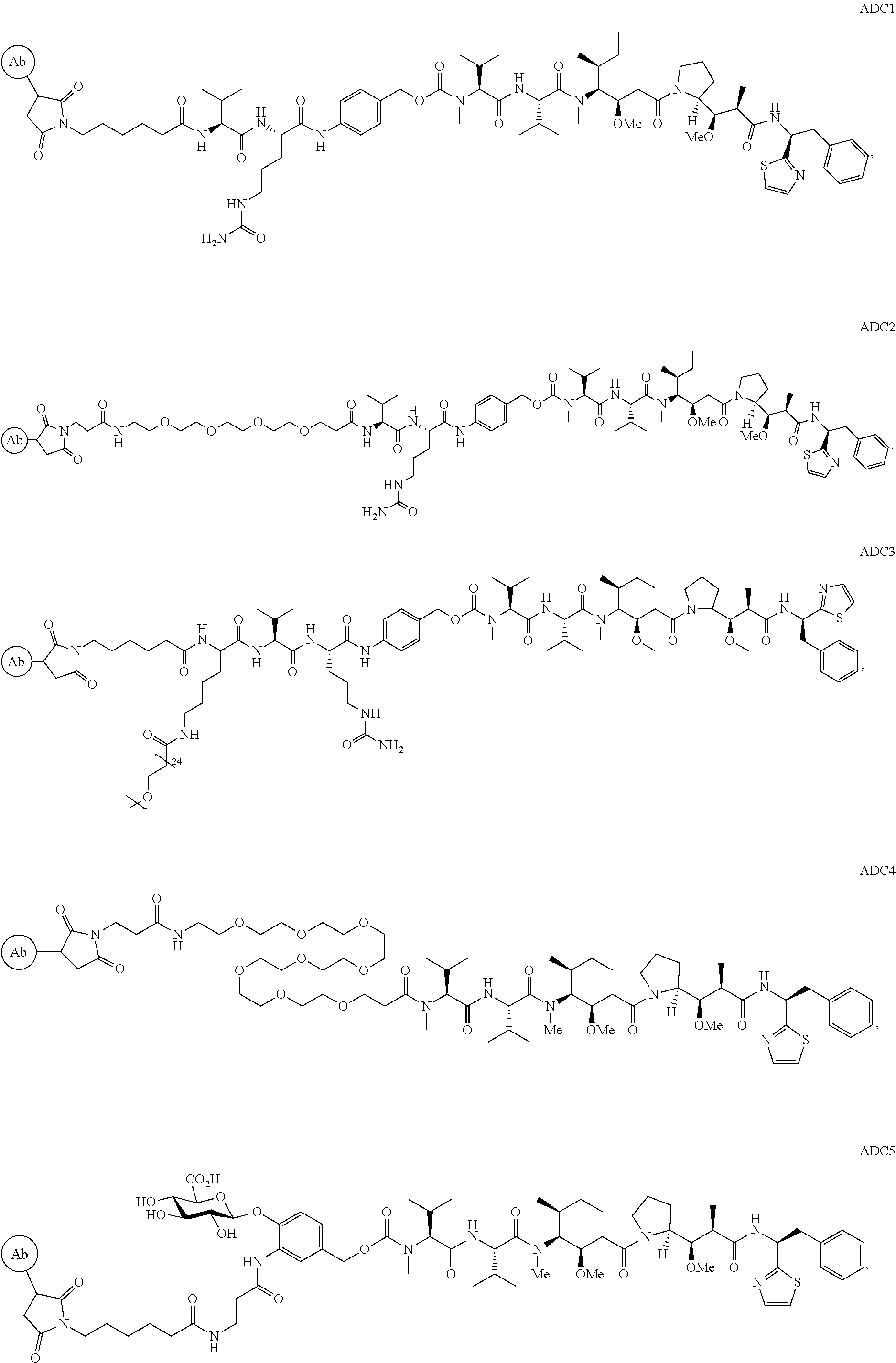 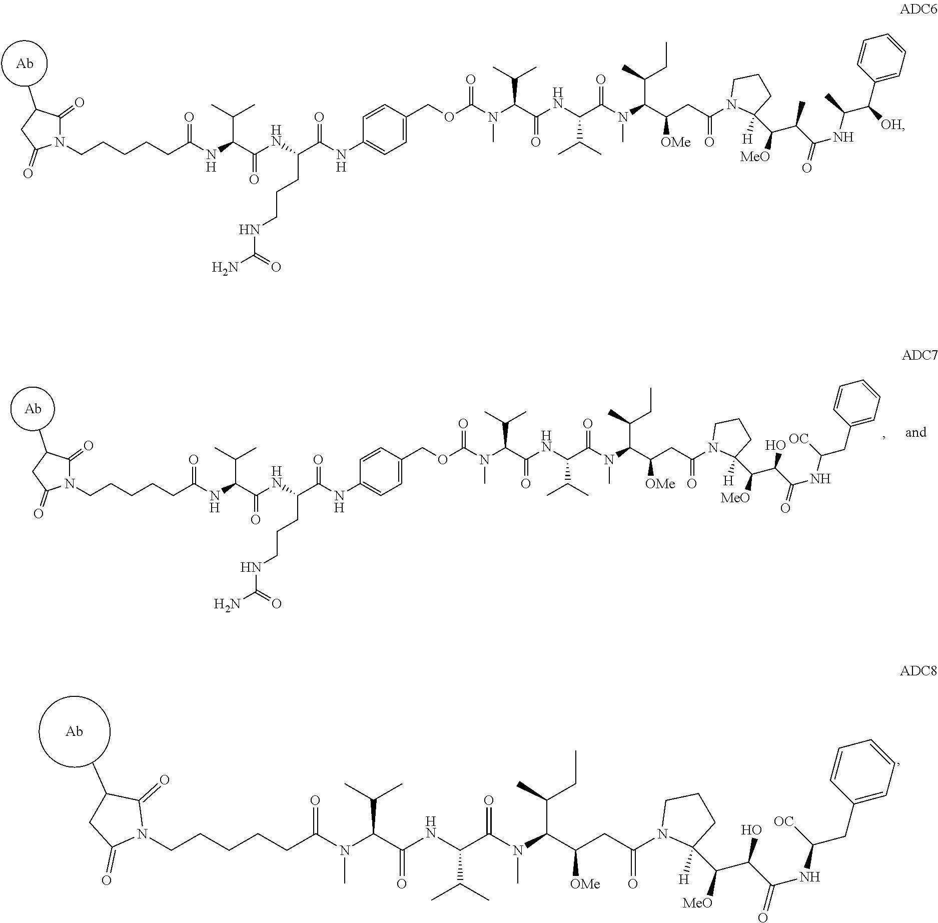
wherein Ab comprises an anti-UPK1B antibody or immunoreactive fragment thereof; and
n is an integer from about 1 to about 20.

41. The ADC of claim 40 wherein the anti-UPK1B antibody comprises a site-specific antibody.

42. The ADC of claim 41 wherein anti-UPK1B antibody comprises hSC115.9ss1 (SEQ ID NOS: 110 and 112).

43. The ADC of claim 42 comprising two unpaired cysteines wherein each cysteine is conjugated to a payload.

44. An ADC of the formula Ab-[L-D]n comprising the structure: 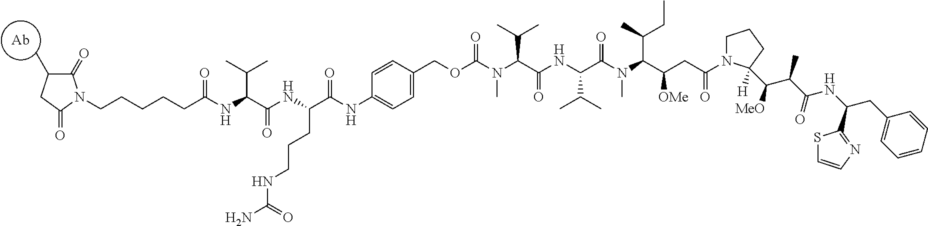
wherein Ab comprises hSC115.9ss1 (SEQ ID NOS: 110 and 112) and n is 2.
